(19) United States
(12) Patent Application Publication
(10) Pub. No.: US 2024/0026386 A1
(43) Pub. Date: Jan. 25, 2024

(54) COMPOSITIONS AND METHODS FOR THE TARGETING OF BCL11A

(71) Applicant: Scribe Therapeutics Inc., Alameda, CA (US)

(72) Inventors: Benjamin OAKES, El Cerrito, CA (US); Sean HIGGINS, Alameda, CA (US); Sarah DENNY, San Francisco, CA (US); Brett T. STAAHL, Tiburon, CA (US); Isabel COLIN, Oakland, CA (US); Maroof ADIL, Davis, CA (US); Cole URNES, Los Angeles, CA (US)

(21) Appl. No.: 18/255,172

(22) PCT Filed: Dec. 2, 2021
(86) PCT No.: PCT/US2021/061672

§ 371 Date: May 31, 2023

Related U.S. Application Data
(60) Provisional application No. 63/120,885, filed on Dec. 3, 2020.

(51) Int. Cl. C12N 15/90; C12N 9/22; C12N 15/11; A61K 35/28; A61K 35/545; A61K 35/34; A61K 35/18

(52) U.S. Cl. CPC C12N 15/907; C12N 9/22; C12N 15/11; C12N 15/111; A61K 35/28; A61K 35/545; A61K 35/34; A61K 35/18; C12N 2310/20

(57) ABSTRACT

Provided herein are systems comprising Class 2, Type V CRISPR polypeptides, guide nucleic acids (gNA), and optionally donor template nucleic acids useful in the modification of a BCL11A gene. The systems are also useful for the modification of cells in subjects with a hemoglobinopathy-related disease. Also provided are methods of treatment of subjects having a hemoglobinopathy-related disease by administration of the systems or nucleic acids encoding such systems that target the BCL11A gene in such subjects.

### CROSS-REFERENCE TO RELATED APPLICATIONS

This application claims priority to U.S. provisional patent application No. 63/120,885, filed on Dec. 3, 2020, the contents of which are incorporated by reference in their entirety herein.

### INCORPORATION BY REFERENCE OF SEQUENCE LISTING

This application contains a Sequence Listing which has been submitted in ASCII format via EFS-WEB and is hereby incorporated by reference in its entirety. Said ASCII copy, created on Dec. 1, 2021 is named SCRB_030_01WO_SeqList_ST25.txt and is 8.78 MB in size.

### BACKGROUND

Fetal hemoglobin (also hemoglobin F, HbF, or α2γ2) is the main oxygen carrier protein in the human fetus. HbF has a different composition from the adult forms of hemoglobin, which allows it to bind oxygen more strongly than the adult form, allowing the developing fetus to retrieve oxygen from the mother's bloodstream. HbF is a tetramer of two adult α-globin polypeptides and two fetal β-like γ-globin polypeptides. During gestation, the duplicated γ-globin genes constitute the predominant genes transcribed in the β-globin cluster. After birth, γ-globin is replaced by adult β-globin, a process referred to as the “fetal switch”, a process that involves expression of BCL11A, a regulator of HbF silencing (Sankaran, V. G., et al. Human Fetal Hemoglobin Expression Is Regulated by the Developmental Stage-Specific Repressor BCL11A. Science 322(5909):1839-1842 (2008); Liu, N., et al. Direct Promoter Repression by BCL11A Controls the Fetal to Adult Hemoglobin Switch. Cell 173(2):430 (2018)). In healthy adults, the composition of hemoglobin is hemoglobin A (˜97%), hemoglobin A2 (2.2-3.5%) and hemoglobin F (<1%) (Thomas, C and Lumb, A. B. Physiology of haemoglobin. Continuing Education in Anaesthesia Critical Care & Pain. 12(5): 251-256 (2012)).

Hemoglobinopathies are inherited single-gene disorders that, in most cases, are inherited as autosomal co-dominant traits. Common hemoglobinopathies include sickle-cell disease and α- and β-thalassemias. Hemoglobinopathies are most common in populations from Africa, the Mediterranean basin and Southeast Asia. Most hemoglobinopathies, including sickle cell anemia, are simply structural abnormalities in the globin proteins themselves. Sickle cell anemia results from a point mutation in the β-globin structural gene, HBB, leading to the production of an abnormal hemoglobin (HbS), which results in a reduced oxygen-carrying capacity of the blood. Thalassemias, in contrast, usually result in underproduction of normal globin proteins, often through mutations in regulatory genes, leading to deficient or absent adult hemoglobin (HbA). In β-thalassemia, where β-globin is deficient, increased γ-globin expression reduces the imbalance of the α- and β-globin chains that underlies the pathophysiology of anemia in this condition (Liu, N., et al. Direct Promoter Repression by BCL11A Controls the Fetal to Adult Hemoglobin Switch. Cell 173(2): 430 (2018)). Both sickle cell disease and thalassemia may cause anemia.

B-cell lymphoma/leukemia 11A (BCL11A) is a protein that in humans is encoded by the BCL11A gene. During hematopoietic cell differentiation, this gene is down-regulated and has been found to play a role in the suppression of fetal hemoglobin production. BCL11A is a major repressor protein of hemoglobin F production, by binding to the gene coding for the γ subunit at the promoter region (Sankaran V G, et al. Human fetal hemoglobin expression is regulated by the developmental stage-specific repressor BCL11A. Science 322:1839 (2008)). As increased γ-globin reduces the clinical severity of the β-hemoglobinopathies, sickle-cell disease, and β-thalassemia caused by mutation or decreased expression of β-globin, respectively, gene editing of BCL11A to increase expression of γ-globin beyond the residual ˜1% fetal hemoglobin has been proposed as an attractive therapeutic strategy in adults with hemoglobinopathies (Smith, E. C., et al. Strict in vivo specificity of the Bc111a erythroid enhancer. Blood 128(19):2338 (2016)).

The advent of CRISPR/Cas systems and the programmable nature of these minimal systems has facilitated their use as a versatile technology for genomic manipulation and engineering. To date, the use of CRISPR/Cas systems for the treatment of hemoglobinopathies have been limited to the editing of cells ex vivo, followed by transplantation into subjects suffering from the underlying hemoglobinopathy. Thus, there is a need for compositions and methods to regulate BCL11A to reduce direct γ-globin gene promoter repression in vivo in subjects with these diseases. Provided herein are compositions and methods for targeting the BCL11A gene to the address this need.

### SUMMARY

The present disclosure relates to compositions of modified Class 2, Type V CRISPR proteins and guide nucleic acids used to alter a target nucleic acid comprising a BCL11A gene in cells. The Class 2, Type V CRISPR proteins and guide nucleic acids are modified for passive entry into target cells. The Class 2, Type V CRISPR proteins and guide nucleic acids are useful in a variety of methods for target nucleic acid modification of BCL11A-related diseases, which methods are also provided.

In one aspect, the present disclosure relates to CasX:guide nucleic acid systems (CasX:gRNA systems) and methods used to knock-down or knock-out a BCL11A gene in order to reduce or eliminate expression of the BCL11A gene product in subjects having a 0-hemoglobinopathy-related disease.

In some embodiments, the CasX:gRNA system gRNA is a gRNA, or a chimera of RNA and DNA, and may be a single-molecule gRNA or a dual-molecule gRNA. In other embodiments, the CasX:gRNA system gRNA has a targeting sequence complementary to a target nucleic acid sequence comprising a region within the BCL11A gene. In some embodiments, the targeting sequence of the gRNA is selected from the group consisting of SEQ ID NOS: 272-2100 and 2286-26789 or a sequence having at least about 50%, at least about 60%, at least about 70%, at least about 80%, at least about 85%, at least about 90%, or at least about 95% identity thereto. The gRNA can comprise a targeting sequence comprising 15 to 20 consecutive nucleotides. In other embodiments, the targeting sequence of the gRNA consists of 20 nucleotides. In other embodiments, the targeting sequence consists of 19 nucleotides. In other embodiments, the targeting sequence consists of 18 nucleotides. In other embodiments, the targeting sequence consists of 17 nucleotides. In other embodiments, the targeting sequence consists of 16 nucleotides. In other embodiments, the targeting sequence consists of 15 nucleotides. In other embodiments, the targeting sequence of the gRNA has a sequence selected from the group consisting of SEQ ID NOS: 272-2100 and 2286-26789. In other embodiments, the targeting sequence of the gRNA has a sequence selected from the group consisting of SEQ ID NOS: 272-2100 and 2286-26789, with a single nucleotide removed from the 3′ end of the sequence. In other embodiments, the targeting sequence consists of 18 nucleotides, has a sequence selected from the group consisting of SEQ ID NOS: 272-2100 and 2286-26789, with two nucleotides removed from the 3′ end of the sequence. In other embodiments, the targeting sequence consists of 17 nucleotides, has a sequence selected from the group consisting of SEQ ID NOS: 272-2100 and 2286-26789, with three nucleotides removed from the 3′ end of the sequence. In other embodiments, the targeting sequence consists of 16 nucleotides, has a sequence selected from the group consisting of SEQ ID NOS: 272-2100 and 2286-26789, with four nucleotides removed from the 3′ end of the sequence. In other embodiments, the targeting sequence consists of 15 nucleotides, has a sequence selected from the group consisting of SEQ ID NOS: 272-2100 and 2286-26789, with five nucleotides removed from the 3′ end of the sequence.

In some embodiments, the gRNA has a scaffold comprising a sequence selected from the group consisting of sequences SEQ ID NOS: 2238-2285, 26794-26839 and 27219-27265, or as set forth in Table 3, or a sequence having at least about 50%, at least about 60%, at least about 70%, at least about 80%, at least about 90%, at least about 95%, at least about 96%, at least about 97%, at least about 98%, at least about 99% sequence identity thereto. In some embodiments, the gRNA has a scaffold comprising a sequence selected from the group consisting of sequences SEQ ID NOS: 2238-2285, 26794-26839 and 27219-27265. In some embodiments, the gRNA has a scaffold comprising a sequence selected from the group consisting of sequences SEQ ID NOS: 2101-2285, 26794-26839 and 27219-27265.

In some embodiments, the CasX:gRNA systems comprise a CasX variant sequence having a sequence selected from the group consisting of SEQ ID NOS: 36-99, 101-148, 26908-27154, or a sequence having at least about 50%, at least about 60%, at least about 70%, at least about 80%, or at least about 90%, or at least about 95%, or at least about 96%, or at least about 97%, or at least about 98%, or at least about 99% sequence identity thereto. In some embodiments, the CasX:gRNA systems comprise a CasX variant sequence having a sequence selected from the group consisting of SEQ ID NOS: 36-99, 101-148, 26908-27154. In some embodiments, the CasX:gRNA systems comprise a CasX variant sequence having a sequence selected from the group consisting of SEQ ID NOS: 59, 72-99, 101-148, and 26908-27154, or as set forth in Table 4, or a sequence having at least about 50%, at least about 60%, at least about 70%, at least about 80%, or at least about 90%, or at least about 95%, or at least about 96%, or at least about 97%, or at least about 98%, or at least about 99% sequence identity thereto. In some embodiments, the CasX:gRNA systems comprise a CasX variant sequence having a sequence selected from the group consisting of SEQ ID NOS: 59, 72-99, 101-148, and 26908-27154. In some embodiments, the CasX:gRNA systems comprise a CasX variant sequence having a sequence selected from the group consisting of SEQ ID NOS: 132-148 and 26908-27154, or a sequence having at least about 50%, at least about 60%, at least about 70%, at least about 80%, or at least about 90%, or at least about 95%, or at least about 96%, or at least about 97%, or at least about 98%, or at least about 99% sequence identity thereto. In some embodiments, the CasX:gRNA systems comprise a CasX variant sequence having a sequence selected from the group consisting of SEQ ID NOS: 132-148 and 26908-27154. In these embodiments, a CasX variant exhibits one or more improved characteristics relative to any one of the reference CasX proteins of SEQ ID NOS: 1-3. In some embodiments, the CasX variant protein has binding affinity for a protospacer adjacent motif (PAM) sequence selected from the group consisting of TTC, ATC, GTC, and CTC. In some embodiments, the CasX variant protein has binding affinity for the PAM sequence that is at least 1.5-fold greater compared to the binding affinity of any one of the reference CasX proteins of SEQ ID NOS: 1-3 for the PAM sequences selected from the group consisting of TTC, ATC, GTC, and CTC.

In other embodiments of the CasX:gRNA system, the CasX molecule and the gRNA molecule are associated together in a ribonuclear protein complex (RNP). In a particular embodiment, the RNP comprising the CasX variant and the gRNA variant exhibits greater editing efficiency and/or binding of a target sequence in the target DNA when any one of the PAM sequences TTC, ATC, GTC, or CTC is located 1 nucleotide 5′ to the non-target strand sequence having identity with the targeting sequence of the gRNA in a cellular assay system compared to the editing efficiency and/or binding of an RNP comprising a reference CasX protein and a reference gRNA in a comparable assay system.

In some embodiments, the CasX:gRNA system further comprises a donor template comprising a nucleic acid comprising at least a portion of a BCL11A gene and having at least 1 to about 5 mutations relative to the wild-type sequence, wherein the BCL11A gene portion is selected from the group consisting of a BCL11A exon, a BCL11A intron, a BCL11A intron-exon junction, a BCL11A regulatory element, or combinations thereof, wherein the donor template is used to knock down or knock out the BCL11A gene. In some cases, the donor sequence is a single-stranded DNA template or a single stranded RNA template. In other cases, the donor template is a double-stranded DNA template.

In other embodiments, the disclosure relates to nucleic acids encoding the CasX:gRNA systems of any of the embodiments described herein, as well as vectors comprising the nucleic acids. In some embodiments, the vector is selected from the group consisting of a retroviral vector, a lentiviral vector, an adenoviral vector, an adeno-associated viral (AAV) vector, a herpes simplex virus (HSV) vector, a plasmid, a minicircle, a nanoplasmid, and an RNA vector. In other embodiments, the vector is a CasX delivery particle (XDP) comprising an RNP of a CasX and gRNA of any of the embodiments described herein and, optionally, a donor template nucleic acid and a targeting moiety such as a viral-derived glycoprotein.

In other embodiments, the disclosure provides a method of modifying a BCL11A target nucleic acid sequence of a cells of a population, wherein said method comprises introducing into the cell: a) CasX:gRNA system of any of the embodiments disclosed herein; b) the nucleic acid of any of the embodiments disclosed herein; c) the vector of any of the embodiments disclosed herein; d) the XDP of any of the embodiments disclosed herein; or e) a combination of the foregoing. In some embodiments of the method, the modifying comprises introducing an insertion, deletion, substitution, duplication, or inversion of one or more nucleotides in the target nucleic acid sequence as compared to the wild-type sequence. The target BCL11A gene includes the GATA1 erythroid-specific enhancer binding site (GATA1) as a regulatory element. In some embodiments, the method of modifying comprises modification of the GATA1 sequence, wherein the BCL11A gene is knocked down or knocked out by the modification. In some cases, the method further comprises contacting the target nucleic acid with a donor template nucleic acid of any of the embodiments disclosed herein. In some embodiments of the method, the donor template comprises a nucleic acid comprising at least a portion of a BCL11A gene but with one or more mutations for knocking out or knocking down the BCL11A gene. In some cases, the modifying of the target nucleic acid sequence occurs in vitro or ex vivo. In some cases, the modifying of the target nucleic acid sequence occurs in vivo. In some embodiments, the cell is a eukaryotic cell selected from the group consisting of a rodent cell, a mouse cell, a rat cell, a primate cell, and a non-human primate cell. In some embodiments, the cell is a human cell. In some embodiments, the cell is a selected from the group consisting of a hematopoietic stem cell (HSC), a hematopoietic progenitor cell (HPC), a CD34+ cell, a mesenchymal stem cell (MSC), induced pluripotent stem cell (iPSC), a common myeloid progenitor cell, a proerythroblast cell, and a erythroblast cell. In some embodiments, the cell is an autologous cell derived from a subject with a β-hemoglobinopathy-related disease. In other embodiments, the cell is allogenic, but of the same species as the subject to be treated.

In other embodiments, the disclosure provides methods of modifying a target nucleic acid sequence of the BCL11A gene wherein the target cells of a population are contacted using vectors encoding the CasX protein and one or more gRNAs comprising a targeting sequence complementary to the BCL11A gene, and optionally further comprising a donor template. In some cases, the vector is an Adeno-Associated Viral (AAV) vector selected from AAV1, AAV2, AAV3, AAV4, AAV5, AAV6, AAV7, AAV8, AAV9, AAV10, AAV-Rh74, or AAVRh10. In other cases, the vector is a lentiviral vector. In other embodiments, the disclosure provides methods wherein the target cells are contacted using a vector, and wherein the vector is a CasX delivery particle (XDP) comprising an RNP of a CasX and gRNA of any of the embodiments described herein and, optionally, a donor template nucleic acid. In some embodiments of the method, the vector is administered to a subject at a therapeutically effective dose. The subject can be a mouse, rat, pig, non-human primate, or a human. The dose can be administered by a route of administration selected from transplantation, local injection, systemic infusion, or combinations thereof.

In other embodiments, the disclosure provides a method of treating a (β-hemoglobinopathy-related disease in a subject in need thereof, comprising modifying a gene encoding BCL11A gene in a cell of the subject, the modifying comprising either contacting said cell with: a) CasX:gRNA system of any of the embodiments disclosed herein; b) the nucleic acid of any of the embodiments disclosed herein; c) the vector of any of the embodiments disclosed herein; d) the XDP of any of the embodiments disclosed herein; or e) a combination of the foregoing. In some embodiments, the β-hemoglobinopathy-related disease is sickle cell anemia or beta-thalassemia. In some cases, the methods of treating a subject with a β-hemoglobinopathy-related disease result in improvement in at least one clinically-relevant parameter. In other cases, the methods of treating a subject with a β-hemoglobinopathy-related disease result in improvement in at least two clinically-relevant parameters.

In other embodiments, the disclosure provides use of the CasX:gRNA systems, nucleic acids, vectors or XDP described herein for treating a β-hemoglobinopathy-related disease in a subject in need thereof. In some embodiments, the use comprises modifying a gene encoding BCL11A gene in a cell of the subject, the modifying comprising either contacting said cell with: a) CasX:gRNA system of any of the embodiments disclosed herein; b) the nucleic acid of any of the embodiments disclosed herein; c) the vector of any of the embodiments disclosed herein; d) the XDP of any of the embodiments disclosed herein; or e) a combination of the foregoing.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

The contents of U.S. provisional applications 63/121,196, filed on Dec. 3, 2020, 63/162,346 filed on Mar. 17, 2021, and 63/208,855, filed on Jun. 9, 2021, which disclose CasX variants and gRNA variants, are hereby incorporated by reference in their entireties. The contents of international application publications WO 2020/247882, published Dec. 10, 2020, WO 2020/247883, published Dec. 10, 2020, and WO 2021/113772, published Jun. 10, 2021 are hereby incorporated by reference in their entireties.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 is a graph of the results of an assay for the quantification of active fractions of RNP formed by sgRNA174 (SEQ ID NO: 2238) and the CasX variants 119 (SEQ ID NO: 59), 457 (SEQ ID NO: 101), 488 (SEQ ID NO: 123) and 491 (SEQ ID NO: 126), as described in Example 8. Equimolar amounts of RNP and target were co-incubated and the amount of cleaved target was determined at the indicated timepoints. Mean and standard deviation of three independent replicates are shown for each timepoint. The biphasic fit of the combined replicates is shown. “2” refers to the reference CasX protein of SEQ ID NO: 2.

FIG. 2 shows the quantification of active fractions of RNP formed by CasX2 (reference CasX protein of SEQ ID NO:2) and the modified sgRNAs, as described in Example 8. Equimolar amounts of RNP and target were co-incubated and the amount of cleaved target was determined at the indicated timepoints. Mean and standard deviation of three independent replicates are shown for each timepoint. The biphasic fit of the combined replicates is shown.

FIG. 3 shows the quantification of active fractions of RNP formed by CasX 491 and the modified sgRNAs under guide-limiting conditions, as described in Example 8. Equimolar amounts of RNP and target were co-incubated and the amount of cleaved target was determined at the indicated timepoints. The biphasic fit of the data is shown.

FIG. 4 shows the quantification of cleavage rates of RNP formed by sgRNA174 and the CasX variants, as described in Example 8. Target DNA was incubated with a 20-fold excess of the indicated RNP and the amount of cleaved target was determined at the indicated time points. Mean and standard deviation of three independent replicates are shown for each timepoint, except for 488 and 491 where a single replicate is shown. The monophasic fit of the combined replicates is shown.

FIG. 5 shows the quantification of cleavage rates of RNP formed by CasX2 and the indicated sgRNA variants, as described in Example 8. Target DNA was incubated with a 20-fold excess of the indicated RNP and the amount of cleaved target was determined at the indicated time points. Mean and standard deviation of three independent replicates are shown for each timepoint. The monophasic fit of the combined replicates is shown.

FIG. 6 shows the quantification of initial velocities of RNP formed by CasX2 and the sgRNA variants, as described in Example 8. The first two time-points of the previous cleavage experiment were fit with a linear model to determine the initial cleavage velocity.

FIG. 7 shows the quantification of cleavage rates of RNP formed by CasX491 and the sgRNA variants, as described in Example 8. Target DNA was incubated with a 20-fold excess of the indicated RNP at 10° C. and the amount of cleaved target was determined at the indicated time points. The monophasic fit of the timepoints is shown.

FIG. 8 shows the quantification of competent fractions of RNP of CasX variant 515 (SEQ ID NO: 133) and 526 (SEQ ID NO: 143) complexed with gRNA variant 174 compared to RNP of reference CasX 2 complexed with gRNA 2 using equimolar amounts of indicated RNP and a complementary target, as described in Example 8. The biphasic fit for each time course or set of combined replicates is shown.

FIG. 9 shows the quantification of cleavage rates of RNP of CasX variant 515 and 526 complexed with gRNA variant 174 compared to RNP of reference CasX 2 complexed with gRNA 2 using with a 20-fold excess of the indicated RNP, as described in Example 8.

FIG. 10A shows the quantification of cleavage rates of CasX variants on TTC PAM, as described in Example 5. Target DNA substrates with identical spacers and the indicated PAM sequence were incubated with a 20-fold excess of the indicated RNP at 37° C. and the amount of cleaved target was determined at the indicated time points. Monophasic fit of a single replicate is shown.

FIG. 10B shows the quantification of cleavage rates of CasX variants on CTC PAM, as described in Example 5. Target DNA substrates with identical spacers and the indicated PAM sequence were incubated with a 20-fold excess of the indicated RNP at 37° C. and the amount of cleaved target was determined at the indicated time points. Monophasic fit of a single replicate is shown.

FIG. 10C shows the quantification of cleavage rates of CasX variants on GTC PAM, as described in Example 5. Target DNA substrates with identical spacers and the indicated PAM sequence were incubated with a 20-fold excess of the indicated RNP at 37° C. and the amount of cleaved target was determined at the indicated time points. Monophasic fit of a single replicate is shown.

FIG. 10D shows the quantification of cleavage rates of CasX variants on ATC PAM, as described in Example 5. Target DNA substrates with identical spacers and the indicated PAM sequence were incubated with a 20-fold excess of the indicated RNP at 37° C. and the amount of cleaved target was determined at the indicated time points. Monophasic fit of a single replicate is shown.

FIG. 11A shows the quantification of cleavage rates of RNP of CasX variant 491 and guide 174 on NTC PAMs, as described in Example 5. Timepoints were taken over the course of 10 minutes and the fraction cleaved was graphed for each target and timepoint, but only the first two minutes of the time course are shown for clarity.

FIG. 11B shows the quantification of cleavage rates of RNP of CasX variant 491 and guide 174 on NTT PAMs, as described in Example 5. Timepoints were taken over the course of 10 minutes and the fraction cleaved was graphed for each target and timepoint.

FIG. 12A shows the quantification of cleavage by RNP formed by sgRNA174 and the CasX variants 515 using spacer lengths of 18, 19, or 20 nucleotides, as described in Example 9.

Target DNA was incubated with a 20-fold excess of the indicated RNP and the amount of cleaved target was determined at the indicated time points. Mean and standard deviation of three independent replicates are shown for each timepoint. The monophasic fit of the combined replicates is shown.

FIG. 12B shows the quantification of cleavage by RNP formed by sgRNA174 and the CasX variant 526 using spacer lengths of 18, 19, or 20 nucleotides, as described in Example 9. Target DNA was incubated with a 20-fold excess of the indicated RNP and the amount of cleaved target was determined at the indicated time points. Mean and standard deviation of three independent replicates are shown for each timepoint. The monophasic fit of the combined replicates is shown.

FIG. 13 is a schematic showing an example of CasX protein and scaffold DNA sequence for packaging in adeno-associated virus (AAV). The DNA segment between the AAV inverted terminal repeats (ITRs), comprised of a CasX-encoding DNA and its promoter, and scaffold-encoding DNA and its promoter gets packaged within an AAV capsid during AAV production.

FIG. 14 shows the results of an editing assay comparing gRNA scaffolds 229-237 (see Table 3 for corresponding sequences and SEQ ID NOs) to scaffold 174 in mouse neural progenitor cells (mNPC) isolated from the Ai9-tdtomato transgenic mice. Cells were nucleofected with the indicated doses of p59 plasmids encoding CasX 491, the scaffold, and spacer 11.30 (5′ AAGGGGCUCCGCACCACGCC 3′, SEQ ID NO: 27197) targeting mRHO. Editing at the mRHO locus was assessed 5 days post-transfection by NGS, and show that editing with constructs with scaffolds 230, 231, 234 and 235 demonstrated greater editing compared to constructs with scaffold 174 at both doses.

FIG. 15 shows the results of an editing assay comparing gRNA scaffolds 229-237 to scaffold 174 in mNPC cells. Cells were nucleofected with the indicated doses of p59 plasmids encoding CasX 491, the scaffold, and spacer 12.7 (5′ CUGCAUUCUAGUUGUGGUUU 3′, SEQ ID NO: 27198) targeting repeat elements preventing expression of the tdTomato fluorescent protein. Editing was assessed 5 days post-transfection by FACS, to quantify the fraction of tdTomato positive cells. Cells nucleofected with scaffolds 231-235 displayed approximately 35% greater editing compared to constructs with scaffold 174 at the high dose, and approximately 25% greater editing at the low dose.

FIG. 16 shows the results of an editing assay comparing CasX nucleases 2, 119, 491, 515, 527, 528, 529, 530, and 531 (see Table 4 for corresponding sequences and SEQ ID NOs) in a custom HEK293 cell line, PASS_V1.01. Cells were lipofected with 2 μg of p67 plasmid encoding the indicated CasX protein. After five days, cell genomic DNA was extracted. PCR amplification and Next-Generation Sequencing was performed to isolate and quantify the fraction of edited cells at custom designed on-target editing sites. For each sample, editing was evaluated at target sites (individual points) consisting of the following PAM sequences: 48 TTC, 14 ATC, 22 CTC, 11 GTC individual sites, and percent editing was normalized to a vehicle control. Cells lipofected with any nuclease displayed higher mean editing at TTC PAM target sites (horizontal bar) than that of the wild-type nuclease CasX 2, except CasX 528. The relative preference of any given nuclease for the four different PAM sequences is also represented by the violin plots. In particular, CasX nucleases 527, 528, and 529 exhibit substantially different PAM preferences than that of the wild-type nuclease CasX 2.

FIG. 17 shows the results of an editing assay comparing improved CasX nuclease 491 to improved nucleases 532 and 533 in a custom HEK293 cell line, PASS_V1.01. Cells were lipofected, in duplicate, with 2 μg of p67 plasmid encoding the indicated CasX protein and a puromycin resistance gene, and grown under puromycin selection. After three days, cell genomic DNA was extracted. PCR amplification and Next-Generation Sequencing was performed to isolate and quantify the fraction of edited cells at custom designed on-target editing sites. For each sample, editing was evaluated at target sites consisting of the following PAM sequences: 48 TTC, 14 ATC, 22 CTC, 11 GTC individual sites, and fraction editing was normalized to a vehicle control. Cells lipofected with CasX 532 or 533 displayed higher mean editing than Cas 491 at each of the PAM sequences, with the exception of CasX 533 at TTC PAM target sites. Error bars represent standard error of the mean for n=2 biological samples.

FIG. 18 shows the results of editing of the BCL11A erythroid enhancer locus in HEK293T cells by CasX protein variant 438 with scaffold 174 compared to a Cas9 system, as described in Example 13.

FIG. 19 shows the results of editing at the GATA1 binding region of the BCL11A erythroid enhancer locus in K562 cells by CasX protein variant 491 with scaffold 174 compared to CasX protein variant 119 with scaffold 174, as described in Example 14.

FIG. 20 shows the results of editing at the GATA1 binding region of the BCL11A erythroid enhancer locus in K562 cells by CasX protein variant 491 with scaffold 174 delivered by various doses of XDP, as described in Example 14.

FIG. 21 shows the results of editing at the GATA1 binding region of the BCL11A erythroid enhancer locus in HSC cells by CasX protein variant 491 with scaffold 174 compared to CasX protein variant 119 with scaffold 174, as described in Example 15.

FIG. 22 shows the results of editing at the GATA1 binding region of the BCL11A erythroid enhancer locus in HSC cells by CasX protein variant 491 with scaffold 174 delivered by various doses of XDP, as described in Example 15.

FIG. 23 is a schematic showing the positioning of the spacer 21.1 (SEQ ID NO: 22) relative to the GATA1 binding site sequence in the target nucleic acid. Top strand: SEQ ID NO: 26790, bottom strand: SEQ ID NO: 26791.

### DETAILED DESCRIPTION

While exemplary embodiments have been shown and described herein, it will be obvious to those skilled in the art that such embodiments are provided by way of example only.

Numerous variations, changes, and substitutions will now occur to those skilled in the art without departing from the inventions claimed herein. It should be understood that various alternatives to the embodiments described herein may be employed in practicing the embodiments of the disclosure. It is intended that the claims define the scope of the invention and that methods and structures within the scope of these claims and their equivalents be covered thereby.

Unless otherwise defined, all technical and scientific terms used herein have the same meaning as commonly understood by one of ordinary skill in the art to which this invention belongs. Although methods and materials similar or equivalent to those described herein can be used in the practice or testing of the present embodiments, suitable methods and materials are described below. In case of conflict, the patent specification, including definitions, will control. In addition, the materials, methods, and examples are illustrative only and not intended to be limiting. Numerous variations, changes, and substitutions will now occur to those skilled in the art without departing from the invention.

### Definitions

The terms “polynucleotide” and “nucleic acid,” used interchangeably herein, refer to a polymeric form of nucleotides of any length, either ribonucleotides or deoxyribonucleotides. Thus, terms “polynucleotide” and “nucleic acid” encompass single-stranded DNA; double-stranded DNA; multi-stranded DNA; single-stranded RNA; double-stranded RNA; multi-stranded RNA; genomic DNA; cDNA; DNA-RNA hybrids; and a polymer comprising purine and pyrimidine bases or other natural, chemically or biochemically modified, non-natural, or derivatized nucleotide bases.

“Hybridizable” or “complementary” are used interchangeably to mean that a nucleic acid (e.g., RNA, DNA) comprises a sequence of nucleotides that enables it to non-covalently bind, i.e., form Watson-Crick base pairs and/or G/U base pairs, “anneal”, or “hybridize,” to another nucleic acid in a sequence-specific, antiparallel, manner (i.e., a nucleic acid specifically binds to a complementary nucleic acid) under the appropriate in vitro and/or in vivo conditions of temperature and solution ionic strength. It is understood that the sequence of a polynucleotide need not be 100% complementary to that of its target nucleic acid to be specifically hybridizable; it can have at least about 70%, at least about 80%, or at least about 90%, or at least about 95% sequence identity and still hybridize to the target nucleic acid. Moreover, a polynucleotide may hybridize over one or more segments such that intervening or adjacent segments are not involved in the hybridization event (e.g., a loop structure or hairpin structure, a ‘bulge’, ‘bubble’ and the like).

A “gene,” for the purposes of the present disclosure, includes a DNA region encoding a gene product (e.g., a protein, RNA), as well as all DNA regions which regulate the production of the gene product, whether or not such regulatory element sequences are adjacent to coding and/or transcribed sequences. Accordingly, a gene may include regulatory sequences including, but not necessarily limited to, promoter sequences, terminators, translational regulatory sequences such as ribosome binding sites and internal ribosome entry sites, enhancers, silencers, insulators, boundary elements, replication origins, matrix attachment sites and locus control regions. Coding sequences encode a gene product upon transcription or transcription and translation; the coding sequences of the disclosure may comprise fragments and need not contain a full-length open reading frame. A gene can include both the strand that is transcribed, e.g. the strand containing the coding sequence, as well as the complementary strand.

The term “downstream” refers to a nucleotide sequence that is located 3′ to a reference nucleotide sequence. In certain embodiments, downstream nucleotide sequences relate to sequences that follow the starting point of transcription. For example, the translation initiation codon of a gene is located downstream of the start site of transcription.

The term “upstream” refers to a nucleotide sequence that is located 5′ to a reference nucleotide sequence. In certain embodiments, upstream nucleotide sequences relate to sequences that are located on the 5′ side of a coding region or starting point of transcription. For example, most promoters are located upstream of the start site of transcription.

The term “adjacent to” with respect to polynucleotide or amino acid sequences refers to sequences that are next to, or adjoining each other in a polynucleotide or polypeptide. The skilled artisan will appreciate that two sequences can be considered to be adjacent to each other and still encompass a limited amount of intervening sequence, e.g., 1, 2, 3, 4, 5, 6, 7, 8, 9 or 10 nucleotides or amino acids.

The term “regulatory element” is used interchangeably herein with the term “regulatory sequence,” and is intended to include promoters, enhancers, and other expression regulatory elements (e.g. transcription termination signals, such as polyadenylation signals and poly-U sequences). Exemplary regulatory elements include a transcription promoter such as, but not limited to, CMV, CMV+ intron A, SV40, RSV, HIV-Ltr, elongation factor 1 alpha (EF11a), MMLV-1tr, internal ribosome entry site (IRES) or P2A peptide to permit translation of multiple genes from a single transcript, metallothionein, a transcription enhancer element, a transcription termination signal, polyadenylation sequences, sequences for optimization of initiation of translation, and translation termination sequences. It will be understood that the choice of the appropriate regulatory element will depend on the encoded component to be expressed (e.g., protein or RNA) or whether the nucleic acid comprises multiple components that require different polymerases or are not intended to be expressed as a fusion protein.

The term “promoter” refers to a DNA sequence that contains an RNA polymerase binding site, transcription start site, TATA box, and/or B recognition element and assists or promotes the transcription and expression of an associated transcribable polynucleotide sequence and/or gene (or transgene). A promoter can be synthetically produced or can be derived from a known or naturally occurring promoter sequence or another promoter sequence. A promoter can be proximal or distal to the gene to be transcribed. A promoter can also include a chimeric promoter comprising a combination of two or more heterologous sequences to confer certain properties. A promoter of the present disclosure can include variants of promoter sequences that are similar in composition, but not identical to, other promoter sequence(s) known or provided herein. A promoter can be classified according to criteria relating to the pattern of expression of an associated coding or transcribable sequence or gene operably linked to the promoter, such as constitutive, developmental, tissue specific, inducible, etc.

The term “enhancer” refers to regulatory element DNA sequences that, when bound by specific proteins called transcription factors, regulate the expression of an associated gene. Enhancers may be located in the intron of the gene, or 5′ or 3′ of the coding sequence of the gene. Enhancers may be proximal to the gene (i.e., within a few tens or hundreds of base pairs (bp) of the promoter), or may be located distal to the gene (i.e., thousands of bp, hundreds of thousands of bp, or even millions of bp away from the promoter). A single gene may be regulated by more than one enhancer, all of which are envisaged as within the scope of the instant disclosure.

As used herein, a “post-transcriptional regulatory element (PRE),” such as a hepatitis PRE, refers to a DNA sequence that, when transcribed creates a tertiary structure capable of exhibiting post-transcriptional activity to enhance or promote expression of an associated gene operably linked thereto.

The term “GATA binding site” refers to a DNA binding site for the GATA family of transcription factors. GATA transcription factors typically recognize a target site conforming to the consensus sequence WGATAR (where W=A or T and R=A or G).

“Recombinant,” as used herein, means that a particular nucleic acid (DNA or RNA) is the product of various combinations of cloning, restriction, and/or ligation steps resulting in a construct having a structural coding or non-coding sequence distinguishable from endogenous nucleic acids found in natural systems. Generally, DNA sequences encoding the structural coding sequence can be assembled from cDNA fragments and short oligonucleotide linkers, or from a series of synthetic oligonucleotides, to provide a synthetic nucleic acid which is capable of being expressed from a recombinant transcriptional unit contained in a cell or in a cell-free transcription and translation system. Such sequences can be provided in the form of an open reading frame uninterrupted by internal non-translated sequences, or introns, which are typically present in eukaryotic genes. Genomic DNA comprising the relevant sequences can also be used in the formation of a recombinant gene or transcriptional unit. Sequences of non-translated DNA may be present 5′ or 3′ from the open reading frame, where such sequences do not interfere with manipulation or expression of the coding regions, and may indeed act to modulate production of a desired product by various mechanisms (see “enhancers” and “promoters”, above).

The term “recombinant polynucleotide” or “recombinant nucleic acid” refers to one which is not naturally occurring, e.g., is made by the artificial combination of two otherwise separated segments of sequence through human intervention. This artificial combination is often accomplished by either chemical synthesis means, or by the artificial manipulation of isolated segments of nucleic acids, e.g., by genetic engineering techniques. Such can be done to replace a codon with a redundant codon encoding the same or a conservative amino acid, while typically introducing or removing a sequence recognition site. Alternatively, it is performed to join together nucleic acid segments of desired functions to generate a desired combination of functions. This artificial combination is often accomplished by either chemical synthesis means, or by the artificial manipulation of isolated segments of nucleic acids, e.g., by genetic engineering techniques.

Similarly, the term “recombinant polypeptide” or “recombinant protein” refers to a polypeptide or protein which is not naturally occurring, e.g., is made by the artificial combination of two otherwise separated segments of amino sequence through human intervention. Thus, e.g., a protein that comprises a heterologous amino acid sequence is recombinant.

As used herein, the term “contacting” means establishing a physical connection between two or more entities. For example, contacting a target nucleic acid sequence with a guide nucleic acid means that the target nucleic acid sequence and the guide nucleic acid are made to share a physical connection; e.g., can hybridize if the sequences share sequence similarity.

“Dissociation constant”, or “K_{d}”, are used interchangeably and mean the affinity between a ligand “L” and a protein “P”; i.e., how tightly a ligand binds to a particular protein. It can be calculated using the formula K_{d}=[L] [P]/[LP], where [P], [L] and [LP] represent molar concentrations of the protein, ligand and complex, respectively.

The disclosure provides compositions and methods useful for editing a target nucleic acid sequence. As used herein “editing” is used interchangeably with “modifying” and includes but is not limited to cleaving, nicking, deleting, knocking in, knocking out, and the like.

The term “knock-out” refers to the elimination of a gene or the expression of a gene. For example, a gene can be knocked out by either a deletion or an addition of a nucleotide sequence that leads to a disruption of the reading frame. As another example, a gene may be knocked out by replacing a part of the gene with an irrelevant sequence. The term “knock-down” as used herein refers to reduction in the expression of a gene or its gene product(s). As a result of a gene knock-down, the protein activity or function may be attenuated or the protein levels may be reduced or eliminated.

As used herein, “homology-directed repair” (HDR) refers to the form of DNA repair that takes place during repair of double-strand breaks in cells. This process requires nucleotide sequence homology, and uses a donor template to repair or knock-out a target DNA, and leads to the transfer of genetic information from the donor (e.g., such as the donor template) to the target. Homology-directed repair can result in an alteration of the sequence of the target nucleic acid sequence by insertion, deletion, or mutation if the donor template differs from the target DNA sequence and part or all of the sequence of the donor template is incorporated into the target DNA at the correct genomic locus.

As used herein, “non-homologous end joining” (NHEJ) refers to the repair of double-strand breaks in DNA by direct ligation of the break ends to one another without the need for a homologous template (in contrast to homology-directed repair, which requires a homologous sequence to guide repair). NHEJ often results in indels; the loss (deletion) or insertion of nucleotide sequence near the site of the double-strand break.

As used herein “micro-homology mediated end joining” (MMEJ) refers to a mutagenic double strand break (DSB) repair mechanism, which always associates with deletions flanking the break sites without the need for a homologous template (in contrast to homology-directed repair, which requires a homologous sequence to guide repair). MMEJ often results in the loss (deletion) of nucleotide sequence near the site of the double-strand break.

A polynucleotide or polypeptide (or protein) has a certain percent “sequence similarity” or “sequence identity” to another polynucleotide or polypeptide, meaning that, when aligned, that percentage of bases or amino acids are the same, and in the same relative position, when comparing the two sequences. Sequence similarity (sometimes referred to as percent similarity, percent identity, or homology) can be determined in a number of different manners. To determine sequence similarity, sequences can be aligned using the methods and computer programs that are known in the art, including BLAST, available over the world wide web at ncbi.nlm.nih.gov/BLAST. Percent complementarity between particular stretches of nucleic acid sequences within nucleic acids can be determined using any convenient method. Example methods include BLAST programs (basic local alignment search tools) and PowerBLAST programs (Altschul et al., J. Mol. Biol., 1990, 215, 403-410; Zhang and Madden, Genome Res., 1997, 7, 649-656) or by using the Gap program (Wisconsin Sequence Analysis Package, Version 8 for Unix, Genetics Computer Group, University Research Park, Madison Wis.), e.g., using default settings, which uses the algorithm of Smith and Waterman (Adv. Appl. Math., 1981, 2, 482-489).

The terms “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones. The term includes fusion proteins, including, but not limited to, fusion proteins with a heterologous amino acid sequence.

A “vector” or “expression vector” is a replicon, such as plasmid, phage, virus, virus-like particle, or cosmid, to which another DNA segment, i.e., an “insert”, may be attached so as to bring about the replication or expression of the attached segment in a cell.

The term “naturally-occurring” or “unmodified” or “wild type” as used herein as applied to a nucleic acid, a polypeptide, a cell, or an organism, refers to a nucleic acid, polypeptide, cell, or organism that is found in nature.

As used herein, a “mutation” refers to an insertion, deletion, substitution, duplication, or inversion of one or more amino acids or nucleotides as compared to a wild-type or reference amino acid sequence or to a wild-type or reference nucleotide sequence.

As used herein the term “isolated” is meant to describe a polynucleotide, a polypeptide, or a cell that is in an environment different from that in which the polynucleotide, the polypeptide, or the cell naturally occurs. An isolated genetically modified host cell may be present in a mixed population of genetically modified host cells.

A “host cell,” as used herein, denotes a eukaryotic cell, a prokaryotic cell, or a cell from a multicellular organism (e.g., a cell line) cultured as a unicellular entity, which eukaryotic or prokaryotic cells are used as recipients for a nucleic acid (e.g., an expression vector), and include the progeny of the original cell which has been genetically modified by the nucleic acid. It is understood that the progeny of a single cell may not necessarily be completely identical in morphology or in genomic or total DNA complement as the original parent, due to natural, accidental, or deliberate mutation. A “recombinant host cell” (also referred to as a “genetically modified host cell”) is a host cell into which has been introduced a heterologous nucleic acid, e.g., an expression vector.

The term “conservative amino acid substitution” refers to the interchangeability in proteins of amino acid residues having similar side chains. For example, a group of amino acids having aliphatic side chains consists of glycine, alanine, valine, leucine, and isoleucine; a group of amino acids having aliphatic-hydroxyl side chains consists of serine and threonine; a group of amino acids having amide-containing side chains consists of asparagine and glutamine; a group of amino acids having aromatic side chains consists of phenylalanine, tyrosine, and tryptophan; a group of amino acids having basic side chains consists of lysine, arginine, and histidine; and a group of amino acids having sulfur-containing side chains consists of cysteine and methionine. Exemplary conservative amino acid substitution groups are: valine-leucine-isoleucine, phenylalanine-tyrosine, lysine-arginine, alanine-valine, and asparagine-glutamine.

As used herein, “treatment” or “treating,” are used interchangeably herein and refer to an approach for obtaining beneficial or desired results, including but not limited to a therapeutic benefit and/or a prophylactic benefit. By therapeutic benefit is meant eradication or amelioration of the underlying disorder or disease being treated. A therapeutic benefit can also be achieved with the eradication or amelioration of one or more of the symptoms or an improvement in one or more clinical parameters associated with the underlying disease such that an improvement is observed in the subject, notwithstanding that the subject may still be afflicted with the underlying disease.

The terms “therapeutically effective amount” and “therapeutically effective dose”, as used herein, refer to an amount of a drug or a biologic, alone or as a part of a composition, that is capable of having any detectable, beneficial effect on any symptom, aspect, measured parameter or characteristics of a disease state or condition when administered in one or repeated doses to a subject such as a human or an experimental animal. Such effect need not be absolute to be beneficial.

As used herein, “administering” is meant as a method of giving a dosage of a composition of the disclosure to a subject.

As used herein, a “subject” is a mammal. Mammals include, but are not limited to, domesticated animals, primates, non-human primates, humans, dogs, porcine (pigs), rabbits, mice, rats and other rodents.

All publications, patents, and patent applications mentioned in this specification are herein incorporated by reference to the same extent as if each individual publication, patent, or patent application was specifically and individually indicated to be incorporated by reference.

### I. General Methods

The practice of the present invention employs, unless otherwise indicated, conventional techniques of immunology, biochemistry, chemistry, molecular biology, microbiology, cell biology, genomics and recombinant DNA, which can be found in such standard textbooks as Molecular Cloning: A Laboratory Manual, 3rd Ed. (Sambrook et al., Harbor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference.

Where a range of values is provided, it is understood that endpoints are included and that each intervening value, to the tenth of the unit of the lower limit unless the context clearly dictates otherwise, between the upper and lower limit of that range and any other stated or intervening value in that stated range, is encompassed. The upper and lower limits of these smaller ranges may independently be included in the smaller ranges, and are also encompassed, subject to any specifically excluded limit in the stated range. Where the stated range includes one or both of the limits, ranges excluding either or both of those included limits are also included.

Unless defined otherwise, all technical and scientific terms used herein have the same meaning as commonly understood by one of ordinary skill in the art to which this invention belongs. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise.

It will be appreciated that certain features of the disclosure, which are, for clarity, described in the context of separate embodiments, may also be provided in combination in a single embodiment. In other cases, various features of the disclosure, which are, for brevity, described in the context of a single embodiment, may also be provided separately or in any suitable sub-combination. It is intended that all combinations of the embodiments pertaining to the disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub-combination was individually and explicitly disclosed herein.

### II Systems for Genetic Editing of BCL11A Genes

In a first aspect, the present disclosure provides systems comprising a Class 2, Type V CRISPR nuclease protein and one or more guide nucleic acids (gRNA) for use in modifying or editing a BCL11A gene in order to reduce or eliminate expression of the BCL11A gene product. Exemplary Class 2, Type V CRISPR nuclease protein and guide nucleic acid systems include the CasX:gRNA system. The CasX:gRNA systems are specifically designed to modify the BCL11A gene in eukaryotic cells. In some cases, the CasX:gRNA systems are designed to knock-down or knock-out the BCL11A gene. Generally, any portion of the BCL11A gene can be targeted using the programable compositions and methods provided herein. In some embodiments, the BCL11A gene to be modified is a wild-type sequence, and the portion to be modified is selected from the group consisting of a BCL11A intron, a BCL11A exon, a BCL11A intron-exon junction, a BCL11A regulatory element, and an intergenic region, or the modification is deletion or mutation of one or more exons.

As used herein, a “system,” such as the systems comprising a CRISPR nuclease protein and one or more gRNAs the disclosure, as well as nucleic acids encoding the CRISPR nuclease proteins and gRNA and vectors comprising the nucleic acids or CRISPR nuclease protein and one or more gRNAs the disclosure, is used interchangeably with term “composition.”

The human BCL11A gene (HGNC: 13221) encodes a protein (Q9H165) having the sequence

MSRRKQGKPQHLSKREFSPEPLEAILTDDEPDHGPLGAPEGDHDLLTCGQCQMNFPLGDILIFIEHKRKQCNGSLCL EKAVDKPPSPSPIEMKKASNPVEVGIQVTPEDDDCLSTSSRGICPKQEHIADKLLHWRGLSSPRSAHGALIPTPGMS AEYAPQGICKDEPSSYTCTTCKQPFTSAWFLLQHAQNTHGLRIYLESEHGSPLTPRVGIPSGLGAECPSQPPLHGIH IADNNPFNLLRIPGSVSREASGLAEGRFPPTPPLFSPPPRHHLDPHRIERLGAEEMALATHHPSAFDRVLRLNPMAM EPPAMDFSRRLRELAGNTSSPPLSPGRPSPMQRLLQPFQPGSKPPFLATPPLPPLQSAPPPSQPPVKSKSCEFCGKT FKFQSNLVVHRRSHTGEKPYKCNLCDHACTQASKLKRHMKTHMHKSSPMTVKSDDGLSTASSPEPGTSDLVGSASSA LKSVVAKFKSENDPNLIPENGDEEEEEDDEEEEEEEEEEEEELTESERVDYGFGLSLEAARHHENSSRGAVVGVGDE SRALPDVMQGMVLSSMQHFSEAFHQVLGEKHKRGHLAEAEGHRDTCDEDSVAGESDRIDDGTVNGRGCSPGESASGG LSKKLLLGSPSSLSPFSKRIKLEKEFDLPPAAMPNTENVYSQWLAGYAASRQLKDPFLSFGDSRQSPFASSSEHSSE NGSLRFSTPPGELDGGISGRSGTGSGGSTPHISGPGPGRPSSKEGRRSDTCEYCGKVFKNCSNLTVHRRSHTGERPY KCELCNYACAQSSKLTRHMKTHGQVGKDVYKCEICKMPFSVYSTLEKHMKKWHSDRVLNNDIKTE (SEQ ID NO: 100). The BCL11A gene is defined as the sequence that spans chr2 60450520-60554467 (GRCh38/hg38 Ensembl 100) of the human genome on chromosome 2.

In some embodiments, the disclosure provides systems specifically designed to modify the BCL11A gene in eukaryotic cells; either in vitro, ex vivo, or in vivo in a subject. Generally, any portion of the BCL11A target nucleic acid can be targeted using the programmable compositions and methods provided herein. In some embodiments, the CRISPR nuclease is a Class 2, Type V nuclease. Although members of Class 2 Type V CRISPR-Cas systems have differences, they share some common characteristics that distinguish them from the Cas9 systems. Firstly, the Type V nucleases possess an RNA-guided single effector containing a RuvC domain but no HNH domain, and they recognize T-rich PAM 5′ upstream to the target region on the non-targeted strand, which is different from Cas9 systems which rely on G-rich PAM at 3′ side of target sequences. Type V nucleases generate staggered double-stranded breaks distal to the PAM sequence, unlike Cas9, which generates a blunt end in the proximal site close to the PAM. In addition, Type V nucleases degrade ssDNA in trans when activated by target dsDNA or ssDNA binding in cis. In some embodiments, the disclosure provides Class 2, Type V nuclease selected from the group consisting of Cas12a, Cas12b, Cas12c, Cas12d (CasY), Cas12j. Cas12k, CasZ, and CasX. In some embodiments, the disclosure provides systems comprising one or more CasX proteins and one or more guide nucleic acids (gRNA) as a CasX:gRNA system. In other embodiments, the CasX:gRNA systems of the disclosure comprise one or more CasX proteins, one or more guide nucleic acids (gRNA) and one or more donor template nucleic acids comprising a nucleic acid encoding a portion of a BCL11A gene wherein the donor template nucleic acid comprises a deletion, an insertion, or a mutation of one or more nucleotides in comparison to a genomic nucleic acid sequence encoding the BCL11A protein. Each of these components and their use in the editing of the BCL11A gene is described herein, below.

In some embodiments, the disclosure provides gene editing pairs of a CasX and a gRNA of any of the embodiments described herein that are capable of being bound together prior to their use for gene editing and, thus, are “pre-complexed” as a ribonuclear protein complex (RNP). The use of a pre-complexed RNP confers advantages in the delivery of the system components to a cell or target nucleic acid sequence for editing of the target nucleic acid sequence.

In some embodiments, the functional RNP can be delivered ex vivo to a cell by electrophoresis or by chemical means. In other embodiments, the functional RNP can be delivered either ex vivo or in vivo by a vector in their functional form. In some embodiments, the RNP can be delivered in vivo to a subject using a CasX delivery particle (XDP). The gRNA can provide target specificity to the complex by including a targeting sequence (or “spacer”) having a nucleotide sequence that is complementary to a sequence of the target nucleic acid sequence while the CasX variant protein of the pre-complexed CasX:gRNA provides the site-specific activity, such as cleavage or nicking of the target sequence, that is guided to a target site (e.g., stabilized at a target site) within a target nucleic acid sequence by virtue of its association with the gRNA.

The systems have utility in the treatment of a subject having a hemoglobinopathy disease, such as sickle cell anemia or β-thalassemia. Each of the components of the CasX:gRNA systems, their functions, and their use in the editing of the target nucleic acids in cells is described more fully, below.

### III. Guide Nucleic Acids of the Systems for Genetic Editing

In another aspect, the disclosure relates to specifically-designed guide ribonucleic acids (gRNA) comprising a targeting sequence complementary to (and are therefore able to hybridize with) a target nucleic acid sequence of a BCL11A gene that have utility, when complexed with a CRISPR nuclease, in genome editing of the BCL11A target nucleic acid in a cell. It is envisioned that in some embodiments, multiple gRNAs are delivered in the systems for the modification of a target nucleic acid. For example, a pair of gRNAs with targeting sequences to different or overlapping regions of the target nucleic acid sequence can be used, when each is complexed with a CRISPR nuclease, in order to bind and cleave at two different or overlapping sites within the gene, which is then edited by non-homologous end joining (NHEJ), homology-directed repair (HDR), homology-independent targeted integration (HITI), micro-homology mediated end joining (MMEJ), single strand annealing (SSA) or base excision repair (BER).

In some embodiments, the disclosure provides gRNAs utilized in the CasX:gRNA systems that have utility in genome editing a BCL11A gene in a eukaryotic cell. In a particular embodiment, the gRNA of the systems are capable of forming a complex with a CasX nuclease. The present disclosure provides specifically-designed gRNAs wherein the targeting sequence (or spacer, described more fully, below) of the gRNA is complementary to (and are therefore able to hybridize with) target nucleic acid sequences when used as a component of the gene editing CasX:gRNA systems. SEQ ID NOs of representative, but non-limiting examples of targeting sequences to the BCL11A target nucleic acid that can be utilized in the gRNA of the embodiments are presented in Table 1, described more fully below.

a. Reference gRNA and gRNA variants

As used herein, a “reference gRNA” refers to a CRISPR guide nucleic acid comprising a wild-type sequence of a naturally-occurring gRNA. In some embodiments, a reference gRNA of the disclosure may be subjected to one or more mutagenesis methods, such as the mutagenesis methods described herein, which may include Deep Mutational Evolution (DME), deep mutational scanning (DMS), error prone PCR, cassette mutagenesis, random mutagenesis, staggered extension PCR, gene shuffling, or domain swapping, in order to generate one or more gRNA variants with enhanced or varied properties relative to the reference gRNA. gRNA variants also include variants comprising one or more exogenous sequences, for example fused to either the 5′ or 3′ end, or inserted internally. The activity of reference gRNAs may be used as a benchmark against which the activity of gRNA variants are compared, thereby measuring improvements in function or other characteristics of the gRNA variants. In other embodiments, a reference gRNA may be subjected to one or more deliberate, specifically-targeted mutations in order to produce a gRNA variant, for example a rationally designed variant.

The gRNAs of the disclosure comprise two segments: a targeting sequence and a protein-binding segment. The targeting segment of a gRNA includes a nucleotide sequence (referred to interchangeably as a guide sequence, a spacer, a targeter, or a targeting sequence) that is complementary to (and therefore hybridizes with) a specific sequence (a target site) within the target nucleic acid sequence (e.g., a target ssRNA, a target ssDNA, a strand of a double stranded target DNA, etc.), described more fully below. The targeting sequence of a gRNA is capable of binding to a target nucleic acid sequence, including a coding sequence, a complement of a coding sequence, a non-coding sequence, and to regulatory elements. The protein-binding segment (or “activator” or “protein-binding sequence”) interacts with (e.g., binds to) a CasX protein as a complex, forming an RNP (described more fully, below). The protein-binding segment is alternatively referred to herein as a “scaffold”, which is comprised of several regions, described more fully, below.

In the case of a dual guide RNA (dgRNA), the targeter and the activator portions each have a duplex-forming segment, where the duplex forming segment of the targeter and the duplex-forming segment of the activator have complementarity with one another and hybridize to one another to form a double stranded duplex (dsRNA duplex for a gRNA). When the gRNA is a gRNA, the term “targeter” or “targeter RNA” is used herein to refer to a crRNA-like molecule (crRNA: “CRISPR RNA”) of a CasX dual guide RNA (and therefore of a CasX single guide RNA when the “activator” and the “targeter” are linked together, e.g., by intervening nucleotides). The crRNA has a 5′ region that anneals with the tracrRNA followed by the nucleotides of the targeting sequence. Thus, for example, a guide RNA (dgRNA or sgRNA) comprises a guide sequence and a duplex-forming segment of a crRNA, which can also be referred to as a crRNA repeat. A corresponding tracrRNA-like molecule (activator) also comprises a duplex-forming stretch of nucleotides that forms the other half of the dsRNA duplex of the protein-binding segment of the guide RNA. Thus, a targeter and an activator, as a corresponding pair, hybridize to form a dual guide NA, referred to herein as a “dual guide NA”, a “dual-molecule gRNA”, a “dgRNA”, a “double-molecule guide NA”, or a “two-molecule guide NA”. Site-specific binding and/or cleavage of a target nucleic acid sequence (e.g., genomic DNA) by the CasX protein can occur at one or more locations (e.g., a sequence of a target nucleic acid) determined by base-pairing complementarity between the targeting sequence of the gRNA and the target nucleic acid sequence. Thus, for example, the gRNA of the disclosure have sequences complementarity to and therefore can hybridize with the target nucleic acid that is adjacent to a sequence complementary to a TC PAM motif or a PAM sequence, such as ATC, CTC, GTC, or TTC. Because the targeting sequence of a guide sequence hybridizes with a sequence of a target nucleic acid sequence, a targeter can be modified by a user to hybridize with a specific target nucleic acid sequence, so long as the location of the PAM sequence is considered. Thus, in some cases, the sequence of a targeter may be a non-naturally occurring sequence. In other cases, the sequence of a targeter may be a naturally-occurring sequence, derived from the gene to be edited. In other embodiments, the activator and targeter of the gRNA are covalently linked to one another (rather than hybridizing to one another) and comprise a single molecule, referred to herein as a “single-molecule gRNA,” “one-molecule guide NA,” “single guide NA”, “single guide RNA”, a “single-molecule guide RNA,” a “one-molecule guide RNA”, or a “sgRNA”. In some embodiments, the sgRNA includes an “activator” or a “targeter” and thus can be an “activator-RNA” and a “targeter-RNA,” respectively. In some embodiments, the gRNA is a ribonucleic acid molecule (“gRNA”), and in other embodiments, the gRNA is a chimera, and comprises both DNA and RNA. As used herein, the term gRNA cover naturally-occurring molecules, as well as sequence variants.

Collectively, the assembled gRNAs of the disclosure comprise four distinct regions, or domains: the RNA triplex, the scaffold stem, the extended stem, and the targeting sequence that, in the embodiments of the disclosure is specific for a target nucleic acid and is located on the 3′ end of the gRNA. The RNA triplex, the scaffold stem, and the extended stem, together, are referred to as the “scaffold” of the gRNA.

b. RNA triplex

In some embodiments of the guide NAs provided herein (including reference sgRNAs), there is a RNA triplex, and the RNA triplex comprises the sequence of a UUU—nX(˜4-15)—UUU (SEQ ID NO: 226) stem loop that ends with an AAAG after 2 intervening stem loops (the scaffold stem loop and the extended stem loop), forming a pseudoknot that may also extend past the triplex into a duplex pseudoknot. The UU-UUU-AAA sequence of the triplex forms as a nexus between the targeting sequence, scaffold stem, and extended stem. In exemplary CasX sgRNAs, the UUU-loop-UUU region is coded for first, then the scaffold stem loop, and then the extended stem loop, which is linked by the tetraloop, and then an AAAG closes off the triplex before becoming the targeting sequence.

c. Scaffold Stem Loop

In some embodiments of sgRNAs of the disclosure, the triplex region is followed by the scaffold stem loop. The scaffold stem loop is a region of the gRNA that is bound by CasX protein (such as a reference or CasX variant protein). In some embodiments, the scaffold stem loop is a fairly short and stable stem loop. In some cases, the scaffold stem loop does not tolerate many changes, and requires some form of an RNA bubble. In some embodiments, the scaffold stem is necessary for CasX sgRNA function. While it is perhaps analogous to the nexus stem of Cas9 as being a critical stem loop, the scaffold stem of a CasX sgRNA, in some embodiments, has a necessary bulge (RNA bubble) that is different from many other stem loops found in CRISPR/Cas systems. In some embodiments, the presence of this bulge is conserved across sgRNA that interact with different CasX proteins. An exemplary sequence of a scaffold stem loop sequence of a gRNA comprises the sequence CCAGCGACUAUGUCGUAUGG (SEQ ID NO: 20).

d. Extended Stem Loop

In some embodiments of the CasX sgRNAs of the disclosure, the scaffold stem loop is followed by the extended stem loop. In some embodiments, the extended stem comprises a synthetic tracr and crRNA fusion that is largely unbound by the CasX protein. In some embodiments, the extended stem loop can be highly malleable. In some embodiments, a single guide gRNA is made with a GAAA tetraloop linker or a GAGAAA linker between the tracr and crRNA in the extended stem loop. In some cases, the targeter and activator of a CasX sgRNA are linked to one another by intervening nucleotides and the linker can have a length of from 3 to 20 nucleotides. In some embodiments of the CasX sgRNAs of the disclosure, the extended stem is a large 32-bp loop that sits outside of the CasX protein in the ribonucleoprotein complex. An exemplary sequence of an extended stem loop sequence of a sgRNA comprises the sequence GCGCUUAUUUAUCGGAGAGAAAUCCGAUAAAUAAGAAGC (SEQ ID NO: 21). In some embodiments, the extended stem loop comprises a GAGAAA spacer sequence.

e. Targeting Sequence

In some embodiments of the gRNAs of the disclosure, the extended stem loop is followed by a region that forms part of the triplex, and then the targeting sequence (or “spacer”) at the 3′ end of the gRNA. The targeting sequence targets the CasX ribonucleoprotein holo complex to a specific region of the target nucleic acid sequence of the gene to be modified. Thus, for example, gRNA targeting sequences of the disclosure have sequences complementarity to, and therefore can hybridize to, a portion of the BCL11A gene in a nucleic acid in a eukaryotic cell (e.g., a eukaryotic chromosome, chromosomal sequence, a eukaryotic RNA, etc.) as a component of the RNP when the TC PAM motif or any one of the PAM sequences TTC, ATC, GTC, or CTC is located 1 nucleotide 5′ to the non-target strand sequence complementary to the target sequence. The targeting sequence of a gRNA can be modified so that the gRNA can target a desired sequence of any desired target nucleic acid sequence, so long as the PAM sequence location is taken into consideration. In some embodiments, the gRNA scaffold is 5′ of the targeting sequence, with the targeting sequence on the 3′ end of the gRNA. In some embodiments, the PAM motif sequence recognized by the nuclease of the RNP is TC. In other embodiments, the PAM sequence recognized by the nuclease of the RNP is NTC.

In some embodiments, the targeting sequence of the gRNA is specific for a portion of a gene encoding a BCL11A protein. In some embodiments, the targeting sequence of a gRNA is specific for a BCL11A exon. In some embodiments, the targeting sequence of a gRNA is specific for a BCL11A intron. In some embodiments, the targeting sequence of the gRNA is specific for a BCL11A intron-exon junction. In some embodiments, the targeting sequence of the gRNA has a sequence that hybridizes with a BCL11A regulatory element, a BCL11A coding region, a BCL11A non-coding region, or combinations thereof (e.g., the intersection of two regions). In some embodiments, the regulatory element comprises a GATA binding sequence. In some embodiments, the targeting sequence of the gRNA is complementary to a sequence comprising one or more single nucleotide polymorphisms (SNPs) of the BCL11A gene or its complement. SNPs that are within BCL11A coding sequence or within BCL11A non-coding sequence are both within the scope of the instant disclosure. In other embodiments, the targeting sequence of the gRNA is complementary to a sequence of an intergenic region of the BCL11A gene or a sequence complementary to an intergenic region of the BCL11A gene.

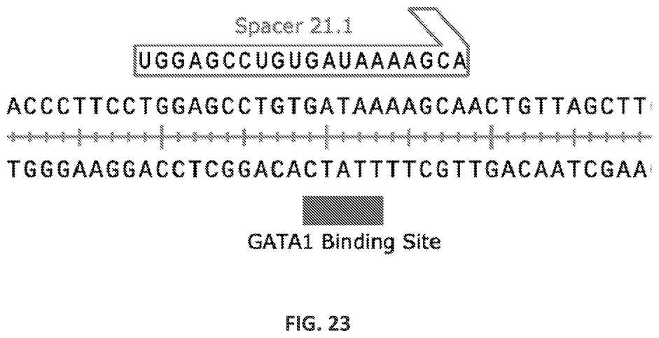

In some embodiments, the targeting sequence of a gRNA is designed to be specific for a regulatory element that regulates expression of the BCL11A gene product. Such regulatory elements include, but are not limited to promoter regions, enhancer regions, intergenic regions, 5′ untranslated regions (5′ UTR), 3′ untranslated regions (3′ UTR), conserved elements, and regions comprising cis-regulatory elements. The promoter region is intended to encompass nucleotides within 5 kb of the initiation point of the encoding sequence or, in the case of gene enhancer elements or conserved elements, can be thousands of base pairs (bp), hundreds of thousands of bp, or even millions of bp away from the encoding sequence of the gene of the target nucleic acid. In particular embodiments, the targeting sequence of the gRNA hybridizes with a sequence that is complementary to a BCL11A regulatory element. In one embodiment, the targeting sequence of the gRNA is UGGAGCCUGUGAUAAAAGCA (SEQ ID NO: 22), which hybridizes with the BCL11A GATA1 erythroid-specific enhancer binding site sequence, or has at least 90% or at least 95% sequence identity thereto (see FIG. 23). In another embodiment, the targeting sequence of the gRNA is UGCUUUUAUCACAGGCUCCA (SEQ ID NO: 23), or has at least 90% or at least 95% sequence identity thereto. In another embodiment, the targeting sequence of the gRNA is UGCUUUUAUCACAGGCUCCA (SEQ ID NO: 23), or has at least 90% or at least 95% sequence identity thereto. In other embodiments, the targeting sequence of the gRNA is selected from the group consisting of CAGGCUCCAGGAAGGGUUUG (SEQ ID NO: 2949), GAGGCCAAACCCUUCCUGGA (SEQ ID NO: 2948), AGUGCAAGCUAACAGUUGCU (SEQ ID NO: 15747), and AUACAACUUUGAAGCUAGUC (SEQ ID NO: 15748).

In subjects that are maturing after birth, GATA1 binding enhances BCL11A expression which, in turn, represses hemoglobin F (HbF) expression, in favor of hemoglobin gamma. However, in subjects with certain hemoglobinopathies, repressing BCL11A expression has been demonstrated to permit HbF expression to resume, which can compensate for otherwise defective hemoglobin in the subject.

In some embodiments, the targeting sequence of the gRNA has between 14 and 35 consecutive nucleotides. In some embodiments, the targeting sequence has 14, 15, 16, 18, 18, 19, 20, 21, 22, 23 24, 25, 26, 27, 28, 29, 30, 31, 32, 33, 34 or 35 consecutive nucleotides. In some embodiments, the targeting sequence consists of 20 consecutive nucleotides. In some embodiments, the targeting sequence consists of 19 consecutive nucleotides. In some embodiments, the targeting sequence consists of 18 consecutive nucleotides. In some embodiments, the targeting sequence consists of 17 consecutive nucleotides. In some embodiments, the targeting sequence consists of 16 consecutive nucleotides. In some embodiments, the targeting sequence consists of 15 consecutive nucleotides. In some embodiments, the targeting sequence has 14, 15, 16, 17, 18, 19, 20, 21, 22, 23, 24, 25, 26, 27, 28, 29, 30, 31, 32, 33, 34 or 35 consecutive nucleotides and the targeting sequence can comprise 0 to 5, 0 to 4, 0 to 3, or 0 to 2 mismatches relative to the target nucleic acid sequence and retain sufficient binding specificity such that the RNP comprising the gRNA comprising the targeting sequence can form a complementary bond with respect to the target nucleic acid.

Representative, but non-limiting examples of targeting sequences to the target nucleic acid sequence contemplated for use in the gRNA of the disclosure are presented as SEQ ID NOS: 272-2100 and 2286-26789 (see Table 1). In some embodiments, the disclosure provides targeting sequences for an ATC PAM comprising a sequence that is at least 50% identical, at least 55% identical, at least 60% identical, at least 65% identical, at least 70% identical, at least 75% identical, at least 80% identical, at least 85% identical, at least 90% identical, at least 95% identical, or 100% identical to a sequence of SEQ ID NOs: 272-2100 or 2286-5625. In some embodiments, the disclosure provides targeting sequences for an ATC PAM comprising a sequence of SEQ ID NOs: 272-2100 or 2286-5625. In some embodiments, the disclosure provides targeting sequences for an CTC PAM comprising a sequence that is at least 50% identical, at least 55% identical, at least 60% identical, at least 65% identical, at least 70% identical, at least 75% identical, at least 80% identical, at least 85% identical, at least 90% identical, at least 95% identical, or 100% identical to a sequence of SEQ ID NOs: 5626-13616. In some embodiments, the disclosure provides targeting sequences for an CTC PAM comprising a sequence of SEQ ID NOs: 5626-13616. In some embodiments, the disclosure provides targeting sequences for an GTC PAM comprising a sequence that is at least 50% identical, at least 55% identical, at least 60% identical, at least 65% identical, at least 70% identical, at least 75% identical, at least 80% identical, at least 85% identical, at least 90% identical, at least 95% identical, or 100% identical to a sequence of SEQ ID NOs: 13617-17903. In some embodiments, the disclosure provides targeting sequences for an GTC PAM comprising a sequence of SEQ ID NOs: 13617-17903. In some embodiments, the disclosure provides targeting sequences for an TTC PAM comprising a sequence that is at least 50% identical, at least 55% identical, at least 60% identical, at least 65% identical, at least 70% identical, at least 75% identical, at least 80% identical, at least 85% identical, at least 90% identical, at least 95% identical, or 100% identical to a sequence of SEQ ID NOs: 17904-26789. In some embodiments, the disclosure provides targeting sequences for an TTC PAM comprising a sequence of SEQ ID NOs: 17904-26789. In some embodiments, the targeting sequence contemplated for use in the gRNA of the disclosure of the gRNA comprises a sequence of SEQ ID NOs: 272-2100 or 2286-26789 with a single nucleotide removed from the 3′ end of the sequence. In other embodiments, the targeting sequence of the gRNA comprises a sequence of SEQ ID NOs: 272-2100 or 2286-26789 with two nucleotides removed from the 3′ end of the sequence. In other embodiments, the targeting sequence of the gRNA comprises a sequence of SEQ ID NOs: 272-2100 or 2286-26789 with three nucleotides removed from the 3′ end of the sequence. In other embodiments, the targeting sequence of the gRNA comprises a sequence of SEQ ID NOs: 272-2100 or 2286-26789 with four nucleotides removed from the 3′ end of the sequence. In other embodiments, the targeting sequence of the gRNA comprises a sequence of SEQ ID NOs: 272-2100 or 2286-26789 with five nucleotides removed from the 3′ end of the sequence. In the foregoing embodiments of the paragraph, thymine (T) nucleotides can be substituted for one or more or all of the uracil (U) nucleotides in any of the targeting sequences such that the gRNA targeting sequence can be a gDNA or a gRNA, or a chimera of RNA and DNA, or in those cases where the encoding sequence for the spacer is incorporated into an expression vector. In some embodiments, a targeting sequence of SEQ ID NOs: 272-2100 or 2286-26789 has at least 1, 2, 3, 4, 5, or 6 or more thymine nucleotides substituted for uracil nucleotides.

TABLE 1
SEQ ID NOs for gRNA Targeting Sequences for BCL11A Gene

| PAM Type | SEQ ID NO |
|---|---|
| ATC | 272-2100, 2286-5625 |
| CTC | 5626-13616 |
| GTC | 13617-17903 |
| TTC | 17904-26789 |

In some embodiments, the CasX:gRNA system comprises a first gRNA and further comprises a second (and optionally a third, fourth, fifth, or more) gRNA, wherein the second gRNA or additional gRNA has a targeting sequence complementary to a different or overlapping portion of the target nucleic acid sequence compared to the targeting sequence of the first gRNA such that multiple points in the target nucleic acid are targeted, and, for example, multiple breaks are introduced in the target nucleic acid by the CasX. It will be understood that in such cases, the second or additional gRNA is complexed with an additional copy of the CasX protein. By selection of the targeting sequences of the gRNA, defined regions of the target nucleic acid sequence bracketing a particular location within the target nucleic acid can be modified or edited using the CasX:gRNA systems described herein, including facilitating the insertion of a donor template comprising a mutation of the BCL11A gene. In a particular embodiment, a second gRNA can comprise a targeting sequence complementary to a sequence that is 5′ or 3′ and adjacent to the GATA1 binding site such that the GATA1 binding site is disrupted.

f gRNA Scaffolds

With the exception of the targeting sequence domain, the remaining components of the gRNA are referred to herein as the scaffold. In some embodiments, the gRNA scaffolds are derived from naturally-occurring sequences, described below as reference gRNA. In other embodiments, the gRNA scaffolds are variants of reference gRNA wherein mutations, insertions, deletions or domain substitutions are introduced to confer desirable or improved properties on the gRNA.

The term “adjacent to” with respect to polynucleotide or amino acid sequences refers to sequences that are next to, or adjoining each other in a polynucleotide or polypeptide. The skilled artisan will appreciate that two sequences can be considered to be adjacent to each other and still encompass a limited amount of intervening sequence, e.g., 1, 2, 3, 4, 5, 6, 7, 8, 9 or 10 nucleotides or amino acids.

Table 2 provides the sequences of reference gRNA tracr and scaffold sequences. In some embodiments, the disclosure provides gRNA sequences wherein the gRNA has a scaffold comprising a sequence of SEQ ID NOs: 4-16 as set forth in Table 2, or a sequence having at least one nucleotide modification relative to a reference gRNA sequence having a sequence of any one of SEQ ID NOS: 4-16 of Table 2. It will be understood that in those embodiments wherein a vector comprises a DNA encoding sequence for a gRNA, or where a gRNA is a chimera of RNA and DNA, that thymine (T) bases can be substituted for the uracil (U) bases of any of the gRNA sequence embodiments described herein.

TABLE 2
Reference gRNA tracr and scaffold sequences

| SEQ | |
|---|---|
| ID | |
| NO. | Nucleotide Sequence |
| 4 | ACAUCUGGCGCGUUUAUUCCAUUACUUUGGAGCCAGUCCCAGCG |
| | ACUAUGUCGUAUGGACGAAGCGCUUAUUUAUCGGAGAGAAACCG |
| | AUAAGUAAAACGCAUCAAAG |
| 5 | UACUGGCGCUUUUAUCUCAUUACUUUGAGAGCCAUCACCAGCGA |
| | CUAUGUCGUAUGGGUAAAGCGCUUAUUUAUCGGAGAGAAAUCCG |
| | AUAAAUAAGAAGCAUCAAAG |
| 6 | ACAUCUGGCGCGUUUAUUCCAUUACUUUGGAGCCAGUCCCAGCG |
| | ACUAUGUCGUAUGGACGAAGCGCUUAUUUAUCGGAGA |
| 7 | ACAUCUGGCGCGUUUAUUCCAUUACUUUGGAGCCAGUCCCAGCG |
| | ACUAUGUCGUAUGGACGAAGCGCUUAUUUAUCGG |
| 8 | UACUGGCGCUUUUAUCUCAUUACUUUGAGAGCCAUCACCAGCGA |
| | CUAUGUCGUAUGGGUAAAGCGCUUAUUUAUCGGAGA |
| 9 | UACUGGCGCUUUUAUCUCAUUACUUUGAGAGCCAUCACCAGCGA |
| | CUAUGUCGUAUGGGUAAAGCGCUUAUUUAUCGG |
| 10 | GUUUACACACUCCCUCUCAUAGGGU |
| 11 | GUUUACACACUCCCUCUCAUGAGGU |
| 12 | UUUUACAUACCCCCUCUCAUGGGAU |
| 13 | GUUUACACACUCCCUCUCAUGGGGG |
| 14 | CCAGCGACUAUGUCGUAUGG |
| 15 | GCGCUUAUUUAUCGGAGAGAAAUCCGAUAAAUAAGAAGC |
| 16 | GGCGCUUUUAUCUCAUUACUUUGAGAGCCAUCACCAGCGACUAU |
| | GUCGUAUGGGUAAAGCGCUUAUUUAUCGGA |

g. gRNA Variants

In another aspect, the disclosure relates to guide nucleic acid variants (referred to herein alternatively as “gRNA variant” or “gRNA variant”), which comprise one or more modifications relative to a reference gRNA scaffold. As used herein, “scaffold” refers to all parts to the gRNA necessary for gRNA function with the exception of the targeting sequence.

In some embodiments, a gRNA variant comprises one or more nucleotide substitutions, insertions, deletions, or swapped or replaced regions relative to a reference gRNA sequence of the disclosure. In some embodiments, a mutation can occur in any region of a reference gRNA scaffold to produce a gRNA variant. In some embodiments, the scaffold of the gRNA variant sequence has at least 20%, at least 30%, at least 40%, at least 50%, at least 60%, or at least 70%, at least 80%, at least 85%, at least about 90%, at least about 95%, at least about 96%, at least about 97%, at least about 98%, or at least about 99% identity to the sequence of SEQ ID NO: 4 or SEQ ID NO: 5.

In some embodiments, a gRNA variant comprises one or more nucleotide changes within one or more regions of the reference gRNA that improve a characteristic relative to the reference gRNA. Exemplary regions include the RNA triplex, the pseudoknot, the scaffold stem loop, and the extended stem loop. In some cases, the variant scaffold stem further comprises a bubble. In other cases, the variant scaffold further comprises a triplex loop region. In still other cases, the variant scaffold further comprises a 5′ unstructured region. In one embodiment, the gRNA variant scaffold comprises a scaffold stem loop having at least 60% sequence identity to SEQ ID NO: 14. In another embodiment, the gRNA variant comprises a scaffold stem loop having the sequence of CCAGCGACUAUGUCGUAGUGG (SEQ ID NO: 25). In another embodiment, the disclosure provides a gRNA scaffold comprising, relative to SEQ ID NO:5, a C18G substitution, a G55 insertion, a U1 deletion, and a modified extended stem loop in which the original 6 nt loop and 13 most-loop-proximal base pairs (32 nucleotides total) are replaced by a Uvsx hairpin (4 nt loop and 5 loop-proximal base pairs; 14 nucleotides total) and the loop-distal base of the extended stem was converted to a fully base-paired stem contiguous with the new Uvsx hairpin by deletion of the A99 and substitution of G64U. In the foregoing embodiment, the gRNA scaffold comprises the sequence

(SEQ ID NO: 2238)
ACUGGCGCUUUUAUCUGAUUACUUUGAGAGCCAUCACCAGCGACUAUGUC
GUAGUGGGUAAAGCUCCCUCUUCGGAGGGAGCAUCAAAG

All gRNA variants that have one or more improved functions or characteristics, or add one or more new functions when the variant gRNA is compared to a reference gRNA described herein, are envisaged as within the scope of the disclosure. A representative example of such a gRNA variant is guide 174 (SEQ ID NO: 2238), the design of which (and the rationale for the design) is described in the Examples. In some embodiments, the gRNA variant adds a new function to the RNP comprising the gRNA variant. In some embodiments, the gRNA variant has an improved characteristic selected from: improved stability; improved solubility; improved transcription of the gRNA; improved resistance to nuclease activity; increased folding rate of the gRNA; decreased side product formation during folding; increased productive folding; improved binding affinity to a CasX protein; improved binding affinity to a target DNA when complexed with a CasX protein; improved gene editing when complexed with a CasX protein; improved specificity of editing when complexed with a CasX protein; and improved ability to utilize a greater spectrum of one or more PAM sequences, including ATC, CTC, GTC, or TTC, in the editing of target DNA when complexed with a CasX protein, or any combination thereof. In some cases, the one or more of the improved characteristics of the gRNA variant is at least about 1.1 to about 100,000-fold improved relative to the reference gRNA of SEQ ID NO: 4 or SEQ ID NO: 5. In other cases, the one or more improved characteristics of the gRNA variant is at least about 1.1, at least about 10, at least about 100, at least about 1000, at least about 10,000, at least about 100,000-fold or more improved relative to the reference gRNA of SEQ ID NO: 4 or SEQ ID NO: 5. In other cases, the one or more of the improved characteristics of the gRNA variant is about 1.1 to 100,00-fold, about 1.1 to 10,00-fold, about 1.1 to 1,000-fold, about 1.1 to 500-fold, about 1.1 to 100-fold, about 1.1 to 50-fold, about 1.1 to 20-fold, about 10 to 100,00-fold, about 10 to 10,00-fold, about 10 to 1,000-fold, about 10 to 500-fold, about 10 to 100-fold, about 10 to 50-fold, about 10 to 20-fold, about 2 to 70-fold, about 2 to 50-fold, about 2 to 30-fold, about 2 to 20-fold, about 2 to 10-fold, about 5 to 50-fold, about 5 to 30-fold, about 5 to 10-fold, about 100 to 100,00-fold, about 100 to 10,00-fold, about 100 to 1,000-fold, about 100 to 500-fold, about 500 to 100,00-fold, about 500 to 10,00-fold, about 500 to 1,000-fold, about 500 to 750-fold, about 1,000 to 100,00-fold, about 10,000 to 100,00-fold, about 20 to 500-fold, about 20 to 250-fold, about 20 to 200-fold, about 20 to 100-fold, about 20 to 50-fold, about 50 to 10,000-fold, about 50 to 1,000-fold, about 50 to 500-fold, about 50 to 200-fold, or about 50 to 100-fold, improved relative to the reference gRNA of SEQ ID NO: 4 or SEQ ID NO: 5. In other cases, the one or more improved characteristics of the gRNA variant is about 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 6-fold, γ-fold, 8-fold, 9-fold, 10-fold, 11-fold, 12-fold, 13-fold, 14-fold, 15-fold, 16-fold, 17-fold, 18-fold, 19-fold, 20-fold, 25-fold, 30-fold, 40-fold, 45-fold, 50-fold, 55-fold, 60-fold, 70-fold, 80-fold, 90-fold, 100-fold, 110-fold, 120-fold, 130-fold, 140-fold, 150-fold, 160-fold, 170-fold, 180-fold, 190-fold, 200-fold, 210-fold, 220-fold, 230-fold, 240-fold, 250-fold, 260-fold, 270-fold, 280-fold, 290-fold, 300-fold, 310-fold, 320-fold, 330-fold, 340-fold, 350-fold, 360-fold, 370-fold, 380-fold, 390-fold, 400-fold, 425-fold, 450-fold, 475-fold, or 500-fold improved relative to the reference gRNA of SEQ ID NO: 4 or SEQ ID NO: 5.

In some embodiments, a gRNA variant can be created by subjecting a reference gRNA to a one or more mutagenesis methods, such as the mutagenesis methods described herein, below, which may include Deep Mutational Evolution (DME), deep mutational scanning (DMS), error prone PCR, cassette mutagenesis, random mutagenesis, staggered extension PCR, gene shuffling, or domain swapping, in order to generate the gRNA variants of the disclosure. The activity of reference gRNAs may be used as a benchmark against which the activity of gRNA variants are compared, thereby measuring improvements in function of gRNA variants compared to the reference gRNA. In other embodiments, a reference gRNA may be subjected to one or more deliberate, targeted mutations, substitutions, or domain swaps in order to produce a gRNA variant, for example a rationally designed variant. Exemplary gRNA variants produced by such methods are described in the Examples and representative sequences of gRNA scaffolds are presented in Table 3.

In some embodiments, the gRNA variant comprises one or more modifications compared to a reference guide nucleic acid scaffold sequence, wherein the one or more modification is selected from: at least one nucleotide substitution in a region of the gRNA variant; at least one nucleotide deletion in a region of the gRNA variant; at least one nucleotide insertion in a region of the gRNA variant; a substitution of all or a portion of a region of the gRNA variant; a deletion of all or a portion of a region of the gRNA variant; or any combination of the foregoing. In some cases, the modification is a substitution of 1 to 15 consecutive or non-consecutive nucleotides in the gRNA variant in one or more regions. In other cases, the modification is a deletion of 1 to 10 consecutive or non-consecutive nucleotides in the gRNA variant in one or more regions. In other cases, the modification is an insertion of 1 to 10 consecutive or non-consecutive nucleotides in the gRNA variant in one or more regions. In other cases, the modification is a substitution of the scaffold stem loop or the extended stem loop with an RNA stem loop sequence from a heterologous RNA source with proximal 5′ and 3′ ends. In some cases, a gRNA variant of the disclosure comprises two or more modifications in one region. In other cases, a gRNA variant of the disclosure comprises modifications in two or more regions. In other cases, a gRNA variant comprises any combination of the foregoing modifications described in this paragraph.

In some embodiments, a 5′ G is added to a gRNA variant sequence for expression in vivo, as transcription from a U6 promoter is more efficient and more consistent with regard to the start site when the +1 nucleotide is a G. In other embodiments, two 5′ Gs are added to a gRNA variant sequence for in vitro transcription to increase production efficiency, as T7 polymerase strongly prefers a G in the +1 position and a purine in the +2 position. In some cases, the 5′ G bases are added to the reference scaffolds of Table 2. In other cases, the 5′ G bases are added to the variant scaffolds SEQ ID NOS: 2238-2285, 26794-26839 and 27219-2726 of Table 3.

Table 3 provides exemplary gRNA variant scaffold sequences of the disclosure. In some embodiments, the gRNA variant scaffold comprises any one of the sequences listed in Table 3, SEQ ID NOS: 2238-2285, 26794-26839 and 27219-27265, or a sequence having at least about 50%, at least about 60%, at least about 70%, at least about 80%, at least about 90%, at least about 95%, at least about 95%, at least about 96%, at least about 97%, at least about 98%, at least about 99% sequence identity thereto. In some embodiments, the gRNA variant scaffold comprises any one of SEQ ID NOS: 2238-2285, 26794-26839 and 27219-27265. In some embodiments, the gRNA variant scaffold comprises any one of SEQ ID NOS: 2281-2285, 26794-26839 and 27219-27265, or a sequence having at least about 50%, at least about 60%, at least about 70%, at least about 80%, at least about 90%, at least about 95%, at least about 95%, at least about 96%, at least about 97%, at least about 98%, at least about 99% sequence identity thereto. In some embodiments, the gRNA variant scaffold comprises any one of SEQ ID NOS: 2281-2285, 26794-26839 and 27219-27265. It will be understood that in those embodiments wherein a vector comprises a DNA encoding sequence for a gRNA, or where a gRNA is a chimera of RNA and DNA, that thymine (T) bases can be substituted for the uracil (U) bases of any of the gRNA sequence embodiments described herein.

TABLE 3
Exemplary gRNA Scaffold Sequences

| SEQ | | |
|---|---|---|
| ID | | |
| NO: | NAME | NUCLEOTIDE SEQUENCE OR DESCRIPTION OF MODIFICATION |
| 2101 | ND | phage replication stable |
| 2102 | ND | Kissing loop_b1 |
| 2103 | ND | Kissing loop_a |
| 2104 | ND | 32, uvsX hairpin |
| 2105 | ND | PP7 |
| 2106 | ND | 64, trip mut, extended stem truncation |
| 2107 | ND | hyperstable tetraloop |
| 2108 | ND | C18G |
| 2109 | ND | U17G |
| 2110 | ND | CUUCGG loop |
| 2111 | ND | MS2 |
| 2112 | ND | −1, A2G, −78, G77U |
| 2113 | ND | QB |
| 2114 | ND | 45,44 hairpin |
| 2115 | ND | U1A |
| 2116 | ND | A14C, U17G |
| 2117 | ND | CUUCGG loop modified |
| 2118 | ND | Kissing loop_b2 |
| 2119 | ND | −76:78, −83:87 |
| 2120 | ND | −4 |
| 2121 | ND | extended stem truncation |
| 2122 | ND | C55 |
| 2123 | ND | trip mut |
| 2124 | ND | −76:78 |
| 2125 | ND | −1:5 |
| 2126 | ND | −83:87 |
| 2127 | ND | =+G28, A82U, −84, |
| 2128 | ND | =+51U |
| 2129 | ND | −1:4, +G5A, +G86, |
| 2130 | ND | =+A94 |
| 2131 | ND | =+G72 |
| 2132 | ND | shorten front, CUUCGG loop modified. extend extended |
| 2133 | ND | A14C |
| 2134 | ND | −1:3, +G3 |
| 2135 | ND | =+C45, +U46 |
| 2136 | ND | CUUCGG loop modified, fun start |
| 2137 | ND | −93:94 |
| 2138 | ND | =+U45 |
| 2139 | ND | −69, −94 |
| 2140 | ND | −94 |
| 2141 | ND | modified CUUCGG, minus U in 1st triplex |
| 2142 | ND | −1:4, +C4, A14C, U17G, +G72, −76:78, −83:87 |
| 2143 | ND | UIC, −73 |
| 2144 | ND | Scaffold uuCG, stem uuCG. Stem swap, t shorten |
| 2145 | ND | Scaffold uuCG, stem uuCG. Stem swap |
| 2146 | ND | =+G60 |
| 2147 | ND | no stem Scaffold uuCG |
| 2148 | ND | no stem Scaffold uuCG, fun start |
| 2149 | ND | Scaffold uuCG, stem uuCG, fun start |
| 2150 | ND | Pseudoknots |
| 2151 | ND | Scaffold uuCG, stem uuCG |
| 2152 | ND | Scaffold uuCG, stem uuCG, no start |
| 2153 | ND | Scaffold uuCG |
| 2154 | ND | =+GCUC36 |
| 2155 | ND | G quadriplex telomere basket+ ends |
| 2156 | ND | G quadriplex M3q |
| 2157 | ND | G quadriplex telomere basket no ends |
| 2158 | ND | 45,44 hairpin (old version) |
| 2159 | ND | Sarcin-ricin loop |
| 2160 | ND | uvsX, C18G |
| 2161 | ND | truncated stem loop, C18G, trip mut (U10C) |
| 2162 | ND | short phage rep, C18G |
| 2163 | ND | phage rep loop, C18G |
| 2164 | ND | =+G18, stacked onto 64 |
| 2165 | ND | truncated stem loop, C18G, −1 A2G |
| 2166 | ND | phage rep loop, C18G, trip mut (U10C) |
| 2167 | ND | short phage rep, C18G, trip mut (U10C) |
| 2168 | ND | uvsX, trip mut (U10C) |
| 2169 | ND | truncated stem loop |
| 2170 | ND | =+A17, stacked onto 64 |
| 2171 | ND | 3′ HDV genomic ribozyme |
| 2172 | ND | phage rep loop, trip mut (U10C) |
| 2173 | ND | −79:80 |
| 2174 | ND | short phage rep, trip mut (U10C) |
| 2175 | ND | extra truncated stem loop |
| 2176 | ND | U17G, C18G |
| 2177 | ND | short phage rep |
| 2178 | ND | uvsX, C18G, −1 A2G |
| 2179 | ND | uvsX, C18G, trip mut (U10C), −1 A2G, HDV −99 G65U |
| 2180 | ND | 3′ HDV antigenomic ribozyme |
| 2181 | ND | uvsX, C18G, trip mut (U10C), −1 A2G, HDV AA(98:99)C |
| 2182 | ND | 3′ HDV ribozyme (Lior Nissim, Timothy Lu) |
| 2183 | ND | TAC(1:3)GA, stacked onto 64 |
| 2184 | ND | uvsX, −1 A2G |
| 2185 | ND | truncated stem loop, C18G, trip mut (U10C), −1 A2G, HDV −99 G65U |
| 2186 | ND | short phage rep, C18G, trip mut (U10C), −1 A2G, HDV −99 G65U |
| 2187 | ND | 3′ sTRSV WT viral Hammerhead ribozyme |
| 2188 | ND | short phage rep, C18G, −1 A2G |
| 2189 | ND | short phage rep, C18G, trip mut (U10C), −1 A2G, 3′ genomic HDV |
| 2190 | ND | phage rep loop, C18G, trip mut (U10C), −1 A2G, HDV −99 G65U |
| 2191 | ND | 3′ HDV ribozyme (Owen Ryan, Jamie Cate) |
| 2192 | ND | phage rep loop, C18G, −1 A2G |
| 2193 | ND | 0.14 |
| 2194 | ND | −78, G77U |
| 2195 | ND | ND |
| 2196 | ND | short phage rep, −1 A2G |
| 2197 | ND | truncated stem loop, C18G, trip mut (U10C), −1 A2G |
| 2198 | ND | −1, A2G |
| 2199 | ND | truncated stem loop, trip mut (U10C), −1 A2G |
| 2200 | ND | uvsX, C18G, trip mut (U10C), −1 A2G |
| 2201 | ND | phage rep loop, −1 A2G |
| 2202 | ND | phage rep loop, trip mut (U10C), −1 A2G |
| 2203 | ND | phage rep loop, C18G, trip mut (U10C), −1 A2G |
| 2204 | ND | truncated stem loop, C18G |
| 2205 | ND | uvsX, trip mut (U10C), −1 A2G |
| 2206 | ND | truncated stem loop, −1 A2G |
| 2207 | ND | short phage rep, trip mut (U10C), −1 A2G |
| 2208 | ND | 5′HDV ribozyme (Owen Ryan, Jamie Cate) |
| 2209 | ND | 5′HDV genomic ribozyme |
| 2210 | ND | truncated stem loop, C18G, trip mut (U10C), −1 A2G, HDV AA(98:99)C |
| 2211 | ND | 5′env25 pistol ribozyme (with an added CUUCGG loop) |
| 2212 | ND | 5′HDV antigenomic ribozyme |
| 2213 | ND | 3′ Hammerhead ribozyme (Lior Nissim, Timothy Lu) guide scaffold scar |
| 2214 | ND | =+A27, stacked onto 64 |
| 2215 | ND | 5′Hammerhead ribozyme (Lior Nissim, Timothy Lu) smaller scar |
| 2216 | ND | phage rep loop, C18G, trip mut (U10C), −1 A2G, HDV AA(98:99)C |
| 2217 | ND | −27, stacked onto 64 |
| 2218 | ND | 3′ Hatchet |
| 2219 | ND | 3′ Hammerhead ribozyme (Lior Nissim, Timothy Lu) |
| 2220 | ND | 5′Hatchet |
| 2221 | ND | 5′HDV ribozyme (Lior Nissim, Timothy Lu) |
| 2222 | ND | 5′Hammerhead ribozyme (Lior Nissim, Timothy Lu) |
| 2223 | ND | 3′ HH15 Minimal Hammerhead ribozyme |
| 2224 | ND | 5′ RBMX recruiting motif |
| 2225 | ND | 3′ Hammerhead ribozyme (Lior Nissim, Timothy Lu) smaller scar |
| 2226 | ND | 3′ env25 pistol ribozyme (with an added CUUCGG loop) |
| 2227 | ND | 3′ Env-9 Twister |
| 2228 | ND | =+AUUAUCUCAUUACU25 |
| 2229 | ND | 5′Env-9 Twister |
| 2230 | ND | 3′ Twisted Sister 1 |
| 2231 | ND | no stem |
| 2232 | ND | 5′HH15 Minimal Hammerhead ribozyme |
| 2233 | ND | 5′Hammerhead ribozyme (Lior Nissim, Timothy Lu) guide scaffold scar |
| 2234 | ND | 5′Twisted Sister 1 |
| 2235 | ND | 5′sTRSV WT viral Hammerhead ribozyme |
| 2236 | ND | 148, =+G55, stacked onto 64 |
| 2237 | ND | 158, 103+148(+G55) −99, G65U |
| 2238 | 174 | ACUGGCGCUUUUAUCUGAUUACUUUGAGAGCCAUCACCAGCGACUAUGUCGUAGUGGGUAA |
| | | AGCUCCCUCUUCGGAGGGAGCAUCAAAG |
| 2239 | 175 | ACUGGCGCCUUUAUCUCAUUACUUUGAGAGCCAUCACCAGCGACUAUGUCGUAUGGGUAAA |
| | | GCGCUUACGGACUUCGGUCCGUAAGAAGCAUCAAAG |
| 2240 | 176 | GCUGGCGCUUUUAUCUGAUUACUUUGAGAGCCAUCACCAGCGACUAUGUCGUAGUGGGUAA |
| | | AGCUCCCUCUUCGGAGGGAGCAUCAAAG |
| 2241 | 177 | ACUGGCGCUUUUAUCUGAUUACUUUGAGAGCCAUCACCAGCGACUAUGUCGUAUGGGUAAA |
| | | GCUCCCUCUUCGGAGGGAGCAUCAAAG |
| 2242 | 181 | ACUGGCGCCUUUAUCUGAUUACUUUGAGAGCCAUCACCAGCGACUAUGUCGUAUGGGUAAA |
| | | GCGCUUACGGACUUCGGUCCGUAAGAAGCAUCAAAG |
| 2243 | 182 | ACUGGCGCUUUUAUCUGAUUACUUUGAGAGCCAUCACCAGCGACUAUGUCGUAUGGGUAAA |
| | | GCGCUUACGGACUUCGGUCCGUAAGAAGCAUCAAAG |
| 2244 | 183 | ACUGGCGCUUUUAUCUGAUUACUUUGAGAGCCAUCACCAGCGACUAUGUCGUAGUGGGUAA |
| | | AGCGCUUACGGACUUCGGUCCGUAAGAAGCAUCAAAG |
| 2245 | 184 | ACUGGCGCUUUUAUCUGAUUACUUUGAGAGCCAUCACCAGCGACUAUGUCGUAUUGGGUAA |
| | | AGCUCCCUCUUCGGAGGGAGCAUCAAAG |
| 2246 | 185 | ACUGGCGCUUUUAUCUGAUUACUUUGAGAGCCAUCACCAGCGACUAUGUCGUAUUGGGUAA |
| | | AGCGCUUACGGACUUCGGUCCGUAAGAAGCAUCAAAG |
| 2247 | 186 | ACUGGCGCCUUUAUCAUCAUUACUUUGAGAGCCAUCACCAGCGACUAUGUCGUAUGGGUAA |
| | | AGCGCUUACGGACUUCGGUCCGUAAGAAGCAUCAAAG |
| 2248 | 187 | ACUGGCGCUUUUAUCUGAUUACUUUGAGAGCCAUCACCAGCGACUAUGUCGUAGUGGGUAA |
| | | AGCGCCCUCUUCGGAGGGAAGCAUCAAAG |
| 2249 | 188 | ACUGGCGCUUUUAUCUGAUUACUUUGAGAGCCAUCACCAGCGACUAUGUCGUAGUGGGUAA |
| | | AGCUCACAUGAGGAUCACCCAUGUGAGCAUCAAAG |
| 2250 | 189 | ACUGGCACUUUUACCUGAUUACUUUGAGAGCCAACACCAGCGACUAUGUCGUAGUGGGUAA |
| | | AGCUCCCUCUUCGGAGGGAGCAUCAAAG |
| 2251 | 190 | ACUGGCACUUUUAUCUGAUUACUUUGAGAGCCAUCACCAGCGACUAUGUCGUAGUGGGUAA |
| | | AGCUCCCUCUUCGGAGGGAGCAUCAAAG |
| 2252 | 191 | ACUGGCCCUUUUAUCUGAUUACUUUGAGAGCCAUCACCAGCGACUAUGUCGUAGUGGGUAA |
| | | AGCUCCCUCUUCGGAGGGAGCAUCAAAG |
| 2253 | 192 | ACUGGCGCUUUUACCUGAUUACUUUGAGAGCCAUCACCAGCGACUAUGUCGUAGUGGGUAA |
| | | AGCUCCCUCUUCGGAGGGAGCAUCAAAG |
| 2254 | 193 | ACUGGCGCUUUUAUCUGAUUACUUUGAGAGCCAACACCAGCGACUAUGUCGUAGUGGGUAA |
| | | AGCUCCCUCUUCGGAGGGAGCAUCAAAG |
| 2255 | 195 | ACUGGCACCUUUACCUGAUUACUUUGAGAGCCAACACCAGCGACUAUGUCGUAUGGGUAAA |
| | | GCGCUUACGGACUUCGGUCCGUAAGAAGCAUCAAAG |
| 2256 | 196 | ACUGGCACCUUUAUCUGAUUACUUUGAGAGCCAUCACCAGCGACUAUGUCGUAUGGGUAAA |
| | | GCGCUUACGGACUUCGGUCCGUAAGAAGCAUCAAAG |
| 2257 | 197 | ACUGGCCCCUUUAUCUGAUUACUUUGAGAGCCAUCACCAGCGACUAUGUCGUAUGGGUAAA |
| | | GCGCUUACGGACUUCGGUCCGUAAGAAGCAUCAAAG |
| 2258 | 198 | ACUGGCGCCUUUAUCUGAUUACUUUGAGAGCCAACACCAGCGACUAUGUCGUAUGGGUAAA |
| | | GCGCUUACGGACUUCGGUCCGUAAGAAGCAUCAAAG |
| 2259 | 199 | GCUGGCGCUUUUAUCUGAUUACUUUGAGAGCCAUCACCAGCGACUAUGUCGUAGUGGGUAA |
| | | AGCUCCCUCUUCGGAGGGAGCAUCAAAG |
| 2260 | 200 | GACUGGCGCUUUUAUCUGAUUACUUUGAGAGCCAUCACCAGCGACUAUGUCGUAGUGGGUA |
| | | AAGCUCCCUCUUCGGAGGGAGCAUCAAAG |
| 2261 | 201 | ACUGGCGCCUUUAUCUGAUUACUUUGGAGAGCCAUCACCAGCGACUAUGUCGUAGUGGGUA |
| | | AAGCUCCCUCUUCGGAGGGAGCAUCAAAG |
| 2262 | 202 | ACUGGCGCAUUUAUCUGAUUACUUUGUGAGCCAUCACCAGCGACUAUGUCGUAGUGGGUAA |
| | | AGCUCCCUCUUCGGAGGGAGCAUCAAAG |
| 2263 | 203 | ACUGGCGCCUUUAUCUGAUUACUUUGAGAGCCAUCACCAGCGACUAUGUCGUAGUGGGUAA |
| | | AGCUCCCUCUUCGGAGGGAGCAUCAAAG |
| 2264 | 204 | ACUGGCGCUUUUAUCUGAUUACUUUGGAGAGCCAUCACCAGCGACUAUGUCGUAGUGGGUA |
| | | AAGCUCCCUCUUCGGAGGGAGCAUCAAAG |
| 2265 | 205 | ACUGGCGCAUUUAUCUGAUUACUUUGAGAGCCAUCACCAGCGACUAUGUCGUAGUGGGUAA |
| | | AGCUCCCUCUUCGGAGGGAGCAUCAAAG |
| 2266 | 206 | ACUGGCGCUUUUAUCUGAUUACUUUGUGAGCCAUCACCAGCGACUAUGUCGUAGUGGGUAA |
| | | AGCUCCCUCUUCGGAGGGAGCAUCAAAG |
| 2267 | 207 | ACUGGCGCUUUUAUUCUGAUUACUUUGAGAGCCAUCACCAGCGACUAUGUCGUAGUGGGUA |
| | | AAGCUCCCUCUUCGGAGGGAGCAUCAAAG |
| 2268 | 208 | ACGGCGCUUUUAUCUGAUUACUUUGAGAGCCAUCACCAGCGACUAUGUCGUAGUGGGUAAA |
| | | GCUCCCUCUUCGGAGGGAGCAUCAAAG |
| 2269 | 209 | ACUGGCGCUUUUAUAUGAUUACUUUGAGAGCCAUCACCAGCGACUAUGUCGUAGUGGGUAA |
| | | AGCUCCCUCUUCGGAGGGAGCAUCAAAG |
| 2270 | 210 | ACUGGCGCUUUUAUCUUGAUUACUUUGAGAGCCAUCACCAGCGACUAUGUCGUAGUGGGUA |
| | | AAGCUCCCUCUUCGGAGGGAGCAUCAAAG |
| 2271 | 211 | ACUGGCGCUUUUAUCUGAUUACUUUGAGAGCCAGCACCAGCGACUAUGUCGUAGUGGGUAA |
| | | AGCUCCCUCUUCGGAGGGAGCAUCAAAG |
| 2272 | 212 | ACUGGCGCUGUUAUCUGAUUACUUCGAGAGCCAUCACCAGCGACUAUGUCGUAGUGGGUAA |
| | | AGCUCCCUCUUCGGAGGGAGCAUCGAAG |
| 2273 | 213 | ACUGGCGCUCUUAUCUGAUUACUUCGAGAGCCAUCACCAGCGACUAUGUCGUAGUGGGUAA |
| | | AGCUCCCUCUUCGGAGGGAGCAUCGAAG |
| 2274 | 214 | ACUGGCGCUUGUAUCUGAUUACUCUGAGAGCCAUCACCAGCGACUAUGUCGUAGUGGGUAA |
| | | AGCUCCCUCUUCGGAGGGAGCAUCAGAG |
| 2275 | 215 | ACUGGCGCUUCUAUCUGAUUACUCUGAGAGCCAUCACCAGCGACUAUGUCGUAGUGGGUAA |
| | | AGCUCCCUCUUCGGAGGGAGCAUCAGAG |
| 2276 | 216 | ACUGGCGCUUUGAUCUGAUUACCUUGAGAGCCAUCACCAGCGACUAUGUCGUAGUGGGUAA |
| | | AGCUCCCUCUUCGGAGGGAGCAUCAAGG |
| 2277 | 217 | ACUGGCGCUUUCAUCUGAUUACCUUGAGAGCCAUCACCAGCGACUAUGUCGUAGUGGGUAA |
| | | AGCUCCCUCUUCGGAGGGAGCAUCAAGG |
| 2278 | 218 | ACUGGCGCUGUUAUCUGAUUACUUUGAGAGCCAUCACCAGCGACUAUGUCGUAGUGGGUAA |
| | | AGCUCCCUCUUCGGAGGGAGCAUCAAAG |
| 2279 | 219 | ACUGGCGCUUUUAUCUGAUUACUUUGAGAGCCAUCACCAGCGACUAUGUCGUAGUGGGUAA |
| | | AGCUCCCUCUUCGGAGGGAGCAUCGAAG |
| 2280 | 220 | ACUGGCGCUUUUAUCUGAUUACUUCGAGAGCCAUCACCAGCGACUAUGUCGUAGUGGGUAA |
| | | AGCUCCCUCUUCGGAGGGAGCAUCAAAG |
| 2281 | 221 | ACUGGCACUUCUAUCUGAUUACUCUGAGAGCCAUCACCAGCGACUAUGUCGUAUGGGUAAA |
| | | GCCGCUUACGGACUUCGGUCCGUAAGAGGCAUCAGAG |
| 2282 | 222 | ACUGGCACUUCUAUCUGAUUACUCUGAGAGCCAUCACCAGCGACUAUGUCGUAGUGGGUAA |
| | | AGCUCCCUCUUCGGAGGGAGCAUCAGAG |
| 2283 | 223 | ACUGGCACCUUUAUCUGAUUACUUUGAGAGCCAUCACCAGCGACUAUGUCGUAUGGGUAAA |
| | | GCCGCUUACGGACUUCGGUCCGUAAGAGGCAUCAAAG |
| 2284 | 224 | ACUGGCACUUGUAUCUGAUUACUCUGAGAGCCAUCACCAGCGACUAUGUCGUAUGGGUAAA |
| | | GCCGCUUACGGACUUCGGUCCGUAAGAGGCAUCAGAG |
| 2285 | 225 | ACUGGCACUUGUAUCUGAUUACUCUGAGAGCCAUCACCAGCGACUAUGUCGUAGUGGGUAA |
| | | AGCUCCCUCUUCGGAGGGAGCAUCAGAG |
| 27219 | 226 | ACUGGCGCUUUUAUCUGAUUACUUUGAGAGCCAUCACCAGCGACUAUGUCGUAGUGGGUAA |
| | | AGCUGCACUAUGGGCGCAGCGUCAAUGACGCUGACGGUACAGGCCAGACAAUUAUUGUCUG |
| | | GUAUAGUGCAGCAUCAAAG |
| 26794 | 229 | ACUGGCACUUUUAUCUGAUUACUUUGAGAGCCAUCACCAGCGACUAUGUCGUAUGGGUAAA |
| | | GCGCUUACGGACUUCGGUCCGUAAGAAGCAUCAAAG |
| 26795 | 230 | ACUGGCACUUCUAUCUGAUUACUCUGAGAGCCAUCACCAGCGACUAUGUCGUAUGGGUAAA |
| | | GCGCUUACGGACUUCGGUCCGUAAGAAGCAUCAGAG |
| 26796 | 231 | ACUGGCGCUUCUAUCUGAUUACUCUGAGAGCCAUCACCAGCGACUAUGUCGUAUGGGUAAA |
| | | GCCGCUUACGGACUUCGGUCCGUAAGAGGCAUCAGAG |
| 26797 | 232 | ACUGGCACUUCUAUCUGAUUACUCUGAGCGCCAUCACCAGCGACUAUGUCGUAUGGGUAAA |
| | | GCCGCUUACGGACUUCGGUCCGUAAGAGGCAUCAGAG |
| 26798 | 233 | ACUGGCGCUUCUAUCUGAUUACUCUGAGCGCCAUCACCAGCGACUAUGUCGUAUGGGUAAA |
| | | GCCGCUUACGGACUUCGGUCCGUAAGAGGCAUCAGAG |
| 26799 | 234 | ACUGGCGCUUCUAUCUGAUUACUCUGAGCGCCAUCACCAGCGACUAUGUCGUAUGGGUAAA |
| | | GCGCCUUACGGACUUCGGUCCGUAAGGAGCAUCAGAG |
| 26800 | 235 | ACUGGCGCUUCUAUCUGAUUACUCUGAGCGCCAUCACCAGCGACUAUGUCGUAGUGGGUAA |
| | | AGCCGCUUACGGACUUCGGUCCGUAAGAGGCAUCAGAG |
| 26801 | 236 | ACGGGACUUUCUAUCUGAUUACUCUGAAGUCCCUCACCAGCGACUAUGUCGUAUGGGUAAA |
| | | GCCGCUUACGGACUUCGGUCCGUAAGAGGCAUCAGAG |
| 26802 | 237 | ACCUGUAGUUCUAUCUGAUUACUCUGACUACAGUCACCAGCGACUAUGUCGUAUGGGUAAA |
| | | GCCGCUUACGGACUUCGGUCCGUAAGAGGCAUCAGAG |
| 26803 | 238 | ACUGGCGCUUUUAUCUGAUUACUUUGAGAGCCAUCACCAGCGACUAUGUCGUAGUGGGUAA |
| | | AGCUGCACGGUGGGCGCAGCUUCGGCUGACGGUACACCGUGCAGCAUCAAAG |
| 26804 | 239 | ACUGGCGCUUUUAUCUGAUUACUUUGAGAGCCAUCACCAGCGACUAUGUCGUAGUGGGUAA |
| | | AGCUGCACGGUGGGCGCAGCUUCGGCUGACGGUACACCGGUGGGCGCAGCUUCGGCUGACG |
| | | GUACACCGUGCAGCAUCAAAG |
| 26805 | 240 | ACUGGCGCUUUUAUCUGAUUACUUUGAGAGCCAUCACCAGCGACUAUGUCGUAGUGGGUAA |
| | | AGCUGCACGGUGGGCGCAGCUUCGGCUGACGGUACACCGGUGGGCGCAGCUUCGGCUGACG |
| | | GUACACCGGUGGGCGCAGCUUCGGCUGACGGUACACCGUGCAGCAUCAAAG |
| 26806 | 241 | ACUGGCGCUUUUAUCUGAUUACUUUGAGAGCCAUCACCAGCGACUAUGUCGUAGUGGGUAA |
| | | AGCUGCACGGUGGGCGCAGCUUCGGCUGACGGUACACCGGUGGGCGCAGCUUCGGCUGACG |
| | | GUACACCGGUGGGCGCAGCUUCGGCUGACGGUACACCGGUGGGCGCAGCUUCGGCUGACGG |
| | | UACACCGUGCAGCAUCAAAG |
| 26807 | 242 | ACUGGCGCUUUUAUCUGAUUACUUUGAGAGCCAUCACCAGCGACUAUGUCGUAGUGGGUAA |
| | | AGCUGCACGGUGGGCGCAGCUUCGGCUGACGGUACACCGGUGGGCGCAGCUUCGGCUGACG |
| | | GUACACCGGUGGGCGCAGCUUCGGCUGACGGUACACCGGUGGGCGCAGCUUCGGCUGACGG |
| | | UACACCGGUGGGCGCAGCUUCGGCUGACGGUACACCGUGCAGCAUCAAAG |
| 26808 | 243 | ACUGGCGCUUUUAUCUGAUUACUUUGAGAGCCAUCACCAGCGACUAUGUCGUAGUGGGUAA |
| | | AGCUGCACCUAGCGGAGGCUAGGUGCAGCAUCAAAG |
| 26809 | 244 | ACUGGCGCUUUUAUCUGAUUACUUUGAGAGCCAUCACCAGCGACUAUGUCGUAGUGGGUAA |
| | | AGCUGCACCUCGGCUUGCUGAAGCGCGCACGGCAAGAGGCGAGGUGCAGCAUCAAAG |
| 26810 | 245 | ACUGGCGCUUUUAUCUGAUUACUUUGAGAGCCAUCACCAGCGACUAUGUCGUAGUGGGUAA |
| | | AGCUGCACCUCUCUCGACGCAGGACUCGGCUUGCUGAAGCGCGCACGGCAAGAGGCGAGGG |
| | | GCGGCGACUGGUGAGUACGCCAAAAAUUUUGACUAGCGGAGGCUAGAAGGAGAGAGGUGCA |
| | | GCAUCAAAG |
| 26811 | 246 | ACUGGCGCUUUUAUCUGAUUACUUUGAGAGCCAUCACCAGCGACUAUGUCGUAGUGGGUAA |
| | | AGCUGCACGGUGCCCGUCUGUUGUGUCGAGAGACGCCAAAAAUUUUGACUAGCGGAGGCUA |
| | | GAAGGAGAGAGAUGGGUGCCGUGCAGCAUCAAAG |
| 26812 | 247 | ACUGGCGCUUUUAUCUGAUUACUUUGAGAGCCAUCACCAGCGACUAUGUCGUAGUGGGUAA |
| | | AGCUGCACAUGGAGAGGAGAUGUGCAGCAUCAAAG |
| 26813 | 248 | ACUGGCGCUUUUAUCUGAUUACUUUGAGAGCCAUCACCAGCGACUAUGUCGUAGUGGGUAA |
| | | AGCUGCACAUGGAGAUGUGCAGCAUCAAAG |
| 26814 | 249 | ACUGGCGCUUUUAUCUGAUUACUUUGAGAGCCAUCACCAGCGACUAUGUCGUAGUGGGUAA |
| | | AGCUUGGGCGCAGCGUCAAUGACGCUGACGGUACAAGCAUCAAAG |
| 26815 | 250 | ACUGGCGCUUUUAUCUGAUUACUUUGAGAGCCAUCACCAGCGACUAUGUCGUAGUGGGUAA |
| | | AGCUGCACUAUGGGCGCAGCGUCAAUGACGCUGACGGUACAGGCCACAUGAGGAUCACCCA |
| | | UGUGGUAUAGUGCAGCAUCAAAG |
| 26816 | 251 | ACUGGCGCUUUUAUCUGAUUACUUUGAGAGCCAUCACCAGCGACUAUGUCGUAGUGGGUAA |
| | | AGCUGCACUAUGGGCGCAGCUCAUGAGGAUCACCCAUGAGCUGACGGUACAGGCCACAUGA |
| | | GGAUCACCCAUGUGGUAUAGUGCAGCAUCAAAG |
| 26817 | 252 | ACUGGCGCUUUUAUCUGAUUACUUUGAGAGCCAUCACCAGCGACUAUGUCGUAGUGGGUAA |
| | | AGCUGCACUAUGGGCGCAGCGUCAAUGACGCUGACGGUACAGGCCACAUGGCAGUCGUAAC |
| | | GACGCGGGUGGUAUAGUGCAGCAUCAAAG |
| 26818 | 253 | ACUGGCGCUUUUAUCUGAUUACUUUGAGAGCCAUCACCAGCGACUAUGUCGUAGUGGGUAA |
| | | AGCUGCACUAUGGGCGCAGCAAACAUGGCAGUCCUAAGGACGCGGGUUUUGCUGACGGUAC |
| | | AGGCCACAUGGCAGUCGUAACGACGCGGGUGGUAUAGUGCAGCAUCAAAG |
| 26819 | 254 | ACUGGCGCUUUUAUCUGAUUACUUUGAGAGCCAUCACCAGCGACUAUGUCGUAGUGGGUAA |
| | | AGCUGCACUAUGGGCGCAGACAUGGCAGUCGUAACGACGCGGGUCUGACGGUACAGGCCAC |
| | | AUGAGGAUCACCCAUGUGGUAUAGUGCAGCAUCAAAG |
| 26820 | 255 | ACUGGCGCUUUUAUCUGAUUACUUUGAGAGCCAUCACCAGCGACUAUGUCGUAGUGGGUAA |
| | | AGCUGCACUAAGGAGUUUAUAUGGAAACCCUUAGUGCAGCAUCAAAG |
| 26821 | 256 | ACUGGCGCUUUUAUCUGAUUACUUUGAGAGCCAUCACCAGCGACUAUGUCGUAGUGGGUAA |
| | | AGCUCAGGAAGCACUAUGGGCGCAGCGUCAAUGACGCUGACGGUACAGGCCAGACAAUUAU |
| | | UGUCUGGUAUAGUGCAGCAGCAGAACAAUUUGCUGAGGGCUAUUGAGGCGCAACAGCAUCU |
| | | GUUGCAACUCACAGUCUGGGGCAUCAAGCAGCUCCAGGCAAGAAUCCUGAGCAUCAAAG |
| 26822 | 257 | ACUGGCGCUUUUAUCUGAUUACUUUGAGAGCCAUCACCAGCGACUAUGUCGUAGUGGGUAA |
| | | AGCUGCACGCCCUGAAGAAGGGCGUGCAGCAUCAAAG |
| 26823 | 258 | ACUGGCGCUUUUAUCUGAUUACUUUGAGAGCCAUCACCAGCGACUAUGUCGUAGUGGGUAA |
| | | AGCUGCACGGCUCGUGUAGCUCAUUAGCUCCGAGCCGUGCAGCAUCAAAG |
| 26824 | 259 | ACUGGCGCUUUUAUCUGAUUACUUUGAGAGCCAUCACCAGCGACUAUGUCGUAGUGGGUAA |
| | | AGCUGCACCCGUGUGCAUCCGCAGUGUCGGAUCCACGGGUGCAGCAUCAAAG |
| 26825 | 260 | ACUGGCGCUUUUAUCUGAUUACUUUGAGAGCCAUCACCAGCGACUAUGUCGUAGUGGGUAA |
| | | AGCUGCACGGAAUCCAUUGCACUCCGGAUUUCACUAGGUGCAGCAUCAAAG |
| 26826 | 261 | ACUGGCGCUUUUAUCUGAUUACUUUGAGAGCCAUCACCAGCGACUAUGUCGUAGUGGGUAA |
| | | AGCUGCACAUGCAUGUCUAAGACAGCAUGUGCAGCAUCAAAG |
| 26827 | 262 | ACUGGCGCUUUUAUCUGAUUACUUUGAGAGCCAUCACCAGCGACUAUGUCGUAGUGGGUAA |
| | | AGCUGCACAAAACAUAAGGAAAACCUAUGUUGUGCAGCAUCAAAG |
| 26828 | 263 | ACUGGCGCUUCUAUCUGAUUACUCUGAGCGCCAUCACCAGCGACUAUGUCGUAGUGGGUAA |
| | | AGCCGCUUACGGACUAUGGGCGCAGCGUCAAUGACGCUGACGGUACAGGCCAGACAAUUAU |
| | | UGUCUGGUAUAGUCCGUAAGAGGCAUCAGAG |
| 26829 | 264 | ACUGGCGCUUCUAUCUGAUUACUCUGAGCGCCAUCACCAGCGACUAUGUCGUAGUGGGUAA |
| | | AGCCGCUUACGGGUGGGCGCAGCGUCAAUGACGCUGACGGUACAGGCCAGACAAUUAUUGU |
| | | CUGGUACCCGUAAGAGGCAUCAGAG |
| 26830 | 265 | ACUGGCGCUUCUAUCUGAUUACUCUGAGCGCCAUCACCAGCGACUAUGUCGUAGUGGGUAA |
| | | AGCCGCUUACGGUAUGGGCGCAGCGUCAAUGACGCUGACGGUACAGGCCACAUGAGGAUCA |
| | | CCCAUGUGGUAUACCGUAAGAGGCAUCAGAG |
| 26831 | 266 | ACUGGCGCUUUUAUCUGAUUACUUUGAGAGCCAUCACCAGCGACUAUGUCGUAGUGGGUAA |
| | | AGCUCCCUAUGGGCGCAGCGUCAAUGACGCUGACGGUACAGGCCACAUGAGGAUCACCCAU |
| | | GUGGUAUAGGGAGCAUCAAAG |
| 26832 | 267 | ACUGGCGCUUCUAUCUGAUUACUCUGAGCGCCAUCACCAGCGACUAUGUCGUAGUGGGUAA |
| | | AGCCGCUUACGGUAUGGGCGCAGCUCAUGAGGAUCACCCAUGAGCUGACGGUACAGGCCAC |
| | | AUGAGGAUCACCCAUGUGGUAUACCGUAAGAGGCAUCAGAG |
| 26833 | 268 | ACUGGCGCUUUUAUCUGAUUACUUUGAGAGCCAUCACCAGCGACUAUGUCGUAGUGGGUAA |
| | | AGCUCCCUAUGGGCGCAGCUCAUGAGGAUCACCCAUGAGCUGACGGUACAGGCCACAUGAG |
| | | GAUCACCCAUGUGGUAUAGGGAGCAUCAAAG |
| 26834 | 269 | ACUGGCGCUUCUAUCUGAUUACUCUGAGCGCCAUCACCAGCGACUAUGUCGUAGUGGGUAA |
| | | AGCCGCUUACGGUAUGGGCGCAGCGUCAAUGACGCUGACGGUACAGGCCACAUGGCAGUCG |
| | | UAACGACGCGGGUGGUAUACCGUAAGAGGCAUCAGAG |
| 26835 | 270 | ACUGGCGCUUUUAUCUGAUUACUUUGAGAGCCAUCACCAGCGACUAUGUCGUAGUGGGUAA |
| | | AGCUCCCUAUGGGCGCAGCGUCAAUGACGCUGACGGUACAGGCCACAUGGCAGUCGUAACG |
| | | ACGCGGGUGGUAUAGGGAGCAUCAAAG |
| 26836 | 271 | ACUGGCGCUUCUAUCUGAUUACUCUGAGCGCCAUCACCAGCGACUAUGUCGUAGUGGGUAA |
| | | AGCCGCUUACGGUAUGGGCGCAGCAAACAUGGCAGUCCUAAGGACGCGGGUUUUGCUGACG |
| | | GUACAGGCCACAUGGCAGUCGUAACGACGCGGGUGGUAUACCGUAAGAGGCAUCAGAG |
| 26837 | 272 | ACUGGCGCUUUUAUCUGAUUACUUUGAGAGCCAUCACCAGCGACUAUGUCGUAGUGGGUAA |
| | | AGCUCCCUAUGGGCGCAGCAAACAUGGCAGUCCUAAGGACGCGGGUUUUGCUGACGGUACA |
| | | GGCCACAUGGCAGUCGUAACGACGCGGGUGGUAUAGGGAGCAUCAAAG |
| 26838 | 273 | ACUGGCGCUUCUAUCUGAUUACUCUGAGCGCCAUCACCAGCGACUAUGUCGUAGUGGGUAA |
| | | AGCCGCUUACGGUAUGGGCGCAGACAUGGCAGUCGUAACGACGCGGGUCUGACGGUACAGG |
| | | CCACAUGAGGAUCACCCAUGUGGUAUACCGUAAGAGGCAUCAGAG |
| 26839 | 274 | ACUGGCGCUUUUAUCUGAUUACUUUGAGAGCCAUCACCAGCGACUAUGUCGUAGUGGGUAA |
| | | AGCUCCCUAUGGGCGCAGACAUGGCAGUCGUAACGACGCGGGUCUGACGGUACAGGCCACA |
| | | UGAGGAUCACCCAUGUGGUAUAGGGAGCAUCAAAG |
| 27220 | 275 | ACUGGCGCUUUUAUCUGAUUACUUUGAGAGCCAUCACCAGCGACUAUGUCG |
| | | UAGUGGGUAAAGCUGCACUAUGGGCGCAGCACCUGAGGAUCACCCAGGUGC |
| | | UGACGGUACAGGCCACCUGAGGAUCACCCAGGUGGUAUAGUGCAGCAUCAA |
| | | AG |
| 27221 | 276 | ACUGGCGCUUUUAUCUGAUUACUUUGAGAGCCAUCACCAGCGACUAUGUCG |
| | | UAGUGGGUAAAGCUGCACUAUGGGCGCAGCGCAUGAGGAUCACCCAUGCGC |
| | | UGACGGUACAGGCCGCAUGAGGAUCACCCAUGCGGUAUAGUGCAGCAUCAA |
| | | AG |
| 27222 | 277 | ACUGGCGCUUUUAUCUGAUUACUUUGAGAGCCAUCACCAGCGACUAUGUCG |
| | | UAGUGGGUAAAGCUGCACUAUGGGCGCAGCGCCUGAGGAUCACCCAGGCGC |
| | | UGACGGUACAGGCCGCCUGAGGAUCACCCAGGCGGUAUAGUGCAGCAUCAA |
| | | AG |
| 27223 | 278 | ACUGGCGCUUUUAUCUGAUUACUUUGAGAGCCAUCACCAGCGACUAUGUCG |
| | | UAGUGGGUAAAGCUGCACUAUGGGCGCAGCGCCUGAGCAUCAGCCAGGCGC |
| | | UGACGGUACAGGCCGCCUGAGCAUCAGCCAGGCGGUAUAGUGCAGCAUCAA |
| | | AG |
| 27224 | 279 | ACUGGCGCUUUUAUCUGAUUACUUUGAGAGCCAUCACCAGCGACUAUGUCG |
| | | UAGUGGGUAAAGCUGCACUAUGGGCGCAGCACAUGAGCAUCAGCCAUGUGC |
| | | UGACGGUACAGGCCACAUGAGCAUCAGCCAUGUGGUAUAGUGCAGCAUCAA |
| | | AG |
| 27225 | 280 | ACUGGCGCUUUUAUCUGAUUACUUUGAGAGCCAUCACCAGCGACUAUGUCG |
| | | UAGUGGGUAAAGCUGCACUAUGGGCGCAGCACAUGAGUAUCAACCAUGUGC |
| | | UGACGGUACAGGCCACAUGAGUAUCAACCAUGUGGUAUAGUGCAGCAUCAA |
| | | AG |
| 27226 | 281 | ACUGGCGCUUUUAUCUGAUUACUUUGAGAGCCAUCACCAGCGACUAUGUCG |
| | | UAGUGGGUAAAGCUGCACUAUGGGCGCAGCACAUGAGAAUCAGCCAUGUGC |
| | | UGACGGUACAGGCCACAUGAGAAUCAGCCAUGUGGUAUAGUGCAGCAUCAA |
| | | AG |
| 27227 | 282 | ACUGGCGCUUUUAUCUGAUUACUUUGAGAGCCAUCACCAGCGACUAUGUCG |
| | | UAGUGGGUAAAGCUGCACUAUGGGCGCAGCCCUUGAGGAUCACCCAUGUGC |
| | | UGACGGUACAGGCCCCUUGAGGAUCACCCAUGUGGUAUAGUGCAGCAUCAA |
| | | AG |
| 27228 | 283 | ACUGGCGCUUUUAUCUGAUUACUUUGAGAGCCAUCACCAGCGACUAUGUCG |
| | | UAGUGGGUAAAGCUGCACUAUGGGCGCAGCACUUGAGGAUCACCCAUGUGC |
| | | UGACGGUACAGGCCACUUGAGGAUCACCCAUGUGGUAUAGUGCAGCAUCAA |
| | | AG |
| 27229 | 284 | ACUGGCGCUUUUAUCUGAUUACUUUGAGAGCCAUCACCAGCGACUAUGUCG |
| | | UAGUGGGUAAAGCUGCACUAUGGGCGCAGCACCUGAGGAUCACCCAUGUGC |
| | | UGACGGUACAGGCCACCUGAGGAUCACCCAUGUGGUAUAGUGCAGCAUCAA |
| | | AG |
| 27230 | 285 | ACUGGCGCUUUUAUCUGAUUACUUUGAGAGCCAUCACCAGCGACUAUGUCGUAGU |
| | | GGGUAAAGCUGCACUAUGGGCGCAGCACAUGAGGAUCACCUAUGUGCUGACGGUA |
| | | CAGGCCACAUGAGGAUCACCUAUGUGGUAUAGUGCAGCAUCAAAG |
| 27231 | 286 | ACUGGCGCUUUUAUCUGAUUACUUUGAGAGCCAUCACCAGCGACUAUGUCGUAGU |
| | | GGGUAAAGCUGCACUAUGGGCGCAGCACAUUAGGAUCACCAAUGUGCUGACGGUA |
| | | CAGGCCACAUUAGGAUCACCAAUGUGGUAUAGUGCAGCAUCAAAG |
| 27232 | 287 | ACUGGCGCUUUUAUCUGAUUACUUUGAGAGCCAUCACCAGCGACUAUGUCGUAGU |
| | | GGGUAAAGCUGCACUAUGGGCGCAGCACAUUAGGAUCACCGAUGUGCUGACGGUA |
| | | CAGGCCACAUUAGGAUCACCGAUGUGGUAUAGUGCAGCAUCAAAG |
| 27233 | 288 | ACUGGCGCUUUUAUCUGAUUACUUUGAGAGCCAUCACCAGCGACUAUGUCGUAGU |
| | | GGGUAAAGCUGCACUAUGGGCGCAGCACAUUAGGAUCACCUAUGUGCUGACGGUA |
| | | CAGGCCACAUUAGGAUCACCUAUGUGGUAUAGUGCAGCAUCAAAG |
| 27234 | 289 | ACUGGCGCUUUUAUCUGAUUACUUUGAGAGCCAUCACCAGCGACUAUGUCGUAGU |
| | | GGGUAAAGCUGCACUAUGGGCGCAGCACAUGAGGAUUACCCAUGUGCUGACGGUA |
| | | CAGGCCACAUGAGGAUUACCCAUGUGGUAUAGUGCAGCAUCAAAG |
| 27235 | 290 | ACUGGCGCUUUUAUCUGAUUACUUUGAGAGCCAUCACCAGCGACUAUGUCGUAGU |
| | | GGGUAAAGCUGCACUAUGGGCGCAGCACAUGAGGAUAACCCAUGUGCUGACGGUA |
| | | CAGGCCACAUGAGGAUAACCCAUGUGGUAUAGUGCAGCAUCAAAG |
| 27236 | 291 | ACUGGCGCUUUUAUCUGAUUACUUUGAGAGCCAUCACCAGCGACUAUGUCGUAGU |
| | | GGGUAAAGCUGCACUAUGGGCGCAGCACAUGAGGAUGACCCAUGUGCUGACGGUA |
| | | CAGGCCACAUGAGGAUGACCCAUGUGGUAUAGUGCAGCAUCAAAG |
| 27237 | 292 | ACUGGCGCUUUUAUCUGAUUACUUUGAGAGCCAUCACCAGCGACUAUGUCGUAGU |
| | | GGGUAAAGCUGCACUAUGGGCGCAGCACAUGAGGACCACCCAUGUGCUGACGGUA |
| | | CAGGCCACAUGAGGACCACCCAUGUGGUAUAGUGCAGCAUCAAAG |
| 27238 | 293 | ACUGGCGCUUUUAUCUGAUUACUUUGAGAGCCAUCACCAGCGACUAUGUCGUAGU |
| | | GGGUAAAGCUGCACUAUGGGCGCAGCAGAUGAGGAUCACCCAUGGGCUGACGGUA |
| | | CAGGCCAGAUGAGGAUCACCCAUGGGGUAUAGUGCAGCAUCAAAG |
| 27239 | 294 | ACUGGCGCUUUUAUCUGAUUACUUUGAGAGCCAUCACCAGCGACUAUGUCGUAGU |
| | | GGGUAAAGCUGCACUAUGGGCGCAGCACAUGGGGAUCACCCAUGUGCUGACGGUA |
| | | CAGGCCACAUGGGGAUCACCCAUGUGGUAUAGUGCAGCAUCAAAG |
| 27240 | 295 | ACUGGCGCUUUUAUCUGAUUACUUUGAGAGCCAUCACCAGCGACUAUGUCGUAGU |
| | | GGGUAAAGCUGCACUAUGGGCGCAGCACAUGAGGAUCACCCAUGUGCUGACGGUA |
| | | CAGGCCACAUGAGGAUCACCCAUGUGGUAUAGUGCAGCAUCAAAG |
| 27241 | 296 | ACUGGCGCUUUUAUCUGAUUACUUUGAGAGCCAUCACCAGCGACUAUGUCGUAGU |
| | | GGGUAAAGCUCACCUGAGGAUCACCCAGGUGAGCAUCAAAG |
| 27242 | 297 | ACUGGCGCUUUUAUCUGAUUACUUUGAGAGCCAUCACCAGCGACUAUGUCGUAGU |
| | | GGGUAAAGCUCGCAUGAGGAUCACCCAUGCGAGCAUCAAAG |
| 27243 | 298 | ACUGGCGCUUUUAUCUGAUUACUUUGAGAGCCAUCACCAGCGACUAUGUCGUAGU |
| | | GGGUAAAGCUCGCCUGAGGAUCACCCAGGCGAGCAUCAAAG |
| 27244 | 299 | ACUGGCGCUUUUAUCUGAUUACUUUGAGAGCCAUCACCAGCGACUAUGUCGUAGU |
| | | GGGUAAAGCUCGCCUGAGCAUCAGCCAGGCGAGCAUCAAAG |
| 27245 | 300 | ACUGGCGCUUUUAUCUGAUUACUUUGAGAGCCAUCACCAGCGACUAUGUCGUAGU |
| | | GGGUAAAGCUCACAUGAGCAUCAGCCAUGUGAGCAUCAAAG |
| 27246 | 301 | ACUGGCGCUUUUAUCUGAUUACUUUGAGAGCCAUCACCAGCGACUAUGUCGUAGU |
| | | GGGUAAAGCUCACAUGAGUAUCAACCAUGUGAGCAUCAAAG |
| 27247 | 302 | ACUGGCGCUUUUAUCUGAUUACUUUGAGAGCCAUCACCAGCGACUAUGUCGUAGU |
| | | GGGUAAAGCUCACAUGAGAAUCAGCCAUGUGAGCAUCAAAG |
| 27248 | 303 | ACUGGCGCUUUUAUCUGAUUACUUUGAGAGCCAUCACCAGCGACUAUGUCGUAGU |
| | | GGGUAAAGCUCCCUUGAGGAUCACCCAUGUGAGCAUCAAAG |
| 27249 | 304 | ACUGGCGCUUUUAUCUGAUUACUUUGAGAGCCAUCACCAGCGACUAUGUCGUAGU |
| | | GGGUAAAGCUCACUUGAGGAUCACCCAUGUGAGCAUCAAAG |
| 27250 | 305 | ACUGGCGCUUUUAUCUGAUUACUUUGAGAGCCAUCACCAGCGACUAUGUCGUAGU |
| | | GGGUAAAGCUCACCUGAGGAUCACCCAUGUGAGCAUCAAAG |
| 27251 | 306 | ACUGGCGCUUUUAUCUGAUUACUUUGAGAGCCAUCACCAGCGACUAUGUCGUAGU |
| | | GGGUAAAGCUCACAUGAGGAUCACCUAUGUGAGCAUCAAAG |
| 27252 | 307 | ACUGGCGCUUUUAUCUGAUUACUUUGAGAGCCAUCACCAGCGACUAUGUCGUAGU |
| | | GGGUAAAGCUCACAUUAGGAUCACCAAUGUGAGCAUCAAAG |
| 27253 | 308 | ACUGGCGCUUUUAUCUGAUUACUUUGAGAGCCAUCACCAGCGACUAUGUCGUAGU |
| | | GGGUAAAGCUCACAUUAGGAUCACCGAUGUGAGCAUCAAAG |
| 27254 | 309 | ACUGGCGCUUUUAUCUGAUUACUUUGAGAGCCAUCACCAGCGACUAUGUCGUAGU |
| | | GGGUAAAGCUCACAUUAGGAUCACCUAUGUGAGCAUCAAAG |
| 27255 | 310 | ACUGGCGCUUUUAUCUGAUUACUUUGAGAGCCAUCACCAGCGACUAUGUCGUAGU |
| | | GGGUAAAGCUCACAUGAGGAUUACCCAUGUGAGCAUCAAAG |
| 27256 | 311 | ACUGGCGCUUUUAUCUGAUUACUUUGAGAGCCAUCACCAGCGACUAUGUCGUAGU |
| | | GGGUAAAGCUCACAUGAGGAUAACCCAUGUGAGCAUCAAAG |
| 27257 | 312 | ACUGGCGCUUUUAUCUGAUUACUUUGAGAGCCAUCACCAGCGACUAUGUCGUAGU |
| | | GGGUAAAGCUCACAUGAGGAUGACCCAUGUGAGCAUCAAAG |
| 27258 | 313 | ACUGGCGCUUUUAUCUGAUUACUUUGAGAGCCAUCACCAGCGACUAUGUCGUAGU |
| | | GGGUAAAGCUCACAUGAGGACCACCCAUGUGAGCAUCAAAG |
| 27259 | 314 | ACUGGCGCUUUUAUCUGAUUACUUUGAGAGCCAUCACCAGCGACUAUGUCGUAGU |
| | | GGGUAAAGCUCAGAUGAGGAUCACCCAUGGGAGCAUCAAAG |
| 27260 | 315 | ACUGGCGCUUUUAUCUGAUUACUUUGAGAGCCAUCACCAGCGACUAUGUCGUAGU |
| | | GGGUAAAGCUCACAUGGGGAUCACCCAUGUGAGCAUCAAAG |
| 27261 | 317 | ACUGGCGCUUCUAUCUGAUUACUCUGAGCGCCAUCACCAGCGACUAUGUCGUAGUGGGUAA |
| | | AGCUCACAUGAGGAUCACCCAUGUGAGCAUCAGAG |
| 27262 | 318 | ACUGGCGCUUCUAUCUGAUUACUCUGAGCGCCAUCACCAGCGACUAUGUCGUAGUGGGUAA |
| | | AGCUGCACUAUGGGCGCAGCGUCAAUGACGCUGACGGUACAGGCCACAUGAGGAUCACCCA |
| | | UGUGGUAUAGUGCAGCAUCAGAG |
| 27263 | 319 | ACUGGCGCUUCUAUCUGAUUACUCUGAGCGCCAUCACCAGCGACUAUGUCGUAGUGGGUAA |
| | | AGCUGCACUAUGGGCGCAGCUCAUGAGGAUCACCCAUGAGCUGACGGUACAGGCCACAUGA |
| | | GGAUCACCCAUGUGGUAUAGUGCAGCAUCAGAG |
| 27264 | 320 | ACUGGCGCUUCUAUCUGAUUACUCUGAGCGCCAUCACCAGCGACUAUGUCGUAGUGGGUAA |
| | | AGCUGCACUAUGGGCGCAGACAUGGCAGUCGUAACGACGCGGGUCUGACGGUACAGGCCAC |
| | | AUGAGGAUCACCCAUGUGGUAUAGUGCAGCAUCAGAG |
| 27265 | 321 | ACUGGCGCUUUUAUCUGAUUACUUUGAGAGCCAUCACCAGCGACUAUGUCGUAGU |
| | | GGGUAAAGCUGCACUAUGGGGCCACAUGAGGAUCACCCAUGUGGUGUACAGCGCA |
| | | GCGUCAAUGACGCUGACGAUAGUGCAGCAUCAAAG |

In some embodiments, a sgRNA variant comprises one or more additional changes to a sequence of SEQ ID NO:2238, SEQ ID NO:2239, SEQ ID NO:2240, SEQ ID NO:2241, SEQ ID NO:2243, SEQ ID NO:2256, SEQ ID NO:2274, SEQ ID NO:2275, SEQ ID NO:2279, SEQ ID NO:2281, SEQ ID NO: 2285, SEQ ID NO: 26797, or SEQ ID NO: 26800 of Table 3.

In some embodiments of the gRNA variants of the disclosure, the gRNA variant comprises at least one modification, wherein the at least one modification compared to the reference guide scaffold of SEQ ID NO:5 is selected from one or more of: (a) a C18G substitution in the triplex loop; (b) a G55 insertion in the stem bubble; (c) a U1 deletion; (d) a modification of the extended stem loop wherein (i) a 6 nt loop and 13 loop-proximal base pairs are replaced by a Uvsx hairpin; and (ii) a deletion of A99 and a substitution of G65U that results in a loop-distal base that is fully base-paired. In exemplary embodiments of the foregoing, the gRNA variant comprises the sequence of any one of SEQ ID NOS: 2238, 2241, 2244, 2248, 2249, 2256, 2259-2285, 26797 or 26800.

In some embodiments, a gRNA variant comprises an exogenous stem loop having a long non-coding RNA (lncRNA). As used herein, a lncRNA refers to a non-coding RNA that is longer than approximately 200 bp in length. In some embodiments, the 5′ and 3′ ends of the exogenous stem loop are base paired; i.e., interact to form a region of duplex RNA. In some embodiments, the 5′ and 3′ ends of the exogenous stem loop are base paired, and one or more regions between the 5′ and 3′ ends of the exogenous stem loop are not base paired.

In some embodiments, the disclosure provide gRNA variants with nucleotide modifications relative to reference gRNA having: (a) substitution of 1 to 15 consecutive or non-consecutive nucleotides in the gRNA variant in one or more regions; (b) a deletion of 1 to 10 consecutive or non-consecutive nucleotides in the gRNA variant in one or more regions; (c) an insertion of 1 to 10 consecutive or non-consecutive nucleotides in the gRNA variant in one or more regions; (d) a substitution of the scaffold stem loop or the extended stem loop with an RNA stem loop sequence from a heterologous RNA source with proximal 5′ and 3′ ends; or any combination of (a)-(d). Any of the substitutions, insertions and deletions described herein can be combined to generate a gNA variant of the disclosure. For example, a gNA variant can comprise at least one substitution and at least one deletion relative to a reference gRNA, at least one substitution and at least one insertion relative to a reference gRNA, at least one insertion and at least one deletion relative to a reference gRNA, or at least one substitution, one insertion and one deletion relative to a reference gRNA.

In some embodiments, a sgRNA variant of the disclosure comprises one or more additional changes to a previously generated variant, the previously generated variant itself serving as the sequence to be modified. In some embodiments, a sgRNA variant comprises one or more additional changes to a sequence of SEQ ID NO: 2238, SEQ ID NO: 2239, SEQ ID NO: 2240, SEQ ID NO: 2241, SEQ ID NO:2241, SEQ ID NO:2274, SEQ ID NO:2275, SEQ ID NO: 2279, or SEQ ID NO: 2285, SEQ ID NO: 26797, or SEQ ID NO: 26800.

In exemplary embodiments, a gRNA variant comprises one or more modification relative to gRNA scaffold variant 174 (SEQ ID NO:2238), wherein the resulting gRNA variant exhibits a functional improvement compared to the parent 174, when assessed in an in vitro or in vivo assay under comparable conditions.

In exemplary embodiments, a gRNA variant comprises one or more modification relative to gRNA scaffold variant 175 (SEQ ID NO:2239), wherein the resulting gRNA variant exhibits a functional improvement compared to the parent 174, when assessed in an in vitro or in vivo assay under comparable conditions.

In exemplary embodiments, a gRNA variant comprises one or more modification relative to gRNA scaffold variant 215 (SEQ ID NO:2275), wherein the resulting gRNA variant exhibits a functional improvement compared to the parent 215, when assessed in an in vitro or in vivo assay under comparable conditions.

In exemplary embodiments, a gRNA variant comprises one or more modification relative to gRNA scaffold variant 221 (SEQ ID NO: 2281), wherein the resulting gRNA variant exhibits a functional improvement compared to the parent 221, when assessed in an in vitro or in vivo assay under comparable conditions.

In exemplary embodiments, a gRNA variant comprises one or more modification relative to gRNA scaffold variant 225 (SEQ ID NO: 2285), wherein the resulting gRNA variant exhibits a functional improvement compared to the parent 225, when assessed in an in vitro or in vivo assay under comparable conditions.

In exemplary embodiments, a gRNA variant comprises one or more modification relative to gRNA scaffold variant 235 (SEQ ID NO: 26800), wherein the resulting gRNA variant exhibits a functional improvement compared to the parent 225, when assessed in an in vitro or in vivo assay under comparable conditions.

In some embodiments, the gRNA variant comprises an exogenous extended stem loop, with such differences from a reference gRNA described as follows. In some embodiments, an exogenous extended stem loop has little or no identity to the reference stem loop regions disclosed herein (e.g., SEQ ID NO:15). In some embodiments, an exogenous stem loop is at least 10 bp, at least 20 bp, at least 30 bp, at least 40 bp, at least 50 bp, at least 60 bp, at least 70 bp, at least 80 bp, at least 90 bp, at least 100 bp, at least 200 bp, at least 300 bp, at least 400 bp, at least 500 bp, at least 600 bp, at least 700 bp, at least 800 bp, at least 900 bp, at least 1,000 bp, at least 2,000 bp, at least 3,000 bp, at least 4,000 bp, at least 5,000 bp, at least 6,000 bp, at least 7,000 bp, at least 8,000 bp, at least 9,000 bp, at least 10,000 bp, at least 12,000 bp, at least 15,000 bp or at least 20,000 bp. In some embodiments, the gRNA variant comprises an extended stem loop region comprising at least 10, at least 100, at least 500, at least 1000, or at least 10,000 nucleotides. In some embodiments, the heterologous stem loop increases the stability of the gRNA. In some embodiments, the heterologous RNA stem loop is capable of binding a protein, an RNA structure, a DNA sequence, or a small molecule. In some embodiments, an exogenous stem loop region replacing the stem loop comprises an RNA stem loop or hairpin in which the resulting gRNA has increased stability and, depending on the choice of loop, can interact with certain cellular proteins or RNA. Such exogenous extended stem loops can comprise, for example a thermostable RNA such as MS2 hairpin (ACAUGAGGAUCACCCAUGU (SEQ ID NO: 27)), QP hairpin (UGCAUGUCUAAGACAGCA (SEQ ID NO: 28)), U1 hairpin II (AAUCCAUUGCACUCCGGAUU (SEQ ID NO: 29)), Uvsx (CCUCUUCGGAGG (SEQ ID NO: 30)), PP7 hairpin (AGGAGUUUCUAUGGAAACCCU (SEQ ID NO: 31)), Phage replication loop (AGGUGGGACGACCUCUCGGUCGUCCUAUCU (SEQ ID NO: 32)), Kissing loop_a (UGCUCGCUCCGUUCGAGCA (SEQ ID NO: 33)), Kissing loop_bl (UGCUCGACGCGUCCUCGAGCA (SEQ ID NO: 34)), Kissing loop_b2 (UGCUCGUUUGCGGCUACGAGCA (SEQ ID NO: 35)), G quadriplex M3q (AGGGAGGGAGGGAGAGG (SEQ ID NO: 149)), G quadriplex telomere basket (GGUUAGGGUUAGGGUUAGG (SEQ ID NO: 150)), Sarcin-ricin loop (CUGCUCAGUACGAGAGGAACCGCAG (SEQ ID NO: 151)) or Pseudoknots (UACACUGGGAUCGCUGAAUUAGAGAUCGGCGUCCUUUCAUUCUAUAUACUUUGG AGUUUUAAAAUGUCUCUAAGUACA (SEQ ID NO: 152)). In some embodiments, one of the foregoing hairpin sequences is incorporated into the stem loop to help traffic the incorporation of the gRNA (and an associated CasX in an RNP complex) into a budding XDP (described more fully, below).

In the embodiments of the gRNA variants, the gRNA variant further comprises a spacer (or targeting sequence) region located at the 3′ end of the gRNA, capable of hybridizing with a target nucleic acid specific to a DMPK sequence described more fully, supra, which comprises at least 14 to about 35 nucleotides wherein the spacer is designed with a sequence that is complementary to a target DNA. In some embodiments, the encoded gRNA variant comprises a targeting sequence of at least 10 to 20 nucleotides complementary to a target DNA. In some embodiments, the targeting sequence has 14, 15, 16, 17, 18, 19, 20, 21, 22, 23, 24, 25, 26, 27, 28, 29, 30, 31, 32, 33, 34 or 35 nucleotides. In some embodiments, the encoded gRNA variant comprises a targeting sequence having 20 nucleotides. In some embodiments, the targeting sequence has 25 nucleotides. In some embodiments, the targeting sequence has 24 nucleotides. In some embodiments, the targeting sequence has 23 nucleotides. In some embodiments, the targeting sequence has 22 nucleotides. In some embodiments, the targeting sequence has 21 nucleotides. In some embodiments, the targeting sequence has 20 nucleotides. In some embodiments, the targeting sequence has 19 nucleotides. In some embodiments, the targeting sequence has 18 nucleotides. In some embodiments, the targeting sequence has 17 nucleotides. In some embodiments, the targeting sequence has 16 nucleotides. In some embodiments, the targeting sequence has 15 nucleotides. In some embodiments, the targeting sequence has 14 nucleotides.

h. Complex Formation with CasX Protein

In some embodiments, upon expression, the gRNA variant is complexed as an RNP with a CasX variant protein comprising any one of the sequences of Table 4 (SEQ ID NOS: 36-99, 101-148, and 26908-27154), or a sequence having at least about 50%, at least about 60%, at least about 70%, at least about 80%, at least about 85%, at least about 90%, at least about 91%, at least about 92%, at least about 93%, at least about 94%, at least about 95%, at least about 96%, at least about 97%, at least about 98%, or at least about 99% identity thereto. In some embodiments, upon expression, the gRNA variant is complexed as an RNP with a CasX variant protein comprising any one of SEQ ID NOS: 59, 72-99, 101-148, or 26908-27154, or a sequence having at least about 50%, at least about 60%, at least about 70%, at least about 80%, at least about 85%, at least about 90%, at least about 91%, at least about 92%, at least about 93%, at least about 94%, at least about 95%, at least about 96%, at least about 97%, at least about 98%, or at least about 99% identity thereto. In some embodiments, upon expression, the gRNA variant is complexed as an RNP with a CasX variant protein comprising any one of SEQ ID NOS 132-148, or 26908-27154 or a sequence having at least about 50%, at least about 60%, at least about 70%, at least about 80%, at least about 85%, at least about 90%, at least about 91%, at least about 92%, at least about 93%, at least about 94%, at least about 95%, at least about 96%, at least about 97%, at least about 98%, or at least about 99% identity thereto.

In some embodiments, a gRNA variant has an improved ability to form a complex with a CasX protein (such as a reference CasX or a CasX variant protein) when compared to a reference gRNA. In some embodiments, a gRNA variant has an improved affinity for a CasX protein (such as a reference or variant protein) when compared to a reference gRNA, thereby improving its ability to form a ribonucleoprotein (RNP) complex with the CasX protein, as described in the Examples. Improving ribonucleoprotein complex formation may, in some embodiments, improve the efficiency with which functional RNPs are assembled. In some embodiments, greater than 90%, greater than 93%, greater than 95%, greater than 96%, greater than 97%, greater than 98% or greater than 99% of RNPs comprising a gRNA variant and its targeting sequence are competent for gene editing of a target nucleic acid.

Exemplary nucleotide changes that can improve the ability of gRNA variants to form a complex with CasX protein may, in some embodiments, include replacing the scaffold stem with a thermostable stem loop. Without wishing to be bound by any theory, replacing the scaffold stem with a thermostable stem loop could increase the overall binding stability of the gRNA variant with the CasX protein. Alternatively, or in addition, removing a large section of the stem loop could change the gRNA variant folding kinetics and make a functional folded gRNA easier and quicker to structurally-assemble, for example by lessening the degree to which the gRNA variant can get “tangled” in itself. In some embodiments, choice of scaffold stem loop sequence could change with different targeting sequences that are utilized for the gRNA. In some embodiments, scaffold sequence can be tailored to the targeting sequence and therefore the target sequence. Biochemical assays can be used to evaluate the binding affinity of CasX protein for the gRNA variant to form the RNP, including the assays of the Examples. For example, a person of ordinary skill can measure changes in the amount of a fluorescently tagged gRNA that is bound to an immobilized CasX protein, as a response to increasing concentrations of an additional unlabeled “cold competitor” gRNA. Alternatively, or in addition, fluorescence signal can be monitored to or seeing how it changes as different amounts of fluorescently labeled gRNA are flowed over immobilized CasX protein. Alternatively, the ability to form an RNP can be assessed using in vitro cleavage assays against a defined target nucleic acid sequence.

### IV. Proteins for Modifying a Target Nucleic Acid

The present disclosure provides systems comprising a CRISPR nuclease that have utility in genome editing of eukaryotic cells. In some embodiments, the CRISPR nuclease employed in the genome editing systems is a Class 2 Type V nuclease. Although members of Class 2, Type V CRISPR-Cas systems have differences, they share some common characteristics that distinguish them from the Cas9 systems. Firstly, the Class 2, Type V nucleases possess a single RNA-guided RuvC domain-containing effector but no HNH domain, and they recognize T-rich PAM 5′ upstream to the target region on the non-targeted strand, which is different from Cas9 systems which rely on G-rich PAM at 3′ side of target sequences. Type V nucleases generate staggered double-stranded breaks distal to the PAM sequence, unlike Cas9, which generates a blunt end in the proximal site close to the PAM. In addition, Type V nucleases degrade ssDNA in trans when activated by target dsDNA or ssDNA binding in cis. In some embodiments, the Type V nucleases of the embodiments recognize a 5′-TC PAM motif and produce staggered ends cleaved solely by the RuvC domain. In some embodiments, the Type V nuclease is selected from the group consisting of Cas12a, Cas12b, Cas12c, Cas12d (CasY), Cas12j, Cas12k, CasZ and CasX. In some embodiments, the present disclosure provides systems comprising a CasX protein and one or more gRNA acids (CasX:gRNA system) that are specifically designed to modify a target nucleic acid sequence in eukaryotic cells.

The term “CasX protein”, as used herein, refers to a family of proteins, and encompasses all naturally occurring CasX proteins, proteins that share at least 50% identity to naturally occurring CasX proteins, as well as CasX variants possessing one or more improved characteristics relative to a naturally-occurring reference CasX protein.

CasX proteins of the disclosure comprise at least one of the following domains: a non-target strand binding (NTSB) domain, a target strand loading (TSL) domain, a helical I domain, a helical II domain, an oligonucleotide binding domain (OBD), and a RuvC DNA cleavage domain.

In some embodiments, a CasX protein can bind and/or modify (e.g., nick, catalyze a double strand break, methylate, demethylate, etc.) a target nucleic acid at a specific sequence targeted by an associated gRNA, which hybridizes to a sequence within the target nucleic acid sequence.

a. Reference CasX Proteins

The disclosure provides naturally-occurring CasX proteins (referred to herein as a “reference CasX protein”), which were subsequently modified to create the CasX variants of the disclosure. For example, reference CasX proteins can be isolated from naturally occurring prokaryotes, such as Deltaproteobacteria, Planctomycetes, or Candidatus Sungbacteria species. A reference CasX protein (interchangeably referred to herein as a reference CasX polypeptide) is a type II CRISPR/Cas endonuclease belonging to the CasX (interchangeably referred to as Cas12e) family of proteins that interacts with a guide RNA to form a ribonucleoprotein (RNP) complex.

In some cases, a reference CasX protein is isolated or derived from Deltaproteobacter having a sequence of:

| (SEQ ID NO: 1) | |
|---|---|
| 1 | MEKRINKIRK KLSADNATKP VSRSGPMKTL LVRVMTDDLK KRLEKRRKKP EVMPQVISNN |
| 61 | AANNLRMLLD DYTKMKEAIL QVYWQEFKDD HVGLMCKFAQ PASKKIDQNK LKPEMDEKGN |
| 121 | LTTAGFACSQ CGQPLFVYKL EQVSEKGKAY TNYFGRCNVA EHEKLILLAQ LKPEKDSDEA |
| 181 | VTYSLGKFGQ RALDFYSIHV TKESTHPVKP LAQIAGNRYA SGPVGKALSD ACMGTIASFL |
| 241 | SKYQDIIIEH QKVVKGNQKR LESLRELAGK ENLEYPSVTL PPQPHTKEGV DAYNEVIARV |
| 301 | RMWVNLNLWQ KLKLSRDDAK PLLRLKGFPS FPVVERRENE VDWWNTINEV KKLIDAKRDM |
| 361 | GRVFWSGVTA EKRNTILEGY NYLPNENDHK KREGSLENPK KPAKRQFGDL LLYLEKKYAG |
| 421 | DWGKVFDEAW ERIDKKIAGL TSHIEREEAR NAEDAQSKAV LTDWLRAKAS FVLERLKEMD |
| 481 | EKEFYACEIQ LQKWYGDLRG NPFAVEAENR VVDISGFSIG SDGHSIQYRN LLAWKYLENG |
| 541 | KREFYLLMNY GKKGRIRFTD GTDIKKSGKW QGLLYGGGKA KVIDLTFDPD DEQLIILPLA |
| 601 | FGTRQGREFI WNDLLSLETG LIKLANGRVI EKTIYNKKIG RDEPALFVAL TFERREVVDP |
| 661 | SNIKPVNLIG VDRGENIPAV IALTDPEGCP LPEFKDSSGG PTDILRIGEG YKEKQRAIQA |
| 721 | AKEVEQRRAG GYSRKFASKS RNLADDMVRN SARDLFYHAV THDAVLVFEN LSRGFGRQGK |
| 781 | RTFMTERQYT KMEDWLTAKL AYEGLTSKTY LSKTLAQYTS KTCSNCGFTI TTADYDGMLV |
| 840 | RLKKTSDGWA TTLNNKELKA EGQITYYNRY KRQTVEKELS AELDRLSEES GNNDISKWTK |
| 901 | GRRDEALFLL KKRFSHRPVQ EQFVCLDCGH EVHADEQAAL NIARSWLFLN SNSTEFKSYK |
| 961 | SGKQPFVGAW QAFYKRRLKE VWKPNA. |

In some cases, a reference CasX protein is isolated or derived from Planctomycetes having a sequence of:

| (SEQ ID NO: 2) | |
|---|---|
| 1 | MQEIKRINKI RRRLVKDSNT KKAGKTGPMK TLLVRVMTPD LRERLENLRK KPENIPQPIS |
| 61 | NTSRANLNKL LTDYTEMKKA ILHVYWEEFQ KDPVGLMSRV AQPAPKNIDQ RKLIPVKDGN |
| 121 | ERLTSSGFAC SQCCQPLYVY KLEQVNDKGK PHTNYFGRCN VSEHERLILL SPHKPEANDE |
| 181 | LVTYSLGKFG QRALDFYSIH VTRESNHPVK PLEQIGGNSC ASGPVGKALS DACMGAVASF |
| 241 | LTKYQDIILE HQKVIKKNEK RLANLKDIAS ANGLAFPKIT LPPQPHTKEG IEAYNNVVAQ |
| 301 | IVIWVNLNLW QKLKIGRDEA KPLQRLKGFP SFPLVERQAN EVDWWDMVCN VKKLINEKKE |
| 361 | DGKVFWQNLA GYKRQEALLP YLSSEEDRKK GKKFARYQFG DLLLHLEKKH GEDWGKVYDE |
| 421 | AWERIDKKVE GLSKHIKLEE ERRSEDAQSK AALTDWLRAK ASFVIEGLKE ADKDEFCRCE |
| 481 | LKLQKWYGDL RGKPFAIEAE NSILDISGFS KQYNCAFIWQ KDGVKKLNLY LIINYFKGGK |
| 541 | LRFKKIKPEA FEANRFYTVI NKKSGEIVPM EVNENFDDPN LIILPLAFGK RQGREFIWND |
| 601 | LLSLETGSLK LANGRVIEKT LYNRRTRQDE PALFVALTFE RREVLDSSNI KPMNLIGIDR |
| 661 | GENIPAVIAL TDPEGCPLSR FKDSLGNPTH ILRIGESYKE KQRTIQAAKE VEQRRAGGYS |
| 721 | RKYASKAKNL ADDMVRNTAR DLLYYAVTQD AMLIFENLSR GFGRQGKRTF MAERQYTRME |
| 781 | DWLTAKLAYE GLPSKTYLSK TLAQYTSKTC SNCGFTITSA DYDRVLEKLK KTATGWMTTI |
| 840 | NGKELKVEGQ ITYYNRYKRQ NVVKDLSVEL DRLSEESVNN DISSWTKGRS GEALSLLKKR |
| 901 | FSHRPVQEKF VCLNCGFETH ADEQAALNIA RSWLELRSQE YKKYQTNKTT GNTDKRAFVE |
| 961 | TWQSFYRKKL KEVWKPAV. |

In some cases, a reference CasX protein is isolated or derived from Candidatus Sungbacteria having a sequence of

| (SEQ ID NO: 3) | |
|---|---|
| 1 | MDNANKPSTK SLVNTTRISD HFGVTPGQVT RVFSFGIIPT KRQYAIIERW FAAVEAARER |
| 61 | LYGMLYAHFQ ENPPAYLKEK FSYETFFKGR PVLNGLRDID PTIMTSAVFT ALRHKAEGAM |
| 121 | AAFHTNHRRL FEEARKKMRE YAECLKANEA LLRGAADIDW DKIVNALRTR LNTCLAPEYD |
| 181 | AVIADFGALC AFRALIAETN ALKGAYNHAL NQMLPALVKV DEPEEAEESP RLRFENGRIN |
| 241 | DLPKFPVAER ETPPDTETII RQLEDMARVI PDTAEILGYI HRIRHKAARR KPGSAVPLPQ |
| 301 | RVALYCAIRM ERNPEEDPST VAGHFLGEID RVCEKRRQGL VRTPEDSQIR ARYMDIISER |
| 361 | ATLAHPDRWT EIQFLRSNAA SRRVRAETIS APFEGFSWTS NRTNPAPQYG MALAKDANAP |
| 421 | ADAPELCICL SPSSAAFSVR EKGGDLIYMR PTGGRRGKDN PGKEITWVPG SFDEYPASGV |
| 481 | ALKLRLYFGR SQARRMLINK TWGLLSDNPR VFAANAELVG KKRNPQDRWK LFFHMVISGP |
| 541 | PPVEYLDFSS DVRSRARTVI GINRGEVNPL AYAVVSVEDG QVLEEGLLGK KEYIDQLIET |
| 601 | RRRISEYQSR EQTPPRDLRQ RVRHLQDTVL GSARAKIHSL IAFWKGILAI ERLDDQFHGR |
| 661 | EQKIIPKKTY LANKTGEMNA LSFSGAVRVD KKGNPWGGMI EIYPGGISRT CTQCGTVWLA |
| 721 | RRPKNPGHRD AMVVIPDIVD DAAATGFDNV DCDAGTVDYG ELFTLSREWV RLTPRYSRVM |
| 781 | RGTLGDLERA IRQGDDRKSR QMLELALEPQ PQWGQFFCHR CGFNGQSDVL AATNLARRAI |
| 841 | SLIRRLPDTD TPPTP. |

b. CasX Variant Proteins

The present disclosure provides variants of a reference CasX protein (interchangeably referred to herein as “CasX variant” or “CasX variant protein”), wherein the CasX variants comprise at least one modification in at least one domain relative to the reference CasX protein, including but not limited to the sequences of SEQ ID NOS:1-3.

The CasX variants of the disclosure have one or more improved characteristics compared to reference CasX proteins. Exemplary improved characteristics of the CasX variant embodiments include, but are not limited to improved folding of the variant, improved binding affinity to the gRNA, improved binding affinity to the target nucleic acid, improved ability to utilize a greater spectrum of PAM sequences in the editing and/or binding of target DNA, improved unwinding of the target DNA, increased editing activity, improved editing efficiency, improved editing specificity, increased percentage of a eukaryotic genome that can be efficiently edited, increased activity of the nuclease, increased target strand loading for double strand cleavage, decreased target strand loading for single strand nicking, decreased off-target cleavage, improved binding of the non-target strand of DNA, improved protein stability, improved protein:gRNA (RNP) complex stability, improved protein solubility, improved protein:gRNA (RNP) complex solubility, improved protein yield, improved protein expression, and improved fusion characteristics, as described more fully, below. Exemplary improved characteristics are described in WO 2020/247882A1 and WO 2020/247883, incorporated by reference herein. In the foregoing embodiments, the one or more of the improved characteristics of the CasX variant is at least about 1.1 to about 100,000-fold improved relative to the reference CasX protein of SEQ ID NO: 1, SEQ ID NO: 2, or SEQ ID NO: 3, when assayed in a comparable fashion. In other embodiments, the improvement is at least about 1.1-fold, at least about 2-fold, at least about 5-fold, at least about 10-fold, at least about 50-fold, at least about 100-fold, at least about 500-fold, at least about 1000-fold, at least about 5000-fold, at least about 10,000-fold, or at least about 100,000-fold compared to the reference CasX protein of SEQ ID NO: 1, SEQ ID NO: 2, or SEQ ID NO: 3, when assayed in a comparable fashion. In other cases, the one or more improved characteristics of an RNP of the CasX variant and the gRNA variant are at least about 1.1, at least about 10, at least about 100, at least about 1000, at least about 10,000, at least about 100,000-fold or more improved relative to an RNP of the reference CasX protein of SEQ ID NO:1, SEQ ID NO:2, or SEQ ID NO:3 and the gRNA of Table 2. In other cases, the one or more of the improved characteristics of an RNP of the CasX variant and the gRNA variant are about 1.1 to 100,00-fold, about 1.1 to 10,00-fold, about 1.1 to 1,000-fold, about 1.1 to 500-fold, about 1.1 to 100-fold, about 1.1 to 50-fold, about 1.1 to 20-fold, about 10 to 100,00-fold, about 10 to 10,00-fold, about 10 to 1,000-fold, about 10 to 500-fold, about 10 to 100-fold, about 10 to 50-fold, about 10 to 20-fold, about 2 to 70-fold, about 2 to 50-fold, about 2 to 30-fold, about 2 to 20-fold, about 2 to 10-fold, about 5 to 50-fold, about 5 to 30-fold, about 5 to 10-fold, about 100 to 100,00-fold, about 100 to 10,00-fold, about 100 to 1,000-fold, about 100 to 500-fold, about 500 to 100,00-fold, about 500 to 10,00-fold, about 500 to 1,000-fold, about 500 to 750-fold, about 1,000 to 100,00-fold, about 10,000 to 100,00-fold, about 20 to 500-fold, about 20 to 250-fold, about 20 to 200-fold, about 20 to 100-fold, about 20 to 50-fold, about 50 to 10,000-fold, about 50 to 1,000-fold, about 50 to 500-fold, about 50 to 200-fold, or about 50 to 100-fold, improved relative to an RNP of the reference CasX protein of SEQ ID NO:1, SEQ ID NO:2, or SEQ ID NO:3 and the gRNA of Table 2, when assayed in a comparable fashion. In other cases, the one or more improved characteristics of an RNP of the CasX variant and the gRNA variant are about 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, 20-fold, 25-fold, 30-fold, 40-fold, 45-fold, 50-fold, 55-fold, 60-fold, 70-fold, 80-fold, 90-fold, 100-fold, 110-fold, 120-fold, 130-fold, 140-fold, 150-fold, 160-fold, 170-fold, 180-fold, 190-fold, 200-fold, 210-fold, 220-fold, 230-fold, 240-fold, 250-fold, 260-fold, 270-fold, 280-fold, 290-fold, 300-fold, 310-fold, 320-fold, 330-fold, 340-fold, 350-fold, 360-fold, 370-fold, 380-fold, 390-fold, 400-fold, 425-fold, 450-fold, 475-fold, or 500-fold improved relative to an RNP of the reference CasX protein of SEQ ID NO:1, SEQ ID NO:2, or SEQ ID NO:3 and the gRNA of Table 2, when assayed in a comparable fashion.

The term CasX variant is inclusive of variants that are fusion proteins; i.e. the CasX is “fused to” a heterologous sequence. This includes CasX variants comprising CasX variant sequences and N-terminal, C-terminal, or internal fusions of the CasX to a heterologous protein or domain thereof.

In some embodiments, the CasX variant comprises at least one modification in the NTSB domain. In some embodiments, the CasX variant comprises at least one modification in the TSL domain. In some embodiments, the CasX variant comprises at least one modification in the helical I domain. In some embodiments, the CasX variant comprises at least one modification in the helical II domain. In some embodiments, the CasX variant comprises at least one modification in the OBD domain. In some embodiments, the CasX variant comprises at least one modification in the RuvC DNA cleavage domain. In some embodiments, the at least one modification in the RuvC DNA cleavage domain comprises an amino acid substitution of one or more of amino acids K682, G695, A708, V711, D732, A739, D733, L742, V747, F755, M771, M779, W782, A788, G791, L792, P793, Y797, M799, Q804, S819, or Y857 or a deletion of amino acid P793 of SEQ ID NO:2.

In some embodiments, the CasX variant protein comprises at least one modification in at least 1 domain, in at least each of 2 domains, in at least each of 3 domains, in at least each of 4 domains or in at least each of 5 domains of the reference CasX protein, including the sequences of SEQ ID NOS: 1-3. In some embodiments, the CasX variant protein comprises two or more modifications in at least one domain of the reference CasX protein. In some embodiments, the CasX variant protein comprises at least two modifications in at least one domain of the reference CasX protein, at least three modifications in at least one domain of the reference CasX protein or at least four modifications in at least one domain of the reference CasX protein. In some embodiments, wherein the CasX variant comprises two or more modifications compared to a reference CasX protein, each modification is made in a domain independently selected from the group consisting of a NTSBD, TSLD, Helical I domain, Helical II domain, OBD, and RuvC DNA cleavage domain. In some embodiments, the at least one modification of the CasX variant protein comprises a deletion of at least a portion of one domain of the reference CasX protein of SEQ ID NOS: 1-3. In some embodiments, the deletion is in the NTSBD, TSLD, Helical I domain, Helical II domain, OBD, or RuvC DNA cleavage domain. In other embodiments, the disclosure provides CasX variants wherein the CasX variants comprise at least one modification relative to another CasX variant; e.g., CasX variant 515 is a variant of CasX variant 491. All variants that improve one or more functions or characteristics of the CasX variant protein when compared to a reference CasX protein (or the variant from which it was derived) described herein are envisaged as being within the scope of the disclosure.

In some embodiments, the modification of the CasX variant is a mutation in one or more amino acids of the reference CasX. In other embodiments, the modification is a substitution of one or more domains of the reference CasX with one or more domains from a different CasX. In some embodiments, insertion includes the insertion of a part or all of a domain from a different CasX protein. Mutations can occur in any one or more domains of the reference CasX protein, and may include, for example, deletion of part or all of one or more domains, or one or more amino acid substitutions, deletions, or insertions in any domain of the reference CasX protein. The domains of CasX proteins include the non-target strand binding (NTSB) domain, the target strand loading (TSL) domain, the helical I domain, the helical II domain, the oligonucleotide binding domain (OBD), and the RuvC DNA cleavage domain. Any change in amino acid sequence of a reference CasX protein that leads to an improved characteristic of the CasX protein is considered a CasX variant protein of the disclosure. For example, CasX variants can comprise one or more amino acid substitutions, insertions, deletions, or swapped domains, or any combinations thereof, relative to a reference CasX protein sequence.

Suitable mutagenesis methods for generating CasX variant proteins of the disclosure may include, for example, Deep Mutational Evolution (DME), deep mutational scanning (DMS), error prone PCR, cassette mutagenesis, random mutagenesis, staggered extension PCR, gene shuffling, or domain swapping. In some embodiments, the CasX variants are designed, for example by selecting one or more desired mutations in a reference CasX. In certain embodiments, the activity of a reference CasX protein is used as a benchmark against which the activity of one or more CasX variants are compared, thereby measuring improvements in function of the CasX variants.

In some embodiments of the CasX variants described herein, the at least one modification comprises: (a) a substitution of 1 to 100 consecutive or non-consecutive amino acids in the CasX variant compared to a reference CasX of SEQ ID NO:1, SEQ ID NO:2, SEQ ID NO:3, CasX variant 491 or CasX variant 515; (b) a deletion of 1 to 100 consecutive or non-consecutive amino acids in the CasX variant compared to a reference CasX or the variant from which it was derived; (c) an insertion of 1 to 100 consecutive or non-consecutive amino acids in the CasX compared to a reference CasX or the variant from which it was derived; or (d) any combination of (a)-(c). In some embodiments, the at least one modification comprises: (a) a substitution of 5-10 consecutive or non-consecutive amino acids in the CasX variant compared to a reference CasX of SEQ ID NO:1, SEQ ID NO:2, SEQ ID NO:3, CasX 491 or CasX 515; (b) a deletion of 1-5 consecutive or non-consecutive amino acids in the CasX variant compared to a reference CasX or the variant from which it was derived; (c) an insertion of 1-5 consecutive or non-consecutive amino acids in the CasX compared to a reference CasX or the variant from which it was derived; or (d) any combination of (a)-(c).

In some embodiments, the CasX variant protein comprises or consists of a sequence that has at least 1, at least 2, at least 3, at least 4, at least 5, at least 6, at least 7, at least 8, at least 9, at least 10, at least 20, at least 30, at least 40, at least 50, at least 60, at least 70, at lease 80, at least 90, or at least 100 alterations relative to the sequence of SEQ ID NO:1, SEQ ID NO:2, SEQ ID NO:3, CasX 491 (with reference to Table 4) or CasX 515 (with reference to Table 4). These alterations can be amino acid insertions, deletions, substitutions, or any combinations thereof. The alterations can be in one domain or in any domain or any combination of domains of the CasX variant. Any amino acid can be substituted for any other amino acid in the substitutions described herein. The substitution can be a conservative substitution (e.g., a basic amino acid is substituted for another basic amino acid). The substitution can be a non-conservative substitution (e.g., a basic amino acid is substituted for an acidic amino acid or vice versa). For example, a proline in a reference CasX protein can be substituted for any of arginine, histidine, lysine, aspartic acid, glutamic acid, serine, threonine, asparagine, glutamine, cysteine, glycine, alanine, isoleucine, leucine, methionine, phenylalanine, tryptophan, tyrosine or valine to generate a CasX variant protein of the disclosure.

Any permutation of the substitution, insertion and deletion embodiments described herein can be combined to generate a CasX variant protein of the disclosure. For example, a CasX variant protein can comprise at least one substitution and at least one deletion relative to a reference CasX protein sequence, at least one substitution and at least one insertion relative to a reference CasX protein sequence, at least one insertion and at least one deletion relative to a reference CasX protein sequence, or at least one substitution, one insertion and one deletion relative to a reference CasX protein sequence.

In some embodiments, the CasX variant comprises at least one modification compared to the reference CasX sequence of SEQ ID NO:2 is selected from one or more of: (a) an amino acid substitution of L379R; (b) an amino acid substitution of A708K; (c) an amino acid substitution of T620P; (d) an amino acid substitution of E385P; (e) an amino acid substitution of Y857R; (f) an amino acid substitution of I658V; (g) an amino acid substitution of F399L; (h) an amino acid substitution of Q252K; (i) an amino acid substitution of L404K; and (j) an amino acid deletion of P793.

In some embodiments, the CasX variant protein comprises between 400 and 2000 amino acids, between 500 and 1500 amino acids, between 700 and 1200 amino acids, between 800 and 1100 amino acids, or between 900 and 1000 amino acids.

In some embodiments, a CasX variant protein comprises a sequence of SEQ ID NOS: 59, 72-99, 101-148, and 26908-27154 as set forth in Table 4. In some embodiments, a CasX variant protein consists of a sequence of SEQ ID NOS: 59, 72-99, 101-148, and 26908-27154 as set forth in Table 4. In other embodiments, a CasX variant protein comprises a sequence at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 86% identical, at least 87% identical, at least 88% identical, at least 89% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical to a sequence of SEQ ID NOS: 59, 72-99, 101-148, or 26908-27154 as set forth in Table 4. In some embodiments, a CasX variant protein comprises or consists of a sequence of SEQ ID NOS: 536-99, 101-148, or 26908-27154. In other embodiments, a CasX variant protein comprises a sequence at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 86% identical, at least 87% identical, at least 88% identical, at least 89% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical to a sequence of SEQ ID NOS: 36-99, 101-148, or 26908-27154. In some embodiments, a CasX variant protein comprises or consists of a sequence of SEQ ID NOS: 132-148, or 26908-27154. In other embodiments, a CasX variant protein comprises a sequence at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 86% identical, at least 87% identical, at least 88% identical, at least 89% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical to a sequence of SEQ ID NOS: 132-148 or 26908-27154.

TABLE 4
CasX Variant Sequences

| SEQ | | |
|---|---|---|
| ID NO | Variant | Description of Variant |
| 36 | ND | TSL, Helical I, Helical II, OBD and RuvC domains from SEQ ID NO: 2 and an |
| | | NTSB domain from SEQ ID NO: 1 |
| 37 | ND | NTSB, Helical I, Helical II, OBD and RuvC domains from SEQ ID NO: 2 and a TSL |
| | | domain from SEQ ID NO: 1. |
| 38 | ND | TSL, Helical I, Helical II, OBD and RuvC domains from SEQ ID NO: 1 and an |
| | | NTSB domain from SEQ ID NO: 2 |
| 39 | ND | NTSB, Helical I, Helical II, OBD and RuvC domains from SEQ ID NO: 1 and an |
| | | TSL domain from SEQ ID NO: 2. |
| 40 | ND | NTSB, TSL, Helical I, Helical II and OBD domains SEQ ID NO: 2 and an exogenous |
| | | RuvC domain or a portion thereof from a second CasX protein. |
| 41 | ND | ND |
| 42 | ND | NTSB, TSL, Helical II, OBD and RuvC domains from SEQ ID NO: 2 and a Helical I |
| | | domain from SEQ ID NO: 1 |
| 43 | ND | NTSB, TSL, Helical I, OBD and RuvC domains from SEQ ID NO: 2 and a Helical II |
| | | domain from SEQ ID NO: 1 |
| 44 | ND | NTSB, TSL, Helical I, Helical II and RuvC domains from a first CasX protein and an |
| | | exogenous OBD or a part thereof from a second CasX protein |
| 45 | ND | ND |
| 46 | ND | ND |
| 47 | ND | substitution of L379R, a substitution of C477K, a substitution of A708K, a deletion of |
| | | P at position 793 and a substitution of T620P of SEQ ID NO: 2 |
| 48 | ND | substitution of M771A of SEQ ID NO: 2. |
| 49 | ND | substitution of L379R, a substitution of A708K, a deletion of P at position 793 and a |
| | | substitution of D732N of SEQ ID NO: 2. |
| 50 | ND | substitution of W782Q of SEQ ID NO: 2. |
| 51 | ND | substitution of M771Q of SEQ ID NO: 2 |
| 52 | ND | substitution of R458I and a substitution of A739V of SEQ ID NO: 2. |
| 53 | ND | L379R, a substitution of A708K, a deletion of P at position 793 and a substitution of |
| | | M771N of SEQ ID NO: 2 |
| 54 | ND | substitution of L379R, a substitution of A708K, a deletion of P at position 793 and a |
| | | substitution of A739T of SEQ ID NO: 2 |
| 55 | ND | substitution of L379R, a substitution of C477K, a substitution of A708K, a deletion of |
| | | P at position 793 and a substitution of D489S of SEQ ID NO: 2. |
| 56 | ND | substitution of L379R, a substitution of C477K, a substitution of A708K, a deletion of |
| | | P at position 793 and a substitution of D732N of SEQ ID NO: 2. |
| 57 | ND | substitution of V711K of SEQ ID NO: 2. |
| 58 | ND | substitution of L379R, a substitution of C477K, a substitution of A708K, a deletion of |
| | | P at position 793 and a substitution of Y797L of SEQ ID NO: 2. |
| 60 | ND | substitution of L379R, a substitution of C477K, a substitution of A708K, a deletion of |
| | | P at position 793 and a substitution of M771N of SEQ ID NO: 2. |
| 61 | ND | substitution of A708K, a deletion of P at position 793 and a substitution of E386S of |
| | | SEQ ID NO: 2. |
| 62 | ND | substitution of L379R, a substitution of C477K, a substitution of A708K and a |
| | | deletion of P at position 793 of SEQ ID NO: 2. |
| 63 | ND | substitution of L792D of SEQ ID NO: 2. |
| 64 | ND | substitution of G791F of SEQ ID NO: 2. |
| 65 | ND | substitution of A708K, a deletion of P at position 793 and a substitution of A739V of |
| | | SEQ ID NO: 2. |
| 66 | ND | substitution of L379R, a substitution of A708K, a deletion of P at position 793 and a |
| | | substitution of A739V of SEQ ID NO: 2. |
| 67 | ND | substitution of C477K, a substitution of A708K and a deletion of P at position 793 of |
| | | SEQ ID NO: 2. |
| 68 | ND | substitution of L2491 and a substitution of M771N of SEQ ID NO: 2. |
| 69 | ND | substitution of V747K of SEQ ID NO: 2. |
| 70 | ND | substitution of L379R, a substitution of C477K, a substitution of A708K, a deletion of |
| | | P at position 793 and a substitution of M779N of SEQ ID NO: 2. |
| 71 | ND | L379R, F755M |
| 59 | 119 | ND |
| 72 | 429 | ND |
| 73 | 430 | ND |
| 74 | 431 | ND |
| 75 | 432 | ND |
| 76 | 433 | ND |
| 77 | 434 | ND |
| 78 | 435 | ND |
| 79 | 436 | ND |
| 80 | 437 | ND |
| 81 | 438 | ND |
| 82 | 439 | ND |
| 83 | 440 | ND |
| 84 | 441 | ND |
| 85 | 442 | ND |
| 86 | 443 | ND |
| 87 | 444 | ND |
| 88 | 445 | ND |
| 89 | 446 | ND |
| 90 | 447 | ND |
| 91 | 448 | ND |
| 92 | 449 | ND |
| 93 | 450 | ND |
| 94 | 451 | ND |
| 95 | 452 | ND |
| 96 | 453 | ND |
| 97 | 454 | ND |
| 98 | 455 | ND |
| 99 | 456 | ND |
| 101 | 457 | ND |
| 102 | 458 | ND |
| 103 | 459 | ND |
| 104 | 460 | ND |
| 105 | 278 | ND |
| 106 | 279 | ND |
| 107 | 280 | ND |
| 108 | 285 | ND |
| 109 | 286 | ND |
| 110 | 287 | ND |
| 111 | 288 | ND |
| 112 | 290 | ND |
| 113 | 291 | ND |
| 114 | 293 | ND |
| 115 | 300 | ND |
| 116 | 492 | ND |
| 117 | 493 | ND |
| 118 | 387 | ND |
| 119 | 395 | ND |
| 120 | 485 | ND |
| 121 | 486 | ND |
| 122 | 487 | ND |
| 123 | 488 | ND |
| 124 | 489 | ND |
| 125 | 490 | ND |
| 126 | 491 | ND |
| 127 | 494 | ND |
| 128 | 328 | ND |
| 129 | 388 | ND |
| 130 | 389 | ND |
| 131 | 390 | ND |
| 132 | 514 | ND |
| 133 | 515 | ND |
| 134 | 516 | ND |
| 135 | 517 | ND |
| 136 | 518 | ND |
| 137 | 519 | ND |
| 138 | 520 | ND |
| 139 | 522 | ND |
| 140 | 523 | ND |
| 141 | 524 | ND |
| 142 | 525 | ND |
| 143 | 526 | ND |
| 144 | 527 | ND |
| 145 | 528 | ND |
| 146 | 529 | ND |
| 147 | 530 | ND |
| 148 | 531 | ND |
| 26908 | 532 | ND |
| 26909 | 533 | ND |
| 26910 | 534 | ND |
| 26911 | 535 | ND |
| 26912 | 536 | ND |
| 26913 | 537 | ND |
| 26914 | 538 | ND |
| 26915 | 539 | ND |
| 26916 | 540 | ND |
| 26917 | 541 | ND |
| 26918 | 542 | ND |
| 26919 | 543 | ND |
| 26920 | 544 | ND |
| 26921 | 545 | ND |
| 26922 | 546 | ND |
| 26923 | 547 | ND |
| 26924 | 548 | ND |
| 26925 | 550 | ND |
| 26926 | 551 | ND |
| 26927 | 552 | ND |
| 26928 | 553 | ND |
| 26929 | 554 | ND |
| 26930 | 555 | ND |
| 26931 | 556 | ND |
| 26932 | 557 | ND |
| 26933 | 558 | ND |
| 26934 | 559 | ND |
| 26935 | 560 | ND |
| 26936 | 561 | ND |
| 26937 | 562 | ND |
| 26938 | 563 | ND |
| 26939 | 564 | ND |
| 26940 | 565 | ND |
| 26941 | 566 | ND |
| 26942 | 567 | ND |
| 26943 | 568 | ND |
| 26944 | 569 | ND |
| 26945 | 570 | ND |
| 26946 | 571 | ND |
| 26947 | 572 | ND |
| 26948 | 573 | ND |
| 26949 | 574 | ND |
| 26950 | 575 | ND |
| 26951 | 576 | ND |
| 26952 | 577 | ND |
| 26953 | 578 | ND |
| 26954 | 579 | ND |
| 26955 | 580 | ND |
| 26956 | 581 | ND |
| 26957 | 582 | ND |
| 26958 | 583 | ND |
| 26959 | 584 | ND |
| 26960 | 585 | ND |
| 26961 | 586 | ND |
| 26962 | 587 | ND |
| 26963 | 588 | ND |
| 26964 | 589 | ND |
| 26965 | 590 | ND |
| 26966 | 591 | ND |
| 26967 | 592 | ND |
| 26968 | 593 | ND |
| 26969 | 594 | ND |
| 26970 | 595 | ND |
| 26971 | 596 | ND |
| 26972 | 597 | ND |
| 26973 | 598 | ND |
| 26974 | 599 | ND |
| 26975 | 600 | ND |
| 26976 | 601 | ND |
| 26977 | 602 | ND |
| 26978 | 603 | ND |
| 26979 | 604 | ND |
| 26980 | 605 | ND |
| 26981 | 606 | ND |
| 26982 | 607 | ND |
| 26983 | 608 | ND |
| 26984 | 609 | ND |
| 26985 | 610 | ND |
| 26986 | 611 | ND |
| 26987 | 612 | ND |
| 26988 | 613 | ND |
| 26989 | 614 | ND |
| 26990 | 615 | ND |
| 26991 | 616 | ND |
| 26992 | 617 | ND |
| 26993 | 618 | ND |
| 26994 | 619 | ND |
| 26995 | 620 | ND |
| 26996 | 621 | ND |
| 26997 | 622 | ND |
| 26998 | 623 | ND |
| 26999 | 624 | ND |
| 27000 | 625 | ND |
| 27001 | 626 | ND |
| 27002 | 627 | ND |
| 27003 | 628 | ND |
| 27004 | 629 | ND |
| 27005 | 630 | ND |
| 27006 | 631 | ND |
| 27007 | 632 | ND |
| 27008 | 633 | ND |
| 27009 | 634 | ND |
| 27010 | 635 | ND |
| 27011 | 636 | ND |
| 27012 | 637 | ND |
| 27013 | 638 | ND |
| 27014 | 639 | ND |
| 27015 | 640 | ND |
| 27016 | 641 | ND |
| 27017 | 642 | ND |
| 27018 | 643 | ND |
| 27019 | 644 | ND |
| 27020 | 645 | ND |
| 27021 | 646 | ND |
| 27022 | 647 | ND |
| 27023 | 648 | ND |
| 27024 | 649 | ND |
| 27025 | 650 | ND |
| 27026 | 651 | ND |
| 27027 | 652 | ND |
| 27028 | 653 | ND |
| 27029 | 654 | ND |
| 27030 | 655 | ND |
| 27031 | 656 | ND |
| 27032 | 657 | ND |
| 27033 | 658 | ND |
| 27034 | 659 | ND |
| 27035 | 660 | ND |
| 27036 | 661 | ND |
| 27037 | 662 | ND |
| 27038 | 663 | ND |
| 27039 | 664 | ND |
| 27040 | 665 | ND |
| 27041 | 666 | ND |
| 27042 | 667 | ND |
| 27043 | 668 | ND |
| 27044 | 669 | ND |
| 27154 | 670 | ND |
| 27045 | 671 | ND |
| 27046 | 672 | ND |
| 27047 | 673 | ND |
| 27048 | 674 | ND |
| 27049 | 675 | ND |
| 27050 | 676 | ND |
| 27051 | 677 | ND |
| 27052 | 678 | ND |
| 27053 | 679 | ND |
| 27054 | 680 | ND |
| 27055 | 681 | ND |
| 27056 | 682 | ND |
| 27057 | 683 | ND |
| 27058 | 684 | ND |
| 27059 | 685 | ND |
| 27060 | 686 | ND |
| 27061 | 687 | ND |
| 27062 | 688 | ND |
| 27063 | 689 | ND |
| 27064 | 690 | ND |
| 27065 | 691 | ND |
| 27066 | 692 | ND |
| 27067 | 693 | ND |
| 27068 | 694 | ND |
| 27069 | 701 | ND |
| 27070 | 702 | ND |
| 27071 | 703 | ND |
| 27072 | 704 | ND |
| 27073 | 705 | ND |
| 27074 | 706 | ND |
| 27075 | 707 | ND |
| 27076 | 708 | ND |
| 27077 | 709 | ND |
| 27078 | 710 | ND |
| 27079 | 711 | ND |
| 27080 | 712 | ND |
| 27081 | 713 | ND |
| 27082 | 714 | ND |
| 27083 | 715 | ND |
| 27084 | 716 | ND |
| 27085 | 717 | ND |
| 27086 | 718 | ND |
| 27087 | 719 | ND |
| 27088 | 720 | ND |
| 27089 | 721 | ND |
| 27090 | 722 | ND |
| 27091 | 723 | ND |
| 27092 | 724 | ND |
| 27093 | 725 | ND |
| 27094 | 726 | ND |
| 27095 | 727 | ND |
| 27096 | 728 | ND |
| 27097 | 729 | ND |
| 27098 | 730 | ND |
| 27099 | 731 | ND |
| 27100 | 732 | ND |
| 27101 | 733 | ND |
| 27102 | 734 | ND |
| 27103 | 735 | ND |
| 27104 | 736 | ND |
| 27105 | 737 | ND |
| 27106 | 738 | ND |
| 27107 | 739 | ND |
| 27108 | 740 | ND |
| 27109 | 741 | ND |
| 27110 | 742 | ND |
| 27111 | 743 | ND |
| 27112 | 744 | ND |
| 27113 | 745 | ND |
| 27114 | 746 | ND |
| 27115 | 747 | ND |
| 27116 | 748 | ND |
| 27117 | 749 | ND |
| 27118 | 750 | ND |
| 27119 | 751 | ND |
| 27120 | 752 | ND |
| 27121 | 753 | ND |
| 27122 | 754 | ND |
| 27123 | 755 | ND |
| 27124 | 756 | ND |
| 27125 | 757 | ND |
| 27126 | 758 | ND |
| 27127 | 759 | ND |
| 27128 | 760 | ND |
| 27129 | 761 | ND |
| 27130 | 762 | ND |
| 27131 | 763 | ND |
| 27132 | 764 | ND |
| 27133 | 765 | ND |
| 27134 | 766 | ND |
| 27135 | 767 | ND |
| 27136 | 768 | ND |
| 27137 | 769 | ND |
| 27138 | 770 | ND |
| 27139 | 777 | ND |
| 27140 | 778 | ND |
| 27141 | 779 | ND |
| 27142 | 780 | ND |
| 27143 | 781 | ND |
| 27144 | 782 | ND |
| 27145 | 783 | ND |
| 27146 | 784 | ND |
| 27147 | 785 | ND |
| 27148 | 786 | ND |
| 27149 | 787 | ND |
| 27150 | 788 | ND |
| 27151 | 789 | ND |
| 27152 | 790 | ND |
| 27153 | 791 | ND |

c. CasX Variant Proteins with Domains from Multiple Source Proteins

In certain embodiments, the disclosure provides a chimeric CasX protein comprising protein domains from two or more different CasX proteins, such as two or more naturally occurring CasX proteins, or two or more CasX variant protein sequences as described herein.

As used herein, a “chimeric CasX protein” refers to a CasX containing at least two domains isolated or derived from different sources, such as two naturally occurring proteins, which may, in some embodiments, be isolated from different species. For example, in some embodiments, a chimeric CasX protein comprises a first domain from a first CasX protein and a second domain from a second, different CasX protein. In some embodiments, the first domain can be selected from the group consisting of the NTSB, TSL, helical I, helical II, OBD and RuvC domains. In some embodiments, the second domain is selected from the group consisting of the NTSB, TSL, helical I, helical II, OBD and RuvC domains with the second domain being different from the foregoing first domain. In the case of split or non-contiguous domains such as helical I, RuvC and OBD, a portion of the non-contiguous domain can be replaced with the corresponding portion from any other source. For example, the helical I-I domain (sometimes referred to as helical I-a) in SEQ ID NO: 2 can be replaced with the corresponding helical I-I sequence from SEQ ID NO: 1, and the like. Domain sequences from reference CasX proteins, and their coordinates, are shown in Table 5. Representative examples of chimeric CasX proteins include the variants of CasX 472-483, 485-491 and 515, the sequences of which are set forth in Table 4.

TABLE 5
Domain coordinates in Reference CasX proteins

| | Coordinates in | Coordinates in |
|---|---|---|
| Domain Name | SEQ ID NO: 1 | SEQ ID NO: 2 |
| OBD a | 1-55 | 1-57 |
| helical I a | 56-99 | 58-101 |
| NTSB | 100-190 | 102-191 |
| helical I b | 191-331 | 192-332 |
| helical II | 332-508 | 333-500 |
| OBD b | 509-659 | 501-646 |
| RuvC a | 660-823 | 647-810 |
| TSL | 824-933 | 811-920 |
| RuvC b | 934-986 | 921-978 |

*OBD a and b, helical I a and b, and RuvC a and b are also referred to herein as OBD I and II, helical I-I and I-II, and RuvC I and II.

d. Protein Affinity for the gRNA

In some embodiments, a CasX variant protein has improved affinity for the gRNA relative to a reference CasX protein, leading to the formation of the ribonucleoprotein complex (RNP). Increased affinity of the CasX variant protein for the gRNA may, for example, result in a lower K_{d} for the generation of a RNP complex, which can, in some cases, result in a more stable ribonucleoprotein complex formation. In some embodiments, increased affinity of the CasX variant protein for the gRNA results in increased stability of the ribonucleoprotein complex when delivered to human cells. This increased stability can affect the function and utility of the complex in the cells of a subject, as well as result in improved pharmacokinetic properties in blood, when delivered to a subject. In some embodiments, increased affinity of the CasX variant protein, and the resulting increased stability of the ribonucleoprotein complex, allows for a lower dose of the CasX variant protein to be delivered to the subject or cells while still having the desired activity, for example in vivo or in vitro gene editing. In some embodiments, a higher affinity (tighter binding) of a CasX variant protein to a gRNA allows for a greater amount of editing events when both the CasX variant protein and the gRNA remain in an RNP complex. Increased editing events can be assessed using editing assays such as the tdTom editing assays described herein. In some embodiments, the K_{d} of a CasX variant protein for a gRNA is increased relative to a reference CasX protein by a factor of at least about 1.1, at least about 1.2, at least about 1.3, at least about 1.4, at least about 1.5, at least about 1.6, at least about 1.7, at least about 1.8, at least about 1.9, at least about 2, at least about 3, at least about 4, at least about 5, at least about 6, at least about 7, at least about 8, at least about 9, at least about 10, at least about 15, at least about 20, at least about 25, at least about 30, at least about 35, at least about 40, at least about 45, at least about 50, at least about 60, at least about 70, at least about 80, at least about 90, or at least about 100. In some embodiments, the CasX variant has about 1.1 to about 10-fold increased binding affinity to the gRNA compared to the reference CasX protein of SEQ ID NO: 2.

In some embodiments, increased affinity of the CasX variant protein for the gRNA results in increased stability of the ribonucleoprotein complex when delivered to mammalian cells, including in vivo delivery to a subject. This increased stability can affect the function and utility of the complex in the cells of a subject, as well as result in improved pharmacokinetic properties in blood, when delivered to a subject. In some embodiments, increased affinity of the CasX variant protein, and the resulting increased stability of the ribonucleoprotein complex, allows for a lower dose of the CasX variant protein to be delivered to the subject or cells while still having the desired activity; for example in vivo or in vitro gene editing. The increased ability to form RNP and keep them in stable form can be assessed using assays such as the in vitro cleavage assays described in the Examples herein. In some embodiments, RNP comprising the CasX variants of the disclosure are able to achieve a k_{cleave} rate when complexed as an RNP that is at last 2-fold, at least 5-fold, or at least 10-fold higher compared to RNP comprising a reference CasX of SEQ ID NOS: 1-3.

In some embodiments, a higher affinity (tighter binding) of a CasX variant protein to a gRNA allows for a greater amount of editing events when both the CasX variant protein and the gRNA remain in an RNP complex. Increased editing events can be assessed using editing assays such as the assays described herein.

Without wishing to be bound by theory, in some embodiments amino acid changes in the Helical I domain can increase the binding affinity of the CasX variant protein with the gRNA targeting sequence, while changes in the Helical II domain can increase the binding affinity of the CasX variant protein with the gRNA scaffold stem loop, and changes in the oligonucleotide binding domain (OBD) increase the binding affinity of the CasX variant protein with the gRNA triplex.

Methods of measuring CasX protein binding affinity for a gRNA include in vitro methods using purified CasX protein and gRNA. The binding affinity for reference CasX and variant proteins can be measured by fluorescence polarization if the gRNA or CasX protein is tagged with a fluorophore. Alternatively, or in addition, binding affinity can be measured by biolayer interferometry, electrophoretic mobility shift assays (EMSAs), or filter binding. Additional standard techniques to quantify absolute affinities of RNA binding proteins such as the reference CasX and variant proteins of the disclosure for specific gRNAs such as reference gRNAs and variants thereof include, but are not limited to, isothermal calorimetry (ITC), and surface plasmon resonance (SPR), as well as the methods of the Examples.

e. Affinity for Target Nucleic Acid

In some embodiments, a CasX variant protein has improved binding affinity for a target nucleic acid sequence relative to the affinity of a reference CasX protein for a target nucleic acid sequence. In some embodiments, affinity of a CasX variant protein of the disclosure for a target nucleic acid molecule is increased relative to a reference CasX protein by a factor of at least about 1.1, at least about 1.2, at least about 1.3, at least about 1.4, at least about 1.5, at least about 1.6, at least about 1.7, at least about 1.8, at least about 1.9, at least about 2, at least about 3, at least about 4, at least about 5, at least about 6, at least about 7, at least about 8, at least about 9, at least about 10, at least about 15, at least about 20, at least about 25, at least about 30, at least about 35, at least about 40, at least about 45, at least about 50, at least about 60, at least about 70, at least about 80, at least about 90, or at least about 100.

CasX variants with higher affinity for their target nucleic acid may, in some embodiments, cleave the target nucleic acid sequence more rapidly than a reference CasX protein that does not have increased affinity for the target nucleic acid. In some embodiments, the improved affinity for the target nucleic acid sequence comprises improved affinity for the target nucleic acid sequence, improved binding affinity to a wider spectrum of PAM sequences, an improved ability to search DNA for the target nucleic acid sequence, or any combinations thereof, resulting in an increased ability to modify the target nucleic acid. In some embodiments, a CasX variant protein with improved target nucleic acid affinity has increased affinity for specific PAM sequences other than the canonical TTC PAM recognized by the reference CasX protein of SEQ ID NO: 2, including binding affinity for PAM sequences selected from the group consisting of TTC, ATC, GTC, and CTC. A higher overall affinity for DNA also, in some embodiments, can increase the frequency at which a CasX protein can effectively start and finish a binding and unwinding step, thereby facilitating target strand invasion and R-loop formation, and ultimately the cleavage of a target nucleic acid sequence.

In some embodiments, a CasX variant protein has improved binding affinity for the non-target strand of the target nucleic acid. As used herein, the term “non-target strand” refers to the strand of the DNA target nucleic acid sequence that does not form Watson and Crick base pairs with the targeting sequence in the gRNA and is complementary to the target DNA strand. In some embodiments, the CasX variant protein has about 1.1 to about 100-fold increased binding affinity to the non-target stand of the target nucleic acid compared to the reference protein of SEQ ID NO: 1, SEQ ID NO: 2, or SEQ ID NO: 3.

Methods of measuring CasX variant protein affinity for a target nucleic acid molecule may include electrophoretic mobility shift assays (EMSAs), filter binding, isothermal calorimetry (ITC), and surface plasmon resonance (SPR), fluorescence polarization and biolayer interferometry (BLI). Further methods of measuring CasX protein affinity for a target include in vitro biochemical assays that measure DNA cleavage events over time; e.g., determination of the k_{cleave} rate, as described in the Examples.

f Improved Specificity for a Target Site

In some embodiments, a CasX variant protein has improved specificity for a target nucleic acid sequence relative to a reference CasX protein. As used herein, “specificity,” interchangeably referred to as “target specificity,” refers to the degree to which a CRISPR/Cas system ribonucleoprotein complex cleaves off-target sequences that are similar, but not identical to the target nucleic acid sequence; e.g., a CasX variant RNP with a higher degree of specificity would exhibit reduced off-target cleavage of sequences relative to a reference CasX protein. The specificity, and the reduction of potentially deleterious off-target effects, of CRISPR/Cas system proteins can be vitally important in order to achieve an acceptable therapeutic index for use in mammalian subjects.

In some embodiments, a CasX variant protein has improved specificity for a target site within the target sequence that is complementary to the targeting sequence of the gRNA relative to a reference CasX protein of SEQ ID NOS: 1-3. Without wishing to be bound by theory, it is possible that amino acid changes in the helical I and II domains that increase the specificity of the CasX variant protein for the target nucleic acid strand can increase the specificity of the CasX variant protein for the target nucleic acid overall. In some embodiments, amino acid changes that increase specificity of CasX variant proteins for target nucleic acid may also result in decreased affinity of CasX variant proteins for DNA.

Methods of testing CasX protein (such as variant or reference) target specificity may include guide and Circularization for In vitro Reporting of Cleavage Effects by Sequencing (CIRCLE-seq), or similar methods. In brief, in CIRCLE-seq techniques, genomic DNA is sheared and circularized by ligation of stem-loop adapters, which are nicked in the stem-loop regions to expose 4 nucleotide palindromic overhangs. This is followed by intramolecular ligation and degradation of remaining linear DNA. Circular DNA molecules containing a CasX cleavage site are subsequently linearized with CasX, and adapter adapters are ligated to the exposed ends followed by high-throughput sequencing to generate paired end reads that contain information about the off-target site. Additional assays that can be used to detect off-target events, and therefore CasX protein specificity include assays used to detect and quantify indels (insertions and deletions) formed at those selected off-target sites such as mismatch-detection nuclease assays and next generation sequencing (NGS). Exemplary mismatch-detection assays include nuclease assays, in which genomic DNA from cells treated with CasX and sgRNA is PCR amplified, denatured and rehybridized to form hetero-duplex DNA, containing one wild-type strand and one strand with an indel. Mismatches are recognized and cleaved by mismatch detection nucleases, such as Surveyor nuclease or T7 endonuclease I.

g. Protospacer and PAM Sequences

Herein, the protospacer is defined as the DNA sequence complementary to the targeting sequence of the guide RNA and the DNA complementary to that sequence, referred to as the target strand and non-target strand, respectively. As used herein, the PAM is a nucleotide sequence located is located 1 nucleotide 5′ of the sequence in the non-target strand that is complementary to the target nucleic acid sequence in the target strand of the target nucleic acid that, in conjunction with the targeting sequence of the gRNA, helps the orientation and positioning of the CasX for the potential cleavage of the protospacer strand(s). PAM sequences may be degenerate, and specific RNP constructs may have different preferred and tolerated PAM sequences that support different efficiencies of cleavage. Following convention, unless stated otherwise, the disclosure refers to both the PAM and the protospacer sequence and their directionality according to the orientation of the non-target strand. This does not imply that the PAM sequence of the non-target strand, rather than the target strand, is determinative of cleavage or mechanistically involved in target recognition. For example, when reference is to a TTC PAM, it may in fact be the complementary GAA sequence that is required for target cleavage, or it may be some combination of nucleotides from both strands. In the case of the CasX proteins disclosed herein, the PAM is located 5′ of the protospacer with a single nucleotide separating the PAM from the first nucleotide of the protospacer. Thus, in the case of reference CasX, a TTC PAM should be understood to mean a sequence following the formula 5′- . . . NNTTCN(protospacer)NNNNNN . . . 3′ where ‘N’ is any DNA nucleotide and ‘(protospacer)’ is a DNA sequence having identity with the targeting sequence of the guide RNA. In the case of a CasX variant with expanded PAM recognition, a TTC, CTC, GTC, or ATC PAM should be understood to mean a sequence following the formulae:

5′- . . . NNTTCN(protospacer)NNNNNN . . . 3′;
5′- . . . NNCTCN(protospacer)NNNNNN . . . 3′;
5′- . . . NNGTCN(protospacer)NNNNNN . . . 3′;
or
5′- . . . NNATCN(protospacer)NNNNNN . . . 3′.

Alternatively, a TC PAM should be understood to mean a sequence following the formula: 5′- . . . NNNTCN(protospacer)NNNNNN . . . 3′ Additionally, the CasX variant proteins of the disclosure have an enhanced ability to efficiently edit and/or bind target DNA, when complexed with a gRNA as an RNP, utilizing a PAM TC motif, including PAM sequences selected from TTC, ATC, GTC, or CTC, (in a 5′ to 3′ orientation), compared to an RNP of a reference CasX protein and reference gRNA. In the foregoing, the PAM sequence is located at least 1 nucleotide 5′ to the non-target strand of the protospacer having identity with the targeting sequence of the gRNA in an assay system compared to the editing efficiency and/or binding of an RNP comprising a reference CasX protein and reference gRNA in a comparable assay system. In one embodiment, an RNP of a CasX variant and gRNA variant exhibits greater editing efficiency and/or binding of a target sequence in the target DNA compared to an RNP comprising a reference CasX protein and a reference gRNA in a comparable assay system, wherein the PAM sequence of the target DNA is TTC. In another embodiment, an RNP of a CasX variant and gRNA variant exhibits greater editing efficiency and/or binding of a target sequence in the target DNA compared to an RNP comprising a reference CasX protein and a reference gRNA in a comparable assay system, wherein the PAM sequence of the target DNA is ATC. In another embodiment, an RNP of a CasX variant and gRNA variant exhibits greater editing efficiency and/or binding of a target sequence in the target DNA compared to an RNP comprising a reference CasX protein and a reference gRNA in a comparable assay system, wherein the PAM sequence of the target DNA is CTC. In another embodiment, an RNP of a CasX variant and gRNA variant exhibits greater editing efficiency and/or binding of a target sequence in the target DNA compared to an RNP comprising a reference CasX protein and a reference gRNA in a comparable assay system, wherein the PAM sequence of the target DNA is GTC. In the foregoing embodiments, the increased editing efficiency and/or binding affinity for the one or more PAM sequences is at least 1.5-fold greater or more compared to the editing efficiency and/or binding affinity of an RNP of any one of the CasX proteins of SEQ ID NOS:1-3 and the gRNA of Table 2 for the PAM sequences. Exemplary assays demonstrating the improved editing are described herein, in the Examples.

h. Unwinding of DNA

In some embodiments, a CasX variant protein has improved ability of unwinding DNA relative to a reference CasX protein. Poor dsDNA unwinding has been shown previously to impair or prevent the ability of CRISPR/Cas system proteins AnaCas9 or Cas14s to cleave DNA. Therefore, without wishing to be bound by any theory, it is likely that increased DNA cleavage activity by some CasX variant proteins of the disclosure is due, at least in part, to an increased ability to find and unwind the dsDNA at a target site.

Without wishing to be bound by theory, it is thought that amino acid changes in the NTSB domain may produce CasX variant proteins with increased DNA unwinding characteristics. Alternatively, or in addition, amino acid changes in the OBD or the helical domain regions that interact with the PAM may also produce CasX variant proteins with increased DNA unwinding characteristics.

Methods of measuring the ability of CasX proteins (such as variant or reference) to unwind DNA include, but are not limited to, in vitro assays that observe increased on rates of dsDNA targets in fluorescence polarization or biolayer interferometry.

i. Catalytic Activity

The ribonucleoprotein complex of the CasX:gRNA systems disclosed herein comprise a CasX variant that bind to a target nucleic acid sequence and cleaves the target nucleic acid sequence. In some embodiments, a CasX variant protein has improved catalytic activity relative to a reference CasX protein. Without wishing to be bound by theory, it is thought that in some cases cleavage of the target strand can be a limiting factor for Cas12-like molecules in creating a dsDNA break. In some embodiments, CasX variant proteins improve bending of the target strand of DNA and cleavage of this strand, resulting in an improvement in the overall efficiency of dsDNA cleavage by the CasX ribonucleoprotein complex.

In some embodiments, a CasX variant protein has increased nuclease activity compared to a reference CasX protein. Variants with increased nuclease activity can be generated, for example, through amino acid changes in the RuvC nuclease domain. In some embodiments, the CasX variant comprises a RuvC nuclease domain having nickase activity. In the foregoing, the CasX nickase of a CasX:gRNA system generates a single-stranded break within 10-18 nucleotides 3′ of a PAM site in the non-target strand. In other embodiments, the CasX variant comprises a RuvC nuclease domain having double-stranded cleavage activity. In the foregoing, the CasX of the CasX:gRNA system generates a double-stranded break within 18-26 nucleotides 5′ of a PAM site on the target strand and 10-18 nucleotides 3′ on the non-target strand. Nuclease activity can be assayed by a variety of methods, including those of the Examples. In some embodiments, a CasX variant has a k_{cleave} constant that is at least 2-fold, or at least 3-fold, or at least 4-fold, or at least 5-fold, or at least 6-fold, or at least 7-fold, or at least 8-fold, or at least 9-fold, or at least 10-fold greater compared to a reference CasX.

In some embodiments, a CasX variant protein has the improved characteristic of forming RNP with gRNA that result in a higher percentage of cleavage-competent RNP compared to an RNP of a reference CasX protein of SEQ ID NO: 1, SEQ ID NO: 2, or SEQ ID NO: 3 and the gRNA, as described in the Examples. By cleavage competent, it is meant that the RNP that is formed has the ability to cleave the target nucleic acid. In some embodiments, the RNP of the CasX variant and the gRNA exhibit at least a 2-fold, or at least a 3-fold, or at least a 4-fold, or at least a 5-fold, or at least a 10-fold cleavage rate compared to an RNP of a reference CasX protein of SEQ ID NO: 1, SEQ ID NO: 2, or SEQ ID NO: 3 and the gRNA of Table 2. In the foregoing embodiment, the improved competency rate can be demonstrated in an in vitro assay, such as described in the Examples.

In some embodiments, a CasX variant protein has increased target strand loading for double strand cleavage compared to a reference CasX. Variants with increased target strand loading activity can be generated, for example, through amino acid changes in the TLS domain. Without wishing to be bound by theory, amino acid changes in the TSL domain may result in CasX variant proteins with improved catalytic activity. Alternatively, or in addition, amino acid changes around the binding channel for the RNA:DNA duplex may also improve catalytic activity of the CasX variant protein. In some embodiments, a CasX variant protein has increased collateral cleavage activity compared to a reference CasX protein. As used herein, “collateral cleavage activity” refers to additional, non-targeted cleavage of nucleic acids following recognition and cleavage of a target nucleic acid sequence. In some embodiments, a CasX variant protein has decreased collateral cleavage activity compared to a reference CasX protein.

In some embodiments, for example those embodiments encompassing applications where cleavage of the target nucleic acid sequence is not a desired outcome, improving the catalytic activity of a CasX variant protein comprises altering, reducing, or abolishing the catalytic activity of the CasX variant protein. In some embodiments, a ribonucleoprotein complex comprising a dCasX variant protein binds to a target nucleic acid sequence and does not cleave the target nucleic acid.

In some embodiments, the CasX ribonucleoprotein complex comprising a CasX variant protein binds a target DNA but generates a single stranded nick in the target DNA. In some embodiments, particularly those embodiments wherein the CasX protein is a nickase, a CasX variant protein has decreased target strand loading for single strand nicking. Variants with decreased target strand loading may be generated, for example, through amino acid changes in the TSL domain.

Exemplary methods for characterizing the catalytic activity of CasX proteins may include, but are not limited to, in vitro cleavage assays, including those of the Examples, below. In some embodiments, electrophoresis of DNA products on agarose gels can interrogate the kinetics of strand cleavage.

j. CasX Fusion Proteins

In some embodiments, the disclosure provides CasX proteins comprising a heterologous protein fused to the CasX. In some cases, the CasX is a reference CasX protein. In other cases, the CasX is a CasX variant of any of the embodiments described herein.

In some embodiments, the CasX variant protein comprises any one of SEQ ID NOS: 59, 72-99, 101-148, and 26908-27154 of the sequences of Table 4 fused to one or more proteins or domains thereof that has a different activity of interest, resulting in a fusion protein. In some embodiments, the CasX variant protein comprises any one of SEQ ID NOS: 36-99, 101-148, 26908-27154 fused to one or more proteins or domains thereof. In some embodiments, the CasX variant protein comprises any one of SEQ ID NOS: 132-148, 26908-2715 fused to one or more proteins or domains thereof. For example, in some embodiments, the CasX variant protein is fused to a protein (or domain thereof) that inhibits transcription, modifies a target nucleic acid sequence, or modifies a polypeptide associated with a nucleic acid (e.g., histone modification).

In some embodiments, a heterologous polypeptide (or heterologous amino acid such as a cysteine residue or a non-natural amino acid) can be inserted at one or more positions within a CasX protein to generate a CasX fusion protein. In other embodiments, a cysteine residue can be inserted at one or more positions within a CasX protein followed by conjugation of a heterologous polypeptide described below. In some alternative embodiments, a heterologous polypeptide or heterologous amino acid can be added at the N- or C-terminus of the CasX variant protein. In other embodiments, a heterologous polypeptide or heterologous amino acid can be inserted internally within the sequence of the CasX protein.

In some embodiments, the CasX variant fusion protein retains RNA-guided sequence specific target nucleic acid binding and cleavage activity. In some cases, the CasX variant fusion protein has (retains) 50% or more of the activity (e.g., cleavage and/or binding activity) of the corresponding CasX variant protein that does not have the insertion of the heterologous protein. In some cases, the CasX variant fusion protein retains at least about 60%, or at least about 70% or more, at least about 80%, or at least about 90%, or at least about 92%, or at least about 95%, or at least about 98%, or at least about 100% of the activity (e.g., cleavage and/or binding activity) of the corresponding CasX protein that does not have the insertion of the heterologous protein.

In some cases, the CasX variant fusion protein retains (has) target nucleic acid binding activity relative to the activity of the CasX protein without the inserted heterologous amino acid or heterologous polypeptide. In some cases, the CasX variant fusion protein retains at least about 60%, or at least about 70% or more, at least about 80%, or at least about 90%, or at least about 92%, or at least about 95%, or at least about 98%, or at least about 100% of the binding activity of the corresponding CasX protein that does not have the insertion of the heterologous protein.

In some cases, the CasX variant fusion protein retains (has) target nucleic acid binding and/or cleavage activity relative to the activity of the parent CasX protein without the inserted heterologous amino acid or heterologous polypeptide. For example, in some cases, the CasX variant fusion protein has (retains) 50% or more of the binding and/or cleavage activity of the corresponding parent CasX protein (the CasX protein that does not have the insertion). For example, in some cases, the CasX variant fusion protein has (retains) 60% or more (70% or more, 80% or more, 90% or more, 92% or more, 95% or more, 98% or more, or 100%) of the binding and/or cleavage activity of the corresponding CasX parent protein (the CasX protein that does not have the insertion). Methods of measuring cleaving and/or binding activity of a CasX protein and/or a CasX fusion protein will be known to one of ordinary skill in the art and any convenient method can be used.

A variety of heterologous polypeptides are suitable for inclusion in a reference CasX or CasX variant fusion protein of the disclosure. In some cases, the fusion partner can modulate transcription (e.g., inhibit transcription, increase transcription) of a target DNA. For example, in some cases the fusion partner is a protein (or a domain from a protein) that inhibits transcription (e.g., a transcriptional repressor, a protein that functions via recruitment of transcription inhibitor proteins, modification of target DNA such as methylation, recruitment of a DNA modifier, modulation of histones associated with target DNA, recruitment of a histone modifier such as those that modify acetylation and/or methylation of histones, and the like).

In some cases the fusion partner is a protein (or a domain from a protein) that increases transcription (e.g., a transcription activator, a protein that acts via recruitment of transcription activator proteins, modification of target DNA such as demethylation, recruitment of a DNA modifier, modulation of histones associated with target DNA, recruitment of a histone modifier such as those that modify acetylation and/or methylation of histones, and the like).In some cases, a fusion partner has enzymatic activity that modifies a target nucleic acid sequence; e.g., nuclease activity, methyltransferase activity, demethylase activity, DNA repair activity, DNA damage activity, deamination activity, dismutase activity, alkylation activity, depurination activity, oxidation activity, pyrimidine dimer forming activity, integrase activity, transposase activity, recombinase activity, polymerase activity, ligase activity, helicase activity, photolyase activity or glycosylase activity. In some embodiments, a CasX variant comprises any one of SEQ ID NOS: 36-99, 101-148, or 26908-27154, or any one of SEQ ID NOS: 59, 72-99, 101-148, or 26908-27154, or any one of SEQ ID NOS 132-148, or 26908-27154, and a polypeptide with methyltransferase activity, demethylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity or demyristoylation activity.

In some embodiments, a CasX variant comprises any one of SEQ ID NOS: 36-99, 101-148, and 26908-27154, or any one of SEQ ID NOS: 59, 72-99, 101-148, or 26908-27154, or any one of SEQ ID NOS 132-148, or 26908-27154, and a fusion partner having enzymatic activity that modifies a polypeptide (e.g., a histone) associated with a target nucleic acid (e.g., methyltransferase activity, demethylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity or demyristoylation activity). Examples of proteins (or fragments thereof) that can be used as a fusion partner to increase transcription include but are not limited to: transcriptional activators such as VP16, VP64, VP48, VP160, p65 subdomain (e.g., from NFkB), and activation domain of EDLL and/or TAL activation domain (e.g., for activity in plants); histone lysine methyltransferases such as SET1A, SET1B, MLL1 to 5, ASH1, SYMD2, NSD1, and the like; histone lysine demethylases such as JHDM2a/b, UTX, JMJD3, and the like; histone acetyltransferases such as GCN5, PCAF, CBP, p300, TAF1, TIP60/PLIP, MOZ/MYST3, MORF/MYST4, SRC1, ACTR, P160, CLOCK, and the like; and DNA demethylases such as Ten-Eleven Translocation (TET) dioxygenase 1 (TET1CD), TET1, DME, DML1, DML2, ROS1, and the like.

Examples of proteins (or fragments thereof) that can be used as a fusion partner to decrease transcription include but are not limited to: transcriptional repressors such as the Kruppel associated box (KRAB or SKD); KOX1 repression domain; the Mad mSIN3 interaction domain (SID); the ERF repressor domain (ERD), the SRDX repression domain (e.g., for repression in plants), and the like; histone lysine methyltransferases such as Pr-SET⅞, SUV4-20H1, RIZ1, and the like; histone lysine demethylases such as JMJD2A/JHDM3A, JMJD2B, JMJD2C/GASC1, JMJD2D, JARID1A/RBP2, JARID1B/PLU-1, JARID 1C/SMCX, JARID1D/SMCY, and the like; histone lysine deacetylases such as HDAC1, HDAC2, HDAC3, HDAC8, HDAC4, HDAC5, HDAC7, HDAC9, SIRT1, SIRT2, HDAC11, and the like; DNA methylases such as HhaI DNA m5c-methyltransferase (M.HhaI), DNA methyltransferase 1 (DNMT1), DNA methyltransferase 3a (DNMT3a), DNA methyltransferase 3b (DNMT3b), METI, DRM3 (plants), ZMET2, CMT1, CMT2 (plants), and the like; and periphery recruitment elements such as Lamin A, Lamin B, and the like.

In some cases, the fusion partner to a CasX variant has enzymatic activity that modifies the target nucleic acid sequence (e.g., ssRNA, dsRNA, ssDNA, dsDNA). Examples of enzymatic activity that can be provided by the fusion partner include but are not limited to: nuclease activity such as that provided by a restriction enzyme (e.g., FokI nuclease), methyltransferase activity such as that provided by a methyltransferase (e.g., Hha1 DNA m5c-methyltransferase (M.Hhal), DNA methyltransferase 1 (DNMT1), DNA methyltransferase 3a (DNMT3a), DNA methyltransferase 3b (DNMT3b), METI, DRM3 (plants), ZMET2, CMT1, CMT2 (plants), and the like); demethylase activity such as that provided by a demethylase (e.g., Ten-Eleven Translocation (TET) dioxygenase 1 (TET 1 CD), TET1, DME, DML1, DML2, ROS1, and the like), DNA repair activity, DNA damage activity, deamination activity such as that provided by a deaminase (e.g., a cytosine deaminase enzyme, e.g., an APOBEC protein such as rat APOBECl), dismutase activity, alkylation activity, depurination activity, oxidation activity, pyrimidine dimer forming activity, integrase activity such as that provided by an integrase and/or resolvase (e.g., Gin invertase such as the hyperactive mutant of the Gin invertase, GinH106Y; human immunodeficiency virus type 1 integrase (IN); Tn3 resolvase; and the like), transposase activity, recombinase activity such as that provided by a recombinase (e.g., catalytic domain of Gin recombinase), polymerase activity, ligase activity, helicase activity, photolyase activity, and glycosylase activity).

In some cases, a CasX variant protein of the present disclosure is fused to a polypeptide selected from a domain for increasing transcription (e.g., a VP16 domain, a VP64 domain), a domain for decreasing transcription (e.g., a KRAB domain, e.g., from the Kox1 protein), a core catalytic domain of a histone acetyltransferase (e.g., histone acetyltransferase p300), a protein/domain that provides a detectable signal (e.g., a fluorescent protein such as GFP), a nuclease domain (e.g., a Fokl nuclease), or a base editor (e.g., cytidine deaminase such as APOBEC1).

In some embodiments, a CasX variant comprises any one of SEQ ID NOS: 36-99, 101-148, or 26908-27154, or any one of SEQ ID NOS: 59, 72-99, 101-148, or 26908-27154, or any one of SEQ ID NOS 132-148, or 26908-27154, and a fusion partner having enzymatic activity that modifies a protein associated with the target nucleic acid (e.g., ssRNA, dsRNA, ssDNA, dsDNA) (e.g., a histone, an RNA binding protein, a DNA binding protein, and the like).

Examples of enzymatic activity (that modifies a protein associated with a target nucleic acid) that can be provided by the fusion partner include but are not limited to: methyltransferase activity such as that provided by a histone methyltransferase (HMT) (e.g., suppressor of variegation β-9 homolog 1 (SUV39H1, also known as KMT1A), euchromatic histone lysine methyltransferase 2 (G9A, also known as KMT1C and EHMT2), SUV39H2, ESET/SETDB 1, and the like, SET1A, SET1B, MLL1 to 5, ASH1, SYMD2, NSD1, DOT1L, Pr-SET⅞, SUV4-20H1, EZH2, RIZ1), demethylase activity such as that provided by a histone demethylase (e.g., Lysine Demethylase 1A (KDM1A also known as LSD1), JHDM2a/b, JMJD2A/JHDM3A, JMJD2B, JMJD2C/GASC1, JMJD2D, JARID1A/RBP2, JARID1B/PLU-1, JARID1C/SMCX, JARID1D/SMCY, UTX, JMJD3, and the like), acetyltransferase activity such as that provided by a histone acetylase transferase (e.g., catalytic core/fragment of the human acetyltransferase p300, GCN5, PCAF, CBP, TAF1, TIP60/PLIP, MOZ/MYST3, MORF/MYST4, HB01/MYST2, HMOF/MYST1, SRC1, ACTR, P160, CLOCK, and the like), deacetylase activity such as that provided by a histone deacetylase (e.g., HDAC1, HDAC2, HDAC3, HDAC8, HDAC4, HDACS, HDAC7, HDAC9, SIRT1, SIRT2, HDAC11, and the like), kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity, and demyristoylation activity.

Additional examples of suitable fusion partners for a CasX variant are (i) a dihydrofolate reductase (DHFR) destabilization domain (e.g., to generate a chemically controllable subject RNA-guided polypeptide or a conditionally active RNA-guided polypeptide), and (ii) a chloroplast transit peptide. In some embodiments, a CasX variant comprises any one of SEQ ID NOS: 36-99, 101-148, or 26908-27154, or any one of SEQ ID NOS: 59, 72-99, 101-148, or 26908-27154, or any one of SEQ ID NOS 132-148, or 26908-27154, or a sequence of Table 4, and a chloroplast transit peptide including, but are not limited to:

(SEQ ID NO: 154)
MASMISSSAVTTVSRASRGQSAAMAPFGGLKSMTGFPVRKVNTDITSIT
SNGGRVKCMQVWPPIGKKKFETLSYLPPLTRDSRA;
(SEQ ID NO: 155)
MASMISSSAVTTVSRASRGQSAAMAPFGGLKSMTGFPVRKVNTDITSIT
SNGGRVKS;
(SEQ ID NO: 156)
MASSMLSSATMVASPAQATMVAPFNGLKSSAAFPATRKANNDITSITSN
GGRVNCMQVWPPIEKKKFETLSYLPDLTDSGGRVNC;
(SEQ ID NO: 157)
MAQVSRICNGVQNPSLISNLSKSSQRKSPLSVSLKTQQHPRAYPISSSW
GLKKSGMTLIGSELRPLKVMSSVSTAC;
(SEQ ID NO: 158)
MAQVSRICNGVWNPSLISNLSKSSQRKSPLSVSLKTQQHPRAYPISSSW
GLKKSGMTLIGSELRPLKVMSSVSTAC;
(SEQ ID NO: 159)
MAQINNMAQGIQTLNPNSNFHKPQVPKSSSFLVFGSKKLKNSANSMLVL
KKDSIFMQLFCSFRISASVATAC;
(SEQ ID NO: 160)
MAALVTSQLATSGTVLSVTDRFRRPGFQGLRPRNPADAALGMRTVGASA
APKQSRKPHRFDRRCLSMVV;
(SEQ ID NO: 161)
MAALTTSQLATSATGFGIADRSAPSSLLRHGFQGLKPRSPAGGDATSLS
VTTSARATPKQQRSVQRGSRRFPSVVVC;
(SEQ ID NO: 162)
MASSVLSSAAVATRSNVAQANMVAPFTGLKSAASFPVSRKQNLDITSIA
SNGGRVQC;
(SEQ ID NO: 163)
MESLAATSVFAPSRVAVPAARALVRAGTVVPTRRTSSTSGTSGVKCSAA
VTPQASPVISRSAAAA;
and
(SEQ ID NO: 164)
MGAAATSMQSLKFSNRLVPPSRRLSPVPNNVTCNNLPKSAAPVRTVKCC
ASSWNSTINGAAATTNGASAASS.

In some cases, a CasX variant protein of the present disclosure can include an endosomal escape peptide. In some cases, an endosomal escape polypeptide comprises the amino acid sequence GLFXALLXLLXSLWXLLLXA (SEQ ID NO: 165), wherein each X is independently selected from lysine, histidine, and arginine. In some cases, an endosomal escape polypeptide comprises the amino acid sequence GLFHALLHLLHSLWHLLLHA (SEQ ID NO: 166), or HHHHHHHHHH (SEQ ID NO: 167).

Non-limiting examples of fusion partners for use with CasX variant proteins when targeting ssRNA target nucleic acid sequences include (but are not limited to): splicing factors (e.g., RS domains); protein translation components (e.g., translation initiation, elongation, and/or release factors; e.g., eIF4G); RNA methylases; RNA editing enzymes (e.g., RNA deaminases, e.g., adenosine deaminase acting on RNA (ADAR), including A to I and/or C to U editing enzymes); helicases; RNA-binding proteins; and the like. It is understood that a heterologous polypeptide can include the entire protein or in some cases can include a fragment of the protein (e.g., a functional domain).

In some embodiments, a CasX variant comprises any one of SEQ ID NOS: 36-99, 101-148, or 26908-27154, or any one of SEQ ID NOS: 59, 72-99, 101-148, or 26908-27154, or any one of SEQ ID NOS 132-148, or 26908-27154 and a fusion partner of any domain capable of interacting with ssRNA (which, for the purposes of this disclosure, includes intramolecular and/or intermolecular secondary structures, e.g., double-stranded RNA duplexes such as hairpins, stem-loops, etc.), whether transiently or irreversibly, directly or indirectly, including but not limited to an effector domain selected from the group comprising; endonucleases (for example RNase III, the CRR22 DYW domain, Dicer, and PIN (PilT N-terminus) domains from proteins such as SMG5 and SMG6); proteins and protein domains responsible for stimulating RNA cleavage (for example CPSF, CstF, CFIm and CFIIm); exonucleases (for example XRN-1 or Exonuclease T); deadenylases (for example HNT3); proteins and protein domains responsible for nonsense mediated RNA decay (for example UPF1, UPF2, UPF3, UPF3b, RNP SI, Y14, DEK, REF2, and SRm160); proteins and protein domains responsible for stabilizing RNA (for example PABP); proteins and protein domains responsible for repressing translation (for example Ago2 and Ago4); proteins and protein domains responsible for stimulating translation (for example Staufen); proteins and protein domains responsible for (e.g., capable of) modulating translation (e.g., translation factors such as initiation factors, elongation factors, release factors, etc., e.g., eIF4G); proteins and protein domains responsible for polyadenylation of RNA (for example PAP1, GLD-2, and Star-PAP); proteins and protein domains responsible for polyuridinylation of RNA (for example CI Dl and terminal uridylate transferase); proteins and protein domains responsible for RNA localization (for example from IMPI, ZBP1, She2p, She3p, and Bicaudal-D); proteins and protein domains responsible for nuclear retention of RNA (for example Rrp6); proteins and protein domains responsible for nuclear export of RNA (for example TAP, NXF1, THO, TREX, REF, and Aly); proteins and protein domains responsible for repression of RNA splicing (for example PTB, Sam68, and hnRNP Al); proteins and protein domains responsible for stimulation of RNA splicing (for example serine/arginine-rich (SR) domains); proteins and protein domains responsible for reducing the efficiency of transcription (for example FUS (TLS)); and proteins and protein domains responsible for stimulating transcription (for example CDK7 and HIV Tat). Alternatively, the effector domain may be selected from the group comprising endonucleases; proteins and protein domains capable of stimulating RNA cleavage; exonucleases; deadenylases; proteins and protein domains having nonsense mediated RNA decay activity; proteins and protein domains capable of stabilizing RNA; proteins and protein domains capable of repressing translation; proteins and protein domains capable of stimulating translation; proteins and protein domains capable of modulating translation (e.g., translation factors such as initiation factors, elongation factors, release factors, etc., e.g., eIF4G); proteins and protein domains capable of polyadenylation of RNA; proteins and protein domains capable of polyuridinylation of RNA; proteins and protein domains having RNA localization activity; proteins and protein domains capable of nuclear retention of RNA; proteins and protein domains having RNA nuclear export activity; proteins and protein domains capable of repression of RNA splicing; proteins and protein domains capable of stimulation of RNA splicing; proteins and protein domains capable of reducing the efficiency of transcription; and proteins and protein domains capable of stimulating transcription. Another suitable heterologous polypeptide is a PUF RNA-binding domain, which is described in more detail in WO2012068627, which is hereby incorporated by reference in its entirety.

Some RNA splicing factors that can be used (in whole or as fragments thereof) as a fusion partner with a CasX variant have modular organization, with separate sequence-specific RNA binding modules and splicing effector domains. For example, members of the serine/arginine-rich (SR) protein family contain N-terminal RNA recognition motifs (RRMs) that bind to exonic splicing enhancers (ESEs) in pre-mRNAs and C-terminal RS domains that promote exon inclusion. As another example, the hnRNP protein hnRNP Al binds to exonic splicing silencers (ESSs) through its RRM domains and inhibits exon inclusion through a C-terminal glycine-rich domain. Some splicing factors can regulate alternative use of splice site (ss) by binding to regulatory sequences between the two alternative sites. For example, ASF/SF2 can recognize ESEs and promote the use of intron proximal sites, whereas hnRNP Al can bind to ESSs and shift splicing towards the use of intron distal sites. One application for such factors is to generate ESFs that modulate alternative splicing of endogenous genes, particularly disease associated genes. For example, Bc1-x pre-mRNA produces two splicing isoforms with two alternative 5′ splice sites to encode proteins of opposite functions. The long splicing isoform Bc1-xL is a potent apoptosis inhibitor expressed in long-lived post mitotic cells and is up-regulated in many cancer cells, protecting cells against apoptotic signals. The short isoform Bc1-xS is a pro-apoptotic isoform and expressed at high levels in cells with a high turnover rate (e.g., developing lymphocytes). The ratio of the two Bc1-x splicing isoforms is regulated by multiple cc-elements that are located in either the core exon region or the exon extension region (i.e., between the two alternative 5′ splice sites). For more examples, see WO2010075303, which is hereby incorporated by reference in its entirety.

Further suitable fusion partners for use with a CasX variant include, but are not limited to proteins (or fragments thereof) that are boundary elements (e.g., CTCF), proteins and fragments thereof that provide periphery recruitment (e.g., Lamin A, Lamin B, etc.), and protein docking elements (e.g., FKBP/FRB, Pill/Abyl, etc.).

In some cases, a heterologous polypeptide (a fusion partner) for use with a CasX variant provides for subcellular localization, i.e., the heterologous polypeptide contains a subcellular localization sequence (e.g., a nuclear localization signal (NLS) for targeting to the nucleus, a sequence to keep the fusion protein out of the nucleus, e.g., a nuclear export sequence (NES), a sequence to keep the fusion protein retained in the cytoplasm, a mitochondrial localization signal for targeting to the mitochondria, a chloroplast localization signal for targeting to a chloroplast, an ER retention signal, and the like). In some embodiments, a subject RNA-guided polypeptide or a conditionally active RNA-guided polypeptide and/or subject CasX fusion protein does not include a NLS so that the protein is not targeted to the nucleus (which can be advantageous, e.g., when the target nucleic acid sequence is an RNA that is present in the cytosol). In some embodiments, a fusion partner can provide a tag (i.e., the heterologous polypeptide is a detectable label) for ease of tracking and/or purification (e.g., a fluorescent protein, e.g., green fluorescent protein (GFP), yellow fluorescent protein (YFP), red fluorescent protein (RFP), cyan fluorescent protein (CFP), mCherry, tdTomato, and the like; a histidine tag, e.g., a 6XHis tag; a hemagglutinin (HA) tag; a FLAG tag; a Myc tag; and the like).

In some cases, non-limiting examples of NLSs suitable for use with a CasX variant include sequences having at least about 80%, at least about 90%, or at least about 95% identity or are identical to sequences derived from: the NLS of the SV40 virus large T-antigen, having the amino acid sequence PKKKRKV (SEQ ID NO: 168); the NLS from nucleoplasmin (e.g., the nucleoplasmin bipartite NLS with the sequence KRPAATKKAGQAKKKK (SEQ ID NO: 169); the c-myc NLS having the amino acid sequence PAAKRVKLD (SEQ ID NO: 170) or RQRRNELKRSP (SEQ ID NO: 171); the hRNPAl M9 NLS having the sequence NQSSNFGPMKGGNFGGRSSGPYGGGGQYFAKPRNQGGY (SEQ ID NO: 172); the sequence RMRIZFKNKGKDTAELRRRRVEVSVELRKAKKDEQILKRRNV (SEQ ID NO: 173) of the IBB domain from importin-alpha; the sequences VSRKRPRP (SEQ ID NO: 174) and PPKKARED (SEQ ID NO: 175) of the myoma T protein; the sequence PQPKKKPL (SEQ ID NO: 176) of human p53; the sequence SALIKKKKKMAP (SEQ ID NO: 177) of mouse c-abl IV; the sequences DRLRR (SEQ ID NO: 178) and PKQKKRK (SEQ ID NO: 179) of the influenza virus NS1; the sequence RKLKKKIKKL (SEQ ID NO: 180) of the Hepatitis virus delta antigen; the sequence REKKKFLKRR (SEQ ID NO: 181) of the mouse Mxl protein; the sequence KRKGDEVDGVDEVAKKKSKK (SEQ ID NO: 182) of the human poly(ADP-ribose) polymerase; the sequence RKCLQAGMNLEARKTKK (SEQ ID NO: 183) of the steroid hormone receptors (human) glucocorticoid; the sequence PRPRKIPR (SEQ ID NO: 184) of Borna disease virus P protein (BDV-P1); the sequence PPRKKRTVV (SEQ ID NO: 185) of hepatitis C virus nonstructural protein (HCV-NS5A);the sequence NLSKKKKRKREK (SEQ ID NO: 186) of LEF1; the sequence RRPSRPFRKP (SEQ ID NO: 187) of ORF57 simirae; the sequence KRPRSPSS (SEQ ID NO: 188) of EBV LANA; the sequence KRGINDRNFWRGENERKTR (SEQ ID NO: 189) of Influenza A protein; the sequence PRPPKMARYDN (SEQ ID NO: 190) of human RNA helicase A (RHA); the sequence KRSFSKAF (SEQ ID NO: 191) of nucleolar RNA helicase II; the sequence KLKIKRPVK (SEQ ID NO: 192) of TUS-protein; the sequence PKKKRKVPPPPAAKRVKLD (SEQ ID NO: 193) associated with importin-alpha; the sequence PKTRRRPRRSQRKRPPT (SEQ ID NO:26792) from the Rex protein in HTLV-1; the sequence SRRRKANPTKLSENAKKLAKEVEN (SEQ ID NO: 194) from the EGL-13 protein of Caenorhabditis elegans; and the sequences KTRRRPRRSQRKRPPT (SEQ ID NO: 195), RRKKRRPRRKKRR (SEQ ID NO: 196), PKKKSRKPKKKSRK (SEQ ID NO: 197), HKKKHPDASVNFSEFSK (SEQ ID NO: 198), QRPGPYDRPQRPGPYDRP (SEQ ID NO: 199), LSPSLSPLLSPSLSPL (SEQ ID NO: 200), RGKGGKGLGKGGAKRHRK (SEQ ID NO: 201), PKRGRGRPKRGRGR (SEQ ID NO: 202), PKKKRKVPPPPAAKRVKLD (SEQ ID NO: 203), PKKKRKVPPPPKKKRKV (SEQ ID NO: 204), PAKRARRGYKC (SEQ ID NO: 27199), KLGPRKATGRW (SEQ ID NO: 27200), PRRKREE (SEQ ID NO: 27201), PYRGRKE (SEQ ID NO: 27202), PLRKRPRR (SEQ ID NO: 27203), PLRKRPRRGSPLRKRPRR (SEQ ID NO: 27204), PAAKRVKLDGGKRTADGSEFESPKKKRKV (SEQ ID NO: 27205), PAAKRVKLDGGKRTADGSEFESPKKKRKVGIHGVPAA (SEQ ID NO: 27206), PAAKRVKLDGGKRTADGSEFESPKKKRKVAEAAAKEAAAKEAAAKA (SEQ ID NO: 207), PAAKRVKLDGGKRTADGSEFESPKKKRKVPG (SEQ ID NO: 27208), KRKGSPERGERKRHW (SEQ ID NO: 27209), KRTADSQHSTPPKTKRKVEFEPKKKRKV (SEQ ID NO: 27210), and PKKKRKVGGSKRTADSQHSTPPKTKRKVEFEPKKKRKV (SEQ ID NO: 27211). In some embodiments, the one or more NLS are linked to the CRISPR protein or to adjacent NLS with a linker peptide wherein the linker peptide is selected from the group consisting of RS, (G)n (SEQ ID NO: 27212), (GS)n (SEQ ID NO: 27213), (GSGGS)n (SEQ ID NO: 214), (GGSGGS)n (SEQ ID NO: 215), (GGGS)n (SEQ ID NO: 216), GGSG (SEQ ID NO: 217), GGSGG (SEQ ID NO: 218), GSGSG (SEQ ID NO: 219), GSGGG (SEQ ID NO: 220), GGGSG (SEQ ID NO: 221), GSSSG (SEQ ID NO: 222), GPGP (SEQ ID NO: 223), GGP, PPP, PPAPPA (SEQ ID NO: 224), PPPG (SEQ ID NO: 27214), PPPGPPP (SEQ ID NO: 225), PPP(GGGS)n (SEQ ID NO: 27215), (GGGS)nPPP (SEQ ID NO: 27216), AEAAAKEAAAKEAAAKA (SEQ ID NO: 27217), and TPPKTKRKVEFE (SEQ ID NO: 27218), where n is 1 to 5. In general, NLS (or multiple NLSs) are of sufficient strength to drive accumulation of a CasX variant fusion protein in the nucleus of a eukaryotic cell. Detection of accumulation in the nucleus may be performed by any suitable technique. For example, a detectable marker may be fused to a CasX variant fusion protein such that location within a cell may be visualized. Cell nuclei may also be isolated from cells, the contents of which may then be analyzed by any suitable process for detecting protein, such as immunohistochemistry, Western blot, or enzyme activity assay. Accumulation in the nucleus may also be determined indirectly.

In general, NLS (or multiple NLSs) are of sufficient strength to drive accumulation of an expressed CasX variant fusion protein in the nucleus of a eukaryotic cell. Detection of accumulation in the nucleus may be performed by any suitable technique. For example, a detectable marker may be fused to a CasX variant fusion protein such that location within a cell may be visualized. Cell nuclei may also be isolated from cells, the contents of which may then be analyzed by any suitable process for detecting protein, such as immunohistochemistry, Western blot, or enzyme activity assay. Accumulation in the nucleus may also be determined indirectly.

In some cases, a CasX variant fusion protein includes a “Protein Transduction Domain” or PTD (also known as a CPP—cell penetrating peptide), which refers to a protein, polynucleotide, carbohydrate, or organic or inorganic compound that facilitates traversing a lipid bilayer, micelle, cell membrane, organelle membrane, or vesicle membrane. A PTD attached to another molecule, which can range from a small polar molecule to a large macromolecule and/or a nanoparticle, facilitates the molecule traversing a membrane, for example going from an extracellular space to an intracellular space, or from the cytosol to within an organelle. In some embodiments, a PTD is covalently linked to the amino terminus of a CasX variant fusion protein. In some embodiments, a PTD is covalently linked to the carboxyl terminus of a CasX variant fusion protein. In some cases, the PTD is inserted internally in the sequence of a CasX variant fusion protein at a suitable insertion site. In some cases, a CasX variant fusion protein includes (is conjugated to, is fused to) one or more PTDs (e.g., two or more, three or more, four or more PTDs). In some cases, a PTD includes one or more nuclear localization signals (NLS). Examples of PTDs include but are not limited to peptide transduction domain of HIV TAT comprising YGRKKRRQRRR (SEQ ID NO: 205), RKKRRQRR (SEQ ID NO: 206); YARAAARQARA (SEQ ID NO: 207); THRLPRRRRRR (SEQ ID NO: 208); and GGRRARRRRRR (SEQ ID NO: 209); a polyarginine sequence comprising a number of arginine residues sufficient to direct entry into a cell (e.g., 3, 4, 5, 6, 7, 8, 9, 10, or 10-50 arginine residues (SEQ ID NO: 26793); a VP22 domain (Zender et al. (2002) Cancer Gene Ther. 9(6):489-96); an Drosophila Antennapedia protein transduction domain (Noguchi et al. (2003) Diabetes 52(7): 1732-1737); a truncated human calcitonin peptide (Trehin et al. (2004) Pharm. Research 21:1248-1256); polylysine (Wender et al. (2000) Proc. Natl. Acad. Sci. USA 97: 13003-13008); RRQRRTSKLMKR (SEQ ID NO: 210); Transportan GWTLNSAGYLLGKINLKALAALAKKIL (SEQ ID NO: 211); KALAWEAKLAKALAKALAKHLAKALAKALKCEA (SEQ ID NO: 212); and RQIKIWFQNRRMKWKK (SEQ ID NO: 213). In some embodiments, the PTD is an activatable CPP (ACPP) (Aguilera et al. (2009) Integr Biol (Camb) June; 1(5-6): 371-381). ACPPs comprise a polycationic CPP (e.g., Arg9 or “R9”) connected via a cleavable linker to a matching polyanion (e.g., Glu9 or “E9”), which reduces the net charge to nearly zero and thereby inhibits adhesion and uptake into cells. Upon cleavage of the linker, the polyanion is released, locally unmasking the polyarginine and its inherent adhesiveness, thus “activating” the ACPP to traverse the membrane.

In some embodiments, a CasX variant fusion protein for use in the systems can include a CasX protein that is linked to an internally inserted heterologous amino acid or heterologous polypeptide (a heterologous amino acid sequence) via a linker polypeptide (e.g., one or more linker polypeptides). In some embodiments, a CasX variant fusion protein can be linked at the C-terminal and/or N-terminal end to a heterologous polypeptide (fusion partner) via a linker polypeptide (e.g., one or more linker polypeptides). The linker polypeptide may have any of a variety of amino acid sequences. Proteins can be joined by a spacer peptide, generally of a flexible nature, although other chemical linkages are not excluded. Suitable linkers include polypeptides of between 4 amino acids and 40 amino acids in length, or between 4 amino acids and 25 amino acids in length. These linkers are generally produced by using synthetic, linker-encoding oligonucleotides to couple the proteins. Peptide linkers with a degree of flexibility can be used. The linking peptides may have virtually any amino acid sequence, bearing in mind that the preferred linkers will have a sequence that results in a generally flexible peptide. The use of small amino acids, such as glycine and alanine, are of use in creating a flexible peptide. The creation of such sequences is routine to those of skill in the art. A variety of different linkers are commercially available and are considered suitable for use. Exemplary linker polypeptides include peptides selected from the group consisting of RS, (G)n (SEQ ID NO: 27212), (GS)n (SEQ ID NO: 27213), (GSGGS)n (SEQ ID NO: 214), (GGSGGS)n (SEQ ID NO: 215), (GGGS)n (SEQ ID NO: 216), where n is an integer of 1 to 5, GGSG (SEQ ID NO: 217), GGSGG (SEQ ID NO: 218), GSGSG (SEQ ID NO: 219), GSGGG (SEQ ID NO: 220), GGGSG (SEQ ID NO: 221), GSSSG (SEQ ID NO: 222), GPGP (SEQ ID NO: 223), GGP, PPP, PPAPPA (SEQ ID NO: 224), PPPG (SEQ ID NO: 27214), PPPGPPP (SEQ ID NO: 225), PPP(GGGS)n (SEQ ID NO: 27215), (GGGS)nPPP (SEQ ID NO: 27216), AEAAAKEAAAKEAAAKA (SEQ ID NO: 27217), and TPPKTKRKVEFE (SEQ ID NO: 27218), where n is 1 to 5. and the like. The ordinarily skilled artisan will recognize that design of a peptide conjugated to any elements described above can include linkers that are all or partially flexible, such that the linker can include a flexible linker as well as one or more portions that confer less flexible structure.

### V. Systems and Methods for Modification of BCL11A Genes

The CRISPR proteins, guide nucleic acids, and variants thereof provided herein are useful for various applications, including as therapeutics, diagnostics, and for research. In some embodiments, to effect the methods of the disclosure for gene editing, provided herein are programmable CasX:gRNA systems. The programmable nature of the systems provided herein allows for the precise targeting to achieve the desired modification at one or more regions of predetermined interest in the BCL11A gene target nucleic acid. A variety of strategies and methods can be employed to modify the target nucleic acid sequence in a cell using the systems provided herein. As used herein “modifying” includes, but is not limited to, cleaving, nicking, editing, deleting, knocking out, knocking down, mutating, correcting, exon-skipping and the like. Depending on the system components utilized, the editing event may be a cleavage event followed by introducing random insertions or deletions (indels) or other mutations (e.g., a substitution, duplication, or inversion of one or more nucleotides), for example by utilizing the imprecise non-homologous DNA end joining (NHEJ) repair pathway, which may generate, for example, a frame shift mutation. Alternatively, the editing event may be a cleavage event followed by homology-directed repair (HDR), homology-independent targeted integration (HITI), micro-homology mediated end joining (MMEJ), single strand annealing (SSA) or base excision repair (BER), resulting in modification of the target nucleic acid sequence.

In some embodiments of the method, the BCL11A gene to be modified comprises a sequence corresponding to a polynucleotide encoding all or a portion of the sequence of SEQ ID NO: 100 or comprises a polynucleotide sequence that spans all or a portion of chr2 60450520-60554467 (GRCh38/hg38 Ensembl 100) of the human genome on chromosome 2. In other embodiments of the method, the target nucleic acid sequence to be modified includes regions of the BCL11A gene encoding the BCL11A protein, a BCL11A regulatory element, a non-coding region of the BCL11A gene, or overlapping portions thereof. In a particular embodiment of the method, the target nucleic acid sequence to be modified comprises the GATA1 binding motif sequence or its complement.

In some embodiments, the disclosure provides methods of modifying a BCL11A target nucleic acid in a cell, the method comprising introducing into the cell a Class 2, Type V CRISPR system. In some embodiments of the methods, the cells to be modified are autologous with respect to a subject to be administered said cell(s). In other embodiments, the cells to be modified are allogeneic with respect to a subject to be administered said cell(s). Thus, the systems and methods described herein can be used to engineer a variety of cells in which mutations exist in the β-globin gene and are associated with disease, e.g., hemoglobinopathies, including sickle-cell disease and α- and β-thalassemias. This approach, therefore, can be used to modify cells for applications in a subject with a hemoglobinopathy-related disease such as, but not limited to sickle-cell disease and α- and β-thalassemias.

In some embodiments, the disclosure provides methods of modifying a BCL11A target nucleic acid in a cell, the method comprising introducing into the cell: i) a CasX:gRNA system comprising a CasX and a gRNA of any one of the embodiments described herein; ii) a CasX:gRNA system comprising a CasX, a gRNA, and a donor template of any one of the embodiments described herein; iii) a nucleic acid encoding the CasX and the gRNA, and optionally comprising the donor template; iv) a vector comprising the nucleic acid of (iii), above; v) an XDP comprising the CasX:gRNA system of any one of the embodiments described herein; or vi) combinations of two or more of (i) to (v), wherein the target nucleic acid sequence of the cells is modified by the CasX protein and, optionally, the donor template. In some embodiments, the vector is an AAV vector. In some embodiments, the disclosure provides CasX:gRNA systems for use in the methods of modifying the BCL11A gene in a cell, wherein the system comprises a CasX variant selected from the group consisting of SEQ ID NOS: 36-99, 101-148, and 26908-27154, or a CasX variant selected from the group consisting of SEQ ID NOS: 59, 72-99, 101-148, and 26908-27154, or a CasX variant selected from the group consisting of SEQ ID NOS 132-148, and 26908-27154, or a variant sequence at least 60% identical, at least 70% identical, at least 80% identical, at least 81% identical, at least 82% identical, at least 83% identical, at least 84% identical, at least 85% identical, at least 86% identical, at least 86% identical, at least 87% identical, at least 88% identical, at least 89% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, or at least 99.5% identical thereto, the gRNA scaffold comprises a sequence selected from the group consisting of SEQ ID NOS: 2101-2285, 26794-26839 and 27219-27265 as set forth in Table 3 or from the group consisting of SEQ ID NOS: 2281-2285, 26794-26839 and 27219-27265, or a sequence at least 65% identical, at least 70% identical, at least 75% identical, at least 80% identical, at least 81% identical, at least 82% identical, at least 83% identical, at least 84% identical, at least 85% identical, at least 86% identical, at least 86% identical, at least 87% identical, at least 88% identical, at least 89% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical thereto, and the gRNA comprises a targeting sequence selected from the group consisting of SEQ ID NOS: 272-2100 or 2286-26789, or a sequence at least 65% identical, at least 70% identical, at least 75% identical, at least 80% identical, at least 85% identical, at least 90% identical, or at least 95% identical thereto and having between 15 and 20 nucleotides. In particular embodiments, the targeting sequence of the gRNA is complementary to, and therefore is capable of hybridizing with, a sequence within the GATA1 binding motif sequence or that is 5′ or 3′ to the GATA1 binding motif sequence. In one embodiment, the targeting sequence of the gRNA is UGGAGCCUGUGAUAAAAGCA (SEQ ID NO: 22), which hybridizes with the BCL11A GATA1 erythroid-specific enhancer binding site sequence, or is a sequence having at least 90% or at least 95% sequence identity thereto. In another embodiment, the targeting sequence of the gRNA is UGCUUUUAUCACAGGCUCCA (SEQ ID NO: 23), which hybridizes with a sequence that is complementary to the reverse complement of the BCL11A GATA1 erythroid-specific enhancer binding site sequence, or is a sequence having at least 90% or at least 95% sequence identity thereto. In another particular embodiment, the targeting sequence of the gRNA is complementary to, and therefore is capable of hybridizing with a sequence within the promoter of the BCL11A gene. In one embodiment of the method, the CasX and gRNA are associated together in a ribonuclear protein complex (RNP). In some embodiments of the method of modifying a BCL11A target nucleic acid sequence in a cell, the modification comprises introducing a single-stranded break in the target nucleic acid sequence. In other embodiments of the method, the modification comprises introducing a double-stranded break in the target nucleic acid sequence. In some embodiments of the method, the modifying comprises introducing an insertion, deletion, substitution, duplication, or inversion of one or more nucleotides in the target nucleic acid sequence. As described herein, a CasX variant introducing double-stranded cleavage of the target nucleic acid generates a double-stranded break within 18-26 nucleotides 5′ of a PAM site on the target strand and 10-18 nucleotides 3′ on the non-target strand. Thus, in some embodiments, the resulting modification by the method can result in random insertions or deletions (indels), or a substitution, duplication, or inversion of one or more nucleotides in those region by non-homologous DNA end joining (NHEJ) repair mechanisms.

In other embodiments of the method of modifying a BCL11A target nucleic acid sequence in a cell, the method comprises contacting the target nucleic acid sequence with a CasX:gRNA system with a first and a second, or a plurality of gRNAs targeted to different or overlapping portions of the BCL11A gene (e.g., wherein the targeting sequence of the second gRNA is complementary to a sequence that is 5′ or 3′ to the GATA1 binding site) wherein the CasX protein introduces multiple breaks in the target nucleic acid that result in a permanent indel or mutation in the target nucleic acid, as described herein, or an excision of the GATA1 binding motif sequence with a corresponding modulation of expression or alteration in the function of the BCL11A gene product, thereby creating an edited cell. In some cases of the foregoing, the plurality of the gRNAs target locations 5′ and 3′ relative to the GATA1 binding motif sequence of the BCL11A gene such that some or all of the GATA1 binding motif sequence is excised from the target gene between the dual cut sites targeted by the two gRNA. It will be understood that the foregoing embodiments of the method can also be effected by use of encoding nucleic acids, vectors comprising the encoding acids, or XDP comprising the CasX:gRNA system components.

In some embodiments, the methods of the disclosure provide CasX protein and gRNA pairs that generate site-specific double strand breaks (DSBs) or single strand breaks (SSBs) (e.g., when the CasX protein is a nickase that can cleave only one strand of a target nucleic acid) within 18-24 nucleotides 3′ of a PAM site, which can then be repaired either by non-homologous end joining (NHEJ), homology-directed repair (HDR), homology-independent targeted integration (HITI), micro-homology mediated end joining (MMEJ), single strand annealing (SSA) or base excision repair (BER), wherein the modification of the BCL11A gene comprises introducing an insertion, a deletion, an inversion, or a duplication mutation of one or more nucleotides as compared to the wild-type sequence, with a corresponding modulation of expression or alteration in the function of the BCL11A gene product, thereby creating an edited cell.

In some cases, the CasX:gRNA system for use in the methods of modifying the BCL11A gene further comprises a donor template nucleic acid of any of the embodiments disclosed herein, wherein the donor template can be inserted by the homology-directed repair (HDR) or homology-independent targeted integration (HITI) repair mechanisms of the host cell. Thus, in some cases, the methods provided herein include contacting the BCL11A gene with a donor template by introducing the donor template (either in vitro inside a cell or in vivo inside a cell), wherein the donor template, a portion of the donor template, a copy of the donor template, or a portion of a copy of the donor template integrates into the BCL11A gene to replace a portion of the BCL11A gene. The donor template can be a short single-stranded or double-stranded oligonucleotide, or a long single-stranded or double-stranded oligonucleotide. In some embodiments, the donor template comprises at least a portion of the BCL11A gene, wherein the BCL11A gene portion is selected from the group consisting of a BCL11A exon, a BCL11A intron, a BCL11A intron-exon junction, a BCL11A regulatory element, or a combination thereof. In some embodiments, the disclosure provides donor templates for use in targeting, or disrupting, the transcriptional activator GATA1 binding site in the BCL11A target sequence wherein the donor template includes sequences that are nonhomologous to regions of DNA within or near GATA1 site in the BCL11A gene, flanked by two regions of homology (“homologous arms”) to the 5′ and 3′ sides of the break site(s) such that the repair mechanisms between the target DNA region and the two flanking sequences results in insertion of the donor template at the target region to facilitate insertion by HDR. The donor template may contain one or more single base changes, insertions, deletions, inversions or rearrangements with respect to the genomic sequence, provided that there is sufficient homology with the target nucleic acid sequence to support its integration into the target nucleic acid, which can result in a frame-shift or other mutation such that the BCL11A protein is not expressed (a knock-out) or is expressed at a lower level (a knock-down). The exogenous donor template inserted by HITI can be any length, for example, a relatively short sequence of between 10 and 50 nucleotides in length, or a longer sequence of about 50-1000 nucleotides in length. The lack of homology can be, for example, having no more than 20-50% sequence identity and/or lacking in specific hybridization at low stringency. In other cases, the lack of homology can further include a criterion of having no more than 5, 6, 7, 8, or 9 bp identity. In some embodiments, the donor template polynucleotide comprises at least about 10, at least about 50, at least about 100, or at least about 200, or at least about 300, or at least about 400, or at least about 500, or at least about 600, or at least about 700, or at least about 800, or at least about 900, or at least about 1000, or at least about 10,000, or at least about 15,000 nucleotides. In other embodiments, the donor template comprises at least about 10 to about 15,000 nucleotides, or at least about 100 to about 10,000 nucleotides, or at least about 400 to about 8,000 nucleotides, or at least about 600 to about 5000 nucleotides, or at least about 1000 to about 2000 nucleotides. The donor template sequence may comprise certain sequence differences as compared to the genomic sequence, e.g., restriction sites, nucleotide polymorphisms, selectable markers (e.g., drug resistance genes, fluorescent proteins, enzymes etc.), etc., which may be used to assess for successful insertion of the donor nucleic acid at the cleavage site or in some cases may be used for other purposes (e.g., to signify expression at the targeted genomic locus). Alternatively, these sequence differences may include flanking recombination sequences such as FLPs, loxP sequences, or the like, that can be activated at a later time for removal of the marker sequence.

In some embodiments of the methods of modifying a BCL11A target nucleic acid of a cell in vitro or ex vivo, to induce cleavage or any desired modification to a target nucleic acid, the gRNA and/or the CasX protein of the present disclosure and, optionally, the donor template sequence, whether they be introduced as nucleic acids or polypeptides, complexed RNP, vectors or XDP, are provided to the cells for about 30 minutes to about 24 hours, or at least about 1 hour, 1.5 hours, 2 hours, 2.5 hours, 3 hours, 3.5 hours 4 hours, 5 hours, 6 hours, 7 hours, 8 hours, 12 hours, 16 hours, 18 hours, 20 hours, or any other period from about 30 minutes to about 24 hours, which may be repeated with a frequency of about every day to about every 4 days, e.g., every 1.5 days, every 2 days, every 3 days, or any other frequency from about every day to about every four days. The agent(s) may be provided to the subject cells one or more times, e.g., one time, twice, three times, or more than three times, and the cells allowed to incubate with the agent(s) for some amount of time following each contacting event e.g., 30 minutes to about 24 hours. In the case of in vitro-based methods, after the incubation period with the CasX and gRNA (and optionally the donor template), the media is replaced with fresh media and the cells are cultured further.

In some embodiments of the methods of modifying a BCL11A target nucleic acid in a cell, the methods further comprises contacting the target nucleic acid sequence of the cell with: a) an additional CRISPR nuclease and a gRNA targeting a different or overlapping portion of the BCL11A target nucleic acid compared to the first gRNA; b) a polynucleotide encoding the additional CRISPR nuclease and the gRNA of (a); c) a vector comprising the polynucleotide of (b); or d) a XDP comprising the additional CRISPR nuclease and the gRNA of (a), wherein the contacting results in modification of the BCL11A target nucleic acid at a different location in the sequence compared to the first gRNA. In some cases, the additional CRISPR nuclease is a CasX protein having a sequence different from the CasX protein of any of the preceding claims. In other cases, the additional CRISPR nuclease is not a CasX protein and is selected from the group consisting of Cas9, Cas12a, Cas12b, Cas12c, Cas12d (CasY), Cas12j, Cas12k, Cas13a, Cas13b, Cas13c, Cas13d, CasY, Cas14, Cpf1, C2cl, Csn2, Cas Phi, and sequence variants thereof.

In those cases where the modification results in a knock-down of the BCL11A gene, expression of the BCL11A protein is reduced by at least about 10%, at least about 20%, at least about 30%, at least about 40%, at least about 50%, at least about 60%, at least about 70%, at least about 80%, or at least about 90% in comparison to cells that have not been modified. In other cases, wherein the modification results in a knock-out of the BCL11A gene, the target nucleic acid of the cells of the population is modified such that expression of the BCL11A protein cannot be detected. Expression of a BCL11A protein can be measured by flow cytometry, ELISA, cell-based assays, Western blot, qRT-PCR, or other methods know in the art, or as described in the Examples.

In some embodiments, the disclosure provides methods of modifying a BCL11A target nucleic acid in a population of cells in vivo in a subject. In some embodiments, the modifying of the target nucleic acid sequence is carried out ex vivo in a eukaryotic cell, wherein the eukaryotic cell is selected from the group consisting of a hematopoietic stem cell (HSC), a hematopoietic progenitor cell (HPC), a CD34+ cell, a mesenchymal stem cell (MSC), induced pluripotent stem cell (iPSC), a common myeloid progenitor cell, a proerythroblast cell, and an erythroblast cell. In the foregoing embodiment, a population of the modified cells can be utilized in a method of treatment in a subject, wherein the modified cells are administered to the subject in need thereof, and wherein the subject is selected from the group consisting of mouse, rat, pig, non-human primate, and human. In some cases, the ex vivo cell is autologous and is isolated from the subject's bone marrow or peripheral blood. In other cases, the ex vivo cell is allogeneic and is isolated from a different subject's bone marrow or peripheral blood. In the methods of treatment, the modified cell can be administered to the subject by a route of administration selected from intraparenchymal, intravenous, intra-arterial, intramuscular, subcuticular, intraarticular, intracardiac, intrapericardial, intravitreal, sub-capsular, or by subcutaneous injection and can be implanted into the subject by transplantation, local injection, systemic infusion, or combinations thereof. In the foregoing embodiment, the method results in the persistence of the modified cell or its progeny for at least about 1 month, at least about 2 months, at least about 3 months, at least about 4 months, at least about 6 months, at least about 7 months, at least about 8 months, at least about 9 months, at least about 10 months, at least about 11 months, at least about 12 months, at least about 18 months, at least about 2 years, at least about 3 years, at least about 4 years, or at least about 5 years.

In some embodiments of the methods of modifying a target nucleic acid sequence, modifying the BCL11A gene comprises binding of the CasX:gRNA complex to the target nucleic acid sequence and is introduced into the cells as an RNP. In some embodiments, the CasX is a catalytically inactive CasX (dCasX) protein that retains the ability to bind to the gRNA and the target nucleic acid sequence. For example, the target nucleic acid sequence comprises a BCL11A sequence comprising a sequence complementary to the GATA1 binding motif sequence, and binding of the dCasX:gRNA complex to the target sequence interferes with or represses transcription of the BCL11A allele. In some embodiments, the dCasX comprises a mutation at residues D672, E769, and/or D935 corresponding to the CasX protein of SEQ ID NO: 1 or D659, E756 and/or D922 corresponding to the CasX protein of SEQ ID NO: 2. In some embodiments of the foregoing, the mutation in the CasX variant protein is a substitution of alanine or glycine for the residue and can be utilized for any of the variants described herein.

Introducing recombinant expression vectors comprising the components or the nucleic acids encoding the components of the system embodiments into a target cell can be carried out in vivo, in vitro or ex vivo. In some embodiments of the method, vectors may be provided directly to a target host cell. Methods of introducing a nucleic acid (e.g., a nucleic acid comprising a donor polynucleotide sequence, one or more nucleic acids (DNA or RNA) encoding a CasX protein and/or gRNA, or a vector comprising same) into a cell are known in the art, and any convenient method can be used to introduce a nucleic acid (e.g., an expression construct) into a cell. Suitable methods include e.g., viral infection, transfection, lipofection, electroporation, calcium phosphate precipitation, polyethyleneimine (PEI)-mediated transfection, DEAE-dextran mediated transfection, liposome-mediated transfection, particle gun technology, nucleofection, electroporation, direct addition by cell penetrating CasX proteins that are fused to or recruit donor DNA, cell squeezing, calcium phosphate precipitation, direct microinjection, nanoparticle-mediated nucleic acid delivery, and the like. Nucleic acids may be introduced into the cells using well-developed commercially-available transfection techniques such as use of TransMessenger® reagents from Qiagen, Stemfect™ RNA Transfection Kit from Stemgent, and TransIT®-mRNA Transfection Kit from Mirus Bio LLC, Lonza nucleofection, Maxagen electroporation and the like. Introducing recombinant expression vectors comprising sequences encoding the CasX:gRNA systems (and, optionally, the donor sequences) of the disclosure into cells under in vitro conditions can occur in any suitable culture media and under any suitable culture conditions that promote the survival of the cells. For example, cells may be contacted with vectors comprising the subject nucleic acids (e.g., recombinant expression vectors having the donor template sequence and nucleic acid encoding the CasX and gRNA) such that the vectors are taken up by the cells. Vectors used for providing the nucleic acids encoding gRNAs and/or CasX proteins to a target host cell can include suitable promoters for driving the expression, that is, transcriptional activation of the nucleic acid of interest. In some cases, the encoding nucleic acid of interest will be operably linked to a promoter. This may include ubiquitously acting promoters, for example, the CMV-beta-actin promoter, or inducible promoters, such as promoters that are active in particular cell populations or that respond to the presence of drugs such as tetracycline or kanamycin. By transcriptional activation, it is intended that transcription will be increased above basal levels in the target host cell comprising the vector by at least about 10-fold, by at least about 100-fold, more usually by at least about 1000-fold. In addition, vectors used for providing a nucleic acid encoding a gRNA and/or a CasX protein to a cell may include nucleic acid sequences that encode for selectable markers in the target cells, so as to identify cells that have taken up the CasX protein and/or the gRNA.

For viral vector delivery, cells can be contacted with viral particles comprising the subject viral expression vectors and the nucleic acid encoding the CasX and gRNA and, optionally, the donor template. In some embodiments, the vector is an Adeno-Associated Viral (AAV) vector, wherein the AAV is selected from AAV1, AAV2, AAV3, AAV4, AAV5, AAV6, AAV7, AAV8, AAV9, AAV10, AAV11, AAV12, AAV44.9, AAV-Rh74, or AAVRh10. In other cases, the AAV is selected from AAV1, AAV2, AAV5, AAV6, AAV7, AAV8, and AAV9, which are efficient for muscle transduction (Gruntman A M, et al. Gene transfer in skeletal and cardiac muscle using recombinant adeno-associated virus. Curr Protoc Microbiol. 14(14D):3 (2013). Embodiments of AAV vectors are described more fully, below. In other embodiments, the vector is a lentiviral vector. Retroviruses, for example, lentiviruses, may be suitable for use in methods of the present disclosure. Commonly used retroviral vectors are “defective”, e.g., are unable to produce viral proteins required for productive infection. Rather, replication of the vector requires growth in a packaging cell line. To generate viral particles comprising nucleic acids of interest, the retroviral nucleic acids comprising the nucleic acid are packaged into viral capsids by a packaging cell line. Different packaging cell lines provide a different envelope protein (ecotropic, amphotropic or xenotropic) to be incorporated into the capsid, and this envelope protein determines the specificity or tropism of the viral particle for the cells (ecotropic for murine and rat; amphotropic for most mammalian cell types including human, dog and mouse; and xenotropic for most mammalian cell types except murine cells). The appropriate packaging cell line may be used to ensure that the cells are targeted by the packaged viral particles. Methods of introducing subject vector expression vectors into packaging cell lines, and of collecting the viral particles that are generated by the packaging lines, are well known in the art, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., J. Virol. 63:03822-3828 (1989). Nucleic acids can also be introduced by direct micro-injection (e.g., injection of RNA).

In other embodiments of the methods of modifying a BCL11A gene, the method utilizes CasX delivery particles (XDP) for the targeted delivery of RNPs to the cells of the subject. XDP are particles that closely resemble viruses, but do not contain viral genetic material and are therefore non-infectious. In some embodiments, the XDP comprise a CasX and gRNA complexed as an RNP and, optionally, a donor template comprising all or a portion of the BCL11A gene to either knock-down or knock-out the BCL11A gene or a portion of the gene by insertion via HDR or HITI mechanisms. Embodiments of XDPs are described more fully, below.

### VI Polynucleotides and Vectors

In another aspect, the present disclosure relates to polynucleotides encoding the Class2, Type V nucleases and gRNA that have utility in the editing of the BCL11A gene. In some embodiments, the disclosure provides polynucleotides encoding the CasX proteins and the polynucleotides of the gRNAs of any of the CasX:gRNA system embodiments described herein. In additional embodiments, the disclosure provides donor template polynucleotides encoding portions or all of a BCL11A gene. In some cases, the donor template comprises a mutation or a heterologous sequence for knocking down or knocking out the BCL11A gene upon its insertion in the target nucleic acid. In yet further embodiments, the disclosure provides vectors comprising polynucleotides encoding the CasX proteins and the CasX gRNAs described herein, as well as the donor templates of the embodiments.

In some embodiments, the disclosure provides a polynucleotide sequence encoding the CasX variants of any of the embodiments described herein, including the CasX protein variants of SEQ ID NOS: 59, 72-99, 101-148, and 26908-27154 as described in Table 4 or sequences having at least about 50%, at least about 60%, at least about 70%, at least about 80%, at least about 90%, at least about 95%, at least about 96%, at least about 97%, at least about 98%, or at least about 99% sequence identity to a sequence of SEQ ID NOS: 59, 72-99, 101-148, and 26908-27154 of Table 4. In some embodiments, the disclosure provides a polynucleotide sequence encoding the CasX variants of any of the embodiments described herein, including the CasX protein variants of SEQ ID NOS: 36-99, 101-148, and 26908-27154 as described in Table 4 or sequences having at least about 50%, at least about 60%, at least about 70%, at least about 80%, at least about 90%, at least about 95%, at least about 96%, at least about 97%, at least about 98%, or at least about 99% sequence identity to a sequence of SEQ ID NOS: 36-99, 101-148, and 26908-27154 of Table 4. In some embodiments, the disclosure provides an isolated polynucleotide sequence encoding a gRNA sequence of any of the embodiments described herein, including the sequences of SEQ ID NOS: 4-16, 2238-2285, 26794-26839 or 27219-27265 of Tables 2 and 3, together with the targeting sequences of SEQ ID NOS: 272-2100 or 2286-26789. In some embodiments, the disclosure provides an isolated polynucleotide sequence encoding a gRNA sequence of any of the embodiments described herein, including the sequences of SEQ ID NOS: 2101-2285, 26794-26839 and 27219-27265, together with the targeting sequences of SEQ ID NOS: 272-2100 or 2286-26789. In some embodiments, the disclosure provides an isolated polynucleotide sequence encoding a gRNA sequence of any of the embodiments described herein, including the sequences of SEQ ID NOS: 2281-2285, 26794-26839 and 27219-27265, together with the targeting sequences of SEQ ID NOS: 272-2100 or 2286-26789. In some embodiments, the sequences encoding the CasX protein are codon optimized for expression in a eukaryotic cell.

In some embodiments, the disclosure provides a polynucleotide encoding a gRNA scaffold sequence of SEQ ID NOS: 4-16, 2238-2285, 26794-26839 or 27219-27265, or as set forth in Table 2 or Table 3, or a sequence having at least about 50%, at least about 60%, at least about 70%, at least about 80%, at least about 90%, at least about 95%, at least about 95%, at least about 96%, at least about 97%, at least about 98%, at least about 99% sequence identity thereto. In other embodiments, the disclosure provides a targeting sequence polynucleotide of Table 1, or a sequence having at least about 65%, at least about 75%, at least about 85%, or at least about 95% identity to a sequence of SEQ ID NOS: 272-2100 or 2286-26789. In some embodiments, the targeting sequence polynucleotide is, in turn, linked to the 3′ end of the gRNA scaffold sequence; either as a sgRNA or a dgRNA. In other embodiments, the disclosure provides gRNAs comprising targeting sequence polynucleotides having one or more single nucleotide polymorphisms (SNP) relative to a sequence of SEQ ID NOS: 272-2100 or 2286-26789.

In other embodiments, the disclosure provides an isolated polynucleotide sequence encoding a gRNA comprising a targeting sequence that is complementary to, and therefore is capable of hybridizing with, the BCL11A gene. In some embodiments, the polynucleotide sequence encodes a gRNA comprising a targeting sequence that hybridizes with a BCL11A exon. In other embodiments, the polynucleotide sequence encodes a gRNA comprising a targeting sequence that hybridizes with a BCL11A intron. In other embodiments, the polynucleotide sequence encodes a gRNA comprising a targeting sequence that hybridizes with a BCL11A intron-exon junction. In other embodiments, the polynucleotide sequence encodes a gRNA comprising a targeting sequence that hybridizes with an intergenic region of the BCL11A gene. In other embodiments, the polynucleotide sequence encodes a gRNA comprising a targeting sequence that hybridizes with a BCL11A regulatory element. In some cases, the BCL11A regulatory element is a BCL11A promoter or enhancer. In some cases, the BCL11A regulatory element is located 5′ of the BCL11A transcription start site, 3′ of the BCL11A transcription start, or in a BCL11A intron. In other embodiments, the polynucleotide sequence encodes a gRNA comprising a targeting sequence that hybridizes with a sequence located 5′ to the GATA1 binding motif sequence. In other embodiments, the polynucleotide sequence encodes a gRNA comprising a targeting sequence that hybridizes with a sequence overlapping the GATA1 binding motif sequence. In a particular embodiment of the foregoing, the polynucleotide sequence encodes a gRNA comprising a targeting sequence having SEQ ID NO: 22. In some cases, the BCL11A regulatory element is in an intron of the BCL11A gene. In other cases, the BCL11A regulatory element comprises the 5′ UTR of the BCL11A gene. In still other cases, the BCL11A regulatory element comprises the 3′ UTR of the BCL11A gene.

In other embodiments, the disclosure provides donor template nucleic acids, wherein the donor template comprises a nucleotide sequence having homology to a BCL11A target nucleic acid sequence. In some embodiments, the BCL11A donor template is intended for gene editing in conjunction with the CasX:gRNA system and comprises at least a portion of a BCL11A gene. In other embodiments, the BCL11A donor sequence comprises a sequence that encodes at least a portion of a BCL11A exon. In other embodiments, the BCL11A donor template has a sequence that encodes at least a portion of a BCL11A intron. In other embodiments, the BCL11A donor template has a sequence that encodes at least a portion of a BCL11A intron-exon junction. In other embodiments, the BCL11A donor template has a sequence that encodes at least a portion of an intergenic region of the BCL11A gene. In other embodiments, the BCL11A donor template has a sequence that encodes at least a portion of a BCL11A regulatory element. In some cases, the BCL11A donor template is a wild-type sequence that encodes at least a portion of SEQ ID NO: 100. In other cases, the BCL11A donor template sequence comprises one or more mutations relative to a wild-type BCL11A gene. In a particular embodiment, the donor template has a sequence that encodes a portion or all of the GATA1 binding motif sequence but with at least 1 to 5 mutations relative to the wild-type sequence. In the foregoing embodiments, the donor template is at least 10 nucleotides, at least 100 nucleotides, at least 200 nucleotides, at least 300 nucleotides, at least 400 nucleotides, at least 500 nucleotides, at least 600 nucleotides, at least 700 nucleotides, at least 800 nucleotides, at least 900 nucleotides, at least 1,000 nucleotides, at least 2,000 nucleotides, at least 3,000 nucleotides, at least 4,000 nucleotides, at least 5,000 nucleotides, at least 6,000 nucleotides, at least 7,000 nucleotides, at least 8,000 nucleotides, at least 9,000 nucleotides, at least 10,000 nucleotides, at least 12,000 nucleotides, or at least 15,000 nucleotides. In some embodiments, the donor template comprises at least about 10 to about 15,000 nucleotides. In some embodiments, the donor template is a single-stranded DNA template. In other embodiments, the donor template is a single stranded RNA template. In other embodiments, the donor template is a double-stranded DNA template. In some embodiments, the donor template can be provided as naked nucleic acid in the systems to edit the BCL11A gene and does not need to be incorporated into a vector. In other embodiments, the donor template can be incorporated into a vector to facilitate its delivery to a cell; e.g., in a viral vector.

In other aspects, the disclosure relates to methods to produce polynucleotide sequences encoding the CasX variants, or the gRNA of any of the embodiments described herein, including homologous variants thereof, as well as methods to express the proteins expressed or RNA transcribed by the polynucleotide sequences. In general, the methods include producing a polynucleotide sequence coding for the CasX variants, or the gRNA of any of the embodiments described herein and incorporating the encoding gene into an expression vector appropriate for a host cell. Standard recombinant techniques in molecular biology can be used to make the polynucleotides and expression vectors of the present disclosure. For production of the encoded reference CasX, the CasX variants, or the gRNA of any of the embodiments described herein, the methods include transforming an appropriate host cell with an expression vector comprising the encoding polynucleotide, and culturing the host cell under conditions causing or permitting the resulting reference CasX, the CasX variants, or the gRNA of any of the embodiments described herein to be expressed or transcribed in the transformed host cell, thereby producing the CasX variants, or the gRNA, which are recovered by methods described herein or by standard purification methods known in the art or as described in the Examples.

In accordance with the disclosure, nucleic acid sequences that encode the CasX variants, or the gRNA of any of the embodiments described herein (or their complement) are used to generate recombinant DNA molecules that direct the expression in appropriate host cells. Several cloning strategies are suitable for performing the present disclosure, many of which are used to generate a construct that comprises a gene coding for a composition of the present disclosure, or its complement. In some embodiments, the cloning strategy is used to create a gene that encodes a construct that comprises nucleotides encoding the CasX variants, or the gRNA that is used to transform a host cell for expression of the composition.

In some approaches, a construct is first prepared containing the DNA sequence encoding a CasX variant or a gRNA. Exemplary methods for the preparation of such constructs are described in the Examples. The construct is then used to create an expression vector suitable for transforming a host cell, such as a prokaryotic or eukaryotic host cell for the expression and recovery of the protein construct, in the case of the CasX, or the gRNA. Where desired, the host cell is an E. coli. In other embodiments, the host cell is a eukaryotic cell. The eukaryotic host cell can be selected from Baby Hamster Kidney fibroblast (BHK) cells, human embryonic kidney 293 (HEK293), human embryonic kidney 293T (HEK293T), NS0 cells, SP2/0 cells, YO myeloma cells, P3X63 mouse myeloma cells, PER cells, PER.C6 cells, hybridoma cells, NIH3T3 cells, CV-1 (simian) in Origin with SV40 genetic material (COS), HeLa, Chinese hamster ovary (CHO), or yeast cells, or other eukaryotic cells known in the art suitable for the production of recombinant products. Exemplary methods for the creation of expression vectors, the transformation of host cells and the expression and recovery of the CasX variants or the gRNA are described in the Examples.

The gene encoding the CasX variant, or the gRNA construct can be made in one or more steps, either fully synthetically or by synthesis combined with enzymatic processes, such as restriction enzyme-mediated cloning, PCR and overlap extension, including methods more fully described in the Examples. The methods disclosed herein can be used, for example, to ligate sequences of polynucleotides encoding the various components (e.g., CasX and gRNA) genes of a desired sequence. Genes encoding polypeptide compositions are assembled from oligonucleotides using standard techniques of gene synthesis.

In some embodiments, the nucleotide sequence encoding a CasX protein is codon optimized for the intended host cell. This type of optimization can entail a mutation of an encoding nucleotide sequence to mimic the codon preferences of the intended host organism or cell while encoding the same CasX protein. Thus, the codons can be changed, but the encoded protein or gRNA remains unchanged. For example, if the intended target cell of the CasX protein was a human cell, a human codon-optimized CasX-encoding nucleotide sequence could be used. As another non-limiting example, if the intended host cell were a mouse cell, then a mouse codon-optimized CasX-encoding nucleotide sequence could be generated. The gene design can be performed using algorithms that optimize codon usage and amino acid composition appropriate for the host cell utilized in the production of the reference CasX or the CasX variants. In one method of the disclosure, a library of polynucleotides encoding the components of the constructs is created and then assembled, as described above. The resulting genes are then assembled and the resulting genes used to transform a host cell and produce and recover the CasX variants, or the gRNA compositions for evaluation of its properties, as described herein.

The disclosure provides for the use of plasmid expression vectors containing replication and control sequences that are compatible with and recognized by the host cell and are operably linked to the gene encoding the polypeptide for controlled expression of the polypeptide or transcription of the RNA. Such vector sequences are well known for a variety of bacteria, yeast, and viruses. Useful expression vectors that can be used include, for example, segments of chromosomal, non-chromosomal and synthetic DNA sequences. “Expression vector” refers to a DNA construct containing a DNA sequence that is operably linked to a suitable control sequence capable of effecting the expression of the DNA encoding the polypeptide in a suitable host. The requirements are that the vectors are replicable and viable in the host cell of choice. Low- or high-copy number vectors may be used as desired. The control sequences of the vector include a promoter to effect transcription, an optional operator sequence to control such transcription, a sequence encoding suitable mRNA ribosome binding sites, and sequences that control termination of transcription and translation. In some embodiments, a nucleotide sequence encoding a gRNA is operably linked to a control element, e.g., a transcriptional control element, such as a promoter. In some embodiments, a nucleotide sequence encoding a CasX protein is operably linked to a control element, e.g., a transcriptional control element, such as a promoter. In other cases, the nucleotide encoding the CasX and gRNA are linked and are operably linked to a single control element. The promoter may be any DNA sequence, which shows transcriptional activity in the host cell of choice and may be derived from genes encoding proteins either homologous or heterologous to the host cell. Exemplary regulatory elements include a transcription promoter, a transcription enhancer element, a transcription termination signal, internal ribosome entry site (IRES) or P2A peptide to permit translation of multiple genes from a single transcript, polyadenylation sequences to promote downstream transcriptional termination, sequences for optimization of initiation of translation, and translation termination sequences. In some cases, the promoter is a constitutively active promoter. In some cases, the promoter is a regulatable promoter. In some cases, the promoter is an inducible promoter. In some cases, the promoter is a tissue-specific promoter. In some cases, the promoter is a cell type-specific promoter. In some cases, the transcriptional control element (e.g., the promoter) is functional in a targeted cell type or targeted cell population. For example, in some cases, the transcriptional control element can be functional in eukaryotic cells, e.g., packaging cells for viral or XDP vectors, hematopoietic stem cells (HSC), hematopoietic progenitor cells (HPC), CD34+ cells, mesenchymal stem cells (MSC), embryonic stem (ES) cells, induced pluripotent stem cells (iPSC), common myeloid progenitor cells, proerythroblast cells, and erythroblast cells.

Non-limiting examples of pol II promoters include, but are not limited to EF-1alpha, EF-1alpha core promoter, Jens Tornoe (JeT), promoters from cytomegalovirus (CMV), CMV immediate early (CMVIE), CMV enhancer, herpes simplex virus (HSV) thymidine kinase, early and late simian virus 40 (SV40), the SV40 enhancer, long terminal repeats (LTRs) from retrovirus, mouse metallothionein-I, adenovirus major late promoter (Ad MLP), CMV promoter full-length promoter, the minimal CMV promoter, the chicken CE<-actin promoter (CBA), CBA hybrid (CBh), chicken CE≤-actin promoter with cytomegalovirus enhancer (CB7), chicken beta-Actin promoter and rabbit beta-Globin splice acceptor site fusion (CAG), the rous sarcoma virus (RSV) promoter, the HIV-Ltr promoter, the hPGK promoter, the HSV TK promoter, a 7SK promoter, the Mini-TK promoter, the human synapsin I (SYN) promoter which confers neuron-specific expression, beta-actin promoter, super core promoter 1 (SCP1), the Mecp2 promoter for selective expression in neurons, the minimal IL-2 promoter, the Rous sarcoma virus enhancer/promoter (single), the spleen focus-forming virus long terminal repeat (LTR) promoter, the TBG promoter, promoter from the human thyroxine-binding globulin gene (Liver specific), the PGK promoter, the human ubiquitin C promoter (UBC), the UCOE promoter (Promoter of HNRPA2B1-CBX3), the synthetic CAG promoter, the Histone H2 promoter, the Histone H3 promoter, the U1a1 small nuclear RNA promoter (226 nt), the U1a1 small nuclear RNA promoter (226 nt), the Ulb2 small nuclear RNA promoter (246 nt) 26, the GUSB promoter, the CBh promoter, rhodopsin (Rho) promoter, silencing-prone spleen focus forming virus (SFFV) promoter, a human H1 promoter (H1), a POL1 promoter, the TTR minimal enhancer/promoter, the b-kinesin promoter, mouse mammary tumor virus long terminal repeat (LTR) promoter, the human eukaryotic initiation factor 4A (EIF4A1) promoter, the ROSA26 promoter, the glyceraldehyde 3-phosphate dehydrogenase (GAPDH) promoter, tRNA promoters, and truncated versions and sequence variants of the foregoing. In a particular embodiment, the pol II promoter is EF-1alpha, wherein the promoter enhances transfection efficiency, the transgene transcription or expression of the CRISPR nuclease, the proportion of expression-positive clones and the copy number of the episomal vector in long-term culture.

Non-limiting examples of pol III promoters include, but are not limited to U6, mini U6, U6 truncated promoters,7SK, and H1 variants, BiH1 (Bidrectional H1 promoter), BiU6, Bi7SK, BiH1 (Bidirectional U6, 7SK, and H1 promoters), gorilla U6, rhesus U6, human 7SK, human H1 promoters, and sequence variants thereof. In the foregoing embodiment, the pol III promoter enhances the transcription of the gRNA.

Selection of the appropriate vector and promoter is well within the level of ordinary skill in the art, as it related to controlling expression, e.g., for modifying a BCL11A gene. The expression vector may also contain a ribosome binding site for translation initiation and a transcription terminator. The expression vector may also include appropriate sequences for amplifying expression. The expression vector may also include nucleotide sequences encoding protein tags (e.g., 6×His tag, hemagglutinin tag, fluorescent protein, etc.) that can be fused to the CasX protein, thus resulting in a chimeric CasX protein that are used for purification or detection.

Recombinant expression vectors of the disclosure can also comprise elements that facilitate robust expression of CasX proteins and the gRNAs of the disclosure. For example, recombinant expression vectors can include one or more of a polyadenylation signal (poly(A)), an intronic sequence or a post-transcriptional regulatory element such as a woodchuck hepatitis post-transcriptional regulatory element (WPRE). Exemplary poly(A) sequences include hGH poly(A) signal (short), HSV TK poly(A) signal, synthetic polyadenylation signals, SV40 poly(A) signal, β-globin poly(A) signal and the like. A person of ordinary skill in the art will be able to select suitable elements to include in the recombinant expression vectors described herein.

In some embodiments, provided herein are one or more recombinant expression vectors comprising one or more of: (i) a nucleotide sequence of a donor template nucleic acid where the donor template comprises a nucleotide sequence having homology to a sequence of the target BCL11A locus of the target nucleic acid (e.g., a target genome); (ii) a nucleotide sequence that encodes a gRNA that hybridizes to a target sequence of the BCL11A locus of the targeted genome (e.g., configured as a single or dual guide RNA) operably linked to a promoter that is operable in a target cell such as a eukaryotic cell; and (iii) a nucleotide sequence encoding a CasX protein operably linked to a promoter that is operable in a target cell such as a eukaryotic cell. In some embodiments, the sequences encoding the donor template, the gRNA and the CasX protein are in different recombinant expression vectors, and in other embodiments one or more polynucleotide sequences (for the donor template, CasX, and the gRNA) are in the same recombinant expression vector. In other cases, the CasX and gRNA are delivered to the target cell as an RNP (e.g., by electroporation or chemical means) and the donor template is delivered by a vector.

The polynucleotide sequence(s) are inserted into the vector by a variety of procedures. In general, DNA is inserted into an appropriate restriction endonuclease site(s) using techniques known in the art. Vector components generally include, but are not limited to, one or more of a signal sequence, an origin of replication, one or more marker genes, an enhancer element, a promoter, and a transcription termination sequence. Construction of suitable vectors containing one or more of these components employs standard ligation techniques which are known to the skilled artisan. Such techniques are well known in the art and well described in the scientific and patent literature. Various vectors are publicly available. The vector may, for example, be in the form of a plasmid, cosmid, viral particle, or phage that may conveniently be subjected to recombinant DNA procedures, and the choice of vector will often depend on the host cell into which it is to be introduced. Thus, the vector may be an autonomously replicating vector, i.e., a vector, which exists as an extrachromosomal entity, the replication of which is independent of chromosomal replication, e.g., a plasmid. Alternatively, the vector may be one which, when introduced into a host cell, is integrated into the host cell genome and replicated together with the chromosome(s) into which it has been integrated. Once introduced into a suitable host cell, expression of the protein involved in antigen processing, antigen presentation, antigen recognition, and/or antigen response can be determined using any nucleic acid or protein assay known in the art. For example, the presence of transcribed mRNA of reference CasX or the CasX variants can be detected and/or quantified by conventional hybridization assays (e.g., Northern blot analysis), amplification procedures (e.g. RT-PCR), SAGE (U.S. Pat. No. 5,695,937), and array-based technologies (see e.g., U.S. Pat. Nos. 5,405,783, 5,412,087 and 5,445,934), using probes complementary to any region of the polynucleotide.

The polynucleotides and recombinant expression vectors can be delivered to the target host cells by a variety of methods. Such methods include, but are not limited to, viral infection, transfection, lipofection, electroporation, calcium phosphate precipitation, polyethyleneimine (PEI)-mediated transfection, DEAE-dextran mediated transfection, microinjection, liposome-mediated transfection, particle gun technology, nucleofection, direct addition by cell penetrating CasX proteins that are fused to or recruit donor DNA, cell squeezing, calcium phosphate precipitation, direct microinjection, nanoparticle-mediated nucleic acid delivery, and using the commercially available TransMessenger® reagents from Qiagen, Stemfect™ RNA Transfection Kit from Stemgent, and TransIT®-mRNA Transfection Kit from Mirus Bio LLC, Lonza nucleofection, Maxagen electroporation and the like.

A recombinant expression vector sequence can be packaged into a virus or virus-like particle (also referred to herein as a “particle” or “virion”) for subsequent infection and transformation of a cell, ex vivo, in vitro or in vivo. Such particles or virions will typically include proteins that encapsidate or package the vector genome. Suitable expression vectors may include viral expression vectors based on vaccinia virus; poliovirus; adenovirus; a retroviral vector (e.g., Murine Leukemia Virus), spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus; and the like. In some embodiments, a recombinant expression vector of the present disclosure is a recombinant adeno-associated virus (AAV) vector. In some embodiments, a recombinant expression vector of the present disclosure is a recombinant lentivirus vector. In some embodiments, a recombinant expression vector of the present disclosure is a recombinant retroviral vector.

In some embodiments, a recombinant expression vector of the present disclosure is a recombinant adeno-associated virus (AAV) vector. In some embodiments, a recombinant expression vector of the present disclosure is a recombinant lentivirus vector. In some embodiments, a recombinant expression vector of the present disclosure is a recombinant retroviral vector.

AAV is a small (20 nm), nonpathogenic virus that is useful in treating human diseases in situations that employ a viral vector for delivery to a cell such as a eukaryotic cell, either in vivo or ex vivo for cells to be prepared for administering to a subject. A construct is generated, for example a construct encoding any of the CasX proteins and/or CasX gRNA embodiments as described herein, and is flanked with AAV inverted terminal repeat (ITR) sequences, thereby enabling packaging of the AAV vector into an AAV viral particle.

An “AAV” vector may refer to the naturally occurring wild-type virus itself or derivatives thereof. The term covers all subtypes, serotypes and pseudotypes, and both naturally occurring and recombinant forms, except where required otherwise. As used herein, the term “serotype” refers to an AAV which is identified by and distinguished from other AAVs based on capsid protein reactivity with defined antisera, e.g., there are many known serotypes of primate AAVs. In some embodiments, the AAV vector is selected from AAV1, AAV2, AAV3, AAV4, AAV5, AAV6, AAV7, AAV8, AAV9, AAV10, AAV11, AAV12, AAV44.9, AAV-Rh74 (Rhesus macaque-derived AAV), and AAVRh10, and modified capsids of these serotypes. For example, serotype AAV-2 is used to refer to an AAV which contains capsid proteins encoded from the cap gene of AAV-2 and a genome containing 5′ and 3′ ITR sequences from the same AAV-2 serotype. Pseudotyped AAV refers to an AAV that contains capsid proteins from one serotype and a viral genome including 5′-3′ ITRs of a second serotype. Pseudotyped rAAV would be expected to have cell surface binding properties of the capsid serotype and genetic properties consistent with the ITR serotype. Pseudotyped recombinant AAV (rAAV) are produced using standard techniques described in the art. As used herein, for example, rAAV1 may be used to refer an AAV having both capsid proteins and 5′-3′ ITRs from the same serotype or it may refer to an AAV having capsid proteins from serotype 1 and 5′-3′ ITRs from a different AAV serotype, e.g., AAV serotype 2. For each example illustrated herein the description of the vector design and production describes the serotype of the capsid and 5′-3′ ITR sequences.

An “AAV virus” or “AAV viral particle” refers to a viral particle composed of at least one AAV capsid protein (preferably by all of the capsid proteins of a wild-type AAV) and an encapsidated polynucleotide. If the particle additionally comprises a heterologous polynucleotide (i.e., a polynucleotide other than a wild-type AAV genome to be delivered to a mammalian cell), it is typically referred to as “rAAV”. An exemplary heterologous polynucleotide is a polynucleotide comprising a CasX protein and/or sgRNA and, optionally, a donor template of any of the embodiments described herein.

By “adeno-associated virus inverted terminal repeats” or “AAV ITRs” is meant the art recognized regions found at each end of the AAV genome which function together in cis as origins of DNA replication and as packaging signals for the virus. AAV ITRs, together with the AAV rep coding region, provide for the efficient excision and rescue from, and integration of a nucleotide sequence interposed between two flanking ITRs into a mammalian cell genome. The nucleotide sequences of AAV ITR regions are known. See, for example Kotin, R. M. (1994) Human Gene Therapy 5:793-801; Berns, K. I. “Parvoviridae and their Replication” in Fundamental Virology, 2^{nd} Edition, (B. N. Fields and D. M. Knipe, eds.). As used herein, an AAV ITR need not have the wild-type nucleotide sequence depicted, but may be altered, e.g., by the insertion, deletion or substitution of nucleotides. Additionally, the AAV ITR may be derived from any of several AAV serotypes, including without limitation, AAV1, AAV2, AAV3, AAV4, AAV5, AAV6, AAV7, AAV8, AAV9, AAV10, AAV-Rh74, and AAVRh10, and modified capsids of these serotypes. Furthermore, 5′ and 3′ ITRs which flank a selected nucleotide sequence in an AAV vector need not necessarily be identical or derived from the same AAV serotype or isolate, so long as they function as intended, i.e., to allow for excision and rescue of the sequence of interest from a host cell genome or vector, and to allow integration of the heterologous sequence into the recipient cell genome when AAV Rep gene products are present in the cell. Use of AAV serotypes for integration of heterologous sequences into a host cell is known in the art (see, e.g., WO2018195555A1 and US20180258424A1, incorporated by reference herein).

By “AAV rep coding region” is meant the region of the AAV genome which encodes the replication proteins Rep 78, Rep 68, Rep 52 and Rep 40. These Rep expression products have been shown to possess many functions, including recognition, binding and nicking of the AAV origin of DNA replication, DNA helicase activity and modulation of transcription from AAV (or other heterologous) promoters. The Rep expression products are collectively required for replicating the AAV genome. By “AAV cap coding region” is meant the region of the AAV genome which encodes the capsid proteins VP1, VP2, and VP3, or functional homologues thereof. These Cap expression products supply the packaging functions which are collectively required for packaging the viral genome.

In some embodiments, AAV capsids utilized for delivery of the encoding sequences for the CasX and gRNA, and, optionally, the DMPK donor template nucleotides to a host cell can be derived from any of several AAV serotypes, including without limitation, AAV1, AAV2, AAV3, AAV4, AAV5, AAV6, AAV7, AAV8, AAV9, AAV10, AAV11, AAV12, AAV44.9, AAV-Rh74 (Rhesus macaque-derived AAV), and AAVRh10, and the AAV ITRs are derived from AAV serotype 2. In a particular embodiment, AAV1, AAV7, AAV6, AAV8, or AAV9 are utilized for delivery of the CasX, gRNA, and, optionally, donor template nucleotides, to a host muscle cell.

In order to produce rAAV viral particles, an AAV expression vector is introduced into a suitable host cell using known techniques, such as by transfection. Packaging cells are typically used to form virus particles; such cells include HEK293 cells (and other cells known in the art), which package adenovirus. A number of transfection techniques are generally known in the art; see, e.g., Sambrook et al. (1989) Molecular Cloning, a laboratory manual, Cold Spring Harbor Laboratories, New York. Particularly suitable transfection methods include calcium phosphate co-precipitation, direct microinjection into cultured cells, electroporation, liposome mediated gene transfer, lipid-mediated transduction, and nucleic acid delivery using high-velocity microprojectiles.

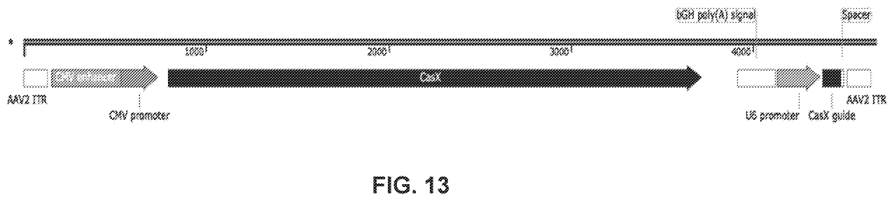

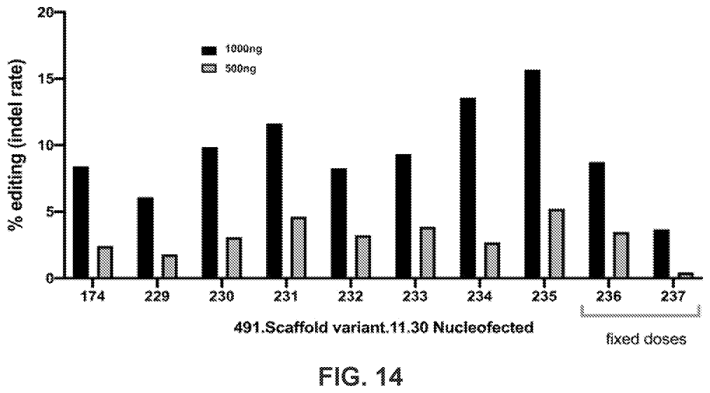

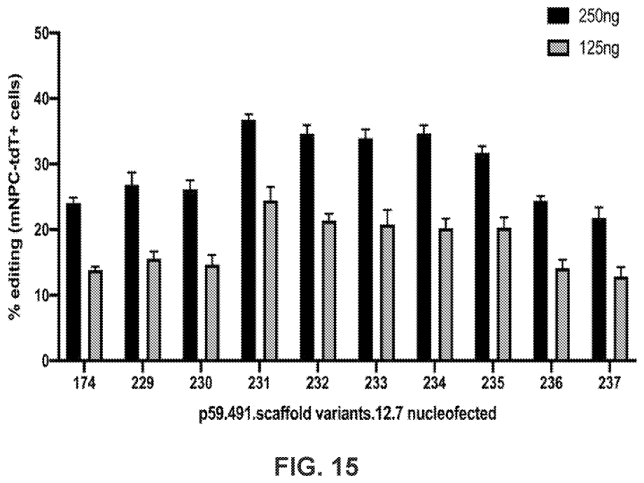

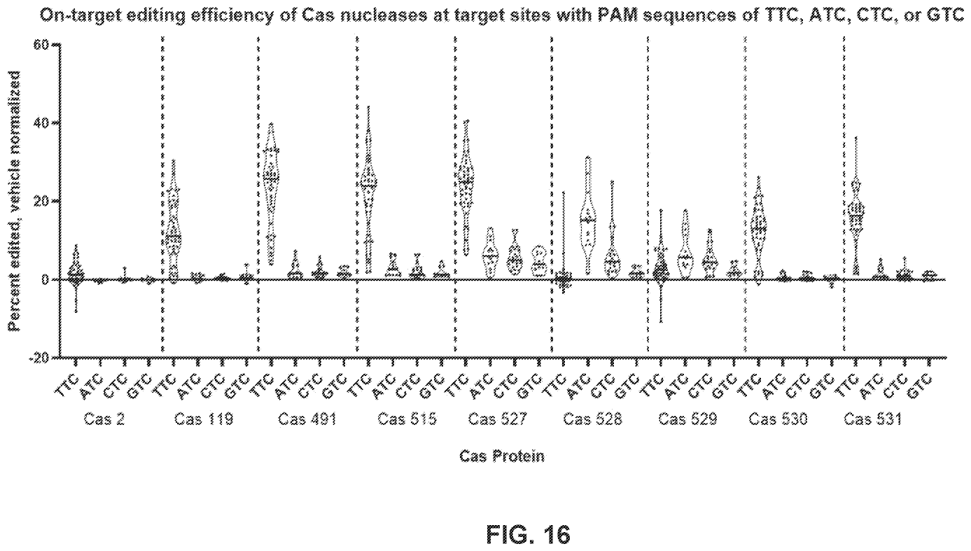

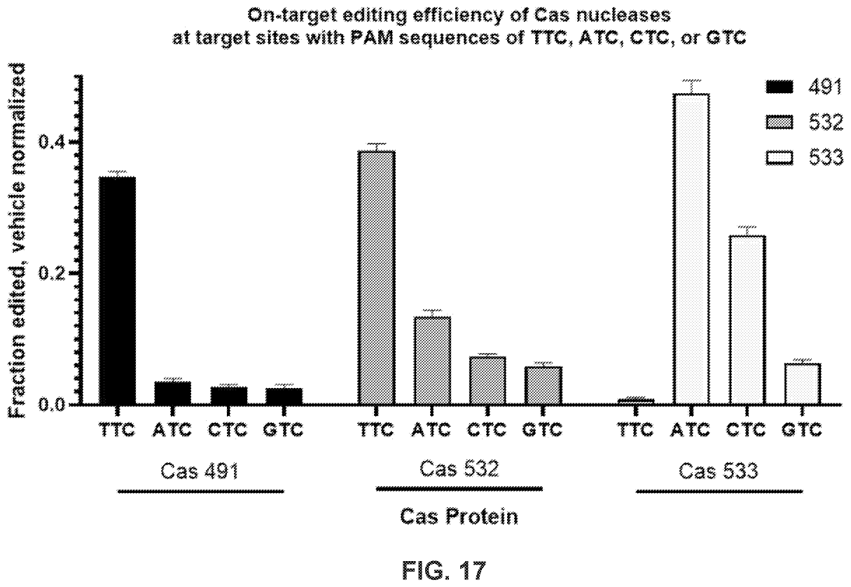

In an advantage of rAAV constructs of the present disclosure, the smaller size of the CRISPR Type V nucleases; e.g., the CasX of the embodiments, permits the inclusion of all the necessary editing and ancillary expression components into the transgene such that a single rAAV particle can deliver and transduce these components into a target cell in a form that results in the expression of the CRISPR nuclease and gRNA that are capable of effectively modifying the target nucleic acid of the target cell. A representative schematic of such a construct is presented in FIG. 13. This stands in marked contrast to other CRISPR systems, such as Cas9, where typically a two-particle system is employed to deliver the necessary editing components to a target cell. Thus, in some embodiments of the rAAV systems, the disclosure provides; i) a first plasmid comprising the ITRs, sequences encoding the CasX variant, sequences encoding one or more gRNA, a first promoter operably linked to the CasX and a second promoter operably linked to the gRNA, and, optionally, one or more enhancer elements; ii) a second plasmid comprising the rep and cap genes; and iii) a third plasmid comprising helper genes, wherein upon transfection of an appropriate packaging cell, the cell is capable of producing an rAAV having the ability to deliver to a target cell, in a single particle, sequences capable of expressing the CasX nuclease and gRNA having the ability to edit the target nucleic acid of the target cell. In some embodiments of the rAAV systems, the sequence encoding the CRISPR protein and the sequence encoding the at least first gRNA are less than about 3100, less than about 3090, less than about 3080, less than about 3070, less than about 3060, less than about 3050, or less than about 3040 nucleotides in length, such that the sequences encoding the first and second promoter and, optionally, one or more enhance elements can have at least about 1300, at least about 1350, at least about 1360, at least about 1370, at least about 1380, at least about 1390, at least about 1400, at least about 1500, at least about 1600 nucleotides, at least 1650, at least about 1700, at least about 1750, at least about 1800, at least about 1850, or at least about 1900 nucleotides in combined length. In some embodiments of the rAAV systems, the sequence encoding the first promoter and the at least one accessory element have greater than at least about 1300, at least about 1350, at least about 1360, at least about 1370, at least about 1380, at least about 1390, at least about 1400, at least about 1500, at least about 1600 nucleotides, at least 1650, at least about 1700, at least about 1750, at least about 1800, at least about 1850, or at least about 1900 nucleotides in combined length. In some embodiments of the rAAV systems, the sequence encoding the first and second promoters and the at least one accessory element have greater than at least about 1300, at least about 1350, at least about 1360, at least about 1370, at least about 1380, at least about 1390, at least about 1400, at least about 1500, at least about 1600 nucleotides, at least 1650, at least about 1700, at least about 1750, at least about 1800, at least about 1850, or at least about 1900 nucleotides in combined length.

In some embodiments, host cells transfected with the above-described AAV expression vectors are rendered capable of providing AAV helper functions in order to replicate and encapsidate the nucleotide sequences flanked by the AAV ITRs to produce rAAV viral particles. AAV helper functions are generally AAV-derived coding sequences which can be expressed to provide AAV gene products that, in turn, function in trans for productive AAV replication. AAV helper functions are used herein to complement necessary AAV functions that are missing from the AAV expression vectors. Thus, AAV helper functions include one, or both of the major AAV ORFs (open reading frames), encoding the rep and cap coding regions, or functional homologues thereof. Accessory functions can be introduced into and then expressed in host cells using methods known to those of skill in the art. Commonly, accessory functions are provided by infection of the host cells with an unrelated helper virus. In some embodiments, accessory functions are provided using an accessory function vector. Depending on the host/vector system utilized, any of a number of suitable transcription and translation control elements, including constitutive and inducible promoters, transcription enhancer elements, transcription terminators, etc., may be used in the expression vector. In some embodiments, the disclosure provides host cells comprising the AAV vectors of the embodiments disclosed herein.

In other embodiments, suitable vectors may include virus-like particles (VLP). Virus-like particles (VLPs) are particles that closely resemble viruses, but do not contain viral genetic material and are therefore non-infectious. In some embodiments, VLPs comprise a polynucleotide encoding a transgene of interest, for example any of the CasX protein and/or a gRNA embodiments, and, optionally, donor template polynucleotides described herein, packaged with one or more viral structural proteins. In other embodiments, the disclosure provides XDPs produced in vitro that comprise a CasX:gRNA RNP complex and, optionally, a donor template. Combinations of structural proteins from different viruses can be used to create XDPs, including components from virus families including Parvoviridae (e.g., adeno-associated virus), Retroviridae (e.g., alpharetrovirus, a betaretrovirus, a gammaretrovirus, a deltaretrovirus, a epsilonretrovirus, or a lentivirus), Flaviviridae (e.g., Hepatitis C virus), Paramyxoviridae (e.g., Nipah) and bacteriophages (e.g., QP, AP205). In some embodiments, the disclosure provides XDP systems designed using components of retrovirus, including lentiviruses (such as HIV) and alpharetrovirus, betaretrovirus, gammaretrovirus, deltaretrovirus, epsilonretrovirus, in which individual plasmids comprising polynucleotides encoding the various components are introduced into a packaging cell that, in turn, produce the XDP. In some embodiments, the disclosure provides XDP comprising one or more components of i) protease, ii) a protease cleavage site, iii) one or more components of a gag polyprotein selected from a matrix protein (MA), a nucleocapsid protein (NC), a capsid protein (CA), a p1 peptide, a p6 peptide, a P2A peptide, a P2B peptide, a P10 peptide, a p12 peptide, a PP 21/24 peptide, a P12/P3/P8 peptide, and a P20 peptide; v) CasX; vi) gRNA, and vi) targeting glycoproteins or antibody fragments wherein the resulting XDP particle encapsidates a CasX:gRNA RNP. The polynucleotides encoding the Gag, CasX and gRNA can further comprise paired components designed to assist the trafficking of the components out of the nucleus of the host cell and into the budding XDP. Non-limiting examples of such trafficking components include hairpin RNA such as MS2 hairpin, PP7 hairpin, QP hairpin, and U1 hairpin II that have binding affinity for MS2 coat protein, PP7 coat protein, QP coat protein, and U1A signal recognition particle, respectively. In other embodiments, the gRNA can comprise Rev response element (RRE) or portions thereof that have binding affinity to Rev, which can be linked to the Gag polyprotein. In other embodiments, the gRNA can comprise one or more RRE and one or more MS2 hairpin sequences. In other embodiments, the gRNA can comprise Rev response element (RRE) or portions thereof that have binding affinity to Rev, which can be linked to the Gag polyprotein. The RRE can be selected from the group consisting of Stem IIB of Rev response element (RRE), Stem II-V of RRE, Stem II of RRE, Rev-binding element (RBE) of Stem IIB, and full-length RRE. In the foregoing embodiment, the components include sequences of

UGGGCGCAGCGUCAAUGACGCUGACGGUACA (Stem IIB; SEQ ID NO: 27266), GCACUAUGGGCGCAGCGUCAAUGACGCUGACGGUACAGGCCAGACAAUUAUUGU CUGGUAUAGUGC (Stem II; SEQ ID NO: 27267), GCUGACGGUACAGGC (RBE, SEQ ID NO: 27268), CAGGAAGCACUAUGGGCGCAGCGUCAAUGACGCUGACGGUACAGGCCAGACAAU UAUUGUCUGGUAUAGUGCAGCAGCAGAACAAUUUGCUGAGGGCUAUUGAGGCGC AACAGCAUCUGUUGCAACUCACAGUCUGGGGCAUCAAGCAGCUCCAGGCAAGAA UCCUG (Stem II-V; SEQ ID NO: 27269), and AGGAGCUUUGUUCCUUGGGUUCUUGGGAGCAGCAGGAAGCACUAUGGGCGCAGC GUCAAUGACGCUGACGGUACAGGCCAGACAAUUAUUGUCUGGUAUAGUGCAGCA GCAGAACAAUUUGCUGAGGGCUAUUGAGGCGCAACAGCAUCUGUUGCAACUCAC AGUCUGGGGCAUCAAGCAGCUCCAGGCAAGAAUCCUGGCUGUGGAAAGAUACCU AAAGGAUCAACAGCUCCU (full-length RRE; SEQ ID NO: 27270). In other embodiments, the gRNA can comprise one or more RRE and one or more MS2 hairpin sequences. In a particular embodiment, the gRNA comprises an MS2 hairpin variant that is optimized to increase the binding affinity to the MS2 coat protein, thereby enhancing the incorporation of the gRNA and associated CasX into the budding XDP. gRNA variants comprising MS2 hairpin variants include gRNA variants 275-315 and 317-320 (SEQ ID NOS: 2722-27264).

The targeting glycoproteins or antibody fragments on the surface that provides tropism of the XDP to the target cell, wherein upon administration and entry into the target cell, the RNP molecule is free to be transported into the nucleus of the cell. The envelope glycoprotein can be derived from any enveloped viruses known in the art to confer tropism to XDP, including but not limited to the group consisting of Argentine hemorrhagic fever virus, Australian bat virus, Autographa californica multiple nucleopolyhedrovirus, Avian leukosis virus, baboon endogenous virus, Bolivian hemorrhagic fever virus, Borna disease virus, Breda virus, Bunyamwera virus, Chandipura virus, Chikungunya virus, Crimean-Congo hemorrhagic fever virus, Dengue fever virus, Duvenhage virus, Eastern equine encephalitis virus, Ebola hemorrhagic fever virus, Ebola Zaire virus, enteric adenovirus, Ephemerovirus, Epstein-Bar virus (EBV), European bat virus 1, European bat virus 2, Fug Synthetic gP Fusion, Gibbon ape leukemia virus, Hantavirus, Hendra virus, hepatitis A virus, hepatitis B virus, hepatitis C virus, hepatitis D virus, hepatitis E virus, hepatitis G Virus (GB virus C), herpes simplex virus type 1, herpes simplex virus type 2, human cytomegalovirus (HHV5), human foamy virus, human herpesvirus (HHV), human Herpesvirus 7, human herpesvirus type 6, human herpesvirus type 8, human immunodeficiency virus 1 (HIV-1), human metapneumovirus, human T-lymphotropic virus 1, influenza A, influenza B, influenza C virus, Japanese encephalitis virus, Kaposi's sarcoma-associated herpesvirus (HHV8), Kaysanur Forest disease virus, La Crosse virus, Lagos bat virus, Lassa fever virus, lymphocytic choriomeningitis virus (LCMV), Machupo virus, Marburg hemorrhagic fever virus, measles virus, Middle eastern respiratory syndrome-related coronavirus, Mokola virus, Moloney murine leukemia virus, monkey pox, mouse mammary tumor virus, mumps virus, murine gammaherpesvirus, Newcastle disease virus, Nipah virus, Nipah virus, Norwalk virus, Omsk hemorrhagic fever virus, papilloma virus, parvovirus, pseudorabies virus, Quaranfil virus, rabies virus, RD 114 Endogenous Feline Retrovirus, respiratory syncytial virus (RSV), Rift Valley fever virus, Ross River virus, rRotavirus, Rous sarcoma virus, rubella virus, Sabia-associated hemorrhagic fever virus, SARS-associated coronavirus (SARS-CoV), Sendai virus, Tacaribe virus, Thogotovirus, tick-borne encephalitis causing virus, varicella zoster virus (HHV3), varicella zoster virus (HHV3), variola major virus, variola minor virus, Venezuelan equine encephalitis virus, Venezuelan hemorrhagic fever virus, vesicular stomatitis virus (VSV), VSV-G, Vesiculovirus, West Nile virus, western equine encephalitis virus, and Zika Virus.

In other embodiments, the disclosure provides XDP of the foregoing and further comprises one or more components of a pol polyprotein (e.g., a protease), and, optionally, a second CasX or a donor template. The disclosure contemplates multiple configurations of the arrangement of the encoded components, including duplicates of some of the encoded components. The foregoing offers advantages over other vectors in the art in that viral transduction to dividing and non-dividing cells is efficient and that the XDP delivers potent and short-lived RNP that escape a subject's immune surveillance mechanisms that would otherwise detect a foreign protein. Non-limiting, exemplary XDP systems are described in PCT/US20/63488 and WO2021113772A1, incorporated by reference herein. In some embodiments, the disclosure provides host cells comprising polynucleotides or vectors encoding any of the foregoing XDP embodiments.

Upon production and recovery of the XDP comprising the CasX:gRNA RNP of any of the embodiments described herein, the XDP can be used in methods to edit target cells of subjects by the administering of such XDP, as described more fully, below.

### VII. Cells

In another aspect, provided herein are populations of cells comprising a BCL11A gene modified ex vivo by embodiments of any of the systems or methods described herein. In some embodiments, cells that have been genetically modified in this way may be administered to a subject for purposes such as gene therapy; e.g., in methods of treatment of a hemoglobinopathy-related disease, such as sickle cell disease or beta-thalassemia wherein the administration results in an increased expression of γ-globin and an increase of fetal hemoglobin (HbF) in the subject. In other embodiments, the disclosure provides compositions of modified cells for use as a medicament in the treatment of a hemoglobinopathy-related disease.

Cells of the present disclosure suitable for ex vivo modification of the BCL11A gene by a Class 2, Type V Cas nuclease and one or more guides targeted to the BCL11A target nucleic acid can be a hematopoietic progenitor cell (HPC), a hematopoietic stem cell (HSC), a CD34+ cell, a mesenchymal stem cell (MSC), an induced pluripotent stem cell (iPSC), a common myeloid progenitor cell, a proerythroblast cell, or an erythroblast cell. In some embodiments, the population of modified cells are animal cells; for example, derived from a rodent, rat, mouse, rabbit, dog cell, or a non-human primate cell; e.g., a cynomolgus monkey cell. In some embodiments, the cell is a human cell. In some cases, the cells to be modified are autologous with respect to the subject to be administered the cells. In other cases, the cells are allogeneic with respect to the subject to be administered the cells. In some cases, the ex vivo cell is isolated from the subject's bone marrow or peripheral blood.

In some embodiments, the disclosure provides methods and populations of cells modified by introducing into each cell of the population: i) a CasX:gRNA system comprising a CasX and a gRNA of any one of the embodiments described herein; ii) a CasX:gRNA system comprising a CasX, a gRNA, and a donor template of any one of the embodiments described herein; iii) a nucleic acid encoding the CasX and the gRNA, and optionally comprising the donor template; iv) a vector comprising the nucleic acid of (iii), above, which can be an AAV of any of the embodiments described herein; v) a XDP comprising the CasX:gRNA system of any one of the embodiments described herein; or vi) combinations of two or more of (i) to (v), wherein the BCL11A target nucleic acid sequence of the cells targeted by the gRNA is modified by the CasX protein and, optionally, the donor template. In some embodiments, the method further comprises administering a second gRNA or a nucleic acid encoding the second gRNA, wherein the second gRNA has a targeting sequence complementary to a different or overlapping portion of the target nucleic acid sequence compared to the first gRNA. In some cases, the CasX and gRNA are delivered to the cells of the population as an RNP (embodiments of which are described herein, supra), and, optionally, the donor template. In some embodiments, the disclosure provides a population of cells modified by the foregoing methods wherein the cells have been modified such that at least 60%, at least 70%, at least 75%, at least 80%, at least 85%, at least 90%, or at least 95% of the modified cells do not express a detectable level of BCL11A protein. In other embodiments, the disclosure provides a population of cells wherein the cells have been modified such that the expression of BCL11A protein is reduced by at least 70%, at least 75%, at least 80%, at least 85%, at least 90%, or at least 95% compared to cells that have not been modified. In still other embodiments, the disclosure provides a population of cells wherein expression of the BCL11A protein cannot be detected in the modified cells of the population. The effects of the modification can be assessed by Western blots, flow cytometry, ELISA, cell-based assays, qRT-PCR, electrochemiluminescence assays, sense transcripts can be analyzed by RNA fluorescence in situ hybridization (FISH) assay, or other methods know in the art, or as described in the Examples.

In some embodiments, the disclosure provides methods of modifying a BCL11A target nucleic acid in a population of cells by in vitro or ex vivo methods. The method provides that the cells can be obtained from a subject using any number of techniques known to the skilled artisan; e.g., a biopsy of the marrow or by obtaining a sample of the peripheral blood. The desired cells may be separated from the remainder of the sample, washed to remove fluids and debris and, optionally, placed in an appropriate buffer or media for subsequent processing steps. The method may include one or more steps of i) introducing into the cells the CasX:gRNA system components for the editing of the target nucleic acids; ii) introducing into the cells a nucleic acid or vector encoding the CasX:gRNA system components to the cells; iii) expansion of the cells in an appropriate medium under conditions suitable for their propagation, and iv) cryopreservation of the cells for subsequent administration to the subject. Thus, the CasX:gRNA systems and methods described herein can be used to modify a variety of cells associated with the hemoglobinopathy to produce populations of cells in which the expression of the BCL11A protein is reduced or eliminated and HbF is increased. This approach, therefore, could be used for methods of treatment in a subject with a hemoglobinopathy such as sickle cell anemia or beta-thalassemia, amongst others. In some cases, the cells are contacted with a CasX and a gRNA wherein the gRNA is a guide RNA (gRNA). In other cases, the cells are contacted with a CasX and a gRNA wherein the gRNA is a chimera comprising DNA and RNA. As described herein, in embodiments of any of the combinations, each of said gRNA molecules (a combination of the scaffold and targeting sequence, which can be configured as a sgRNA or a dgRNA) can be provided as an RNP with a CasX embodiment described herein for incorporation into the cells to be modified. In one embodiment, the target nucleic acid of the cell is modified by contacting the cells with a CasX protein, a guide nucleic acid (gRNA) comprising a targeting sequence complementary to the BCL11A target nucleic acid, and a donor template wherein the donor template is inserted into or replaces a portion of the target nucleic acid sequence of the cell such that the BCL11A protein is not expressed or is expressed at a reduced level. In other cases, the CasX and gRNA are delivered to the cells of the population in a vector (embodiments of which are described herein, supra), wherein the target nucleic acid is modified such that the BCL11A protein is not expressed or is expressed at a reduced level.

In some embodiments, the cells of the population are contacted with a CasX variant comprising a sequence of Table 4 or a sequence at least 65% identical, at least 70% identical, at least 75% identical, at least 80% identical, at least 81% identical, at least 82% identical, at least 83% identical, at least 84% identical, at least 85% identical, at least 86% identical, at least 86% identical, at least 87% identical, at least 88% identical, at least 89% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, or at least 99.5% identical thereto, the gRNA scaffold comprises a sequence of Table 3 or a sequence at least 65% identical, at least 70% identical, at least 75% identical, at least 80% identical, at least 81% identical, at least 82% identical, at least 83% identical, at least 84% identical, at least 85% identical, at least 86% identical, at least 86% identical, at least 87% identical, at least 88% identical, at least 89% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical thereto, and the gRNA comprises a targeting sequence selected from the group consisting of SEQ ID NOS: 272-2100 and 2286-26789 of Table 1 or a sequence at least 65% identical, at least 70% identical, at least 75% identical, at least 80% identical, at least 85% identical, at least 90% identical, or at least 95% identical thereto and having between 15 and 20 amino acids. In other cases, the CasX and the one or more gRNA are introduced into the population of cells as encoding polynucleotides using a vector; embodiments of which are described herein. Additional methods of modification of the cells using the CasX:gRNA system components include viral infection, transfection, conjugation, protoplast fusion, particle gun technology, calcium phosphate precipitation, direct microinjection, and the like. The choice of method is generally dependent on the type of cell being transformed and the circumstances under which the transformation is taking place; e.g., in vitro, ex vivo, or in vivo. A general discussion of these methods can be found in Ausubel, et al, Short Protocols in Molecular Biology, 3rd ed., Wiley & Sons, 1995.

Upon hybridization with the target nucleic acid by the CasX and the gRNA, the CasX introduces one or more single-strand breaks or double-strand breaks within the BCL11A gene that results in a modification of the target nucleic acid such as a permanent indel (deletion or insertion) or other mutation (e.g., substitution, duplication, or inversion) in the target nucleic acid that, in connection with the repair mechanisms of the host cell, results in a corresponding reduction or elimination of the expression of functional BCL11A protein, thereby creating the modified population of cells. As described herein, a CasX variant introducing double-stranded cleavage of the target nucleic acid generates a double-stranded break within 18-26 nucleotides 5′ of a PAM site on the target strand and 10-18 nucleotides 3′ on the non-target strand. Thus, in some embodiments, the resulting modification by the method can result in random insertions or deletions (indels), or a substitution, duplication, or inversion of one or more nucleotides in those region by non-homologous DNA end joining (NHEJ) repair mechanisms.

In some embodiments of the method of modifying a population of cells, the first gRNA comprises a targeting sequence complementary to a sequence proximal to or within any one of BCL11A gene exons. In one embodiment, the first gRNA comprises a targeting sequence complementary to a sequence proximal to or within or adjacent to any one of the regulatory elements of the BCL11A gene. In a particular embodiment, the first gRNA comprises a targeting sequence complementary to a sequence within or 5′ adjacent to the GATA1 binding motif sequence of the BCL11A gene. In a particular embodiment, the targeting sequence is SEQ ID NO: 22. By the foregoing, disruption of the target nucleic acid sequence results in a modification of the BCL11A gene such that expression of functional BCL11A protein is reduced or eliminated in the modified cells of the population.

In some embodiments of the method, the target nucleic acid of the cells of the population is modified using a plurality of gRNAs (e.g., two, three, four or more) targeted to different or overlapping portions of the BCL11A gene wherein the CasX protein introduces multiple breaks in the target nucleic acid sequence that result in a permanent indel (deletion or insertion) or other mutations (e.g., a substitution, duplication, or inversion of one or more nucleotides) such that expression of functional BCL11A protein is reduced or eliminated in the modified cells of the population.

In other embodiments, disclosure provides populations of cells modified by contacting the cell with a CasX protein, one or more gRNA comprising a targeting sequence, and a donor template of any of the embodiments described herein wherein the donor template is inserted into the break sites introduced by the nuclease, replacing all or a portion of the target nucleic acid sequence of the BCL11A gene to be modified. In one embodiment of the foregoing, the donor template comprises at least a portion of a BCL11A exon and one or more mutations and the modification of the cell results in a modification of the gene such that expression of functional BCL11A protein is reduced or eliminated in the modified cells of the population. In another embodiment of the foregoing, the donor template comprises a sequence within or 5′ adjacent to the GATA1 binding motif sequence but having one or more mutations relative to the wild-type sequence and the modification of the cell results in a reduction or elimination of expression of functional BCL11A protein in the modified cells of the population. It will be understood that in such cases, the donor template replacements are larger in the 5′ and 3′ direction than the location of the cleavage sites introduced by the nuclease in the specific portions of the target nucleic acid to be replaced and would further comprise homologous arms that are 5′ and 3′ to the cleavage sites introduced by the nuclease to facilitate its insertion. In some cases, the donor template is a single-stranded DNA template or a single stranded RNA template. In other cases, the donor template is a double-stranded DNA template. The insertion of the donor template at the target region which can be mediated by homology-directed repair (HDR, as described, supra) or homology-independent targeted integration (HITI). The exogenous sequence inserted by HITI can be any length, for example, a relatively short sequence of between 10 and 50 nucleotides in length, or a longer sequence of about 50-1000 nucleotides in length. The donor template sequence may comprise certain sequence differences as compared to the genomic sequence, e.g., restriction sites, nucleotide polymorphisms, barcodes, selectable markers (e.g., drug resistance genes, fluorescent proteins, enzymes etc.), etc., which may be used to assess for successful insertion of the donor nucleic acid at the cleavage site or, in some cases, may be used for other purposes (e.g., to signify expression at the targeted genomic locus). Alternatively, these sequence differences may include flanking recombination sequences such as FLPs, loxP sequences, or the like, that can be activated at a later time for removal of the marker sequence.

In some embodiments of the method of modifying the population of cells, the method further comprises contacting the BCL11A gene target nucleic acid sequence of the population of cells with: i) an additional CRISPR nuclease and a gRNA targeting a different or overlapping portion of the BCL11A target nucleic acid compared to the first gRNA; ii) a polynucleotide encoding the additional CRISPR nuclease and the gRNA of (i); iii) a vector comprising the polynucleotide of (ii); or iv) a XDP comprising the additional CRISPR nuclease and the gRNA of (i), wherein the contacting results in modification of the BCL11A gene at a different location in the sequence compared to the sequence targeted by the first gRNA. In one embodiment of the foregoing, the additional CRISPR nuclease is a CasX protein having a sequence different from the CasX protein of the previous embodiments. In another embodiment of the foregoing, the additional CRISPR nuclease is not a CasX protein and is selected from the group consisting of Cas9, Cas12a, Cas12b, Cas12c, Cas12d (CasY), Cas12j, Cask, Cas13a, Cas13b, Cas13c, Cas13d, Cas14, Cpf1, C2cl, Csn2, and sequence variants thereof.

In other embodiments, the disclosure provides methods of modifying a BCL11A target nucleic acid in a population of cells in vivo in a subject. In one embodiment of the method of in vivo modification, the method comprises administration of a vector of the embodiments described herein to the subject at a therapeutically effective dose. In some embodiments, the vector is administered to the subject at a dose of at least about 1×10⁵ vector genomes (vg/kg), at least about 1×10⁶ vg/kg, at least about 1×10⁷ vg/kg, at least about 1×10⁸ vg/kg, at least about 1×10⁹ vg/kg, at least about 1×10¹⁰ vg/kg, at least about 1×10¹¹ vg/kg, at least about 1×10¹² vg/kg, at least about 1×10¹³ vg/kg, at least about 1×10¹⁴ vg/kg, at least about 1×10¹⁵ vg/kg, or at least about 1×10¹⁶ vg/kg. In other embodiments, the vector is administered to the subject at a dose of at least about 1×10⁵ vg/kg to at least about 1×10¹⁶ vg/kg, or at least about 1×10⁶ vg/kg to about 1×10¹⁵ vg/kg, or at least about 1×10⁷ vg/kg to about 1×10¹⁴ vg/kg, or at least about 1×10⁸ vg/kg to about 1×10¹⁴ vg/kg. In another embodiment of the method of in vivo modification, the method comprises administration of a XDP to the subject at a therapeutically effective dose, wherein the XDP comprises a CasX and gRNA complexed in an RNP and, optionally, a donor template (described more fully, supra), wherein the XDP has tropism for the target cells and is able to deliver the RNP for the editing of the BCL11A gene, as described herein. The XDP embodiments utilized in the foregoing method of editing are described herein. In one embodiment, the XDP is administered to the subject at a dose of at least about 1×10⁵ particles/kg, at least about 1×10⁶ particles/kg, at least about 1×10⁷ particles/kg at least about 1×10⁸ particles/kg, at least about 1×10⁹ particles/kg, at least about 1×10¹⁰ particles/kg, at least about 1×10¹¹ particles/kg, at least about 1×10¹² particles/kg, at least about 1×10¹³ particles/kg, at least about 1×10¹⁴ particles/kg, at least about 1×10¹⁵ particles/kg, at least about 1×10¹⁶ particles/kg, or at least about 1×10⁶ particles/kg to about 1×10¹⁵ particles/kg, or at least about 1×10⁷ particles/kg to about 1×10¹⁴ particles/kg. In the foregoing embodiments of the paragraph, the vector or XDP is administered to the subject by a route of administration selected from intraparenchymal, intravenous, intra-arterial, intraperitoneal, intracapsular, subcutaneously, intramuscularly, intraabdominally, or combinations thereof, wherein the administering method is injection, transfusion, or implantation.

### VIII Therapeutic Methods

In another aspect, the present disclosure relates to methods of treating a hemoglobinopathy-related disease in a subject in need thereof, including but not limited to sickle-cell disease or beta-thalassemia in which repression or elimination of expression of the BCL11A protein by modifying the BCL11A gene in target cells of the subject ameliorates the signs, symptoms, or effects of the disease, notwithstanding that the subject may still be afflicted with the underlying disease.

A number of therapeutic strategies have been used to design the compositions for use in the methods of treatment of a subject with a hemoglobinopathy-related disease. In some embodiments, the method comprises administering to the subject having a hemoglobinopathy (e.g., sickle cell anemia or beta-thalassemia) a therapeutically effective dose of a Class 2, Type V CRISPR nuclease and guide RNA disclosed herein. In some embodiments, the method of treatment comprises administering to the subject a therapeutically effective dose of: i) the CasX:gRNA system comprising a first CasX protein and a first gRNA with a targeting sequence complementary to the target nucleic acid; ii) the CasX:gRNA system comprising a first CasX protein and a first gRNA with a targeting sequence complementary to the target nucleic acid and a donor template; iii) a nucleic acid encoding the CasX:gRNA system of (i) or (ii); iv) a vector comprising the nucleic acid of (iii), which can be an AAV of any of the embodiments described herein; v) a XDP comprising the CasX:gRNA system of (i) or (ii); or vi) combinations of two or more of (i)-(v), wherein 1) the BCL11A gene of the cells of the subject targeted by the first gRNA is modified (e.g., knocked-down or knocked-out) by the CasX protein and, optionally, the donor template; and 2) an increase in production of hemoglobin F (HbF) results in the subject. In some embodiments, the method of treating further comprises administering a second gRNA or a nucleic acid encoding the second gRNA, wherein the second gRNA has a targeting sequence complementary to a different or overlapping portion of the target nucleic acid sequence compared to the first gRNA. In some cases, the cells targeted for modification are selected from the group consisting of hematopoietic stem cells (HSC), hematopoietic progenitor cells (HPC), CD34+ cells, mesenchymal stem cells (MSC), induced pluripotent stem cells (iPSC), common myeloid progenitor cells, proerythroblast cells, and erythroblast cells. In some embodiments, the subject to be treated is selected from the group consisting of rodent, mouse, rat, and non-human primate. In another embodiment, the subject is a human.

In some embodiments of the method of treatment, the vector is an AAV vector encoding the CasX:gRNA system, and is administered to the subject at a dose of at least about 1×10⁵ vector genomes/kg (vg/kg), at least about 1×10⁶ vg/kg, at least about 1×10⁷ vg/kg, at least about 1×10⁸ vg/kg, at least about 1×10⁹ vg/kg, at least about 1×10¹⁰ vg/kg, at least about 1×10¹¹ vg/kg, at least about 1×10¹² vg/kg, at least about 1×10¹³ vg/kg, at least about 1×10¹⁴ vg/kg, at least about 1×10¹⁵ vg/kg, or at least about 1×10¹⁶ vg/kg. In other embodiments of the method, the AAV vector is administered to the subject at a dose of at least about 1×10⁵ vg/kg to about 1×10¹⁶ vg/kg, at least about 1×10⁶ vg/kg to about 1×10¹⁵ vg/kg, or at least about 1×10⁷ vg/kg to about 1×10¹⁴ vg/kg. In other embodiments, the method of treatment comprises administering a therapeutically effective dose of a XDP comprising the CasX:gRNA system to the subject. In one embodiment, the XDP is administered to the subject at a dose of at least about 1×10⁵ particles/kg, at least about 1×10⁶ particles/kg, at least about 1×10⁷ particles/kg at least about 1×10⁸ particles/kg, at least about 1×10⁹ particles/kg, at least about 1×10¹⁰ particles/kg, at least about 1×10¹¹ particles/kg, at least about 1×10¹² particles/kg, at least about 1×10¹³ particles/kg, at least about 1×10¹⁴ particles/kg, at least about 1×10¹⁵ particles/kg, at least about 1×10¹⁶ particles/kg. In another embodiment, the XDP is administered to the subject at a dose of at least about 1×10⁵ particles/kg to about 1×10¹⁶ particles/kg, or at least about 1×10⁶ particles/kg to about 1×10¹⁵ particles/kg, or at least about 1×10⁷ particles/kg to about 1×10¹⁴ particles/kg. In the foregoing embodiments of the paragraph, the vector or XDP is administered to the subject by a route of administration selected from intraparenchymal, intravenous, intra-arterial, intraperitoneal, intracapsular, subcutaneously, intramuscularly, intraabdominally, or combinations thereof, wherein the administering method is injection, transfusion, or implantation. The administration can be once, twice, or can be administered multiple times using a regimen schedule of weekly, every two weeks, monthly, quarterly, or every six months.

In some embodiments, the method of treatment comprises administering a vector comprising a polynucleotide encoding a CasX and a plurality of gRNAs targeted to different or overlapping regions of the BCL11A gene to a subject, wherein the administration results in contacting the subject target nucleic acid sequence with the expression product(s) of the vectors within a cell of the subject, and wherein the BCL11A gene is modified in the cell of the subject. In other embodiments of the methods of treatment, the methods comprise administering to a subject a vector encoding the CasX protein and the gRNA, and further comprising a donor template, wherein said administering results in modification of the target nucleic acid sequence of a cell of the subject by cleavage by the CasX protein and insertion of the donor template into the target nucleic acid. In other embodiments, the methods comprise administering a first vector comprising a polynucleotide encoding a CasX and a plurality of gRNAs targeted to different or overlapping sequences of the BCL11A gene and a second vector comprising a donor template polynucleotide encoding at least a portion of or the entirety of a BCL11A gene to a subject, wherein the administration of the vectors results in contacting the subject target nucleic acid sequence within a cell of the subject with the expression product(s) of the CasX and gRNA vectors and the donor template, wherein the BCL11A gene is modified in the cell of the subject, as described herein. In some embodiments of the methods of treatment, the vector administered to the subject is an AAV vector as described herein. In the foregoing, the AAV vector is selected from AAV1, AAV2, AAV3, AAV4, AAV5, AAV6, AAV7, AAV8, AAV9, AAV10, AAV-Rh74, or AAVRh10. In some embodiments of the methods of treatment, the vector administered to the subject is a XDP as described herein, comprising an RNP of a CasX:gRNA system.

In some embodiments of the method, the modifying comprises introducing a single-stranded break in the BCL11A gene of the cells of a population. In other cases, the modifying comprises introducing a double-stranded break in the BCL11A gene of the cells of a population. In some embodiments, the modifying introduces one or more mutations in the BCL11A target nucleic acid, such as an insertion, deletion, substitution, duplication, or inversion of one or more nucleotides in the BCL11A gene, wherein expression of BCL11A protein in the cells of the subject is reduced by at least about 10%, at least about 20%, at least about 30%, at least about 40%, at least about 50%, at least about 60%, at least about 70%, at least about 80%, or at least about 90% in comparison to a cell that has not been modified. In some cases, the BCL11A gene of the cells of the subject are modified such that at least 70%, at least 75%, at least 80%, at least 85%, at least 90%, or at least 95% of the modified cells do not express a detectable level of BCL11A protein. In other cases of the method of treatment, the modifying results in an increased production of HbF in the circulating blood of the subject of at least about 5%, at least about 10%, at least about 20%, at least about 30%, at least about 40%, or at least about 50% compared to the levels of HbF in the subject prior to treatment. In other embodiments, the method results in a ratio of HbF to hemoglobin S (HbS) in the circulating blood of the subject of at least 0.01:1.0, at least 0.025:1.0, at least 0.05:1.0, at least 0.075:1.0 at least 0.1:1.0, at least 0.2:1.0, at least 0.3:1.0, at least 0.4:1.0, at least 0.5:1:0, at least 0.75:1.0, at least 1.0:1.0, at least 1.25:1.0, at least 1.5:1.0, or at least 1.75:1.0. In other embodiments, the method results in HbF levels of at least about 5%, or at least about 10%, or at least about 20%, or at least about 30% of total hemoglobin in the circulating blood of the subject. In the foregoing embodiments, the subject is selected from the group consisting of mouse, rat, pig, non-human primate, and human. Methods of obtaining samples from treated subjects for analysis to determine the effectiveness of the treatment, such as body fluids or tissues, and methods of preparation of the samples to allow for analysis are well known to those skilled in the art. Methods for analysis of RNA and protein levels are discussed above and are well known to those skilled in the art. The effects of treatment can also be assessed by measuring biomarkers associated with the target gene expression in the aforementioned fluids, tissues or organs, collected from an animal contacted with one or more compounds of the invention, by routine clinical methods known in the art. Biomarkers of hemoglobinopathy diseases include, but are not limited to, percentage of sickle cells in circulating blood, BCL11A levels, BCL11A RNA, hemoglobin S levels, hemoglobin-gamma levels, and hemoglobin F levels.

In some cases, the method of treating a hemoglobinopathy in a subject further comprises administering a therapeutically effective dose of an additional CRISPR nuclease, or a polynucleotide encoding the additional CRISPR nuclease. In one embodiment, the additional CRISPR nuclease is a CasX protein having a sequence different from the first CasX. In another embodiment, the additional CRISPR nuclease is not a CasX protein; i.e., is a Cas9, Cas12a, Cas12b, Cas12c, Cas12d (CasY), Cas12j, Cas12k, Cas13a, Cas13b, Cas13c, Cas13d, Cas14, Cpf1, C2cl, Csn2, or is a sequence variant thereof. In some embodiments, the method of treating a hemoglobinopathy in a subject further comprises administering a chemotherapeutic agent.

In other embodiments, the disclosure provides methods of treating a hemoglobinopathy-related disease in a subject in need thereof by the administration to the subject of a therapeutically effective amount of a population of cells modified in vitro or ex vivo by CasX:gRNA system compositions of the embodiments described herein, including i) the CasX:gRNA system comprising a first CasX protein and a first gRNA with a targeting sequence complementary to the target nucleic acid; ii) the CasX:gRNA system comprising a first CasX protein and a first gRNA with a targeting sequence complementary to the target nucleic acid and a donor template; iii) a nucleic acid encoding the CasX:gRNA system of (i) or (ii); iv) a vector comprising the nucleic acid of (iii), which can be an AAV of any of the embodiments described herein; v) a XDP comprising the CasX:gRNA system of (i) or (ii); or vi) combinations of two or more of (i)-(v). In one embodiment, the method of treatment comprises: i) isolating induced pluripotent stem cells (iPSC) or hematopoietic stem cells (HSC) from a subject; ii) modifying the BCL11A target nucleic acid of the iPSC or HSC by the methods of any of the embodiments described herein; iii) differentiating the modified iPSC or HSC into a hematopoietic progenitor cell; and iv) implanting the hematopoietic progenitor cell into the subject with the hemoglobinopathy, wherein the method results in an increased levels of hemoglobin F (HbF) in circulating blood of the subject of at least about 5%, at least about 10%, at least about 20%, at least about 30%, at least about 40%, or at least about 50% compared to the levels of HbF in the subject prior to treatment. In some cases, the cells are autologous with respect to the subject to be administered the cells and are isolated from the subject's bone marrow or peripheral blood. In other cases, the cells are allogeneic with respect to the subject to be administered the cells and are isolated from a different subject's bone marrow or peripheral blood. The modified cells can be implanted into the subject by transplantation, local injection, systemic infusion, or combinations thereof. The methods to modify the cells for administration to a subject have been described herein, but, briefly, the modifying comprises contacting the cells with: i) the CasX:gRNA system comprising a first CasX protein and a first gRNA with a targeting sequence complementary to the target nucleic acid; ii) the CasX:gRNA system comprising a first CasX protein and a first gRNA with a targeting sequence complementary to the target nucleic acid and a donor template; iii) a nucleic acid encoding the CasX:gRNA system of (i) or (ii); iv) a vector comprising the nucleic acid of (iii), which can be an AAV of any of the embodiments described herein; v) a XDP comprising the CasX:gRNA system of (i) or (ii); or vi) combinations of two or more of (i)-(v), wherein expression of the BCL11A protein is reduced or the cell does not express a detectable level of the BCL11A protein. In some embodiments, the method further comprises administering a second gRNA or a nucleic acid encoding the second gRNA, wherein the second gRNA has a targeting sequence complementary to a different or overlapping portion of the target nucleic acid sequence compared to the first gRNA. In some cases, the CasX and gRNA is delivered to the cells of the population as an RNP (embodiments of which are described herein, supra), and, optionally, the donor template, wherein the target nucleic acid is modified such that the BCL11A protein is not expressed or is expressed at a reduced level. In other cases, the CasX and gRNA is delivered to the cells of the population in a vector (embodiments of which are described herein, supra), wherein the target nucleic acid is modified such that the BCL11A protein is not expressed or is expressed at a reduced level. In some embodiments, the cells of the population to be modified by the administration of the compositions are eukaryotic cells selected from the group consisting of rodent cells, mouse cells, rat cells, and non-human primate cells. In some embodiments, the eukaryotic cells are human cells. In some embodiments, the eukaryotic cell is selected from the group consisting of a hematopoietic stem cell (HSC), a hematopoietic progenitor cell (HPC), a CD34+ cell, a mesenchymal stem cell (MSC), induced pluripotent stem cell (iPSC), a common myeloid progenitor cell, a proerythroblast cell, and an erythroblast cell. In some embodiments of the method, the cells or their progeny administered to the subject persist in the subject for at least one month, two month, three months, four months, five months, six months, seven months, eight months, nine months, ten months, eleven months, twelve months, thirteen months, fourteen month, fifteen months, sixteen months, seventeen months, eighteen months, nineteen months, twenty months, twenty-one months, twenty-two months, twenty-three months, two years, three years, four years, or five years after administration to the subject. In some embodiments, the methods of treatment of the disclosure result in an increased levels of hemoglobin F (HbF) in circulating blood of at least about 5%, at least about 10%, at least about 20%, at least about 30%, at least about 40%, or at least about 50% compared to the levels of HbF in the subject prior to treatment. In other embodiments, the method results in a ratio of HbF to hemoglobin S (HbS) in the subject of at least 0.01:1.0, at least 0.025:1.0, at least 0.05:1.0, at least 0.075:1.0 at least 0.1:1.0, at least 0.2:1.0, at least 0.3:1.0, at least 0.4:1.0, at least 0.5:1:0, at least 0.75:1.0, at least 1.0:1.0, at least 1.25:1.0, at least 1.5:1.0, or at least 1.75:1.0. In other embodiments, the method results in HbF levels of at least about 5%, or at least about 10%, or at lease about 20%, or at least about 30% of total circulating hemoglobin in the subject.

In other embodiments, the disclosure provides methods of increasing fetal hemoglobin (HbF) in a subject having a hemoglobinopathy by genome editing, the method comprising: i) administering to the subject an effective dose of a vector or a XDP embodiment described herein, wherein the vector or XDP delivers the CasX:gRNA system to cells of the subject; ii) the BCL11A target nucleic acid of cells of the subject are edited by the CasX targeted by the first gRNA; iii) the editing comprises introducing an insertion, deletion, substitution, duplication, or inversion of one or more nucleotides in the target nucleic acid sequence such that expression of BCL11A protein is reduced or eliminated, wherein the method results in an increased levels of hemoglobin F (HbF) in circulating blood of the subject of at least about 5%, at least about 10%, at least about 20%, at least about 30%, at least about 40%, or at least about 50% compared to the levels of HbF in the subject prior to treatment. In the foregoing, the cells are selected from the group consisting of hematopoietic stem cells (HSC), hematopoietic progenitor cells (HPC), CD34+ cells, mesenchymal stem cells (MSC), induced pluripotent stem cells (iPSC), common myeloid progenitor cells, proerythroblast cells, and erythroblast cells. In one embodiment of the method, the target nucleic acid of the cells has been edited such that expression of the BCL11A protein is reduced by at least about 10%, at least about 20%, at least about 30%, at least about 40%, at least about 50%, at least about 60%, at least about 70%, at least about 80%, or at least about 90% in comparison to target nucleic acid of cells that have not been edited. In some cases, the subject is selected from the group consisting of mouse, rat, pig, and non-human primate. In other cases, the subject is a human.

In some embodiments of the method of treating a hemoglobinopathy in a subject, the method results in improvement in at least one clinically-relevant parameter selected from the group consisting of occurrence of end-organ disease, albuminuria, hypertension, hyposthenia, hyposthenuria, diastolic dysfunction, functional exercise capacity, acute coronary syndrome, pain events, pain severity, anemia, hemolysis, tissue hypoxia, organ dysfunction, abnormal hematocrit values, childhood mortality, incidence of strokes, hemoglobin levels compared to baseline, HbF levels, reduced incidence of pulmonary embolisms, incidence of vaso-occlusive crises, concentration of hemoglobin S in erythrocytes, rate of hospitalizations, liver iron concentration, required blood transfusions, and quality of life score. In other embodiments of the method of treating a hemoglobinopathy in a subject, the method results in improvement in at least two clinically-relevant parameters selected from the group consisting of occurrence of end-organ disease, albuminuria, hypertension, hyposthenia, hyposthenuria, diastolic dysfunction, functional exercise capacity, acute coronary syndrome, pain events, pain severity, anemia, hemolysis, tissue hypoxia, organ dysfunction, abnormal hematocrit values, childhood mortality, incidence of strokes, hemoglobin levels compared to baseline, HbF levels, reduced incidence of pulmonary embolisms, incidence of vaso-occlusive crises, concentration of hemoglobin S in erythrocytes, rate of hospitalizations, liver iron concentration, required blood transfusions, and quality of life score.

In some embodiments, the method of treatment comprises administering to the subject a liposome or lipid nanoparticle comprising the CasX protein and the gRNA. In some embodiments, the liposome or lipid nanoparticle further comprises a donor template of any of the embodiments described herein.

In some embodiments, the disclosure provides a method of treatment of a subject having a hemoglobinopathy-related disease, the method comprising administering to the subject a CasX:gRNA composition, or a vector, or a XDP comprising an RNP of the CasX:gRNA composition of any of the embodiments disclosed herein according to a treatment regimen comprising one or more consecutive doses using a therapeutically effective dose. In some embodiments of the treatment regimen, the therapeutically effective dose of the composition or vector is administered as a single dose. In other embodiments of the treatment regimen, the therapeutically effective dose is administered to the subject as two or more doses over a period of at least two weeks, or at least one month, or at least two months, or at least three months, or at least four months, or at least five months, or at least six months. In some embodiments of the treatment regimen, the effective doses are administered by a route selected from the group consisting of transplantation, local injection, systemic infusion, or combinations thereof.

In some embodiments, the methods of treatment further comprise administering a chemotherapeutic agent wherein the agent is effective in improving the signs or symptoms associated with a hemoglobinopathy-related disease, including but not limited to hydroxyurea, L-glutamine oral powder, voxelotor, and analgesics.

In some embodiments, the present disclosure provides a CasX:gRNA composition, a nucleic acid encoding a CasX:gRNA composition, a vector comprising the nucleic acid, or a XDP comprising an RNP of the CasX:gRNA for use as a medicament for the treatment of a hemoglobinopathy, including sickle-cell disease or beta-thalassemia.

### XIV. Kits and Compositions

In other embodiments, provided herein are kits comprising a CasX protein, one or a plurality of gRNA of any of the embodiments of the disclosure comprising a targeting sequence specific for a BCL11A gene, and a suitable container (for example a tube, vial or plate). In some embodiments, the kit further comprises a buffer, a nuclease inhibitor, a protease inhibitor, a liposome, a therapeutic agent, a label, a label visualization reagent, or any combination of the foregoing. In some embodiments, the kit further comprises a pharmaceutically acceptable carrier, diluent or excipient. In some embodiments, the kit comprises appropriate control compositions for gene modifying applications, and instructions for use. In some embodiments, the kit comprises a vector comprising a sequence encoding a CasX protein of the disclosure, a gRNA of the disclosure, optionally a donor template, or a combination thereof.

In other embodiments of the kits of the disclosure, the kit comprises a composition for the treatment of a hemoglobinopathy in a subject by modifying a BCL11A target nucleic acid in isolated cells of the subject, the modifying comprising contacting the target nucleic acid sequence of the cells with an embodiment disclosed herein of: i) a CasX:gRNA system; ii) a nucleic acid encoding the components of the CasX:gRNA system; iii) a vector comprising the nucleic acid; iv) a XDP comprising a CasX protein and a guide nucleic acid (gRNA); or v) combinations of any of (i)-(iv), wherein i) said contacting results in modification of the BCL11A target nucleic acid sequence by the CasX protein; ii) reduced expression of the BCL11A protein; and iii) increased production of hemoglobin F (HbF) upon maturation of the cells. In some cases, the cell is an induced pluripotent stem cell (iPSC). In other cases, the cell is a hematopoietic stem cell (HSC). In one embodiment, the use of the composition results in reduction of expression of the BCL11A protein by the matured cells is reduced by at least about 10%, at least about 20%, at least about 30%, at least about 40%, at least about 50%, at least about 60%, at least about 70%, at least about 80%, or at least about 90% in comparison to target nucleic acid that has not been modified. In another embodiment, expression of the BCL11A protein by the matured cells cannot be detected.

In some embodiments, the kit comprises a plurality of cells edited using the CasX:gRNA systems described herein.

The present description sets forth numerous exemplary configurations, methods, parameters, and the like. It should be recognized, however, that such description is not intended as a limitation on the scope of the present disclosure, but is instead provided as a description of exemplary embodiments.

### Enumerated Embodiments

The invention may be defined by reference to the following enumerated, illustrative embodiments.

### Set I

Embodiment 1. A composition comprising a Class 2 Type V CRISPR protein and a first guide nucleic acid (gNA), wherein the gNA comprises a targeting sequence complementary to a polypyrimidine tract-binding protein 1 (BCL11A) gene target nucleic acid sequence.

Embodiment 2. The composition of embodiment 1, wherein the gNA comprises a targeting sequence complementary to a target nucleic acid sequence selected from the group consisting of:
a. a BCL11A intron;
b. a BCL11A exon;
c. a BCL11A intron-exon junction;
d. a BCL11A regulatory element; and
e. an intergenic region.

Embodiment 3. The composition of embodiment 1, wherein the BCL11A gene comprises a wild-type sequence.

Embodiment 4. The composition of any one of embodiments 1-3, wherein the gNA is a guide RNA (gRNA).

Embodiment 5. The composition of any one of embodiments 1-3, wherein the gNA is a guide DNA (gDNA).

Embodiment 6. The composition of any one of embodiments 1-3, wherein the gNA is a chimera comprising DNA and RNA.

Embodiment The composition of any one of embodiments 1-6, wherein the gNA is a single-molecule gNA (sgNA).

Embodiment 8. The composition of any one of embodiments 1-6, wherein the gNA is a dual-molecule gNA (dgNA).

Embodiment 9. The composition of any one of embodiments 1-8, wherein the targeting sequence of the gNA comprises a sequence selected from the group consisting of SEQ ID NOS: 272-2100 and 2286-26789, or a sequence having at least about 65%, at least about 75%, at least about 85%, or at least about 95% identity thereto.

Embodiment 10. The composition of any one of embodiments 1-8, wherein the targeting sequence of the gNA comprises a sequence selected from the group consisting of the SEQ ID NOS: 272-2100 and 2286-26789.

Embodiment 11. The composition of any one of embodiments 1-8, wherein the targeting sequence of the gNA comprises a sequence of SEQ ID NOS: 272-2100 and 2286-26789 with a single nucleotide removed from the 3′ end of the sequence.

Embodiment 12. The composition of any one of embodiments 1-8, wherein the targeting sequence of the gNA comprises a sequence of SEQ ID NOS: 272-2100 and 2286-26789 with two nucleotides removed from the 3′ end of the sequence.

Embodiment 13. The composition of any one of embodiments 1-8, wherein the targeting sequence of the gNA comprises a sequence of SEQ ID NOS: 272-2100 and 2286-26789 with three nucleotides removed from the 3′ end of the sequence.

Embodiment 14. The composition of any one of embodiments 1-8, wherein the targeting sequence of the gNA comprises a sequence of SEQ ID NOS: 272-2100 and 2286-26789 with four nucleotides removed from the 3′ end of the sequence.

Embodiment 15. The composition of any one of embodiments 1-8, wherein the targeting sequence of the gNA comprises a sequence of SEQ ID NOS: 272-2100 and 2286-26789 with five nucleotides removed from the 3′ end of the sequence.

Embodiment 16. The composition of any one of embodiments 1-15, wherein the targeting sequence of the gNA is complementary to a sequence of a BCL11A exon.

Embodiment 17. The composition of embodiment 16, wherein the targeting sequence of the gNA is complementary to a sequence selected from the group consisting of a BCL11A exon 1 sequence, BCL11A exon 2 sequence, BCL11A exon 3 sequence, BCL11A exon 4 sequence, BCL11A exon 5 sequence, BCL11A exon 6 sequence, BCL11A exon 7 sequence, BCL11A exon 8 sequence, and a BCL11A exon 9 sequence.

Embodiment 18. The composition of embodiment 17, wherein the targeting sequence of the gNA is complementary to a sequence selected from the group consisting of a BCL11A exon 1 sequence, BCL11A exon 2 sequence, and a BCL11A exon 3 sequence.

Embodiment 19. The composition of any one of embodiments 1-15, wherein the targeting sequence of the gNA is complementary to a sequence of a BCL11A regulatory element.

Embodiment 20. The composition of embodiment 19, wherein the targeting sequence of the gNA is complementary to a sequence of a promoter of the BCL11A gene.

Embodiment 21. The composition of embodiment 19, wherein the targeting sequence of the gNA is complementary to a sequence of an enhancer regulatory element.

Embodiment 22. The composition of embodiment 21, wherein the targeting sequence of the gNA is complementary to a sequence that comprises a GATA1 erythroid-specific enhancer binding site (GATA1) of the BCL11A gene.

Embodiment 23. The composition of embodiment 22, wherein the targeting sequence of the gNA has the sequence UGGAGCCUGUGAUAAAAGCA (SEQ ID NO: 22), or a sequence having at least 90% or 95% sequence identity thereto.

Embodiment 24. The composition of embodiment 22, wherein the targeting sequence of the gNA consists of the sequence UGGAGCCUGUGAUAAAAGCA (SEQ ID NO: 22).

Embodiment 25. The composition of embodiment 21, wherein the targeting sequence of the gNA has the sequence UGCUUUUAUCACAGGCUCCA (SEQ ID NO: 23), or a sequence having at least 90% or 95% sequence identity thereto.

Embodiment 26. The composition of embodiment 21, wherein the targeting sequence of the gNA consists of the sequence UGCUUUUAUCACAGGCUCCA (SEQ ID NO: 23).

Embodiment 27. The composition of any one of embodiments 1-26, further comprising a second gNA, wherein the second gNA has a targeting sequence complementary to a different or overlapping portion of the BCL11A target nucleic acid compared to the targeting sequence of the gNA of the first gNA.

Embodiment 28. The composition of embodiment 27, wherein the targeting sequence of the second gNA is complementary to a sequence of the target nucleic acid that is 5′ or 3′ to the GATA1 binding site sequence.

Embodiment 29. The composition of embodiment 27, wherein the second gNA has a targeting sequence complementary to the same exon targeted by the first gNA.

Embodiment 30. The composition of any one of embodiments 1-29, wherein the first or second gNA has a scaffold comprising a sequence having at least about 50%, at least about 60%, at least about 70%, at least about 80%, at least about 90%, at least about 95%, at least about 96%, at least about 97%, at least about 98%, at least about 99% sequence identity to a sequence selected from the group consisting of SEQ ID NOS: 4-16 and 2101-2285 as set forth in Tables 1 and 2.

Embodiment 31. The composition of any one of embodiments 1-30, wherein the first or second gNA has a scaffold comprising a sequence selected from the group consisting of SEQ ID NOs:2101-2285.

Embodiment 32. The composition of any one of embodiments 1-30, wherein the first or second gNA has a scaffold consisting of a sequence selected from the group consisting of SEQ ID NOs:2101-2285.

Embodiment 33. The composition of any one of embodiments 1-30, wherein the first or second gNA scaffold comprises a sequence having at least one modification relative to a reference gNA sequence selected from the group consisting of SEQ ID NOS: 4-16.

Embodiment 34. The composition of embodiment 33, wherein the at least one modification of the reference gNA comprises at least one substitution, deletion, or substitution of a nucleotide of the reference gNA sequence.

Embodiment 35. The composition of any one of embodiments 1-34, wherein the first or second gNA variant comprises a targeting sequence of UGGAGCCUGUGAUAAAAGCA (SEQ ID NO: 22).

Embodiment 36. The composition of any one of embodiments 1-35, wherein the first or second gNA is chemically modified.

Embodiment 37. The composition of any one of embodiments 1-36, wherein the Class 2 Type V CRISPR protein is a reference CasX protein having a sequence of any one of SEQ ID NOS: 1-3, a CasX variant protein having a sequence SEQ ID NOS: 36-99 or 101-148 as set forth in Table 4, or a sequence having at least about 50%, at least about 60%, at least about 70%, at least about 80%, at least about 90%, or at least about 95%, or at least about 95%, or at least about 96%, or at least about 97%, or at least about 98%, or at least about 99% sequence identity thereto.

Embodiment 38. The composition of embodiment 37, wherein the Class 2 Type V CRISPR protein is a CasX variant protein having a sequence of SEQ ID NOS: 36-99 or 101-148.

Embodiment 39. The composition of embodiment 37, wherein the CasX variant protein consists of a sequence of SEQ ID NOS: 36-99 or 101-148.

Embodiment 40. The composition of embodiment 37, wherein the CasX variant protein comprises at least one modification relative to a reference CasX protein having a sequence selected from SEQ ID NOS:1-3.

Embodiment 41. The composition of embodiment 40, wherein the at least one modification comprises at least one amino acid substitution, deletion, or substitution in a domain of the CasX variant protein relative to the reference CasX protein.

Embodiment 42. The composition of embodiment 41, wherein the domain is selected from the group consisting of a non-target strand binding (NTSB) domain, a target strand loading (TSL) domain, a helical I domain, a helical II domain, an oligonucleotide binding domain (OBD), and a RuvC DNA cleavage domain.

Embodiment 43. The composition of any one of embodiments 37-42, wherein the CasX protein further comprises one or more nuclear localization signals (NLS).

Embodiment 44. The composition of embodiment 43, wherein the one or more NLS are selected from the group of sequences consisting of PKKKRKV (SEQ ID NO: 168), KRPAATKKAGQAKKKK (SEQ ID NO: 169), PAAKRVKLD (SEQ ID NO: 170), RQRRNELKRSP (SEQ ID NO: 171), NQSSNFGPMKGGNFGGRSSGPYGGGGQYFAKPRNQGGY (SEQ ID NO: 172), RMRIZFKNKGKDTAELRRRRVEVSVELRKAKKDEQILKRRNV (SEQ ID NO: 173), VSRKRPRP (SEQ ID NO: 174), PPKKARED (SEQ ID NO: 175), PQPKKKPL (SEQ ID NO: 176), SALIKKKKKMAP (SEQ ID NO: 177), DRLRR (SEQ ID NO: 178), PKQKKRK (SEQ ID NO: 179), RKLKKKIKKL (SEQ ID NO: 180), REKKKFLKRR (SEQ ID NO: 181), KRKGDEVDGVDEVAKKKSKK (ESQ ID NO: 182), RKCLQAGMNLEARKTKK (SEQ ID NO: 183), PRPRKIPR (SEQ ID NO: 184), PPRKKRTVV (SEQ ID NO: 185), NLSKKKKRKREK (SEQ ID NO: 186), RRPSRPFRKP (SEQ ID NO: 187), KRPRSPSS (SEQ ID NO: 188), KRGINDRNFWRGENERKTR (SEQ ID NO: 189), PRPPKMARYDN (SEQ ID NO: 190), KRSFSKAF (SEQ ID NO: 191), KLKIKRPVK (SEQ ID NO: 192), PKTRRRPRRSQRKRPPT (SEQ ID NO: 26792), RRKKRRPRRKKRR (SEQ ID NO: 196), PKKKSRKPKKKSRK (SEQ ID NO: 197), HKKKHPDASVNFSEFSK (SEQ ID NO: 198), QRPGPYDRPQRPGPYDRP (SEQ ID NO: 199), LSPSLSPLLSPSLSPL (SEQ ID NO: 200), RGKGGKGLGKGGAKRHRK (SEQ ID NO: 201), PKRGRGRPKRGRGR (SEQ ID NO: 202), MSRRRKANPTKLSENAKKLAKEVEN (SEQ ID NO: 194), PKKKRKVPPPPAAKRVKLD (SEQ ID NO: 193), and PKKKRKVPPPPKKKRKV (SEQ ID NO: 204).

Embodiment 45. The composition of embodiment 43 or embodiment 44, wherein the one or more NLS are expressed at or near the C-terminus of the CasX protein.

Embodiment 46. The composition of embodiment 43 or embodiment 44, wherein the one or more NLS are expressed at or near the N-terminus of the CasX protein.

Embodiment 47. The composition of embodiment 43 or embodiment 44, comprising one or more NLS located at or near the N-terminus and at or near the C-terminus of the CasX protein.

Embodiment 48. The composition of any one of embodiments 37-47, wherein the CasX variant is capable of forming a ribonuclear protein complex (RNP) with a guide nucleic acid (gNA).

Embodiment 49. The composition of embodiment 48, wherein an RNP of the CasX variant protein and the gNA variant exhibit at least one or more improved characteristics as compared to an RNP of a reference CasX protein of SEQ ID NO: 1, SEQ ID NO: 2, or SEQ ID NO: 3 and a gNA comprising a sequence of SEQ ID NOs: 4-16.

Embodiment 50. The composition of embodiment 49, wherein the improved characteristic is selected from one or more of the group consisting of improved folding of the CasX variant; improved binding affinity to a guide nucleic acid (gNA); improved binding affinity to a target DNA; improved ability to utilize a greater spectrum of one or more PAM sequences, including ATC, CTC, GTC, or TTC, in the editing of target DNA; improved unwinding of the target DNA; increased editing activity; improved editing efficiency; improved editing specificity; increased nuclease activity; increased target strand loading for double strand cleavage; decreased target strand loading for single strand nicking; decreased off-target cleavage; improved binding of non-target DNA strand; improved protein stability; improved protein solubility; improved protein:gNA complex (RNP) stability; improved protein:gNA complex solubility; improved protein yield; improved protein expression; and improved fusion characteristics.

Embodiment 51. The composition of embodiment 49 or embodiment 50, wherein the improved characteristic of the RNP of the CasX variant protein and the gNA variant is at least about 1.1 to about 100-fold or more improved relative to the RNP of the reference CasX protein of SEQ ID NO: 1, SEQ ID NO: 2, or SEQ ID NO: 3 and the gNA comprising a sequence of SEQ ID NOs: 4-16.

Embodiment 52. The composition of embodiment 49 or embodiment 50, wherein the improved characteristic of the CasX variant protein is at least about 1.1, at least about 2, at least about 10, at least about 100-fold or more improved relative to the reference CasX protein of SEQ ID NO: 1, SEQ ID NO: 2, or SEQ ID NO: 3 and the gNA comprising a sequence of SEQ ID NOs: 4-16.

Embodiment 53. The composition of any one of embodiments 49-52, wherein the improved characteristic comprises editing efficiency, and the RNP of the CasX variant protein and the gNA variant comprises a 1.1 to 100-fold improvement in editing efficiency compared to the RNP of the reference CasX protein of SEQ ID NO: 2 and the gNA of SEQ ID NOs: 4-16.

Embodiment 54. The composition of any one of embodiments 48-53, wherein the RNP comprising the CasX variant and the gNA variant exhibits greater editing efficiency and/or binding of a target sequence in the target DNA when any one of the PAM sequences TTC, ATC, GTC, or CTC is located 1 nucleotide 5′ to the non-target strand of the protospacer having identity with the targeting sequence of the gNA in a cellular assay system compared to the editing efficiency and/or binding of an RNP comprising a reference CasX protein and a reference gNA in a comparable assay system.

Embodiment 55. The composition of embodiment 54, wherein the PAM sequence is TTC.

Embodiment 56. The composition of embodiment 54, wherein the PAM sequence is ATC.

Embodiment 57. The composition of embodiment 54, wherein the PAM sequence is CTC.

Embodiment 58. The composition of embodiment 54, wherein the PAM sequence is GTC.

Embodiment 59. The composition of any one of embodiments 54-58, wherein the increased binding affinity for the one or more PAM sequences is at least 1.5-fold greater compared to the binding affinity of any one of the CasX proteins of SEQ ID NOS: 1-3 for the PAM sequences.

Embodiment 60. The composition of any one of embodiments 48-59, wherein the RNP has at least a 5%, at least a 10%, at least a 15%, or at least a 20% higher percentage of cleavage-competent RNP compared to an RNP of the reference CasX and the gNA of SEQ ID NOs: 4-16.

Embodiment 61. The composition of any one of embodiments 37-60, wherein the CasX variant protein comprises a RuvC DNA cleavage domain having nickase activity.

Embodiment 62. The composition of any one of embodiments 37-60, wherein the CasX variant protein comprises a RuvC DNA cleavage domain having double-stranded cleavage activity.

Embodiment 63. The composition of any one of embodiments 1-48, wherein the CasX protein is a catalytically inactive CasX (dCasX) protein, and wherein the dCasX and the gNA retain the ability to bind to the BCL11A target nucleic acid.

Embodiment 64. The composition of embodiment 63, wherein the dCasX comprises a mutation at residues:
a. D672, E769, and/or D935 corresponding to the CasX protein of SEQ ID NO:1; or
b. D659, E756 and/or D922 corresponding to the CasX protein of SEQ ID NO: 2.

Embodiment 65. The composition of embodiment 64, wherein the mutation is a substitution of alanine for the residue.

Embodiment 66. The composition of any one of embodiments 1-62, further comprising a donor template nucleic acid.

Embodiment 67. The composition of embodiment 66, wherein the donor template comprises a nucleic acid comprising at least a portion of a BCL11A gene selected from the group consisting of a BCL11A exon, a BCL11A intron, a BCL11A intron-exon junction, a BCL11A regulatory element, and the GATA1 binding site sequence.

Embodiment 68. The composition of embodiment 67, wherein the donor template sequence comprises one or more mutations relative to a corresponding portion of a wild-type BCL11A gene.

Embodiment 69. The composition of embodiment 67 or embodiment 68, wherein the donor template comprises a nucleic acid comprising at least a portion of a BCL11A exon selected from the group consisting of BCL11A exon 1, BCL11A exon 2, BCL11A exon 3, BCL11A exon 4, BCL11A exon 5, BCL11A exon 6, BCL11A exon 7, BCL11A exon 8, and BCL11A exon 9.

Embodiment 70. The composition of embodiment 69, wherein the donor template comprises a nucleic acid comprising at least a portion of a BCL11A exon selected from the group consisting of BCL11A exon 1, BCL11A exon 2, and BCL11A exon 3.

Embodiment 71. The composition of any one of embodiments 66-70, wherein the donor template ranges in size from 10-15,000 nucleotides.

Embodiment 72. The composition of any one of embodiments 66-71, wherein the donor template is a single-stranded DNA template or a single stranded RNA template.

Embodiment 73. The composition of any one of embodiments 66-71, wherein the donor template is a double-stranded DNA template.

Embodiment 74. The composition of any one of embodiments 66-73, wherein the donor template comprises homologous arms at or near the 5′ and 3′ ends of the donor template that are complementary to sequences flanking cleavage sites in the BCL11A target nucleic acid introduced by the Class 2 Type V CRISPR protein.

Embodiment 75. A nucleic acid comprising the donor template of any one of embodiments 66-74.

Embodiment 76. A nucleic acid comprising a sequence that encodes the CasX of any one of embodiments 37-65.

Embodiment 77. A nucleic acid comprising a sequence that encodes the gNA of any one of embodiments 1-36.

Embodiment 78. The nucleic acid of embodiment 76, wherein the sequence that encodes the CasX protein is codon optimized for expression in a eukaryotic cell.

Embodiment 79. A vector comprising the gNA of any one of embodiments 1-36, the CasX protein of any one of embodiments 37-65, or the nucleic acid of any one of embodiments 75-78.

Embodiment The vector of embodiment 79, wherein the vector further comprises a promoter.

Embodiment 81. The vector of embodiment 79 or embodiment 80, wherein the vector is selected from the group consisting of a retroviral vector, a lentiviral vector, an adenoviral vector, an adeno-associated viral (AAV) vector, a herpes simplex virus (HSV) vector, a virus-like particle (VLP), a plasmid, a minicircle, a nanoplasmid, a DNA vector, and an RNA vector.

Embodiment 82. The vector of embodiment 81, wherein the vector is an AAV vector.

Embodiment 83. The vector of embodiment 82, wherein the AAV vector is selected from AAV1, AAV2, AAV3, AAV4, AAV5, AAV6, AAV7, AAV8, AAV9, AAV10, AAV-Rh74, or AAVRh10.

Embodiment 84. The vector of embodiment 81, wherein the vector is a retroviral vector.

Embodiment 85. The vector of embodiment 81, wherein the vector is a VLP comprising one or more components of a gag polyprotein.

Embodiment 86. The vector of embodiment 85, wherein the one or more components of the gag polyprotein are selected from the group consisting of matrix protein (MA), nucleocapsid protein (NC), capsid protein (CA), and p1-p6 protein.

Embodiment 87. The vector of embodiment 85 or embodiment 86, comprising the CasX protein and the gNA.

Embodiment 88. The vector of embodiment 87, wherein the CasX protein and the gNA are associated together in an RNP.

Embodiment 89. The VLP of any one of embodiments 85-88, further comprising a pseudotyping viral envelope glycoprotein or antibody fragment that provides for binding and fusion of the VLP to a target cell.

Embodiment 90. The VLP of embodiment of embodiment 89, wherein the target cell is selected from the group consisting of a hematopoietic stem cell (HSC), a hematopoietic progenitor cell (HPC), a CD34+ cell, a mesenchymal stem cell (MSC), an embryonic stem (ES) cell, an induced pluripotent stem cell (iPSC), a common myeloid progenitor cell, a proerythroblast cell, and an erythroblast cell.

Embodiment 91. The vector of any one of embodiments 85-90, further comprising the donor template.

Embodiment 92. A host cell comprising the vector of any one of embodiments 79-91.

Embodiment 93. The host cell of embodiment 92, wherein the host cell is selected from the group consisting of BHK, HEK293, HEK293T, NS0, SP2/0, YO myeloma cells, P3X63 mouse myeloma cells, PER, PER.C6, NIH3T3, COS, HeLa, CHO, and yeast cells.

Embodiment 94. A method of modifying a BCL11A target nucleic acid sequence in a population of cells, the method comprising introducing into cells of the population:
a. the composition of any one of embodiments 1-74;
b. the nucleic acid of any one of embodiments 75-78;
c. the vector as in any one of embodiments 79-84;
d. the VLP of any one of embodiments 85-91; or
e. combinations of two or more of (a)-(d),
   wherein the BCL11A gene target nucleic acid sequence of the cells targeted by the first gNA is modified by the CasX protein.

Embodiment 95. The method of embodiment 94, wherein the modifying comprises introducing a single-stranded break in the BCL11A gene target nucleic acid sequence of the cells of the population.

Embodiment 96. The method of embodiment 94, wherein the modifying comprises introducing a double-stranded break in the BCL11A gene target nucleic acid sequence of the cells of the population.

Embodiment 97. The method of any one of embodiments 94-96, further comprising introducing into the cells of the population a second gNA or a nucleic acid encoding the second gNA, wherein the second gNA has a targeting sequence complementary to a different or overlapping portion of the BCL11A gene target nucleic acid compared to the first gNA, resulting in an additional break in the BCL11A target nucleic acid of the cells of the population.

Embodiment 98. The method of any one of embodiments 94-97, wherein the modifying comprises introducing an insertion, deletion, substitution, duplication, or inversion of one or more nucleotides in the BCL11A gene of the cells of the population.

Embodiment 99. The method of embodiment 94-98, wherein a GATA1 binding site sequence of the target nucleic acid is modified.

Embodiment 100. The method of any one of embodiments 94-97, wherein the method comprises insertion of the donor template into the break site(s) of the BCL11A gene target nucleic acid sequence of the cells of the population.

Embodiment 101. The method of embodiment 98, wherein the insertion of the donor template is mediated by homology-directed repair (HDR) or homology-independent targeted integration (HITI).

Embodiment 102. The method of embodiment 100 or embodiment 101, wherein the GATA1 binding site sequence of the target nucleic acid is modified.

Embodiment 103. The method of any one of embodiments 100-102, wherein insertion of the donor template results in a knock-down or knock-out of the BCL11A gene in the cells of the population.

Embodiment 104. The method of any one of embodiments 94-103, wherein the BCL11A gene of the cells of the population is modified such that expression of the BCL11A protein is reduced by at least about 10%, at least about 20%, at least about 30%, at least about 40%, at least about 50%, at least about 60%, at least about 70%, at least about 80%, or at least about 90% in comparison to cells in which the BCL11A gene has not been modified.

Embodiment 105. The method of any one of embodiments 94-103, wherein the BCL11A gene of the cells of the population is modified such that at least about 10%, at least about 20%, at least about 30%, at least about 40%, at least about 50%, at least about 60%, at least about 70%, at least about 80%, or at least about 90% of the cells do not express a detectable level of BCL11A protein.

Embodiment 106. The method of any one of embodiments 94-105, wherein the cells are eukaryotic.

Embodiment 107. The method of embodiment 106, wherein the eukaryotic cells are selected from the group consisting of rodent cells, mouse cells, rat cells, and non-human primate cells.

Embodiment 108. The method of embodiment 106, wherein the eukaryotic cells are human cells.

Embodiment 109. The method of any one of embodiments 106-108, wherein the eukaryotic cell is selected from the group consisting of a hematopoietic stem cell (HSC), a hematopoietic progenitor cell (HPC), a CD34+ cell, a mesenchymal stem cell (MSC), induced pluripotent stem cell (iPSC), a common myeloid progenitor cell, a proerythroblast cell, and an erythroblast cell.

Embodiment 110. The method of any one of embodiment 94-109, wherein the modification of the BCL11A gene target nucleic acid sequence of the population of cells occurs in vitro or ex vivo.

Embodiment 111. The method of any one of embodiment 94-109, wherein the modification of the BCL11A gene target nucleic acid sequence of the population of cells occurs in vivo in a subject.

Embodiment 112. The method of embodiment 111, wherein the subject is selected from the group consisting of a rodent, a mouse, a rat, and a non-human primate.

Embodiment 113. The method of embodiment 111, wherein the subject is a human.

Embodiment 114. The method of any one of embodiments 111-113, wherein the method comprises administering a therapeutically effective dose of an AAV vector to the subject.

Embodiment 115. The method of embodiment 114, wherein the AAV vector is administered to the subject at a dose of at least about 1×10⁵ vector genomes/kg (vg/kg), at least about 1×10⁶ vg/kg, at least about 1×10⁷ vg/kg, at least about 1×10⁸ vg/kg, at least about 1×109 vg/kg, at least about 1×10¹⁰ vg/kg, at least about 1×10¹¹ vg/kg, at least about 1×10¹² vg/kg, at least about 1×10¹³ vg/kg, at least about 1×10¹⁴ vg/kg, at least about 1×10¹⁵ vg/kg, or at least about 1×10¹⁶ vg/kg.

Embodiment 116. The method of embodiment 114, wherein the AAV vector is administered to the subject at a dose of at least about 1×10⁵ vg/kg to about 1×10¹⁶ vg/kg, at least about 1×10⁶ vg/kg to about 1×10¹⁵ vg/kg, or at least about 1×10⁷ vg/kg to about 1×10¹⁴ vg/kg.

Embodiment 117. The method of any one of embodiments 111-113, wherein the method comprises administering a therapeutically effective dose of a VLP to the subject.

Embodiment 118. The method of embodiment 117, wherein the VLP is administered to the subject at a dose of at least about 1×10⁵ particles/kg, at least about 1×10⁶ particles/kg, at least about 1×10⁷ particles/kg at least about 1×10⁸ particles/kg, at least about 1×10⁹ particles/kg, at least about 1×10¹⁰ particles/kg, at least about 1×10¹¹ particles/kg, at least about 1×10¹² particles/kg, at least about 1×10¹³ particles/kg, at least about 1×10¹⁴ particles/kg, at least about 1×10¹⁵ particles/kg, at least about 1×10¹⁶ particles/kg.

Embodiment 119. The method of embodiment 117, wherein the VLP is administered to the subject at a dose of at least about 1×10⁵ particles/kg to about 1×10¹⁶ particles/kg, or at least about 1×10⁶ particles/kg to about 1×10¹⁵ particles/kg, or at least about 1×10⁷ particles/kg to about 1×10¹⁴ particles/kg

Embodiment 120. The method of any one of embodiments 112-119, wherein the vector or VLP is administered to the subject by a route of administration selected from transplantation, local injection, systemic infusion, or combinations thereof.

Embodiment 121. The method of any one of embodiments 94-120, further comprising contacting the BCL11A gene target nucleic acid sequence of the population of cells with:
a. an additional CRISPR nuclease and a gNA targeting a different or overlapping portion of the BCL11A target nucleic acid compared to the first gNA;
b. a polynucleotide encoding the additional CRISPR nuclease and the gNA of (a);
c. a vector comprising the polynucleotide of (b); or
d. a VLP comprising the additional CRISPR nuclease and the gNA of (a) wherein the contacting results in modification of the BCL11A gene at a different location in the sequence compared to the sequence targeted by the first gNA.

Embodiment 122. The method of embodiment 121, wherein the additional CRISPR nuclease is a CasX protein having a sequence different from the CasX protein of any of the preceding embodiments.

Embodiment 123. The method of embodiment 121, wherein the additional CRISPR nuclease is not a CasX protein.

Embodiment 124. The method of embodiment 123, wherein the additional CRISPR nuclease is selected from the group consisting of Cas9, Cas12a, Cas12b, Cas12c, Cas12d (CasY), Cas12J, Cas13a, Cas13b, Cas13c, Cas13d, CasX, CasY, Cas14, Cpf1, C2cl, Csn2, and sequence variants thereof.

Embodiment 125. A population of cells modified by the method of any one of embodiments 94-124, wherein the cells have been modified such that at least 70%, at least 75%, at least 80%, at least 85%, at least 90%, or at least 95% of the modified cells do not express a detectable level of BCL11A protein.

Embodiment 126. A population of cells modified by the method of any one of embodiments 94-124, wherein the cells have been modified such that the expression of BCL11A protein is reduced by at least 70%, at least 75%, at least 80%, at least 85%, at least 90%, or at least 95% compared to cells where the BCL11A gene has not been modified.

Embodiment 127. A method of treating a hemoglobinopathy in a subject in need thereof, the method comprising administering to the subject a therapeutically effective amount of the cells of embodiment 125 or embodiment 126.

Embodiment 128. The method of embodiment 127, wherein the hemoglobinopathy is a sickle cell disease or beta-thalassemia.

Embodiment 129. The method of any one of embodiments 127 or embodiment 128, wherein the cells are autologous with respect to the subject to be administered the cells.

Embodiment 130. The method of any one of embodiments 127 or embodiment 128, wherein the cells are allogeneic with respect to the subject to be administered the cells.

Embodiment 131. The method of any one of embodiments 127-130, wherein the cells or their progeny persist in the subject for at least one month, two month, three months, four months, five months, six months, seven months, eight months, nine months, ten months, eleven months, twelve months, thirteen months, fourteen month, fifteen months, sixteen months, seventeen months, eighteen months, nineteen months, twenty months, twenty-one months, twenty-two months, twenty-three months, two years, three years, four years, or five years after administration of the modified cells to the subject.

Embodiment 132. The method of any one of embodiments 127-131, wherein the method results in an increased levels of hemoglobin F (HbF) in circulating blood of at least about 5%, at least about 10%, at least about 20%, at least about 30%, at least about 40%, or at least about 50% compared to the levels of HbF in the subject prior to treatment.

Embodiment 133. The method of any one of embodiments 127-131, wherein the method results in a ratio of HbF to hemoglobin S (HbS) in the subject of at least 0.01:1.0, at least 0.025:1.0, at least 0.05:1.0, at least 0.075:1.0 at least 0.1:1.0, at least 0.2:1.0, at least 0.3:1.0, at least 0.4:1.0, at least 0.5:1:0, at least 0.75:1.0, at least 1.0:1.0, at least 1.25:1.0, at least 1.5:1.0, or at least 1.75:1.0.

Embodiment 134. The method of any one of embodiments 127-131, wherein the method results in HbF levels of at least about 5%, or at least about 10%, or at least about 20%, or at least about 30% of total circulating hemoglobin in the subject.

Embodiment 135. The method of any one of embodiments 127-134, wherein the subject is selected from the group consisting of a rodent, a mouse, a rat, and a non-human primate.

Embodiment 136. The method of any one of embodiments 127-134, wherein the subject is a human.

Embodiment 137. A method of treating a hemoglobinopathy in a subject in need thereof, comprising modifying a BCL11A gene in cells of the subject, the modifying comprising contacting said cells with a therapeutically effective dose of:
a. the composition of any one of embodiments 1-74;
b. the nucleic acid of any one of embodiments 75-78;
c. the vector as in any one of embodiments 79-84;
d. the VLP of any one of embodiments 85-88; or
e. combinations of two or more of (a)-(d),
   wherein the BCL11A gene of the cells targeted by the first gNA is modified by the CasX protein.

Embodiment 138. The method of embodiment 137, wherein the hemoglobinopathy is sickle cell disease or beta-thalassemia.

Embodiment 139. The method of any one of embodiments 137 or embodiment 138, wherein the cell is selected from the group consisting of hematopoietic stem cells (HSC), hematopoietic progenitor cells (HPC), CD34+ cells, mesenchymal stem cells (MSC), induced pluripotent stem cells (iPSC), common myeloid progenitor cells, proerythroblast cells, and erythroblast cells.

Embodiment 140. The method of any one of embodiments 137-139, wherein the modifying comprises introducing a single-stranded break in the BCL11A gene of the cells.

Embodiment 141. The method of any one of embodiments 137-139, wherein the modifying comprises introducing a double-stranded break in the BCL11A gene of the cells.

Embodiment 142. The method of any one of embodiments 137-141, further comprising introducing into the cells of the subject a second gNA or a nucleic acid encoding the second gNA, wherein the second gNA has a targeting sequence complementary to a different or overlapping portion of the target nucleic acid compared to the first gNA, resulting in an additional break in the BCL11A target nucleic acid of the cells of the subject.

Embodiment 143. The method of any one of embodiments 137-142, wherein the modifying comprises introducing an insertion, deletion, substitution, duplication, or inversion of one or more nucleotides in the BCL11A gene of the cells.

Embodiment 144. The method of embodiment 143, wherein the modifying results in a knock-down or knock-out of the BCL11A gene in the modified cells of the subject.

Embodiment 145. The method of any one of embodiments 137-144, wherein the BCL11A gene of the cells are modified such that expression of the BCL11A protein by the modified cells is reduced by at least about 10%, at least about 20%, at least about 30%, at least about 40%, at least about 50%, at least about 60%, at least about 70%, at least about 80%, or at least about 90% in comparison to cells that have not been modified.

Embodiment 146. The method of any one of embodiments 137-144, wherein the BCL11A gene of the cells of the subject are modified such that at least 70%, at least 75%, at least 80%, at least 85%, at least 90%, or at least 95% of the modified cells do not express a detectable level of BCL11A protein.

Embodiment 147. The method of any one of embodiments 137-146, wherein the method results in an increased levels of hemoglobin F (HbF) in circulating blood of the subject of at least about 5%, at least about 10%, at least about 20%, at least about 30%, at least about 40%, or at least about 50% compared to the levels of HbF in the subject prior to treatment.

Embodiment 148. The method of any one of embodiments 137-147, wherein the method results in a ratio of HbF to hemoglobin S (HbS) in circulating blood of the subject of at least 0.01:1.0, at least 0.025:1.0, at least 0.05:1.0, at least 0.075:1.0 at least 0.1:1.0, at least 0.2:1.0, at least 0.3:1.0, at least 0.4:1.0, at least 0.5:1:0, at least 0.75:1.0, at least 1.0:1.0, at least 1.25:1.0, at least 1.5:1.0, or at least 1.75:1.0.

Embodiment 149. The method of any one of embodiments 137-146, wherein the method results in HbF levels of at least about 5%, or at least about 10%, or at least about 20%, or at least about 30% of total hemoglobin in circulating blood of the subject.

Embodiment 150. The method of any one of embodiments 137-142, wherein the method comprises insertion of the donor template into the break site(s) of the BCL11A gene target nucleic acid sequence of the cells.

Embodiment 151. The method of embodiment 149, wherein the insertion of the donor template is mediated by homology-directed repair (HDR) or homology-independent targeted integration (HITI).

Embodiment 152. The method of embodiment 149 or embodiment 151, wherein insertion of the donor template results in a knock-down or knock-out of the BCL11A gene in the modified cells of the subject.

Embodiment 153. The method of any one of embodiments 147-152, wherein the BCL11A gene of the cells are modified such that expression of the BCL11A protein by the modified cells is reduced by at least about 10%, at least about 20%, at least about 30%, at least about 40%, at least about 50%, at least about 60%, at least about 70%, at least about 80%, or at least about 90% in comparison to cells that have not been modified.

Embodiment 154. The method of any one of embodiments 147-152, wherein the BCL11A gene of the cells of the subject are modified such that at least 70%, at least 75%, at least 80%, at least 85%, at least 90%, or at least 95% of the modified cells do not express a detectable level of BCL11A protein.

Embodiment 155. The method of any one of embodiments 147-154, wherein the method results in an increased levels of hemoglobin F (HbF) in circulating blood of the subject of at least about 5%, at least about 10%, at least about 20%, at least about 30%, at least about 40%, or at least about 50% compared to the levels of HbF in the subject prior to treatment.

Embodiment 156. The method of any one of embodiments 147-154, wherein the method results in a ratio of HbF to hemoglobin S (HbS) in circulating blood of the subject of at least 0.01:1.0, at least 0.025:1.0, at least 0.05:1.0, at least 0.075:1.0 at least 0.1:1.0, at least 0.2:1.0, at least 0.3:1.0, at least 0.4:1.0, at least 0.5:1:0, at least 0.75:1.0, at least 1.0:1.0, at least 1.25:1.0, at least 1.5:1.0, or at least 1.75:1.0.

Embodiment 157. The method of any one of embodiments 147-154, wherein the method results in HbF levels of at least about 5%, or at least about 10%, or at least about 20%, or at least about 30% of total hemoglobin in circulating blood of the subject.

Embodiment 158. The method of any one of embodiments 137-156, wherein the subject is selected from the group consisting of rodent, mouse, rat, and non-human primate.

Embodiment 159. The method of any one of embodiments 137-156, wherein the subject is a human.

Embodiment 160. The method of any one of embodiments 137-159, wherein the vector is AAV and is administered to the subject at a dose of at least about 1×10⁵ vector genomes/kg (vg/kg), at least about 1×10⁶ vg/kg, at least about 1×10⁷ vg/kg, at least about 1×10⁸ vg/kg, at least about 1×10⁹ vg/kg, at least about 1×10¹⁰ vg/kg, at least about 1×10¹¹ vg/kg, at least about 1×10¹² vg/kg, at least about 1×10¹³ vg/kg, at least about 1×10¹⁴ vg/kg, at least about 1×10¹⁵ vg/kg, or at least about 1×10¹⁶ vg/kg.

Embodiment 161. The method of any one of embodiments 137-159, wherein the vector is AAV and is administered to the subject at a dose of at least about 1×10⁵ vg/kg to about 1×10¹⁶ vg/kg, at least about 1×10⁶ vg/kg to about 1×10¹⁵ vg/kg, or at least about 1×10⁷ vg/kg to about 1×10¹⁴ vg/kg.

Embodiment 162. The method of any one of embodiments 137-159, wherein the VLP is administered to the subject at a dose of at least about 1×10⁵ particles/kg, at least about 1×10⁶ particles/kg, at least about 1×10⁷ particles/kg at least about 1×10⁸ particles/kg, at least about 1×10⁹ particles/kg, at least about 1×10¹⁰ particles/kg, at least about 1×10¹¹ particles/kg, at least about 1×10¹² particles/kg, at least about 1×10¹³ particles/kg, at least about 1×10¹⁴ particles/kg, at least about 1×10¹5 particles/kg, at least about 1×10¹⁶ particles/kg.

Embodiment 163. The method of any one of embodiments 137-159, wherein the VLP is administered to the subject at a dose of at least about 1×10⁵ particles/kg to about 1×10¹⁶ particles/kg, or at least about 1×10⁶ particles/kg to about 1×10¹⁵ particles/kg, or at least about 1×10⁷ particles/kg to about 1×10¹⁴ particles/kg

Embodiment 164. The method of any one of embodiments 137-163, wherein the vector or VLP is administered to the subject by a route of administration selected from transplantation, local injection, systemic infusion, or combinations thereof.

Embodiment 165. The method of any one of embodiments 137-164, wherein the method results in improvement in at least one clinically-relevant endpoint in the subject.

Embodiment 166. The method of embodiment 165, wherein the method results in improvement in at least one clinically-relevant parameter selected from the group consisting of occurrence of end-organ disease, albuminuria, hypertension, hyposthenia, hyposthenuria, diastolic dysfunction, functional exercise capacity, acute coronary syndrome, pain events, pain severity, anemia, hemolysis, tissue hypoxia, organ dysfunction, abnormal hematocrit values, childhood mortality, incidence of strokes, hemoglobin levels compared to baseline, HbF levels, reduced incidence of pulmonary embolisms, incidence of vaso-occlusive crises, concentration of hemoglobin S in erythrocytes, rate of hospitalizations, liver iron concentration, required blood transfusions, and quality of life score.

Embodiment 167. The method of embodiment 165, wherein the method results in improvement in at least two clinically-relevant parameters selected from the group consisting of occurrence of end-organ disease, albuminuria, hypertension, hyposthenia, hyposthenuria, diastolic dysfunction, functional exercise capacity, acute coronary syndrome, pain events, pain severity, anemia, hemolysis, tissue hypoxia, organ dysfunction, abnormal hematocrit values, childhood mortality, incidence of strokes, hemoglobin levels compared to baseline, HbF levels, reduced incidence of pulmonary embolisms, incidence of vaso-occlusive crises, concentration of hemoglobin S in erythrocytes, rate of hospitalizations, liver iron concentration, required blood transfusions, and quality of life score.

Embodiment 168. A method for treating a subject with a hemoglobinopathy, the method comprising:
a. isolating induced pluripotent stem cells (iPSC) or hematopoietic stem cells (HSC) from a subject;
b. modifying the BCL11A target nucleic acid of the iPSC or HSC by the method of any one of embodiments 94-110;
c. differentiating the modified iPSC or HSC into a hematopoietic progenitor cell; and
d. implanting the hematopoietic progenitor cell into the subject with the hemoglobinopathy, wherein the method results in an increased levels of hemoglobin F (HbF) in circulating blood of the subject of at least about 5%, at least about 10%, at least about 20%, at least about 30%, at least about 40%, or at least about 50% compared to the levels of HbF in the subject prior to treatment.

Embodiment 169. The method of embodiment 168, wherein the iPSC or HSC is autologous and is isolated from the subject's bone marrow or peripheral blood.

Embodiment 170. The method of embodiment 168, wherein the iPSC or HSC is allogeneic and is isolated from a different subject's bone marrow or peripheral blood.

Embodiment 171. The method of any one of embodiments 168-170, wherein the implanting comprises administering the hematopoietic progenitor cell to the subject by transplantation, local injection, systemic infusion, or combinations thereof.

Embodiment 172. The method of any one of embodiments 168-171, wherein the hemoglobinopathy is sickle cell disease or beta-thalassemia.

Embodiment 173. A method of increasing fetal hemoglobin (HbF) in a subject by genome editing, the method comprising: a. administering to the subject an effective dose of the vector of any one of embodiments 79-84 or the VLP of any one of embodiments 85-90, wherein the vector or VLP delivers the CasX:gNA system to cells of the subject;
b. the BCL11A target nucleic acid of cells of the subject are edited by the CasX targeted by the first gNA;
c. the editing comprises introducing an insertion, deletion, substitution, duplication, or inversion of one or more nucleotides in the target nucleic acid sequence such that expression of BCL11A protein is reduced or eliminated,
wherein the method results in an increased levels of hemoglobin F (HbF) in circulating blood of the subject of at least about 5%, at least about 10%, at least about 20%, at least about 30%, at least about 40%, or at least about 50% compared to the levels of HbF in the subject prior to treatment.

Embodiment 174. The method of embodiment 173, wherein the cells are selected from the group consisting of hematopoietic stem cells (HSC), hematopoietic progenitor cells (HPC), CD34+ cells, mesenchymal stem cells (MSC), induced pluripotent stem cells (iPSC), common myeloid progenitor cells, proerythroblast cells, and erythroblast cells.

Embodiment 175. The method of embodiment 173 or embodiment 174, wherein the target nucleic acid of the cells has been edited such that expression of the BCL11A protein is reduced by at least about 10%, at least about 20%, at least about 30%, at least about 40%, at least about 50%, at least about 60%, at least about 70%, at least about 80%, or at least about 90% in comparison to target nucleic acid of cells that have not been edited.

Embodiment 176. The method of any one of embodiments 173-175, wherein the subject is selected from the group consisting of mouse, rat, pig, and non-human primate.

Embodiment 177. The method of any one of embodiments 173-175, wherein the subject is a human.

Embodiment 178. The method of any one of embodiments 173-177, wherein the vector is administered at a dose of at least about 1×10⁵ vector genomes/kg (vg/kg), at least about 1×10⁶ vg/kg, at least about 1×10⁷ vg/kg, at least about 1×10⁸ vg/kg, at least about 1×10⁹ vg/kg, at least about 1×10¹⁰ vg/kg at least about 1×10¹¹ vg/kg, at least about 1×10¹² vg/kg, at least about 1×10¹³ vg/kg, at least about 1×10¹⁴vg/kg, at least about 1×10¹⁵ vg/kg, or at least about 1×10¹⁶ vg/kg.

Embodiment 179. The method of any one of embodiments 173-177, wherein the VLP is administered at a dose of at least about 1×10⁵ particles/kg, at least about 1×10⁶ particles/kg, at least about 1×10⁷ particles/kg, at least about 1×10⁸ particles/kg, at least about 1×10⁹ particles/kg, at least about 1×10¹⁰ particles/kg at least about 1×10¹¹ particles/kg, at least about 1×10¹² particles/kg, at least about 1×10¹³ particles/kg, at least about 1×10¹⁴ particles/kg, at least about 1×10¹⁵ particles/kg, or at least about 1×10¹⁶ particles/kg.

Embodiment 180. The method of any one of embodiments 173-179, wherein the vector or VLP is administered by a route of administration selected from transplantation, local injection, systemic infusion, or combinations thereof.

Embodiment 181. The composition of any one of embodiments 1-74, the nucleic acid of any one of embodiments 75-78, the vector of any one of 79-84, the VLP of any one of embodiments 85-88, the host cell of embodiment 92 or embodiment 93, or the population of cells of embodiment 125 or embodiment 126, for use as a medicament for the treatment of a hemoglobinopathy.

Embodiment 182. The composition of embodiment 1, wherein the target nucleic acid sequence is complementary to a non-target strand sequence located 1 nucleotide 3′ of a protospacer adjacent motif (PAM) sequence.

Embodiment 183. The composition of embodiment 182, wherein the PAM sequence comprises a TC motif.

Embodiment 184. The composition of embodiment 183, wherein the PAM sequence comprises ATC, GTC, CTC or TTC.

Embodiment 185. The composition of any one of embodiments 182-184, wherein the Class 2 Type V CRISPR protein comprises a RuvC domain.

Embodiment 186. The composition of embodiment 185, wherein the RuvC domain generates a staggered double-stranded break in the target nucleic acid sequence.

Embodiment 187. The composition of any one of embodiments 182-186, wherein the Class 2 Type V CRISPR protein does not comprise an HNH nuclease domain.

Set II

Embodiment 1. A system comprising a Class 2 Type V CRISPR protein and a first guide ribonucleic acid (gRNA), wherein the gRNA comprises a targeting sequence complementary to a target nucleic acid sequence comprising a polypyrimidine tract-binding protein 1 (BCL11A) gene.

Embodiment 2. The system of embodiment 1, wherein the gRNA comprises a targeting sequence complementary to a target nucleic acid sequence selected from the group consisting of:
a. a BCL11A intron;
b. a BCL11A exon;
c. a BCL11A intron-exon junction;
d. a BCL11A regulatory element; and e. an intergenic region.

Embodiment 3. The system of embodiment 1 or embodiment 2, wherein the BCL11A gene comprises a wild-type sequence.

Embodiment 4. The system of any one of embodiments 1-3, wherein the gRNA is a single-molecule gRNA (sgRNA).

Embodiment 5. The system of any one of embodiments 1-4, wherein the gRNA is a dual-molecule gRNA (dgRNA).

Embodiment 6. The system of any one of embodiments 1-5, wherein the targeting sequence of the gRNA comprises a sequence selected from the group consisting of SEQ ID NOS: 272-2100 and 2286-26789, or a sequence having at least about 65%, at least about 75%, at least about 85%, or at least about 95% identity thereto.

Embodiment 7. The system of any one of embodiments 1-5, wherein the targeting sequence of the gRNA comprises a sequence selected from the group consisting of SEQ ID NOS: 272-2100 and 2286-26789.

Embodiment 8. The system of embodiment 7, wherein the targeting sequence has a single nucleotide removed from the 3′ end of the sequence.

Embodiment 9. The system of embodiment 7, wherein the targeting sequence has two nucleotides removed from the 3′ end of the sequence.

Embodiment 10. The system of embodiment 7, wherein the targeting sequence has three nucleotides removed from the 3′ end of the sequence.

Embodiment 11. The system of embodiments 7, wherein the targeting sequence has four nucleotides removed from the 3′ end of the sequence.

Embodiment 12. The system of embodiment 7, wherein the targeting sequence has five nucleotides removed from the 3′ end of the sequence.

Embodiment 13. The system of any one of embodiments 1-12, wherein the targeting sequence of the gRNA is complementary to a sequence of a BCL11A exon.

Embodiment 14. The system of embodiment 13, wherein the targeting sequence of the gRNA is complementary to a sequence selected from the group consisting of a BCL11A exon 1 sequence, BCL11A exon 2 sequence, BCL11A exon 3 sequence, BCL11A exon 4 sequence, BCL11A exon 5 sequence, BCL11A exon 6 sequence, BCL11A exon 7 sequence, BCL11A exon 8 sequence, and a BCL11A exon 9 sequence.

Embodiment 15. The system of embodiment 14, wherein the targeting sequence of the gRNA is complementary to a sequence selected from the group consisting of a BCL11A exon 1 sequence, BCL11A exon 2 sequence, and a BCL11A exon 3 sequence.

Embodiment 16. The system of any one of embodiments 1-12, wherein the targeting sequence of the gRNA is complementary to a sequence of a BCL11A regulatory element.

Embodiment 17. The system of embodiment 16, wherein the targeting sequence of the gRNA is complementary to a sequence of a promoter of the BCL11A gene.

Embodiment 18. The system of embodiment 16, wherein the targeting sequence of the gRNA is complementary to a sequence of an enhancer regulatory element.

Embodiment 19. The system of embodiment 18, wherein the targeting sequence of the gRNA is complementary to a sequence that comprises a GATA1 erythroid-specific enhancer binding site (GATA1) of the BCL11A gene.

Embodiment 20. The system of embodiment 16, wherein the targeting sequence of the gRNA is complementary to a sequence that is 5′ to the GATA1 binding site of the BCL11A gene.

Embodiment 21. The system of embodiment 19 or embodiment 20, wherein the targeting sequence of the gRNA comprises a sequence of UGGAGCCUGUGAUAAAAGCA (SEQ ID NO: 22), or a sequence having at least 90% or 95% sequence identity thereto.

Embodiment 22. The system of embodiment 19, wherein the targeting sequence of the gRNA consists of a sequence of UGGAGCCUGUGAUAAAAGCA (SEQ ID NO: 22).

Embodiment 23. The system of embodiment 18, wherein the targeting sequence of the gRNA comprises a sequence of UGCUUUUAUCACAGGCUCCA (SEQ ID NO: 23), or a sequence having at least 90% or 95% sequence identity thereto.

Embodiment 24. The system of embodiment 18, wherein the targeting sequence of the gRNA consists of a sequence of UGCUUUUAUCACAGGCUCCA (SEQ ID NO: 23).

Embodiment 25. The system of embodiment 19 or embodiment 20, wherein the targeting sequence of the gRNA comprises a sequence of CAGGCUCCAGGAAGGGUUUG (SEQ ID NO: 2949), or a sequence having at least 90% or 95% sequence identity thereto.

Embodiment 26. The system of embodiment 19 or embodiment 20, wherein the targeting sequence of the gRNA consists of a sequence of CAGGCUCCAGGAAGGGUUUG (SEQ ID NO: 2949).

Embodiment 27. The system of embodiment 19 or embodiment 20, wherein the targeting sequence of the gRNA comprises a sequence of GAGGCCAAACCCUUCCUGGA (SEQ ID NO: 2948), or a sequence having at least 90% or 95% sequence identity thereto.

Embodiment 28. The system of embodiment 19 or embodiment 20, wherein the targeting sequence of the gRNA consists of a sequence of CAGGCUCCAGGAAGGGUUUG (SEQ ID NO: 2948).

Embodiment 29. The system of embodiment 19 or embodiment 20, wherein the targeting sequence of the gRNA comprises a sequence of AGUGCAAGCUAACAGUUGCU (SEQ ID NO: 15747), or a sequence having at least 90% or 95% sequence identity thereto.

Embodiment 30. The system of embodiment 19 or embodiment 20, wherein the targeting sequence of the gRNA consists of a sequence of AGUGCAAGCUAACAGUUGCU (SEQ ID NO: 15747).

Embodiment 31. The system of embodiment 19 or embodiment 20, wherein the targeting sequence of the gRNA comprises a sequence of AUACAACUUUGAAGCUAGUC (SEQ ID NO: 15748), or a sequence having at least 90% or 95% sequence identity thereto.

Embodiment 32. The system of embodiment 19 or embodiment 20, wherein the targeting sequence of the gRNA consists of a sequence of AUACAACUUUGAAGCUAGUC (SEQ ID NO: 15748).

Embodiment 33. The system of any one of embodiments 1-32, further comprising a second gRNA, wherein the second gRNA has a targeting sequence complementary to a different or overlapping portion of the BCL11A target nucleic acid compared to the targeting sequence of the gRNA of the first gRNA.

Embodiment 34. The system of embodiment 33, wherein the targeting sequence of the second gRNA is complementary to a sequence of the target nucleic acid that is 5′ or 3′ to the GATA1 binding site sequence.

Embodiment 35. The system of embodiment 33, wherein the first and the second gRNA each have a targeting sequence complementary to a sequence within the promoter of the BCL11A gene.

Embodiment 36. The system of any one of embodiments 1-35, wherein the first or second gRNA has a scaffold comprising a sequence selected from the group consisting of SEQ ID NOS: 2238-2285, 26794-26839 and 27219-27265 or a sequence having at least about 50%, at least about 60%, at least about 70%, at least about 80%, at least about 90%, at least about 95%, at least about 96%, at least about 97%, at least about 98%, at least about 99% sequence identity thereto.

Embodiment 37. The system of any one of embodiments 1-36, wherein the first or second gRNA has a scaffold comprising a sequence selected from the group consisting of SEQ ID NOs: 2238-2285, 26794-26839 and 27219-27265.

Embodiment 38. The system of any one of embodiments 1-36, wherein the first or second gRNA has a scaffold consisting of a sequence selected from the group consisting of SEQ ID NOs: 2238-2285, 26794-26839 and 27219-27265.

Embodiment 39. The system of embodiment 38, wherein the first or second gRNA has a scaffold consisting of the sequence of SEQ ID NO: 2238 or SEQ ID NO: 26800.

Embodiment 40. The system of any one of embodiments 36-39, wherein targeting sequence is linked to the 3′ end of the scaffold of the gRNA.

Embodiment 41. The system of any one of embodiments 1-40, wherein the Class 2 Type V CRISPR protein is a CasX variant protein comprising a sequence selected from the group consisting of SEQ ID NOS: 59, 72-99, 101-148, and 26908-27154, or a sequence having at least about 50%, at least about 60%, at least about 70%, at least about 80%, at least about 90%, or at least about 95%, or at least about 95%, or at least about 96%, or at least about 97%, or at least about 98%, or at least about 99% sequence identity thereto.

Embodiment 42. The system of embodiment 41, wherein the Class 2 Type V CRISPR protein is a CasX variant protein comprising a sequence selected from the group consisting of SEQ ID NOS: 59, 72-99, 101-148, and 26908-27154.

Embodiment 43. The system of embodiment 41, wherein the CasX variant protein consists of a sequence selected from the group consisting of SEQ ID NOS: 59, 72-99, 101-148, and 26908-27154.

Embodiment 44. The system of embodiment 42, wherein the CasX variant protein consists of a sequence selected from the group consisting of SEQ ID NOS: 126, 27043, 27046, 27050.

Embodiment 45. The system of embodiment 41, wherein the CasX variant protein comprises at least one modification relative to a reference CasX protein having a sequence selected from SEQ ID NOS:1-3.

Embodiment 46. The system of embodiment 45, wherein the at least one modification comprises at least one amino acid substitution, deletion, or substitution in a domain of the CasX variant protein relative to the reference CasX protein.

Embodiment 47. The system of embodiment 46, wherein the domain is selected from the group consisting of a non-target strand binding (NTSB) domain, a target strand loading (TSL) domain, a helical I domain, a helical II domain, an oligonucleotide binding domain (OBD), and a RuvC DNA cleavage domain.

Embodiment 48. The system of any one of embodiments 41-47, wherein the CasX variant protein does not comprise an HNH domain.

Embodiment 49. The system of any one of embodiments 41-48, wherein the CasX variant protein further comprises one or more nuclear localization signals (NLS).

Embodiment 50. The system of embodiment 49, wherein the one or more NLS are selected from the group of sequences consisting of PKKKRKV (SEQ ID NO: 168), KRPAATKKAGQAKKKK (SEQ ID NO: 169), PAAKRVKLD (SEQ ID NO: 170), RQRRNELKRSP (SEQ ID NO: 171), NQSSNFGPMKGGNFGGRSSGPYGGGGQYFAKPRNQGGY (SEQ ID NO: 172), RMRIZFKNKGKDTAELRRRRVEVSVELRKAKKDEQILKRRNV (SEQ ID NO: 173), VSRKRPRP (SEQ ID NO: 174), PPKKARED (SEQ ID NO: 175), PQPKKKPL (SEQ ID NO: 176), SALIKKKKKMAP (SEQ ID NO: 177), DRLRR (SEQ ID NO: 178), PKQKKRK (SEQ ID NO: 179), RKLKKKIKKL (SEQ ID NO: 180), REKKKFLKRR (SEQ ID NO: 181), KRKGDEVDGVDEVAKKKSKK (SEQ ID NO: 182), RKCLQAGMNLEARKTKK (SEQ ID NO: 183), PRPRKIPR (SEQ ID NO: 184), PPRKKRTVV (SEQ ID NO: 185), NLSKKKKRKREK (SEQ ID NO: 186), RRPSRPFRKP (SEQ ID NO: 187), KRPRSPSS (SEQ ID NO: 188), KRGINDRNFWRGENERKTR (SEQ ID NO: 189), PRPPKMARYDN (SEQ ID NO: 190), KRSFSKAF (SEQ ID NO: 191), KLKIKRPVK (SEQ ID NO: 192), PKKKRKVPPPPAAKRVKLD (SEQ ID NO: 193), PKTRRRPRRSQRKRPPT (SEQ ID NO:26792), SRRRKANPTKLSENAKKLAKEVEN (SEQ ID NO: 194), KTRRRPRRSQRKRPPT (SEQ ID NO: 195), RRKKRRPRRKKRR (SEQ ID NO: 196), PKKKSRKPKKKSRK (SEQ ID NO: 197), HKKKHPDASVNFSEFSK (SEQ ID NO: 198), QRPGPYDRPQRPGPYDRP (SEQ ID NO: 199), LSPSLSPLLSPSLSPL (SEQ ID NO: 200), RGKGGKGLGKGGAKRHRK (SEQ ID NO: 201), PKRGRGRPKRGRGR (SEQ ID NO: 202), PKKKRKVPPPPAAKRVKLD (SEQ ID NO: 203), PKKKRKVPPPPKKKRKV (SEQ ID NO: 204), PAKRARRGYKC (SEQ ID NO: 27199), KLGPRKATGRW (SEQ ID NO: 27200), PRRKREE (SEQ ID NO: 27201), PYRGRKE (SEQ ID NO: 27202), PLRKRPRR (SEQ ID NO: 27203), PLRKRPRRGSPLRKRPRR (SEQ ID NO: 27204), PAAKRVKLDGGKRTADGSEFESPKKKRKV (SEQ ID NO: 27205), PAAKRVKLDGGKRTADGSEFESPKKKRKVGIHGVPAA (SEQ ID NO: 27206), PAAKRVKLDGGKRTADGSEFESPKKKRKVAEAAAKEAAAKEAAAKA (SEQ ID NO: 207), PAAKRVKLDGGKRTADGSEFESPKKKRKVPG (SEQ ID NO: 27208), KRKGSPERGERKRHW (SEQ ID NO: 27209), KRTADSQHSTPPKTKRKVEFEPKKKRKV (SEQ ID NO: 27210), and PKKKRKVGGSKRTADSQHSTPPKTKRKVEFEPKKKRKV (SEQ ID NO: 27211), wherein the one or more NLS are linked to the CRISPR protein or to adjacent NLS with a linker peptide wherein the linker peptide is selected from the group consisting of RS, (G)n (SEQ ID NO: 27212), (GS)n (SEQ ID NO: 27213), (GSGGS)n (SEQ ID NO: 214), (GGSGGS)n (SEQ ID NO: 215), (GGGS)n (SEQ ID NO: 216), GGSG (SEQ ID NO: 217), GGSGG (SEQ ID NO: 218), GSGSG (SEQ ID NO: 219), GSGGG (SEQ ID NO: 220), GGGSG (SEQ ID NO: 221), GSSSG (SEQ ID NO: 222), GPGP (SEQ ID NO: 223), GGP, PPP, PPAPPA (SEQ ID NO: 224), PPPG (SEQ ID NO: 27214), PPPGPPP (SEQ ID NO: 225), PPP(GGGS)n (SEQ ID NO: 27215), (GGGS)nPPP (SEQ ID NO: 27216), AEAAAKEAAAKEAAAKA (SEQ ID NO: 27217), and TPPKTKRKVEFE (SEQ ID NO: 27218), wherein n is 1 to 5.

Embodiment 51. The system of embodiment 49 or embodiment 50, wherein the one or more NLS are located at or near the C-terminus of the CasX variant protein.

Embodiment 52. The system of embodiment 49 or embodiment 50, wherein the one or more NLS are located at or near the N-terminus of the CasX variant protein.

Embodiment 53. The system of embodiment 49 or embodiment 50, comprising one or more NLS located at or near the N-terminus and at or near the C-terminus of the CasX variant protein.

Embodiment 54. The system of any one of embodiments 41-53, wherein the CasX variant is capable of forming a ribonuclear protein complex (RNP) with a guide nucleic acid (gRNA).

Embodiment 55. The system of embodiment 54, wherein an RNP of the CasX variant protein and the gRNA variant exhibit at least one or more improved characteristics as compared to an RNP of a reference CasX protein of SEQ ID NO: 1, SEQ ID NO: 2, or SEQ ID NO: 3 and a gRNA comprising a sequence of any one of SEQ ID NOs: 4-16.

Embodiment 56. The system of embodiment 55, wherein the improved characteristic is selected from one or more of the group consisting of improved folding of the CasX variant; improved binding affinity to a guide nucleic acid (gRNA); improved binding affinity to a target DNA; improved ability to utilize a greater spectrum of one or more protospacer adjacent motif (PAM) sequences, including ATC, CTC, GTC, or TTC, in the editing of target DNA; improved unwinding of the target DNA; increased editing activity; improved editing efficiency; improved editing specificity; increased nuclease activity; improved target nucleic acid sequence cleavage rate; increased target strand loading for double strand cleavage; decreased target strand loading for single strand nicking; decreased off-target cleavage; improved binding of non-target DNA strand; improved protein stability; improved protein solubility; improved ribonuclear protein complex (RNP) formation; higher percentage of cleavage-competent RNP; improved protein:gRNA complex (RNP) stability; improved protein:gRNA complex solubility; improved protein yield; improved protein expression; and improved fusion characteristics.

Embodiment 57. The system of embodiment 55 or embodiment 56, wherein the improved characteristic of the RNP of the CasX variant protein and the gRNA variant is at least about 1.1 to about 100-fold or more improved relative to the RNP of the reference CasX protein of SEQ ID NO: 1, SEQ ID NO: 2, or SEQ ID NO: 3 and the gRNA comprising a sequence of any one of SEQ ID NOs: 4-16.

Embodiment 58. The system of embodiment 55 or embodiment 56, wherein the improved characteristic of the CasX variant protein is at least about 1.1, at least about 2, at least about 10, at least about 100-fold or more improved relative to the reference CasX protein of SEQ ID NO: 1, SEQ ID NO: 2, or SEQ ID NO: 3 and the gRNA comprising a sequence of any one of SEQ ID NOs: 4-16.

Embodiment 59. The system of embodiment 55 or embodiment 56, wherein the improved characteristic of the CasX variant protein is at least about 1.1, at least about 2, at least about 10, at least about 100-fold or more improved relative to the reference CasX protein of SEQ ID NO: 1, SEQ ID NO: 2, or SEQ ID NO: 3 and the gNA comprising a sequence of any one of SEQ ID NOS: 4-16.

Embodiment 60. The system of any one of embodiments 55-59, wherein the improved characteristic comprises editing efficiency, and the RNP of the CasX variant protein and the gRNA variant comprises a 1.1 to 100-fold improvement in editing efficiency compared to the RNP of the reference CasX protein of SEQ ID NO: 2 and the gRNA of any one of SEQ ID NOs: 4-16.

Embodiment 61. The system of any one of embodiments 54-59, wherein the RNP comprising the CasX variant and the gRNA variant exhibits greater editing efficiency and/or binding of a target nucleic acid sequence when any one of the PAM sequences TTC, ATC, GTC, or CTC is located 1 nucleotide 5′ to the non-target strand of a protospacer having identity with the targeting sequence of the gRNA in a cellular assay system compared to the editing efficiency and/or binding of an RNP comprising a reference CasX protein and a reference gRNA in a comparable assay system.

Embodiment 62. The system of embodiment 61, wherein the PAM sequence is TTC.

Embodiment 63. The system of embodiment 62, wherein the targeting sequence of the gRNA comprises a sequence selected from the group consisting of SEQ ID NOS: 17904-26789.

Embodiment 64. The system of embodiment 61, wherein the PAM sequence is ATC.

Embodiment 65. The system of embodiment 64, wherein the targeting sequence of the gRNA comprises a sequence selected from the group consisting of SEQ ID NOS: 272-2100 and 2286-5625.

Embodiment 66. The system of embodiment 61, wherein the PAM sequence is CTC.

Embodiment 67. The system of embodiment 66, wherein the targeting sequence of the gRNA comprises a sequence selected from the group consisting of SEQ ID NOS: 5626-13616.

Embodiment 68. The system of embodiment 61, wherein the PAM sequence is GTC.

Embodiment 69. The system of embodiment 66, wherein the targeting sequence of the gRNA comprises a sequence selected from the group consisting of SEQ ID NOS: 13617-17903.

Embodiment 70. The system of any one of embodiments 61-68, wherein the increased binding affinity for the one or more PAM sequences is at least 1.5-fold greater compared to the binding affinity of any one of the reference CasX proteins of SEQ ID NOS: 1-3 for the PAM sequences.

Embodiment 71. The system of any one of embodiments 54-70, wherein the RNP has at least a 5%, at least a 10%, at least a 15%, or at least a 20% higher percentage of cleavage-competent RNP compared to an RNP of the reference CasX protein and the gRNA of SEQ ID NOs: 4-16.

Embodiment 72. The system of any one of embodiments 41-71, wherein the CasX variant protein comprises a RuvC DNA cleavage domain having nickase activity.

Embodiment 73. The system of any one of embodiments 41-71, wherein the CasX variant protein comprises a RuvC DNA cleavage domain having double-stranded cleavage activity.

Embodiment 74. The system of any one of embodiments 1-54, wherein the CasX protein is a catalytically inactive CasX (dCasX) protein, and wherein the dCasX and the gRNA retain the ability to bind to the BCL11A target nucleic acid.

Embodiment 75. The system of embodiment 74, wherein the dCasX comprises a mutation at residues:
a. D672, E769, and/or D935 corresponding to the CasX protein of SEQ ID NO:1; or b. D659, E756 and/or D922 corresponding to the CasX protein of SEQ ID NO: 2.

Embodiment 76. The system of embodiment 75, wherein the mutation is a substitution of alanine for the residue.

Embodiment 77. The system of any one of embodiments 1-73, further comprising a donor template nucleic acid.

Embodiment 78. The system of embodiment 77, wherein the donor template comprises a nucleic acid comprising at least a portion of a BCL11A gene selected from the group consisting of a BCL11A exon, a BCL11A intron, a BCL11A intron-exon junction, a BCL11A regulatory element, and the GATA1 binding site sequence.

Embodiment 79. The system of embodiment 78, wherein the donor template sequence comprises one or more mutations relative to a corresponding portion of a wild-type BCL11A gene.

Embodiment 80. The system of embodiment 78 or embodiment 79, wherein the donor template comprises a nucleic acid comprising at least a portion of a BCL11A exon selected from the group consisting of BCL11A exon 1, BCL11A exon 2, BCL11A exon 3, BCL11A exon 4, BCL11A exon 5, BCL11A exon 6, BCL11A exon 7, BCL11A exon 8, and BCL11A exon 9.

Embodiment 81. The system of embodiment 80, wherein the donor template comprises a nucleic acid comprising at least a portion of a BCL11A exon selected from the group consisting of BCL11A exon 1, BCL11A exon 2, and BCL11A exon 3.

Embodiment 82. The system of any one of embodiments 77-81, wherein the donor template ranges in size from 10-15,000 nucleotides.

Embodiment 83. The system of any one of embodiments 77-82, wherein the donor template is a single-stranded DNA template or a single stranded RNA template.

Embodiment 84. The system of any one of embodiments 77-82, wherein the donor template is a double-stranded DNA template.

Embodiment 85. The system of any one of embodiments 77-84, wherein the donor template comprises homologous arms at or near the 5′ and 3′ ends of the donor template that are complementary to sequences flanking cleavage sites in the BCL11A target nucleic acid introduced by the Class 2 Type V CRISPR protein.

Embodiment 86. A nucleic acid comprising the donor template of any one of embodiments 77-85.

Embodiment 87. A nucleic acid comprising a sequence that encodes the CasX of any one of embodiments 41-76.

Embodiment 88. A nucleic acid comprising a sequence that encodes the gRNA of any one of embodiments 1-39.

Embodiment 89. The nucleic acid of embodiment 87, wherein the sequence that encodes the CasX protein is codon optimized for expression in a eukaryotic cell.

Embodiment A vector comprising the gRNA of any one of embodiments 1-39, the CasX protein of any one of embodiments 41-76, or the nucleic acid of any one of embodiments 86-89.

Embodiment 91. The vector of embodiment 90, wherein the vector further comprises one or more promoters.

Embodiment 92. The vector of embodiment 90 or embodiment 91, wherein the vector is selected from the group consisting of a retroviral vector, a lentiviral vector, an adenoviral vector, an adeno-associated viral (AAV) vector, a herpes simplex virus (HSV) vector, a virus-like particle (VLP), a CasX delivery particle (XDP), a plasmid, a minicircle, a nanoplasmid, a DNA vector, and an RNA vector.

Embodiment 93. The vector of embodiment 92, wherein the vector is an AAV vector.

Embodiment 94. The vector of embodiment 93, wherein the AAV vector is selected from AAV1, AAV2, AAV3, AAV4, AAV5, AAV6, AAV7, AAV8, AAV9, AAV10, AAV-Rh74, or AAVRh10.

Embodiment 95. The vector of embodiment 94, wherein the AAV vector is selected from AAV1, AAV2, AAV5, AAV8, or AAV9.

Embodiment 96. The vector of embodiment 94 or embodiment 95, wherein the AAV vector comprises a nucleic acid comprising the following components:
a. 5′ ITR;
b. a 3′ ITR; and
c. a transgene comprising the nucleic acid of embodiment 87 operably linked to a first promoter and the nucleic acid of embodiment 88 operably linked to a second promoter.

Embodiment 97. The vector of embodiment 96, wherein the nucleic acid further comprises a poly(A) sequence 3′ to the sequence encoding the CasX protein.

Embodiment 98. The vector of embodiment 96 or embodiment 97, wherein the nucleic acid further comprises one or more enhancer elements.

Embodiment 99. The vector of any one of embodiments 96-98, wherein a single AAV particle is capable of containing the nucleic acid, wherein the AAV particle has all the components necessary to transduce and effectively modify a target nucleic in a target cell.

Embodiment 100. The vector of embodiment 92, wherein the vector is a retroviral vector.

Embodiment 101. The vector of embodiment 92, wherein the vector is a XDP comprising one or more components of a gag polyprotein.

Embodiment 102. The vector of embodiment 101, wherein the one or more components of the gag polyprotein are selected from the group consisting of a matrix protein (MA), a nucleocapsid protein (NC), a capsid protein (CA), a pl peptide, a p6 peptide, a P2A peptide, a P2B peptide, a P10 peptide, a p12 peptide, a PP 21/24 peptide, a P12/P3/P8 peptide, and a P20 peptide.

Embodiment 103. The vector of embodiment 101 or embodiment 102, wherein the XDP comprises the one or more components of the gag polyprotein, the CasX protein, and the gRNA.

Embodiment 104. The vector of embodiment 103, wherein the CasX protein and the gRNA are associated together in an RNP.

Embodiment 105. The vector of any one of embodiments 101-104, further comprising the donor template.

Embodiment 106. The vector of any one of embodiments 101-104, further comprising a pseudotyping viral envelope glycoprotein or antibody fragment that provides for binding and fusion of the XDP to a target cell.

Embodiment 107. The vector of embodiment of embodiment, wherein the target cell is selected from the group consisting of a hematopoietic stem cell (HSC), a hematopoietic progenitor cell (HPC), a CD34+ cell, a mesenchymal stem cell (MSC), an embryonic stem (ES) cell, an induced pluripotent stem cell (iPSC), a common myeloid progenitor cell, a proerythroblast cell, and an erythroblast cell.

Embodiment 108. A host cell comprising the vector of any one of embodiments 90-107..

Embodiment 109. The host cell of embodiment 108, wherein the host cell is selected from the group consisting of BHK, HEK293, HEK293T, NS0, SP2/0, YO myeloma cells, P3X63 mouse myeloma cells, PER, PER.C6, NIH3T3, COS, HeLa, CHO, and yeast cells.

Embodiment 110. A method of modifying a BCL11A target nucleic acid sequence in a population of cells, the method comprising introducing into cells of the population:
a. the system of any one of embodiments 1-85;
b. the nucleic acid of any one of embodiments 86-89;
c. the vector as in any one of embodiments 90-95;
d. the XDP of any one of embodiments 101-107; or
e. combinations of two or more of (a)-(d),
   wherein the BCL11A gene target nucleic acid sequence of the cells targeted by the first gRNA is modified by the CasX variant protein.

Embodiment 111. The method of embodiment 110, wherein the modifying comprises introducing a single-stranded break in the BCL11A gene target nucleic acid sequence of the cells of the population.

Embodiment 112. The method of embodiment 110, wherein the modifying comprises introducing a double-stranded break in the BCL11A gene target nucleic acid sequence of the cells of the population.

Embodiment 113. The method of any one of embodiments 110-112, further comprising introducing into the cells of the population a second gRNA or a nucleic acid encoding the second gRNA, wherein the second gRNA has a targeting sequence complementary to a different or overlapping portion of the BCL11A gene target nucleic acid compared to the first gRNA, resulting in an additional break in the BCL11A target nucleic acid of the cells of the population.

Embodiment 114. The method of any one of embodiments 110-113, wherein the modifying comprises introducing an insertion, deletion, substitution, duplication, or inversion of one or more nucleotides in the BCL11A gene of the cells of the population.

Embodiment 115. The method of embodiment 110-114, wherein a GATA1 binding site sequence of the target nucleic acid is modified.

Embodiment 116. The method of any one of embodiments 110-113, wherein the method comprises insertion of the donor template into the break site(s) of the BCL11A gene target nucleic acid sequence of the cells of the population.

Embodiment 117. The method of embodiment 114, wherein the insertion of the donor template is mediated by homology-directed repair (HDR) or homology-independent targeted integration (HITI).

Embodiment 118. The method of embodiment 116 or embodiment 117, wherein the GATA1 binding site sequence of the target nucleic acid is modified.

Embodiment 119. The method of any one of embodiments 116-118, wherein insertion of the donor template results in a knock-down or knock-out of the BCL11A gene in the cells of the population.

Embodiment 120. The method of any one of embodiments 110-119, wherein the BCL11A gene of the cells of the population is modified such that expression of the BCL11A protein is reduced by at least about 10%, at least about 20%, at least about 30%, at least about 40%, at least about 50%, at least about 60%, at least about 70%, at least about 80%, or at least about 90% in comparison to cells in which the BCL11A gene has not been modified.

Embodiment 121. The method of any one of embodiments 110-119, wherein the BCL11A gene of the cells of the population is modified such that at least about 10%, at least about 20%, at least about 30%, at least about 40%, at least about 50%, at least about 60%, at least about 70%, at least about 80%, or at least about 90% of the modified cells do not express a detectable level of BCL11A protein.

Embodiment 122. The method of any one of embodiments 110-121, wherein the cells are eukaryotic.

Embodiment 123. The method of embodiment 122, wherein the eukaryotic cells are selected from the group consisting of rodent cells, mouse cells, rat cells, and non-human primate cells.

Embodiment 124. The method of embodiment 122, wherein the eukaryotic cells are human cells.

Embodiment 125. The method of any one of embodiments 122-124, wherein the eukaryotic cell is selected from the group consisting of a hematopoietic stem cell (HSC), a hematopoietic progenitor cell (HPC), a CD34+ cell, a mesenchymal stem cell (MSC), induced pluripotent stem cell (iPSC), a common myeloid progenitor cell, a proerythroblast cell, and an erythroblast cell.

Embodiment 126. The method of any one of embodiment 110-125, wherein the modification of the BCL11A gene target nucleic acid sequence of the population of cells occurs in vitro or ex vivo.

Embodiment 127. The method of any one of embodiment 110-125, wherein the modification of the BCL11A gene target nucleic acid sequence of the population of cells occurs in vivo in a subject.

Embodiment 128. The method of embodiment 127, wherein the subject is selected from the group consisting of a rodent, a mouse, a rat, and a non-human primate.

Embodiment 129. The method of embodiment 127, wherein the subject is a human.

Embodiment 130. The method of any one of embodiments 127-129, wherein the method comprises administering a therapeutically effective dose of the AAV vector to the subject.

Embodiment 131. The method of embodiment 130, wherein the AAV vector is administered to the subject at a dose of at least about 1×10⁵ vector genomes/kg (vg/kg), at least about 1×10⁶ vg/kg, at least about 1×10⁷ vg/kg, at least about 1×10⁸ vg/kg, at least about 1×109 vg/kg, at least about 1×10¹⁰ vg/kg, at least about 1×10¹¹ vg/kg, at least about 1×10¹² vg/kg, at least about 1×10¹³ vg/kg, at least about 1×10¹⁴ vg/kg, at least about 1×10¹⁵ vg/kg, or at least about 1×10¹⁶ vg/kg.

Embodiment 132. The method of embodiment 130, wherein the AAV vector is administered to the subject at a dose of at least about 1×10⁵ vg/kg to about 1×10¹⁶ vg/kg, at least about 1×10⁶ vg/kg to about 1×10¹⁵ vg/kg, or at least about 1×10⁷ vg/kg to about 1×10¹⁴ vg/kg.

Embodiment 133. The method of any one of embodiments 127-129, wherein the method comprises administering a therapeutically effective dose of a XDP to the subject.

Embodiment 134. The method of embodiment 133, wherein the XDP is administered to the subject at a dose of at least about 1×10⁵ particles/kg, at least about 1×10⁶ particles/kg, at least about 1×10⁷ particles/kg at least about 1×10⁸ particles/kg, at least about 1×10⁹ particles/kg, at least about 1×10¹⁰ particles/kg, at least about 1×10¹¹ particles/kg, at least about 1×10¹² particles/kg, at least about 1×10¹³ particles/kg, at least about 1×10¹⁴ particles/kg, at least about 1×10¹⁵ particles/kg, at least about 1×10¹⁶ particles/kg.

Embodiment 135. The method of embodiment 133, wherein the XDP is administered to the subject at a dose of at least about 1×10⁵ particles/kg to about 1×10¹⁶ particles/kg, or at least about 1×10⁶ particles/kg to about 1×10¹⁵ particles/kg, or at least about 1×10⁷ particles/kg to about 1×10¹⁴ particles/kg

Embodiment 136. The method of any one of embodiments 128-135, wherein the vector or XDP is administered to the subject by a route of administration selected from transplantation, local injection, systemic infusion, or combinations thereof.

Embodiment 137. The method of any one of embodiments 128-136, wherein the method results in an increased levels of hemoglobin F (HbF) in circulating blood of the subject of at least about 5%, at least about 10%, at least about 20%, at least about 30%, at least about 40%, or at least about 50% compared to the levels of HbF in the subject prior to treatment.

Embodiment 138. The method of any one of embodiments 128-137, wherein the method results in a ratio of HbF to hemoglobin S (HbS) in circulating blood of the subject of at least 0.01:1.0, at least 0.025:1.0, at least 0.05:1.0, at least 0.075:1.0 at least 0.1:1.0, at least 0.2:1.0, at least 0.3:1.0, at least 0.4:1.0, at least 0.5:1:0, at least 0.75:1.0, at least 1.0:1.0, at least 1.25:1.0, at least 1.5:1.0, or at least 1.75:1.0.

Embodiment 139. The method of any one of embodiments 128-138, wherein the method results in HbF levels of at least about 5%, or at least about 10%, or at least about 20%, or at least about 30% of total hemoglobin in circulating blood of the subject.

Embodiment 140. The method of any one of embodiments 110-139, further comprising contacting the BCL11A gene target nucleic acid sequence of the population of cells with:
a. an additional CRISPR nuclease and a gRNA targeting a different or overlapping portion of the BCL11A target nucleic acid compared to the first gRNA;
b. a polynucleotide encoding the additional CRISPR nuclease and the gRNA of (a);
c. a vector comprising the polynucleotide of (b); or
d. a XDP comprising the additional CRISPR nuclease and the gRNA of (a)
   wherein the contacting results in modification of the BCL11A gene at a different location in the sequence compared to the sequence targeted by the first gRNA.

Embodiment 141. The method of embodiment 140, wherein the additional CRISPR nuclease is a CasX protein having a sequence different from the CasX protein of any of the preceding embodiments.

Embodiment 142. The method of embodiment 140, wherein the additional CRISPR nuclease is not a CasX protein.

Embodiment 143. The method of embodiment 142, wherein the additional CRISPR nuclease is selected from the group consisting of Cas9, Cas12a, Cas12b, Cas12c, Cas12d (CasY), Cas12j, Cas12k, Cas13a, Cas13b, Cas13c, Cas13d, Cas14, Cpf1, C2cl, Csn2, and sequence variants thereof.

Embodiment 144. A population of cells modified by the method of any one of embodiments 110-143, wherein the cells have been modified such that at least 70%, at least 75%, at least 80%, at least 85%, at least 90%, or at least 95% of the modified cells do not express a detectable level of BCL11A protein.

Embodiment 145. A population of cells modified by the method of any one of embodiments 110-143, wherein the cells have been modified such that the expression of BCL11A protein is reduced by at least 70%, at least 75%, at least 80%, at least 85%, at least 90%, or at least 95% compared to cells where the BCL11A gene has not been modified.

Embodiment 146. A method of treating a hemoglobinopathy in a subject in need thereof, the method comprising administering to the subject a therapeutically effective amount of the cells of embodiment 144 or embodiment 145.

Embodiment 147. The method of embodiment 146, wherein the hemoglobinopathy is a sickle cell disease or beta-thalassemia.

Embodiment 148. The method of embodiment 146 or embodiment 147, wherein the cells are autologous with respect to the subject to be administered the cells.

Embodiment 149. The method of embodiments 146 or embodiment 147, wherein the cells are allogeneic with respect to the subject to be administered the cells.

Embodiment 150. The method of any one of embodiments 146-149, wherein the cells or their progeny persist in the subject for at least one month, two month, three months, four months, five months, six months, seven months, eight months, nine months, ten months, eleven months, twelve months, thirteen months, fourteen month, fifteen months, sixteen months, seventeen months, eighteen months, nineteen months, twenty months, twenty-one months, twenty-two months, twenty-three months, two years, three years, four years, or five years after administration of the modified cells to the subject.

Embodiment 151. The method of any one of embodiments 146-150, wherein the method results in an increased levels of hemoglobin F (HbF) in circulating blood of at least about 5%, at least about 10%, at least about 20%, at least about 30%, at least about 40%, or at least about 50% compared to the levels of HbF in the subject prior to treatment.

Embodiment 152. The method of any one of embodiments 146-150, wherein the method results in a ratio of HbF to hemoglobin S (HbS) in the subject of at least 0.01:1.0, at least 0.025:1.0, at least 0.05:1.0, at least 0.075:1.0 at least 0.1:1.0, at least 0.2:1.0, at least 0.3:1.0, at least 0.4:1.0, at least 0.5:1:0, at least 0.75:1.0, at least 1.0:1.0, at least 1.25:1.0, at least 1.5:1.0, or at least 1.75:1.0.

Embodiment 153. The method of any one of embodiments 146-150, wherein the method results in HbF levels of at least about 5%, or at least about 10%, or at least about 20%, or at least about 30% of total circulating hemoglobin in the subject.

Embodiment 154. The method of any one of embodiments 146-153, wherein the subject is selected from the group consisting of a rodent, a mouse, a rat, and a non-human primate.

Embodiment 155. The method of any one of embodiments 146-153, wherein the subject is a human.

Embodiment 156. A method of treating a hemoglobinopathy in a subject in need thereof, comprising modifying a BCL11A gene in cells of the subject, the modifying comprising contacting said cells with a therapeutically effective dose of:
a. the system of any one of embodiments 1-85;
b. the nucleic acid of any one of embodiments 86-89;
c. the vector as in any one of embodiments 90-95;
d. the XDP of any one of embodiments 101-104; or
e. combinations of two or more of (a)-(d),
   wherein the BCL11A gene of the cells targeted by the first gRNA is modified by the CasX protein.

Embodiment 157. The method of embodiment 156, wherein the hemoglobinopathy is sickle cell disease or beta-thalassemia.

Embodiment 158. The method of any one of embodiments 156 or embodiment 157, wherein the cell is selected from the group consisting of hematopoietic stem cells (HSC), hematopoietic progenitor cells (HPC), CD34+ cells, mesenchymal stem cells (MSC), induced pluripotent stem cells (iPSC), common myeloid progenitor cells, proerythroblast cells, and erythroblast cells.

Embodiment 159. The method of any one of embodiments 156-158, wherein the modifying comprises introducing a single-stranded break in the BCL11A gene of the cells.

Embodiment 160. The method of any one of embodiments 156-158, wherein the modifying comprises introducing a double-stranded break in the BCL11A gene of the cells.

Embodiment 161. The method of any one of embodiments 156-160, further comprising introducing into the cells of the subject a second gRNA or a nucleic acid encoding the second gRNA, wherein the second gRNA has a targeting sequence complementary to a different or overlapping portion of the target nucleic acid compared to the first gRNA, resulting in an additional break in the BCL11A target nucleic acid of the cells of the subject.

Embodiment 162. The method of any one of embodiments 156-161, wherein the modifying comprises introducing an insertion, deletion, substitution, duplication, or inversion of one or more nucleotides in the BCL11A gene of the cells.

Embodiment 163. The method of embodiment 162, wherein the modifying results in a knock-down or knock-out of the BCL11A gene in the modified cells of the subject.

Embodiment 164. The method of any one of embodiments 156-163, wherein the BCL11A gene of the cells are modified such that expression of the BCL11A protein by the modified cells is reduced by at least about 10%, at least about 20%, at least about 30%, at least about 40%, at least about 50%, at least about 60%, at least about 70%, at least about 80%, or at least about 90% in comparison to cells that have not been modified.

Embodiment 165. The method of any one of embodiments 156-163, wherein the BCL11A gene of the cells of the subject are modified such that at least 70%, at least 75%, at least 80%, at least 85%, at least 90%, or at least 95% of the modified cells do not express a detectable level of BCL11A protein.

Embodiment 166. The method of any one of embodiments 156-165, wherein the method results in an increased levels of hemoglobin F (HbF) in circulating blood of the subject of at least about 5%, at least about 10%, at least about 20%, at least about 30%, at least about 40%, or at least about 50% compared to the levels of HbF in the subject prior to treatment.

Embodiment 167. The method of any one of embodiments 156-166, wherein the method results in a ratio of HbF to hemoglobin S (HbS) in circulating blood of the subject of at least 0.01:1.0, at least 0.025:1.0, at least 0.05:1.0, at least 0.075:1.0 at least 0.1:1.0, at least 0.2:1.0, at least 0.3:1.0, at least 0.4:1.0, at least 0.5:1:0, at least 0.75:1.0, at least 1.0:1.0, at least 1.25:1.0, at least 1.5:1.0, or at least 1.75:1.0.

Embodiment 168. The method of any one of embodiments 156-165, wherein the method results in HbF levels of at least about 5%, or at least about 10%, or at least about 20%, or at least about 30% of total hemoglobin in circulating blood of the subject.

Embodiment 169. The method of any one of embodiments 156-161, wherein the method comprises insertion of the donor template into the break site(s) of the BCL11A gene target nucleic acid sequence of the cells.

Embodiment 170. The method of embodiment 168, wherein the insertion of the donor template is mediated by homology-directed repair (HDR) or homology-independent targeted integration (HITI).

Embodiment 171. The method of embodiment 168 or embodiment 170, wherein insertion of the donor template results in a knock-down or knock-out of the BCL11A gene in the modified cells of the subject.

Embodiment 172. The method of any one of embodiments 166-171, wherein the BCL11A gene of the cells are modified such that expression of the BCL11A protein by the modified cells is reduced by at least about 10%, at least about 20%, at least about 30%, at least about 40%, at least about 50%, at least about 60%, at least about 70%, at least about 80%, or at least about 90% in comparison to cells that have not been modified.

Embodiment 173. The method of any one of embodiments 166-171, wherein the BCL11A gene of the cells of the subject are modified such that at least 70%, at least 75%, at least 80%, at least 85%, at least 90%, or at least 95% of the modified cells do not express a detectable level of BCL11A protein.

Embodiment 174. The method of any one of embodiments 166-173, wherein the method results in an increased levels of hemoglobin F (HbF) in circulating blood of the subject of at least about 5%, at least about 10%, at least about 20%, at least about 30%, at least about 40%, or at least about 50% compared to the levels of HbF in the subject prior to treatment.

Embodiment 175. The method of any one of embodiments 166-173, wherein the method results in a ratio of HbF to hemoglobin S (HbS) in circulating blood of the subject of at least 0.01:1.0, at least 0.025:1.0, at least 0.05:1.0, at least 0.075:1.0 at least 0.1:1.0, at least 0.2:1.0, at least 0.3:1.0, at least 0.4:1.0, at least 0.5:1:0, at least 0.75:1.0, at least 1.0:1.0, at least 1.25:1.0, at least 1.5:1.0, or at least 1.75:1.0.

Embodiment 176. The method of any one of embodiments 166-173, wherein the method results in HbF levels of at least about 5%, or at least about 10%, or at least about 20%, or at least about 30% of total hemoglobin in circulating blood of the subject.

Embodiment 177. The method of any one of embodiments 156-175, wherein the subject is selected from the group consisting of rodent, mouse, rat, and non-human primate.

Embodiment 178. The method of any one of embodiments 156-175, wherein the subject is a human.

Embodiment 179. The method of any one of embodiments 156-178, wherein the vector is AAV and is administered to the subject at a dose of at least about 1×10⁵ vector genomes/kg (vg/kg), at least about 1×10⁶ vg/kg, at least about 1×10⁷ vg/kg, at least about 1×10⁸ vg/kg, at least about 1×10⁹ vg/kg, at least about 1×10¹⁰ vg/kg, at least about 1×10¹¹ vg/kg, at least about 1×10¹² vg/kg, at least about 1×10¹³ vg/kg, at least about 1×10¹⁴ vg/kg, at least about 1×10¹⁵ vg/kg, or at least about 1×10¹⁶ vg/kg.

Embodiment 180. The method of any one of embodiments 156-178, wherein the vector is AAV and is administered to the subject at a dose of at least about 1×10⁵ vg/kg to about 1×10¹⁶ vg/kg, at least about 1×10⁶ vg/kg to about 1×10¹⁵ vg/kg, or at least about 1×10⁷ vg/kg to about 1×10¹⁴ vg/kg.

Embodiment 181. The method of any one of embodiments 156-178, wherein the XDP is administered to the subject at a dose of at least about 1×10⁵ particles/kg, at least about 1×10⁶ particles/kg, at least about 1×10⁷ particles/kg at least about 1×10⁸ particles/kg, at least about 1×10⁹ particles/kg, at least about 1×10¹⁰ particles/kg, at least about 1×10¹¹ particles/kg, at least about 1×10¹² particles/kg, at least about 1×10¹³ particles/kg, at least about 1×10¹⁴ particles/kg, at least about 1×10¹⁵ particles/kg, at least about 1×10¹⁶ particles/kg.

Embodiment 182. The method of any one of embodiments 156-178, wherein the XDP is administered to the subject at a dose of at least about 1×10⁵ particles/kg to about 1×10¹⁶ particles/kg, or at least about 1×10⁶ particles/kg to about 1×10¹⁵ particles/kg, or at least about 1×10⁷ particles/kg to about 1×10¹⁴ particles/kg.

Embodiment 183. The method of any one of embodiments 156-182, wherein the vector or XDP is administered to the subject by a route of administration selected from intraparenchymal, intravenous, intra-arterial, intraperitoneal, intracapsular, subcutaneously, intramuscularly, intraabdominally, or combinations thereof, wherein the administering method is injection, transfusion, or implantation.

Embodiment 184. The method of any one of embodiments 156-183, wherein the method results in improvement in at least one clinically-relevant endpoint in the subject.

Embodiment 185. The method of embodiment 184, wherein the method results in improvement in at least one clinically-relevant parameter selected from the group consisting of occurrence of end-organ disease, albuminuria, hypertension, hyposthenia, hyposthenuria, diastolic dysfunction, functional exercise capacity, acute coronary syndrome, pain events, pain severity, anemia, hemolysis, tissue hypoxia, organ dysfunction, abnormal hematocrit values, childhood mortality, incidence of strokes, hemoglobin levels compared to baseline, HbF levels, reduced incidence of pulmonary embolisms, incidence of vaso-occlusive crises, concentration of hemoglobin S in erythrocytes, rate of hospitalizations, liver iron concentration, required blood transfusions, and quality of life score.

Embodiment 186. The method of embodiment 184, wherein the method results in improvement in at least two clinically-relevant parameters selected from the group consisting of occurrence of end-organ disease, albuminuria, hypertension, hyposthenia, hyposthenuria, diastolic dysfunction, functional exercise capacity, acute coronary syndrome, pain events, pain severity, anemia, hemolysis, tissue hypoxia, organ dysfunction, abnormal hematocrit values, childhood mortality, incidence of strokes, hemoglobin levels compared to baseline, HbF levels, reduced incidence of pulmonary embolisms, incidence of vaso-occlusive crises, concentration of hemoglobin S in erythrocytes, rate of hospitalizations, liver iron concentration, required blood transfusions, and quality of life score.

Embodiment 187. A method for treating a subject with a hemoglobinopathy, the method comprising:
a. isolating induced pluripotent stem cells (iPSC) or hematopoietic stem cells (HSC) from a subject;
b. modifying the BCL11A target nucleic acid of the iPSC or HSC by the method of any one of embodiments 110-126;
c. differentiating the modified iPSC or HSC into a hematopoietic progenitor cell; and
d. implanting the hematopoietic progenitor cell into the subject with the hemoglobinopathy,
   wherein the method results in an increased levels of hemoglobin F (HbF) in circulating blood of the subject of at least about 5%, at least about 10%, at least about 20%, at least about 30%, at least about 40%, or at least about 50% compared to the levels of HbF in the subject prior to treatment.

Embodiment 188. The method of embodiment 187, wherein the iPSC or HSC is autologous and is isolated from the subject's bone marrow or peripheral blood.

Embodiment 189. The method of embodiment 187, wherein the iPSC or HSC is allogeneic and is isolated from a different subject's bone marrow or peripheral blood.

Embodiment 190. The method of any one of embodiments 187-189, wherein the implanting comprises administering the hematopoietic progenitor cell to the subject by transplantation, local injection, systemic infusion, or combinations thereof.

Embodiment 191. The method of any one of embodiments 187-190, wherein the hemoglobinopathy is sickle cell disease or beta-thalassemia.

Embodiment 192. A method of increasing fetal hemoglobin (HbF) in a subject by genome editing, the method comprising:
a. administering to the subject an effective dose of the vector of any one of embodiments 90-95 or the XDP of any one of embodiments 101-107, wherein the vector or XDP delivers the CasX:gRNA system to cells of the subject;
b. the BCL11A target nucleic acid of cells of the subject are edited by the CasX targeted by the first gRNA;
c. the editing comprises introducing an insertion, deletion, substitution, duplication, or inversion of one or more nucleotides in the target nucleic acid sequence such that expression of BCL11A protein is reduced or eliminated,
   wherein the method results in an increased levels of hemoglobin F (HbF) in circulating blood of the subject of at least about 5%, at least about 10%, at least about 20%, at least about 30%, at least about 40%, or at least about 50% compared to the levels of HbF in the subject prior to treatment.

Embodiment 193. The method of embodiment 192, wherein the method results in a ratio of HbF to hemoglobin S (HbS) in the subject of at least 0.01:1.0, at least 0.025:1.0, at least 0.05:1.0, at least 0.075:1.0 at least 0.1:1.0, at least 0.2:1.0, at least 0.3:1.0, at least 0.4:1.0, at least 0.5:1:0, at least 0.75:1.0, at least 1.0:1.0, at least 1.25:1.0, at least 1.5:1.0, or at least 1.75:1.0.

Embodiment 194. The method of embodiment 192 or embodiment 193, wherein the method results in HbF levels of at least about 5%, or at least about 10%, or at least about 20%, or at least about 30% of total circulating hemoglobin in the subject.

Embodiment 195. The method of any one of embodiments 192-194, wherein the cells are selected from the group consisting of hematopoietic stem cells (HSC), hematopoietic progenitor cells (HPC), CD34+ cells, mesenchymal stem cells (MSC), induced pluripotent stem cells (iPSC), common myeloid progenitor cells, proerythroblast cells, and erythroblast cells.

Embodiment 196. The method of any one of embodiments 192-195, wherein the target nucleic acid of the cells has been edited such that expression of the BCL11A protein is reduced by at least about 10%, at least about 20%, at least about 30%, at least about 40%, at least about 50%, at least about 60%, at least about 70%, at least about 80%, or at least about 90% in comparison to target nucleic acid of cells that have not been edited.

Embodiment 197. The method of any one of embodiments 192-196, wherein the subject is selected from the group consisting of mouse, rat, pig, and non-human primate.

Embodiment 198. The method of any one of embodiments 192-196, wherein the subject is a human.

Embodiment 199. The method of any one of embodiments 192-198, wherein the vector is administered at a dose of at least about 1×10⁵ vector genomes/kg (vg/kg), at least about 1×106 vg/kg, at least about 1×10⁷ vg/kg, at least about 1×10⁸ vg/kg, at least about 1×10⁹ vg/kg, at least about 1×10¹⁰ vg/kg at least about 1×10¹¹ vg/kg, at least about 1×10¹² vg/kg, at least about 1×10¹³ vg/kg, at least about 1×10¹⁴ vg/kg, at least about 1×10¹⁵ vg/kg, or at least about 1×10¹⁶ vg/kg.

Embodiment 200. The method of any one of embodiments 192-198, wherein the XDP is administered at a dose of at least about 1×10⁵ particles/kg, at least about 1×10⁶ particles/kg, at least about 1×10⁷ particles/kg, at least about 1×10⁸ particles/kg, at least about 1×10⁹ particles/kg, at least about 1×10¹⁰ particles/kg at least about 1×10¹¹ particles/kg, at least about 1×10¹² particles/kg, at least about 1×10¹³ particles/kg, at least about 1×10¹⁴ particles/kg, at least about 1×10¹⁵ particles/kg, or at least about 1×10¹⁶ particles/kg.

Embodiment 201. The method of any one of embodiments 192-200, wherein the vector or XDP is administered by a route of administration selected from transplantation, local injection, systemic infusion, or combinations thereof.

Embodiment 202. The system of any one of embodiments 1-85, the nucleic acid of any one of embodiments 86-89, the vector of any one of 90-95, the XDP of any one of embodiments 101-104, the host cell of embodiment 108 or embodiment 109, or the population of cells of embodiment 144 or embodiment 145, for use as a medicament for the treatment of a hemoglobinopathy.

Embodiment 203. The system of embodiment 1, wherein the target nucleic acid sequence is complementary to a non-target strand sequence located 1 nucleotide 3′ of a protospacer adjacent motif (PAM) sequence.

Embodiment 204. The system of embodiment 203, wherein the PAM sequence comprises a TC motif.

Embodiment 205. The system of embodiment 204, wherein the PAM sequence comprises ATC, GTC, CTC or TTC.

Embodiment 206. The system of any one of embodiments 203-205, wherein the Class 2 Type V CRISPR protein comprises a RuvC domain.

Embodiment 207. The system of embodiment 206, wherein the RuvC domain generates a staggered double-stranded break in the target nucleic acid sequence.

Embodiment 208. The system of any one of embodiments 203-207, wherein the Class 2 Type V CRISPR protein does not comprise an HNH nuclease domain.

### EXAMPLES

### Example 1: Generating CasX Variant Constructs

In order to generate the CasX 488 construct (sequences in Table 6), the codon-optimized CasX 119 construct (based on the CasX Stx2 construct, encoding Planctomycetes CasX SEQ ID NO: 2, with amino acid substitutions and deletions) was cloned into a destination plasmid (pStX) using standard cloning methods. In order to generate the CasX 491 construct (sequences in Table 6), the codon-optimized CasX 484 construct (based on the CasX Stx2 construct, encoding Planctomycetes CasX SEQ ID NO: 2, with substitutions and deletions of certain amino acids, with fused NLS, and linked guide and non-targeting sequences) was cloned into a destination plasmid (pStX) using standard cloning methods. Construct CasX 1 (CasX SEQ ID NO: 1) was cloned into a destination vector using standard cloning methods. To build CasX 488, the CasX 119 construct DNA was PCR amplified in two reactions using Q5 DNA polymerase according to the manufacturer's protocol, using universal appropriate primers. To build CasX 491, the codon optimized CasX 484 construct DNA was PCR amplified in two reactions using Q5 DNA polymerase according to the manufacturer's protocol, using appropriate primers. The CasX 1 construct was PCR amplified in two reactions using Q5 DNA polymerase according to the manufacturer's protocol, universal appropriate primers. Each of the PCR products were purified by gel extraction from a 1% agarose gel (Gold Bio Cat #A-201-500) using Zymoclean Gel DNA Recovery Kit according to the manufacturer's protocol. The corresponding fragments were then pieced together using Gibson assembly (New England BioLabs Cat #E2621S) following the manufacturer's protocol. Assembled products in pStx1 were transformed into chemically-competent Turbo Competent E. coli bacterial cells, plated on LB-Agar plates containing kanamycin. Individual colonies were picked and miniprepped using Qiagen spin Miniprep Kit following the manufacturer's protocol. The resultant plasmids were sequenced using Sanger sequencing to ensure correct assembly. The correct clones were then subcloned into the mammalian expression vector pStx34 using restriction enzyme cloning. The pStx34 backbone and the CasX 488 and 491 clones in pStx1 were digested with XbaI and BamHI respectively. The digested backbone and respective insert fragments were purified by gel extraction from a 1% agarose gel (Gold Bio Cat #A-201-500) using Zymoclean Gel DNA Recovery Kit according to the manufacturer's protocol. The clean backbone and insert were then ligated together using T4 Ligase (New England Biolabs Cat #M0202L) according to the manufacturer's protocol. The ligated products were transformed into chemically-competent Turbo Competent E. coli bacterial cells, plated on LB-Agar plates containing carbenicillin. Individual colonies were picked and miniprepped using Qiagen spin Miniprep Kit following the manufacturer's protocol. The resultant plasmids were sequenced using Sanger sequencing to ensure correct assembly.

To build CasX 515 (sequences in Table 6), the CasX 491 construct DNA was PCR amplified in two reactions using Q5 DNA polymerase according to the manufacturer's protocol, using appropriate primers. To build CasX 527 (sequences in Table 6), the CasX 491 construct DNA was PCR amplified in two reactions using Q5 DNA polymerase according to the manufacturer's protocol, using appropriate primers. The PCR products were purified by gel extraction from a 1% agarose gel using Zymoclean Gel DNA Recovery Kit according to the manufacturer's protocol. The pStX backbone was digested using XbaI and SpeI in order to remove the 2931 base pair fragment of DNA between the two sites in plasmid pStx56. The digested backbone fragment was purified by gel extraction from a 1% agarose gel using Zymoclean Gel DNA Recovery Kit according to the manufacturer's protocol. The insert and backbone fragments were then pieced together using Gibson assembly (New England BioLabs Cat #E2621S) following the manufacturer's protocol. Assembled products in the pStx56 were transformed into chemically-competent Turbo Competent E. coli bacterial cells, plated on LB-Agar plates containing kanamycin. Individual colonies were picked and miniprepped using Qiagen spin Miniprep Kit following the manufacturer's protocol. The resultant plasmids were sequenced using Sanger sequencing to ensure correct assembly. pStX34 includes an EF-1a promoter for the protein as well as a selection marker for both puromycin and carbenicillin. pStX56 includes an EF-1a promoter for the protein as well as a selection marker for both puromycin and kanamycin Sequences encoding the targeting sequences that target the gene of interest were designed based on CasX PAM locations. Targeting sequence DNA was ordered as single-stranded DNA (ssDNA) oligos (Integrated DNA Technologies) consisting of the targeting sequence and the reverse complement of this sequence. These two oligos were annealed together and cloned into pStX individually or in bulk by Golden Gate assembly using T4 DNA Ligase and an appropriate restriction enzyme for the plasmid. Golden Gate products were transformed into chemically or electro-competent cells such as NEB Turbo competent E. coli (NEB Cat #C2984I), plated on LB-Agar plates containing the appropriate antibiotic. Individual colonies were picked and miniprepped using Qiaprep spin Miniprep Kit and following the manufacturer's protocol. The resultant plasmids were sequenced using Sanger sequencing to ensure correct ligation.

To build CasX 535-537 (sequences in Table 6), the CasX 515 construct DNA was PCR amplified in two reactions for each construct using Q5 DNA polymerase according to the manufacturer's protocol. For CasX 535, appropriate primers were used for the amplification. For CasX 536 appropriate primers were used. For CasX 537, appropriate primers were used. The PCR products were purified by gel extraction from a 1% agarose gel using Zymoclean Gel DNA Recovery Kit according to the manufacturer's protocol. The pStX backbone was digested using XbaI and SpeI in order to remove the 2931 base pair fragment of DNA between the two sites in plasmid pStx56. The digested backbone fragment was purified by gel extraction from a 1% agarose gel using Zymoclean Gel DNA Recovery Kit according to the manufacturer's protocol. The insert and backbone fragments were then pieced together using Gibson assembly following the manufacturer's protocol. Assembled products in pStx56 were transformed into chemically-competent Turbo Competent E. coli bacterial cells, plated on LB-Agar plates containing kanamycin. Individual colonies were picked and miniprepped using Qiagen spin Miniprep Kit following the manufacturer's protocol. The resultant plasmids were sequenced using Sanger sequencing to ensure correct assembly. pStX34 includes an EF-1a promoter for the protein as well as a selection marker for both puromycin and carbenicillin. pStX56 includes an EF-1a promoter for the protein as well as a selection marker for both puromycin and kanamycin. Sequences encoding the targeting sequences that target the gene of interest were designed based on CasX PAM locations. Targeting sequence DNA was ordered as single-stranded DNA (ssDNA) oligos (Integrated DNA Technologies) consisting of the targeting sequence and the reverse complement of this sequence. These two oligos were annealed together and cloned into pStX individually or in bulk by Golden Gate assembly using T4 DNA Ligase and an appropriate restriction enzyme for the plasmid. Golden Gate products were transformed into chemically or electro-competent cells such as NEB Turbo competent E. coli, plated on LB-Agar plates containing the appropriate antibiotic. Individual colonies were picked and miniprepped using Qiaprep spin Miniprep Kit and following the manufacturer's protocol. The resultant plasmids were sequenced using Sanger sequencing to ensure correct ligation.

All subsequent CasX variants, such as CasX 544 and CasX 660-664, 668, 670, 672, 676, and 677 were cloned using the same methodology as described above using Gibson assembly with mutation-specific internal primers and universal forward and reverse primers (the differences between them were the mutation specific primers designed as well as which CasX base construct was used). SaCas9 and SpyCas9 control plasmids were prepared similarly to pStX plasmids described above, with the protein and guide regions of pStX exchanged for the respective protein and guide. Targeting sequences for SaCas9 and SpyCas9 were either obtained from the literature or were rationally designed according to established methods.

The expression and recovery of the CasX constructs was performed using standard

### Methodologies and are Summarized as Follows:

### Purification:

Frozen samples were thawed overnight at 4° C. with magnetic stirring. The viscosity of the resulting lysate was reduced by sonication and lysis was completed by homogenization in two passes at 20k PSI using a NanoDeBEE (BEE International). Lysate was clarified by centrifugation at 50,000×g, 4° C., for 30 minutes and the supernatant was collected. The clarified supernatant was applied to a Heparin 6 Fast Flow column (Cytiva) using an AKTA Pure FPLC (Cytiva). The column was washed with 5 CV of Heparin Buffer A (50 mM HEPES-NaOH, 250 mM NaCl, 5 mM MgCl2, 0.5 mM TCEP, 10% glycerol, pH 8), then with 3 CV of Heparin Buffer B (Buffer A with the NaCl concentration adjusted to 500 mM). Protein was eluted with 1.75 CV of Heparin Buffer C (Buffer A with the NaCl concentration adjusted to 1 M). The eluate was applied to a StrepTactin HP column (Cytiva) using the FPLC. The column was washed with 10 CV of Strep Buffer (50 mM HEPES-NaOH, 500 mM NaCl, 5 mM MgCl2, 0.5 mM TCEP, 10% glycerol, pH 8). Protein was eluted from the column using 1.65 CV of Strep Buffer with 2.5 mM Desthiobiotin added. CasX-containing fractions were pooled, concentrated at 4° C. using a 50 kDa cut-off spin concentrator (Amicon), and purified by size exclusion chromatography on a Superdex 200 μg column (Cytiva). The column was equilibrated with SEC Buffer (25 mM sodium phosphate, 300 mM NaCl, 1 mM TCEP, 10% glycerol, pH 7.25) and operated by FPLC. CasX-containing fractions that eluted at the appropriate molecular weight were pooled, concentrated at 4° C. using a 50 kDa cut-off spin concentrator, aliquoted, and snap-frozen in liquid nitrogen before being stored at −80° C.

CasX variant 488: The average yield was 2.7 mg of purified CasX protein per liter of culture at 98.8% purity, as evaluated by colloidal Coomassie staining.

CasX Variant 491: The average yield was 12.4 mg of purified CasX protein per liter of culture at 99.4% purity, as evaluated by colloidal Coomassie staining.

CasX variant 515: The average yield was 7.8 mg of purified CasX protein per liter of culture at 90% purity, as evaluated by colloidal Coomassie staining.

CasX variant 526: The average yield was 13.79 mg per liter of culture, at 93% purity.

Purity was evaluated by colloidal Coomassie staining.

TABLE 6
CasX variant DNA and amino acid sequences

| | SEQ ID NO of | SEQ ID NO of |
|---|---|---|
| Construct | DNA Sequence | Amino Acid Sequence |
| CasX 488 | 27155 | 123 |
| CasX 491 | 27156 | 126 |
| CasX 515 | 27157 | 133 |
| CasX 527 | 27158 | 144 |
| CasX 535 | 27159 | 26911 |
| CasX 536 | 27160 | 26912 |
| CasX 537 | 27161 | 26913 |
| CasX 583 | 27162 | 26958 |
| CasX 660 | 27163 | 27035 |
| CasX 661 | 27164 | 27036 |
| CasX 662 | 27165 | 27037 |
| CasX 663 | 27166 | 27038 |
| CasX 664 | 27167 | 27039 |
| CasX 668 | 27168 | 27043 |
| CasX 670 | 27169 | 27154 |
| CasX 672 | 27170 | 27046 |
| CasX 676 | 27171 | 27050 |
| CasX 677 | 27172 | 27051 |

### Example 2: Generation of RNA Guides

For the generation of RNA single guides and targeting sequences, templates for in vitro transcription were generated by performing PCR with Q5 polymerase, template primers for each backbone, and amplification primers with the T7 promoter and the targeting sequence. The DNA primer sequences for the T7 promoter, guide and targeting sequence for guides and targeting sequences are presented in Table 7, below. The sgl, sg2, sg32, sg64, sg174, and sg235 guides correspond to SEQ ID NOS: 4, 5, 2104, 2106, 2238, and 26800, respectively, with the exception that sg2, sg32, and sg64 were modified with an additional 5′ G to increase transcription efficiency (compare sequences in Table 7 to Table 3). The 7.37 targeting sequence targets beta2-microglobulin (B2M). Following PCR amplification, templates were cleaned and isolated by phenol-chloroform-isoamyl alcohol extraction followed by ethanol precipitation.

In vitro transcriptions were carried out in buffer containing 50 mM Tris pH 8.0, 30 mM MgCl2, 0.01% Triton X-100, 2 mM spermidine, 20 mM DTT, 5 mM NTPs, 0.5 μM template, and 100 μg/mL T7 RNA polymerase. Reactions were incubated at 37° C. overnight. 20 units of DNase I (Promega #M6101)) were added per 1 mL of transcription volume and incubated for one hour. RNA products were purified via denaturing PAGE, ethanol precipitated, and resuspended in 1× phosphate buffered saline. To fold the sgRNAs, samples were heated to 70° C. for 5 min and then cooled to room temperature. The reactions were supplemented to 1 mM final MgCl2 concentration, heated to 50° C. for 5 min and then cooled to room temperature. Final RNA guide products were stored at −80° C.

TABLE 7
DNA primer sequences for the T7 promoter, guide and targeting sequence for guides

| Primer | SEQ ID NO | RNA product | SEQ ID NO |
|---|---|---|---|
| T7 promoter primer | 234 | Used for all | |
| sg2 backbone fwd | 238 | GGUACUGGCGCUUUUAUCUCAUUACUUUGAGAGCCAUCA | 251 |
| sg2 backbone rev | 239 | CCAGCGACUAUGUCGUAUGGGUAAAGCGCUUAUUUAUCG | |
| sg2.7.37 spacer primer | 240 | GAGAGAAAUCCGAUAAAUAAGAAGCAUCAAAGGGCCGAG | |
| | | AUGUCUCGCUCCG | |
| sg32 backbone fwd | 241 | GGUACUGGCGCUUUUAUCUCAUUACUUUGAGAGCCAUCA | 252 |
| sg32 backbone rev | 242 | CCAGCGACUAUGUCGUAUGGGUAAAGCGCCCUCUUCGGA | |
| sg32.7.37 spacer primer | 243 | GGGAAGCAUCAAAGGGCCGAGAUGUCUCG | |
| sg64 backbone fwd | 244 | GGUACUGGCGCCUUUAUCUCAUUACUUUGAGAGCCAUCA | 253 |
| | | CCAGCGACUAUGUCGUAUGGGUAAAGCGCUUACGGACUU | |
| sg64 backbone rev | 245 | CGGUCCGUAAGAAGCAUCAAAGGGCCGAGAUGUCUCGCU | |
| sg64.7.37 spacer primer | 246 | CCG | |
| sg174 backbone fwd | 247 | ACUGGCGCUUUUAUCUgAUUACUUUGAGAGCCAUCACCA | 254 |
| sg174 backbone rev | 248 | GCGACUAUGUCGUAgUGGGUAAAGCUCCCUCUUCGGAGG | |
| sg174.7.37 spacer | 249 | GAGCAUCAAAGGGCCGAGAUGUCUCGCUCCG | |
| primer | | | |
| sg235 backbone fwd | ND | ACUGGCGCUUCUAUCUGAUUACUCUGAGCGCCAUCACCA | 26800 |
| sg235 backbone rev | ND | GCGACUAUGUCGUAGUGGGUAAAGCCGCUUACGGACUUC | |
| sg235.7.37 spacer | ND | GGUCCGUAAGAGGCAUCAGAG | |
| primer | | | |

### Example 3: Assessing Binding Affinity to the Guide RNA

Purified wild-type and improved CasX will be incubated with synthetic single-guide RNA containing a 3′ Cy7.5 moiety in low-salt buffer containing magnesium chloride as well as heparin to prevent non-specific binding and aggregation. The sgRNA will be maintained at a concentration of 10 pM, while the protein will be titrated from 1 pM to 100 μM in separate binding reactions. After allowing the reaction to come to equilibrium, the samples will be run through a vacuum manifold filter-binding assay with a nitrocellulose membrane and a positively charged nylon membrane, which bind protein and nucleic acid, respectively. The membranes will be imaged to identify guide RNA, and the fraction of bound vs unbound RNA will be determined by the amount of fluorescence on the nitrocellulose vs nylon membrane for each protein concentration to calculate the dissociation constant of the protein-sgRNA complex. The experiment will also be carried out with improved variants of the sgRNA to determine if these mutations also affect the affinity of the guide for the wild-type and mutant proteins. We will also perform electromobility shift assays to qualitatively compare to the filter-binding assay and confirm that soluble binding, rather than aggregation, is the primary contributor to protein-RNA association.

### Example 4: Assessing Binding Affinity to the Target DNA

Purified wild-type and improved CasX will be complexed with single-guide RNA bearing a targeting sequence complementary to the target nucleic acid. The RNP complex will be incubated with double-stranded target DNA containing a PAM and the appropriate target nucleic acid sequence with a 5′ Cy7.5 label on the target strand in low-salt buffer containing magnesium chloride as well as heparin to prevent non-specific binding and aggregation. The target DNA will be maintained at a concentration of 1 nM, while the RNP will be titrated from 1 μM to 100 μM in separate binding reactions. After allowing the reaction to come to equilibrium, the samples will be run on a native 5% polyacrylamide gel to separate bound and unbound target DNA. The gel will be imaged to identify mobility shifts of the target DNA, and the fraction of bound vs unbound DNA will be calculated for each protein concentration to determine the dissociation constant of the RNP-target DNA ternary complex.

### Example 5: Assessing Differential PAM Recognition In Vitro

### 1. Comparison of Reference and CasX Variants

In vitro cleavage assays were performed using CasX2, CasX119, and CasX438 complexed with sg174.7.37, essentially as describe in Example 8. Fluorescently labeled dsDNA targets with a 7.37 spacer and either a TTC, CTC, GTC, or ATC PAM were used (sequences are in Table 8). Time points were taken at 0.25, 0.5, 1, 2, 5, 10, 30, and 60 minutes. Gels were imaged with an Cytiva Typhoon and quantified using the IQTL 8.2 software. Apparent first-order rate constants for non-target strand cleavage (k_{cleave}) were determined for each CasX:sgRNA complex on each target. Rate constants for targets with non-TTC PAM were compared to the TTC PAM target to determine whether the relative preference for each PAM was altered in a given protein variant.

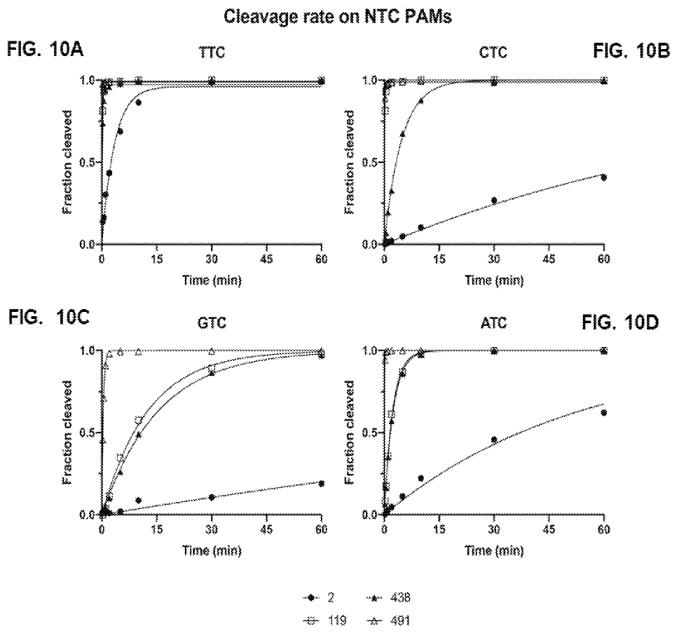

For all variants, the TTC target supported the highest cleavage rate, followed by the ATC, then the CTC, and finally the GTC target (FIGS. 10A-D, Table 9). For each combination of CasX variant and NTC PAM, the cleavage rate k_{cleave} is shown. For all non-NTC PAMs, the relative cleavage rate as compared to the TTC rate for that variant is shown in parentheses. All non-TTC PAMs exhibited substantially decreased cleavage rates (>10-fold for all). The ratio between the cleavage rate of a given non-TTC PAM and the TTC PAM for a specific variant remained generally consistent across all variants. The CTC target supported cleavage 3.5-4.3% as fast as the TTC target; the GTC target supported cleavage 1.0-1.4% as fast; and the ATC target supported cleavage 6.5-8.3% as fast. The exception is for 491, where the kinetics of cleavage at TTC PAMs are too fast to allow accurate measurement, which artificially decreases the apparent difference between TTC and non-TTC PAMs. Comparing the relative rates of 491 on GTC, CTC, and ATC PAMs, which fall within the measurable range, results in ratios comparable to those for other variants when comparing across non-TTC PAMs, consistent with the rates increasing in tandem. Overall, differences between the variants are not substantial enough to suggest that the relative preference for the various NTC PAMs have been altered. However, the higher basal cleavage rates of the variants allow targets with ATC or CTC PAMs to be cleaved nearly completely within 10 minutes, and the apparent k_{cleaves} are comparable to or greater than the k_{cleave} of CasX2 on a TTC PAM (Table 9). This increased cleavage rate may cross the threshold necessary for effective genome editing in a human cell, explaining the apparent increase in PAM flexibility for these variants.

TABLE 8
Sequences of DNA substrates used in in
vitro PAM cleavage assay

| Guide* | DNA Sequence | SEQ ID NO |
|---|---|---|
| 7.37 | AGCGCGAGCACAGCTAAGGCCACGGAGCGAGACATCTCGGCCCGAATGCTGTCAGCTTCA | 27176 |
| TTC | | |
| PAM TS | | |
| 7.37 | TGAAGCTGACAGCATTCGGGCCGAGATGTCTCGCTCCGTGGCCTTAGCTGTGCTCGCGCT | 27177 |
| TTC | | |
| PAM NTS | | |
| 7.37 | AGCGCGAGCACAGCTAAGGCCACGGAGCGAGACATCTCGGCCCGAGTGCTGTCAGCTTCA | 27178 |
| CTC | | |
| PAM TS | | |
| 7.37 | TGAAGCTGACAGCACTCGGGCCGAGATGTCTCGCTCCGTGGCCTTAGCTGTGCTCGCGCT | 27179 |
| CTC | | |
| PAM NTS | | |
| 7.37 | AGCGCGAGCACAGCTAAGGCCACGGAGCGAGACATCTCGGCCCGACTGCTGTCAGCTTCA | 27180 |
| GTC | | |
| PAM TS | | |
| 7.37 | TGAAGCTGACAGCAGTCGGGCCGAGATGTCTCGCTCCGTGGCCTTAGCTGTGCTCGCGCT | 27181 |
| GTC | | |
| PAM NTS | | |
| 7.37 | AGCGCGAGCACAGCTAAGGCCACGGAGCGAGACATCTCGGCCCGATTGCTGTCAGCTTCA | 27182 |
| ATC | | |
| PAM TS | | |
| 7.37 | TGAAGCTGACAGCAATCGGGCCGAGATGTCTCGCTCCGTGGCCTTAGCTGTGCTCGCGCT | 27183 |
| ATC | | |
| PAM NTS | | |

*The PAM sequences for each are bolded. TS-target strand. NTS-Non-target strand.

TABLE 9
Apparent cleavage rates of CasX variants against NTC PAMs

| Variant | TTC | CTC | GTC | ATC |
|---|---|---|---|---|
| 2 | 0.267 min⁻¹ | 9.29E−3 min⁻¹ | 3.75E−3 min⁻¹ | 1.87E−2 min⁻¹ |
| | | (0.035) | (0.014) | (0.070) |
| 119 | 8.33 min⁻¹ | 0.303 min⁻¹ | 8.64E−2 min⁻¹ | 0.540 min⁻¹ |
| | | (0.036) | (0.010) | (0.065) |
| 438 | 4.94 min⁻¹ | 0.212 min⁻¹ | 1.31E−2 min⁻¹ | 0.408 min⁻¹ |
| | | (0.043) | (0.013) | (0.083) |
| 491 | 16.42 min⁻¹ | 8.605 min⁻¹ | 2.447 min⁻¹ | 11.33 min⁻¹ |
| | | (0.524) | (0.149) | (0.690) |

### 2. Comparison of PAM Recognition Using Single CasX Variant

### Materials and Methods:

Fluorescently labeled dsDNA targets with a 7.37 spacer and either a TTC, CTC, GTC, ATC, TTT, CTT, GTT, or ATT PAM were used (sequences are in Table 10). Oligos were ordered with a 5′ amino modification and labeled with a Cy7.5 NHS ester for target strand oligos and a Cy5.5 NHS ester for non-target strand oligos. dsDNA targets were formed by mixing the oligos in a 1:1 ratio in 1× cleavage buffer (20 mM Tris HCl pH 7.5, 150 mM NaCl, 1 mM TCEP, 5% glycerol, 10 mM MgCl2), heating to 95° C. for 10 minutes, and allowing the solution to cool to room temperature.

CasX variant 491 was complexed with sg174.7.37. The guide was diluted in 1× cleavage buffer to a final concentration of 1.5 μM, and then protein was added to a final concentration of 1 μM. The RNP was incubated at 37° C. for 10 minutes and then put on ice.

Cleavage assays were carried out by diluting RNP in cleavage buffer to a final concentration of 200 nM and adding dsDNA target to a final concentration of 10 nM. Time points were taken at 0.25, 0.5, 1, 2, 5, and 10 minutes and quenched by adding to an equal volume of 95% formamide and 20 mM EDTA. Cleavage products were resolved by running on a 10% urea-PAGE gel. Gels were imaged with an Amersham Typhoon and quantified using the IQTL 8.2 software. Apparent first-order rate constants for non-target strand cleavage (k_{cleave}) were determined for each target using GraphPad Prism.

### Results:

The relative cleavage rate of the 491.174 RNP on various PAMs was investigated. In addition to aiding in the prediction of cleavage efficiencies of targets and potential off-targets in cells, these data will also allow us to adjust the cleavage rate of synthetic targets. In the case of self-limiting AAV vectors, where new protospacers can be added within the vector to allow for self-targeting, we reasoned that the rate of episome cleavage could be adjusted up or down by changing the PAM.

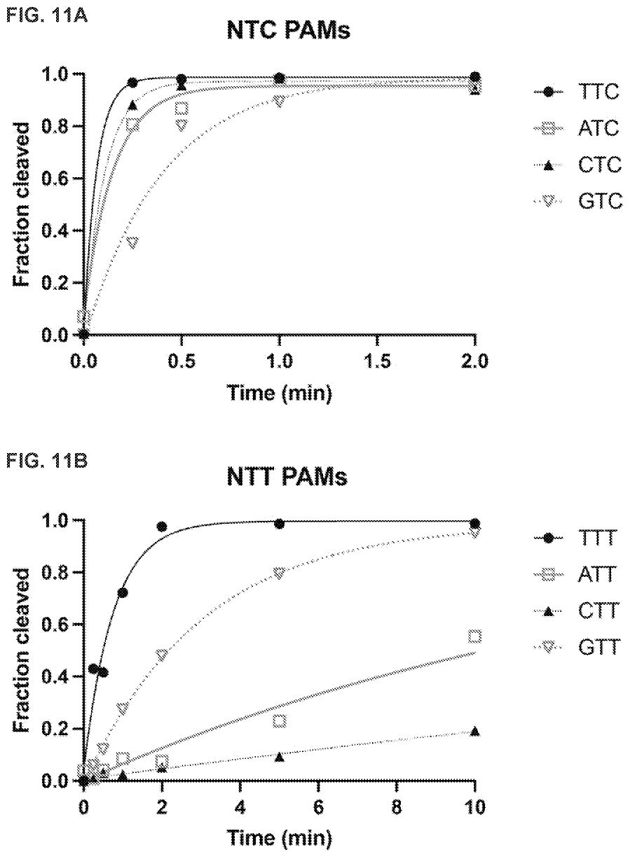

We tested the cleavage rate of the RNP against various dsDNA substrates that were identical in sequence aside from the PAM. This experimental setup should allow for the isolation of the effects of the PAM itself, rather than convoluting PAM recognition with effects resulting from spacer sequence and genomic context. All NTC and NTT PAMs were tested. As expected, the RNP cleaved the target with the TTC PAM most quickly, converting essentially all of it to product by the first time point (FIG. 11A). CTC was cleaved roughly half as quickly, though the rapid cleavage of TTC makes determining an accurate k_{cleave} difficult under these assay conditions, which are optimized to capture a broader array of cleavage rates (FIG. 11A, Table 11). The GTC target was cleaved most slowly of the NTC PAMs, with a cleavage rate roughly six-fold slower than the TTC target. All NTT PAMs were cleaved more slowly than all NTC PAMs, with TTT cut most efficiently, followed by GTT (FIG. 111B, Table 11). The relative efficiency of GTT cleavage among all NTT PAMs, compared to the low rate of GTC cleavage compared to all NTC PAMs, demonstrates that recognition of individual PAM nucleotides is context-dependent, with nucleotide identity at one position in the PAM affecting sequence preference at the other positions.

The PAM sequences tested here yield cleavage rates spanning three orders of magnitude while still maintaining cleavage activity at the same spacer sequence. These data demonstrate that cleavage rates at a given synthetic target can be readily modified by changing the associated PAM, allowing for adjustment of self-cleavage activity to allow for efficient targeting of the genomic target prior to cleavage and elimination of the AAV episome.

TABLE 10
Sequences of DNA substrates used in in vitro PAM cleavage assay*

| PAM & Strand | Spacer and PAM Sequence | SEQ ID NO |
|---|---|---|
| 7.37 TTC PAM TS | AGCGCGAGCACAGCTAAGGCCACGGAGCGAGACATCTCGGCCCGAATGCT | 27176 |
| | GTCAGCTTCA | |
| 7.37 TTC PAM NTS | TGAAGCTGACAGCATTCGGGCCGAGATGTCTCGCTCCGTGGCCTTAGCTG | 27177 |
| | TGCTCGCGCT | |
| 7.37 CTC PAM TS | AGCGCGAGCACAGCTAAGGCCACGGAGCGAGACATCTCGGCCCGAGTGCT | 27178 |
| | GTCAGCTTCA | |
| 7.37 CTC PAM NTS | TGAAGCTGACAGCACTCGGGCCGAGATGTCTCGCTCCGTGGCCTTAGCTG | 27179 |
| | TGCTCGCGCT | |
| 7.37 GTC PAM TS | AGCGCGAGCACAGCTAAGGCCACGGAGCGAGACATCTCGGCCCGACTGCT | 27180 |
| | GTCAGCTTCA | |
| 7.37 GTC PAM NTS | TGAAGCTGACAGCAGTCGGGCCGAGATGTCTCGCTCCGTGGCCTTAGCTG | 27181 |
| | TGCTCGCGCT | |
| 7.37 ATC PAM TS | AGCGCGAGCACAGCTAAGGCCACGGAGCGAGACATCTCGGCCCGATTGCT | 27182 |
| | GTCAGCTTCA | |
| 7.37 ATC PAM NTS | TGAAGCTGACAGCAATCGGGCCGAGATGTCTCGCTCCGTGGCCTTAGCTG | 27183 |
| | TGCTCGCGCT | |
| 7.37 TTT PAM TS | AGCGCGAGCACAGCTAAGGCCACGGAGCGAGACATCTCGGCCCAAATGCT | 27184 |
| | GTCAGCTTCA | |
| 7.37 TTT PAM NTS | TGAAGCTGACAGCATTTGGGCCGAGATGTCTCGCTCCGTGGCCTTAGCTG | 27185 |
| | TGCTCGCGCT | |
| 7.37 CTT PAM TS | AGCGCGAGCACAGCTAAGGCCACGGAGCGAGACATCTCGGCCCTAGTGCT | 27186 |
| | GTCAGCTTCA | |
| 7.37 CTT PAM NTS | TGAAGCTGACAGCACTTGGGCCGAGATGTCTCGCTCCGTGGCCTTAGCTG | 27187 |
| | TGCTCGCGCT | |
| 7.37 GTT PAM TS | AGCGCGAGCACAGCTAAGGCCACGGAGCGAGACATCTCGGCCCTACTGCT | 27188 |
| | GTCAGCTTCA | |
| 7.37 GTT PAM NTS | TGAAGCTGACAGCAGTTGGGCCGAGATGTCTCGCTCCGTGGCCTTAGCTG | 27189 |
| | TGCTCGCGCT | |
| 7.37 ATT PAM TS | AGCGCGAGCACAGCTAAGGCCACGGAGCGAGACATCTCGGCCCTATTGCT | 27190 |
| | GTCAGCTTCA | |
| 7.37 ATT PAM NTS | TGAAGCTGACAGCAATTGGGCCGAGATGTCTCGCTCCGTGGCCTTAGCTG | 27191 |
| | TGCTCGCGCT | |

*The DNA sequences used to generate each dsDNA substrate are shown. The PAM sequences for
each are bolded. TS-target strand. NTS-Non-target strand.

TABLE 11
Apparent cleavage rates of CasX
491.174 against NTC and NTT PAMs

| PAM | TTC | ATC | CTC | GTC | TTT | ATT | CTT | GTT |
|---|---|---|---|---|---|---|---|---|
| k_{cleave} | 15.6* | 6.66 | 9.45 | 2.52 | 1.33 | 0.0675 | 0.0204 | 0.330 |
| (min⁻¹) | | | | | | | | |

*The rate of TTC cleavage exceeds the resolution of this assay, so the resulting k_{cleave} should be taken as a lower bound.

### Example 6: Assessing Nuclease Activity for Double-Strand Cleavage

Purified wild-type and engineered CasX variants will be complexed with single-guide RNA bearing a fixed HRS targeting sequence. The RNP complexes will be added to buffer containing MgCl2 at a final concentration of 100 nM and incubated with double-stranded target DNA with a 5′ Cy7.5 label on either the target or non-target strand at a concentration of 10 nM. Aliquots of the reactions will be taken at fixed time points and quenched by the addition of an equal volume of 50 mM EDTA and 95% formamide. The samples will be run on a denaturing polyacrylamide gel to separate cleaved and uncleaved DNA substrates. The results will be visualized and the cleavage rates of the target and non-target strands by the wild-type and engineered variants will be determined. To more clearly differentiate between changes to target binding vs the rate of catalysis of the nucleolytic reaction itself, the protein concentration will be titrated over a range from 10 nM to 1 uM and cleavage rates will be determined at each concentration to generate a pseudo-Michaelis-Menten fit and determine the kcat* and KM*. Changes to KM* are indicative of altered binding, while changes to kcat* are indicative of altered catalysis.

### Example 7: The PASS Assay Identifies CasX Protein Variants of Differing PAM Sequence Specificity

Experiments were conducted to identify the PAM sequence specificities of CasX proteins 2 (SEQ ID NO: 2), 491 (SEQ ID NO: 126), 515 (SEQ ID NO: 133), 533 (SEQ ID NO: 26909), 535 (SEQ ID NO: 26911), 668 (SEQ ID NO: 27043), and 672 (SEQ ID NO: 27046). To accomplish this, the HEK293 cell line PASS_V1.01 or PASS_V1.02 was treated with the above CasX proteins in at least two replicate experiments, and Next-generation sequencing (NGS) was performed to calculate the percent editing using a variety of spacers at their intended target sites.

Materials and Methods: A multiplexed pooled approach was taken to assay clonal protein variants using the PASS system. Briefly, two pooled HEK293 cell lines were generated and termed PASS_V1.01 and PASS_V1.02. Each cell within the pool contained a genome-integrated single-guide RNA (sgRNA), paired with a specific target site. After transfection of protein-expression constructs, editing at a specific target by a specific spacer could be quantified by NGS. Each guide-target pair was designed to provide data related to activity, specificity, and targetability of the CasX-guide RNP complex.

Paired spacer-target sequences were synthesized by Twist Biosciences and obtained as an equimolar pool of oligonucleotides. This pool was amplified by PCR and cloned by Golden Gate cloning to generate a final library of plasmids named p77. Each plasmid contained a sgRNA expression element and a target site, along with a GFP expression element. The sgRNA expression element consisted of a U6 promoter driving transcription of gRNA scaffold 174 (SEQ ID NO:2238), followed by a spacer sequence which would target the RNP of the guide and CasX variant to the intended target site. 250 possible unique, paired spacer-target synthetic sequences were designed and synthesized. A pool of lentivirus was then produced from this plasmid library using the LentiX production system (Takara Bio USA, Inc) according to the manufacturer's instructions. The resulting viral preparation was then quantified by qPCR and transduced into a standard HEK293 cell line at a low multiplicity of infection so as to generate single copy integrations. The resulting cell line was then purified by fluorescence-activated cell sorting (FACS) to complete the production of PASS_V1.01 or PASS_V1.02. A cell line was then seeded in six-well plate format and treated in duplicate with either water or was transfected with 2 μg of plasmid p67, delivered by Lipofectamine Transfection Reagent (ThermoFisher) according to the manufacturer's instructions. Plasmid p67 contains an EF-1alpha promoter driving expression of a CasX protein tagged with the SV40 Nuclear Localization Sequence. After two days, treated cells were collected, lysed, and genomic DNA was extracted using a genomic DNA isolation kit (Zymo Research). Genomic DNA was then PCR amplified with custom primers to generate amplicons compatible with Illumina NGS and sequenced on a NextSeq instrument. Sample reads were demultiplexed and filtered for quality. Editing outcome metrics (fraction of reads with indels) were then quantified for each spacer-target synthetic sequence across treated samples.

To assess the PAM sequence specificity for a CasX protein, editing outcome metrics for four different PAM sequences were categorized. For TTC PAM target sites, 48 different spacer-target pairs were quantified; for ATC, CTC, and GTC PAM target sites, 14, 22, and 11 individual target sites were quantified, respectively. For some CasX proteins, replicate experiments were repeated dozens of times over several months. For each of these experiments, the average editing efficiency was calculated for each of the above described spacers. The average editing efficiency across the four categories of PAM sequence was then calculated from all such experiments, along with the standard deviation of these measurements.

### Results:

Table 12 lists the average editing efficiency across PAM categories and across CasX protein variants, along with the standard deviation of these measurements. The number of measurements for each category is also indicated. These data indicate that the engineered CasX variants 491 and 515 are specific for the canonical PAM sequence TTC, while other engineered variants of CasX performed more or less efficiently at the PAM sequences tested. In particular, the average rank order of PAM preferences for CasX 491 is TTC»ATC>CTC>GTC, or TTC»ATC>GTC>CTC for CasX 515, while the wild-type CasX 2 exhibits an average rank order of TTC»GTC>CTC>ATC. Note that for the lower editing PAM sequences the error of these average measurements is high. In contrast, CasX variants 535, 668, and 672 have considerably broader PAM recognition, with a rank order of TTC>CTC>ATC>GTC. Finally, CasX 533 exhibits a completely re-ordered ranking relative to the WT CasX, ATC>CTC»GTC>TTC. These data can be used to engineer maximally-active therapeutic CasX molecules for a target DNA sequence of interest.

Under the conditions of the experiments, a set of CasX proteins was identified that are improved for double-stranded DNA cleavage in human cells at target DNA sequences associated with a PAM of sequence TTC, ATC, CTC, or GTC, supporting that CasX variants with an altered spectrum of PAM specificity, relative to CasX 491, for non-canonical PAM (i.e., ATC, CTC, and GTC).

TABLE 12
Average editing of selected CasX Proteins at spacers associated
with PAM sequences of TTC, ATC, CTC, or GTC

| | PAM | Average Percent | Standard | Number of |
|---|---|---|---|---|
| CasX Name | Sequence | Editing | Deviation | Measurements |
| 2 | ATC | 0.40 | 1.35 | 336 |
| 2 | CTC | 0.46 | 2.29 | 528 |
| 2 | GTC | 0.69 | 6.27 | 264 |
| 2 | TTC | 5.28 | 7.34 | 1152 |
| 491 | ATC | 6.86 | 8.29 | 364 |
| 491 | CTC | 4.54 | 6.40 | 572 |
| 491 | GTC | 3.40 | 6.68 | 286 |
| 491 | TTC | 40.41 | 23.13 | 1248 |
| 515 | ATC | 4.47 | 5.49 | 252 |
| 515 | CTC | 3.36 | 4.80 | 396 |
| 515 | GTC | 3.65 | 10.75 | 198 |
| 515 | TTC | 36.75 | 24.89 | 864 |
| 533 | ATC | 47.50 | 15.86 | 96 |
| 533 | CTC | 25.90 | 14.74 | 28 |
| 533 | GTC | 6.34 | 8.36 | 44 |
| 533 | TTC | 0.87 | 3.05 | 22 |
| 535 | ATC | 9.70 | 10.20 | 56 |
| 535 | CTC | 11.77 | 13.59 | 88 |
| 535 | GTC | 7.62 | 15.04 | 44 |
| 535 | TTC | 29.29 | 18.78 | 192 |
| 668 | ATC | 44.69 | 24.40 | 56 |
| 668 | CTC | 46.14 | 26.57 | 88 |
| 668 | GTC | 30.48 | 24.06 | 44 |
| 668 | TTC | 55.34 | 28.59 | 192 |
| 672 | ATC | 25.51 | 20.85 | 56 |
| 672 | CTC | 30.05 | 22.95 | 88 |
| 672 | GTC | 14.21 | 13.38 | 44 |
| 672 | TTC | 52.36 | 27.64 | 192 |

### Example 8: CasX:gRNA In Vitro Cleavage Assays

### 1. Assembly of RNP

Purified wild-type and RNP of CasX and single guide RNA (sgRNA) were either prepared immediately before experiments or prepared and snap-frozen in liquid nitrogen and stored at −80° C. for later use. To prepare the RNP complexes, the CasX protein was incubated with sgRNA at 1:1.2 molar ratio. Briefly, sgRNA was added to Buffer #1 (25 mM NaPi, 150 mM NaCl, 200 mM trehalose, 1 mM MgCl2), then the CasX was added to the sgRNA solution, slowly with swirling, and incubated at 37° C. for 10 min to form RNP complexes. RNP complexes were filtered before use through a 0.22 tm Costar 8160 filters that were pre-wet with 200 μl Buffer #1. If needed, the RNP sample was concentrated with a 0.5 ml Ultra 100-Kd cutoff filter, (Millipore part #UFC510096), until the desired volume was obtained. Formation of competent RNP was assessed as described below.

### 2. Determining Cleavage-Competent Fractions for Protein Variants Compared to Wild-Type Reference CasX

The ability of CasX variants to form active RNP compared to reference CasX was determined using an in vitro cleavage assay. The beta-2 microglobulin (B2M) 7.37 target for the cleavage assay was created as follows. DNA oligos with the sequence TGAAGCTGACAGCATTCGGGCCGAGATGTCTCGCTCCGTGGCCTTAGCTGTGCTCGC GCT (non-target strand, NTS (SEQ ID NO: 27177)) and AGCGCGAGCACAGCTAAGGCCACGGAGCGAGACATCTCGGCCCGAATGCTGTCAGC TTCA (target strand, TS (SEQ ID NO: 27176)) were purchased with 5′ fluorescent labels (LI-COR IRDye 700 and 800, respectively). dsDNA targets were formed by mixing the oligos in a 1:1 ratio in 1× cleavage buffer (20 mM Tris HCl pH 7.5, 150 mM NaCl, 1 mM TCEP, 5% glycerol, 10 mM MgCl2), heating to 95° C. for 10 minutes, and allowing the solution to cool to room temperature.

CasX RNPs were reconstituted with the indicated CasX and guides (see graphs) at a final concentration of 1 μM with 1.5-fold excess of the indicated guide unless otherwise specified in 1x cleavage buffer (20 mM Tris HCl pH 7.5, 150 mM NaCl, 1 mM TCEP, 5% glycerol, 10 mM MgCl2) at 37° C. for 10 min before being moved to ice until ready to use. The 7.37 target was used, along with sgRNAs having spacers complementary to the 7.37 target.

Cleavage reactions were prepared with final RNP concentrations of 100 nM and a final target concentration of 100 nM. Reactions were carried out at 37° C. and initiated by the addition of the 7.37 target DNA. Aliquots were taken at 5, 10, 30, 60, and 120 minutes and quenched by adding to 95% formamide, 20 mM EDTA. Samples were denatured by heating at 95° C. for 10 minutes and run on a 10% urea-PAGE gel. The gels were either imaged with a LI-COR Odyssey CLx and quantified using the LI-COR Image Studio software or imaged with a Cytiva Typhoon and quantified using the Cytiva IQTL software. The resulting data were plotted and analyzed using Prism. We assumed that CasX acts essentially as a single-turnover enzyme under the assayed conditions, as indicated by the observation that sub-stoichiometric amounts of enzyme fail to cleave a greater-than-stoichiometric amount of target even under extended time-scales and instead approach a plateau that scales with the amount of enzyme present. Thus, the fraction of target cleaved over long time-scales by an equimolar amount of RNP is indicative of what fraction of the RNP is properly formed and active for cleavage. The cleavage traces were fit with a biphasic rate model, as the cleavage reaction clearly deviates from monophasic under this concentration regime, and the plateau was determined for each of three independent replicates. The mean and standard deviation were calculated to determine the active fraction (Table 13).

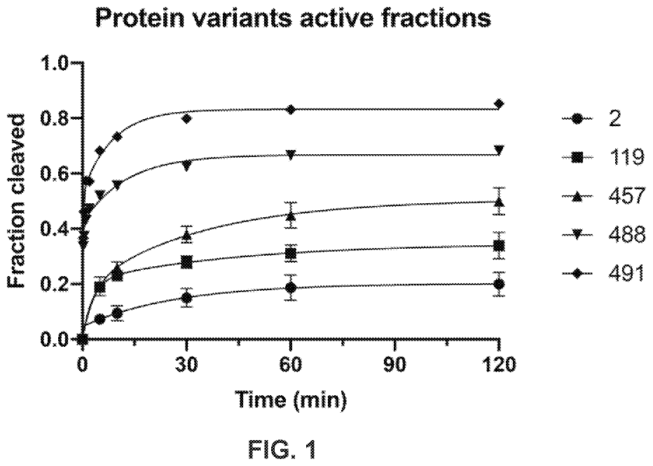

Apparent active (competent) fractions were determined for RNPs formed for CasX2+ guide 174+7.37 spacer, CasX119+ guide 174+7.37 spacer, CasX457+ guide 174+7.37 spacer, CasX488+ guide 174+7.37 spacer, and CasX491+ guide 174+7.37 spacer as shown in FIG. 1. The determined active fractions are shown in Table 13. All CasX variants had higher active fractions than the wild-type CasX2, indicating that the engineered CasX variants form significantly more active and stable RNP with the identical guide under tested conditions compared to wild-type CasX. This may be due to an increased affinity for the sgRNA, increased stability or solubility in the presence of sgRNA, or greater stability of a cleavage-competent conformation of the engineered CasX:sgRNA complex. An increase in solubility of the RNP was indicated by a notable decrease in the observed precipitate formed when CasX457, CasX488, or CasX491 was added to the sgRNA compared to CasX2.

3. In Vitro Cleavage Assays—Determining Cleavage-Competent Fractions for Single Guide Variants relative to reference single guides

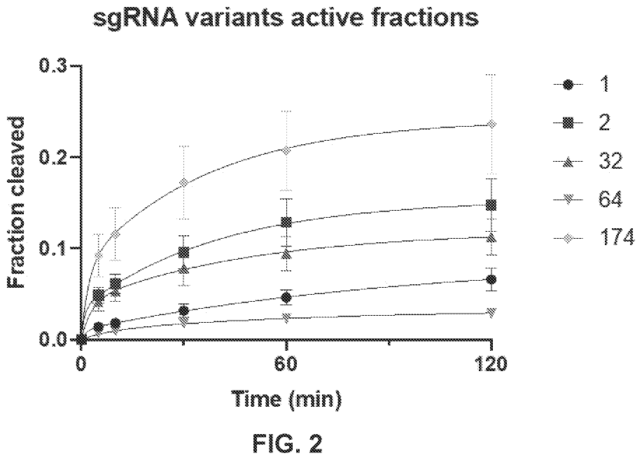

Cleavage-competent fractions were also determined using the same protocol for CasX2.2.7.37, CasX2.32.7.37, CasX2.64.7.37, and CasX2.174.7.37 to be 16±3%, 13±3%, 5+2%, and 22±5%, as shown in FIG. 2 and Table 10.

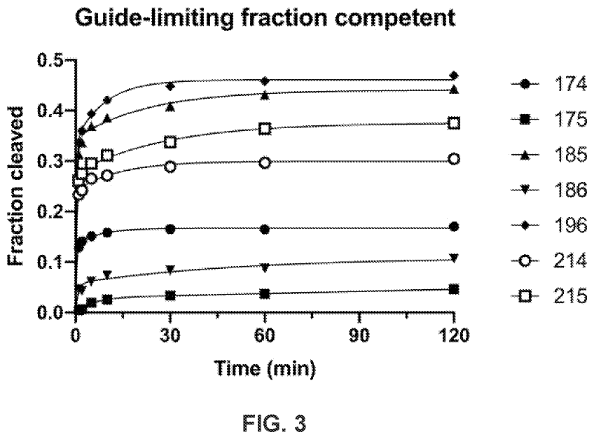

A second set of guides were tested under different conditions to better isolate the contribution of the guide to RNP formation. Guides 174, 175, 185, 186, 196, 214, and 215 with 7.37 spacer were mixed with CasX 491 at final concentrations of 1 μM for the guide and 1.5 μM for the protein, rather than with excess guide as before. Results are shown in FIG. 3 and Table 10. Many of these guides exhibited additional improvement over 174, with 185 and 196 achieving 91±4% and 91±1% competent fractions, respectively, compared with 80±9% for 174 under these guide-limiting conditions.

The data indicate that both CasX variants and sgRNA variants are able to form a higher degree of active RNP with guide RNA compare to wild-type CasX and wild-type sgRNA. The apparent cleavage rates of CasX variants 119, 457, 488, and 491 compared to wild-type reference CasX were determined using an in vitro fluorescent assay for cleavage of the target 7.37.

4. In Vitro Cleavage Assays—Determining k_{cleave} for CasX Variants Compared to Wild-Type Reference CasX

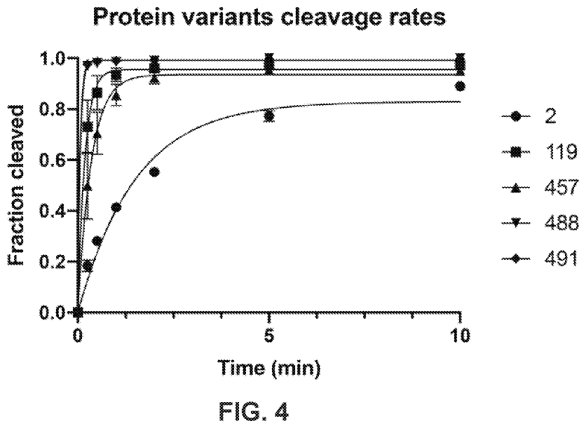

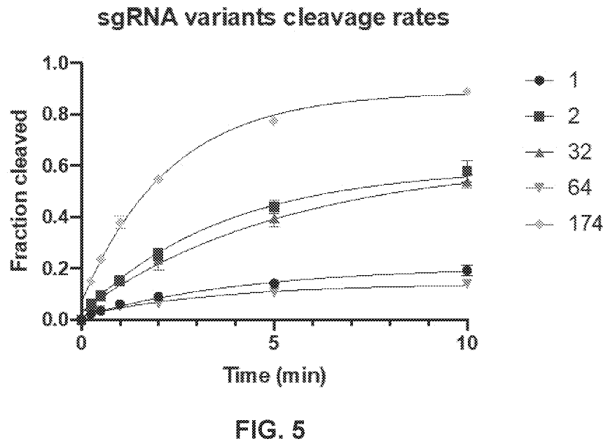

CasX RNPs were reconstituted with the indicated CasX (see FIG. 4) at a final concentration of 1 μM with 1.5-fold excess of the indicated guide in 1× cleavage buffer (20 mM Tris HCl pH 7.5, 150 mM NaCl, 1 mM TCEP, 5% glycerol, 10 mM MgCl₂) at 37° C. for 10 min before being moved to ice until ready to use. Cleavage reactions were set up with a final RNP concentration of 200 nM and a final target concentration of 10 nM. Reactions were carried out at 37° C. except where otherwise noted and initiated by the addition of the target DNA. Aliquots were taken at 0.25, 0.5, 1, 2, 5, and 10 minutes and quenched by adding to 95% formamide, 20 mM EDTA. Samples were denatured by heating at 95° C. for 10 minutes and run on a 10% urea-PAGE gel. The gels were imaged with a LI-COR Odyssey CLx and quantified using the LI-COR Image Studio software or imaged with a Cytiva Typhoon and quantified using the Cytiva IQTL software. The resulting data were plotted and analyzed using Prism, and the apparent first-order rate constant of non-target strand cleavage (k_{cleave}) was determined for each CasX:sgRNA combination replicate individually. The mean and standard deviation of three replicates with independent fits are presented in Table 10, and the cleavage traces are shown in FIG. 5.

Apparent cleavage rate constants were determined for wild-type CasX2, and CasX variants 119, 457, 488, and 491 with guide 174 and spacer 7.37 utilized in each assay (see Table 10 and FIG. 4). All CasX variants had improved cleavage rates relative to the wild-type CasX2. CasX 457 cleaved more slowly than 119, despite having a higher competent fraction as determined above. CasX488 and CasX491 had the highest cleavage rates by a large margin; as the target was almost entirely cleaved in the first timepoint, the true cleavage rate exceeds the resolution of this assay, and the reported k_{cleave} should be taken as a lower bound.

The data indicate that the CasX variants have a higher level of activity, with k_{cleave} rates reaching at least 30-fold higher compared to wild-type CasX2.

### 5. In Vitro Cleavage Assays: Comparison of Guide Variants to Wild-Type Guides

Cleavage assays were also performed with wild-type reference CasX2 and reference guide 2 compared to guide variants 32, 64, and 174 to determine whether the variants improved cleavage. The experiments were performed as described above. As many of the resulting RNPs did not approach full cleavage of the target in the time tested, we determined initial reaction velocities (V₀) rather than first-order rate constants. The first two timepoints (15 and 30 seconds) were fit with a line for each CasX:sgRNA combination and replicate. The mean and standard deviation of the slope for three replicates were determined.

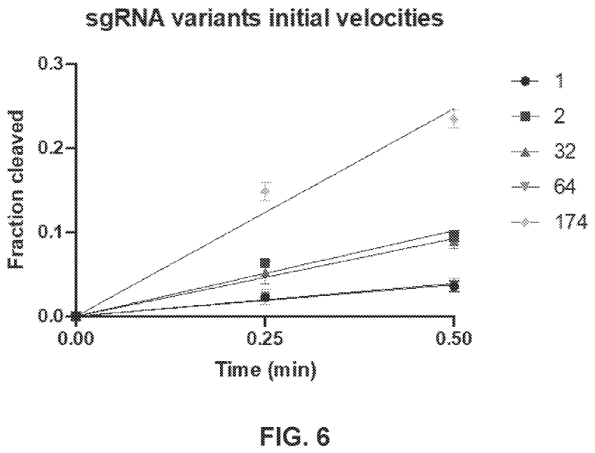

Under the assayed conditions, the V₀ for CasX2 with guides 2, 32, 64, and 174 were 20.4±1.4 nM/min, 18.4 2.4 nM/min, 7.8 1.8 nM/min, and 49.3±1.4 nM/min (see Table 13 and FIG. 5 and FIG. 6). Guide 174 showed substantial improvement in the cleavage rate of the resulting RNP (˜2.5-fold relative to 2, see FIG. 6), while guides 32 and 64 performed similar to or worse than guide 2. Notably, guide 64 supports a cleavage rate lower than that of guide 2 but performs much better in vivo (data not shown). Some of the sequence alterations to generate guide 64 likely improve in vivo transcription at the cost of a nucleotide involved in triplex formation. Improved expression of guide 64 likely explains its improved activity in vivo, while its reduced stability may lead to improper folding in vitro.

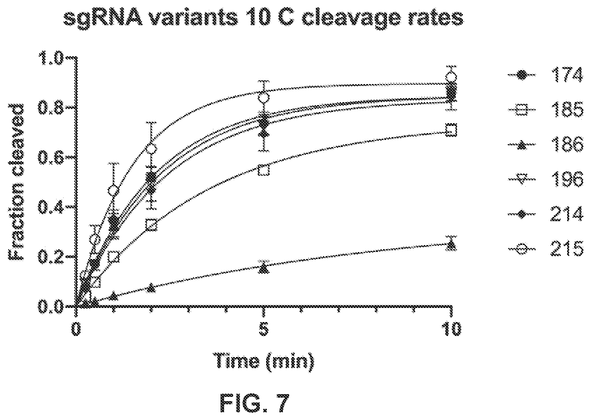

Additional experiments were carried out with guides 174, 175, 185, 186, 196, 214, and 215 with spacer 7.37 and CasX 491 to determine relative cleavage rates. To reduce cleavage kinetics to a range measurable with our assay, the cleavage reactions were incubated at 10° C. Results are in FIG. 7 and Table 13. Under these conditions, 215 was the only guide that supported a faster cleavage rate than 174. 196, which exhibited the highest active fraction of RNP under guide-limiting conditions, had kinetics essentially the same as 174, again highlighting that different variants result in improvements of distinct characteristics.

The data support that, under the conditions of the assay, use of the majority of the guide variants with CasX results in RNP with a higher level of activity than one with the wild-type guide, with improvements in initial cleavage velocity ranging from −2-fold to >6-fold. Numbers in Table 13 indicate, from left to right, CasX variant, sgRNA scaffold, and spacer sequence of the RNP construct. In the RNP construct names in the table below, CasX protein variant, guide scaffold and spacer are indicated from left to right.

### 6. In Vitro Cleavage Assays: Comparing Cleavage Rate and Competent Fraction of 515.174 and 526.174 Against Reference 2.2.

We wished to compare engineered protein CasX variants 515 and 526 in complex with engineered single-guide variant 174 against the reference wild-type protein 2 (SEQ ID NO:2) and minimally-engineered guide variant 2 (SEQ ID NO: 5). RNP complexes were assembled as described above, with 1.5-fold excess guide. Cleavage assays to determine k_{cleave} and competent fraction were performed as described above, with both performed at 37° C., and with different timepoints used to determine the competent fraction for the wild-type vs engineered RNPs due to the significantly different times needed for the reactions to near completion.

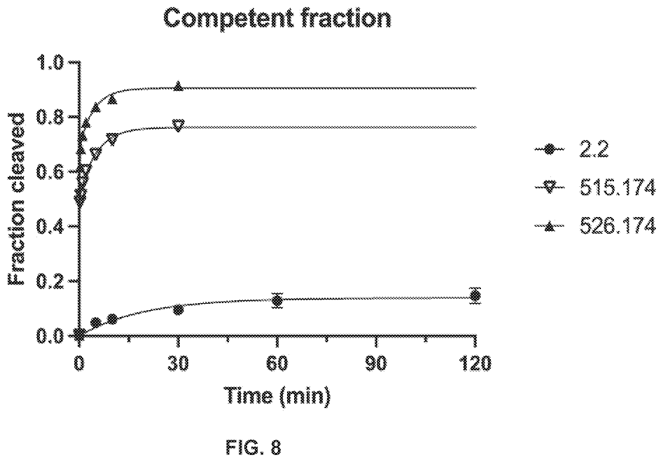

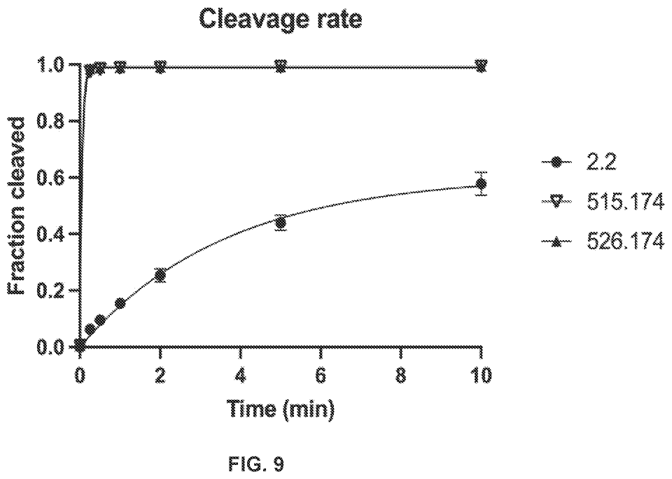

The resulting data clearly demonstrate the dramatic improvements made to RNP activity by engineering both protein and guide. RNPs of 515.174 and 526.174 had competent fractions of 76% and 91%, respectively, as compared to 16% for 2.2 (FIG. 8, Table 13). In the kinetic assay, both 515.174 and 526.174 cut essentially all of the target DNA by the first timepoint, exceeding the resolution of the assay and resulting in estimated cleavage rates of 17.10 and 19.87 min⁻¹, respectively (FIG. 9, Table 13). AnRNP of 2.2, by contrast, cut on average less than 60. of 5the target DNA by the final 10-minute timepoint and has an estimated k_{cleave} nearly two orders of magnitude lower than the engineered RNPs. The modifications made to the protein and guide have resulted in RNPs that are more stable, more likely to form active particles, and cut DNA much more efficiently on a per-particle basis as well.

TABLE 13
Results of cleavage and RNP formation assays

| RNP Construct | k_{cleave}* | | Initial velocity* | Competent fraction |
|---|---|---|---|---|
| 2.2.7.37 | | | 20.4 ± 1.4 nM/min | 16 ± 3% |
| 2.32.7.37 | | | 18.4 ± 2.4 nM/min | 13 ± 3% |
| 2.64.7.37 | | | 7.8 ± 1.8 nM/min | 5 ± 2% |
| 2.174.7.37 | 0.51 ± 0.01 | min⁻¹ | 49.3 ± 1.4 nM/min | 22 ± 5% |
| 119.174.7.37 | 6.29 ± 2.11 | min⁻¹ | | 35 ± 6% |
| 457.174.7.37 | 3.01 ± 0.90 | min⁻¹ | | 53 ± 7% |
| 488.174.7.37 | 15.19 | min⁻¹ | | 67% |
| 491.174.7.37 | 16.59 min⁻¹/ | | | 83%/17% (guide-limited) |
| | 0.293 min⁻¹ (10° C.) | | | |
| 491.175.7.37 | 0.089 min⁻¹ (10° C.) | | | 5% (guide-limited) |
| 491.185.7.37 | 0.227 min⁻¹ (10° C.) | | | 44% (guide-limited) |
| 491.186.7.37 | 0.099 min⁻¹ (10° C.) | | | 11% (guide-limited) |
| 491.196.7.37 | 0.292 min⁻¹ (10° C.) | | | 46% (guide-limited) |
| 491.214.7.37 | 0.284 min⁻¹ (10° C.) | | | 30% (guide-limited) |
| 491.215.7.37 | 0.398 min⁻¹ (10° C.) | | | 38% (guide-limited) |
| 515.174.7.37 | 17.10 | min⁻¹** | | 76% |
| 526.174.7.37 | 19.87 | min⁻¹** | | 91% |

*Mean and standard deviation
**Rate exceeds resolution of assay

### Example 9: Testing Effects of Spacer Length on In Vitro Cleavage Kinetics

Ribonuclear protein complexes (RNP) of two CasX variants and guide RNA with spacers of varying length were tested for in vitro cleavage activity to determine what spacer length supports the most efficient cleavage of a target nucleic acid and whether spacer length preference changes with the protein.

### Methods:

Ribonuclear protein complexes (RNP) of CasX and guide RNA with spacers of varying length were tested for in vitro cleavage activity to determine what spacer length supports the most efficient cleavage of a target nucleic acid.

CasX variant 515 and 526 were purified as described above. Guides with scaffold 174 (SEQ ID NO: 2238) were prepared by in vitro transcription (IVT). IVT templates were generated by PCR using Q5 polymerase (NEB M0491) according to the recommended protocol, template oligos for each scaffold backbone, and amplification primers with the T7 promoter and the 7.37 spacer (GGCCGAGATGTCTCGCTCCG; targeting tdTomato (SEQ ID NO: 27192)) of 20 nucleotides or truncated from the 3′ end to 18 or 19 nucleotides. Spacer sequences as well as the oligonucleotides used to generate each template are shown in Table 14. The resulting templates were then used with T7 RNA polymerase to produce RNA guides according to standard protocols. The guides were purified using denaturing polyacrylamide gel electrophoresis and refolded prior to use.

CasX RNPs were reconstituted by diluting CasX to 1 μM in 1× cleavage buffer (20 mM Tris HCl pH 7.5, 150 mM NaCl, 1 mM TCEP, 5% glycerol, 10 mM MgCl2) and adding sgRNA to 1.2 μM and incubating at 37° C. for 10 min before being moved to ice until ready to use. Fluorescently-labeled 7.37 target DNA was purchased as individual oligonucleotides from Integrated DNA Technologies (full sequences in Table 14), and dsDNA target was prepared by heating an equimolar mix of the two complementary strands in 1× cleavage buffer and slow-cooling to room temperature.

RNPs were diluted in cleavage buffer to a final concentration of 200 nM and incubated at 10° C. without shaking. Cleavage reactions were initiated by the addition of 7.37 target DNA to a final concentration of 10 nM. Timepoints were taken at 0.25, 0.5, 1, 2, 5, 10, and 30 minutes. Timepoints were quenched by adding to an equal volume of 95% formamide, 20 mM EDTA. Samples were denatured by heating at 95° C. for 10 minutes and run on a 10% urea-PAGE gel. Gels were imaged with an Amersham Typhoon and analyzed with IQTL software. The resulting data were plotted and analyzed using Prism. The cleavage of the non-target strand was fit with a single exponential function to determine the apparent first-order rate constant (k_{cleave}).

### Results:

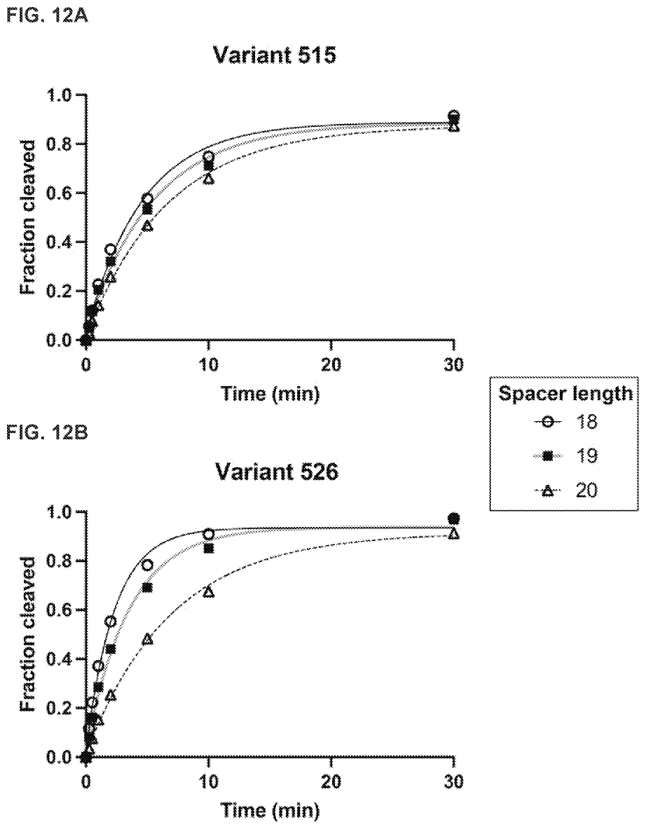

Cleavage rates were compared for CasX variants 515 and 526 in complex with sgRNAs with 18, 19, or 20 nucleotide spacers to determine which spacer length resulted in the most efficient cleavage for each protein variant. Consistent with other experiments performed with in vitro-transcribed sgRNA, the 18-nt spacer guide performed best for both protein variants (FIGS. 12A and B, Table 14). The 18-nt spacer was 1.4-fold faster than the 20-nt spacer for protein 515, and it was 3-fold faster than the 20-nt spacer for protein 526. The 19-nt spacer had intermediate activity for both proteins, though again the difference was more pronounced for variant 526. In general, spacers shorter than 20-nt have been observed to have increased activity across a range of proteins, spacers, and delivery methods, but the degree of improvement and the optimal spacer length have varied. These data show that two engineered proteins that are quite similar in sequence (different in only two residues) can have changes in activity as a result of spacer length that are similar in direction but substantially different in degree.

TABLE 14
Relevant sequences and oligonucleotides

| Description | Sequence | SEQ ID NO |
|---|---|---|
| 7.37 target sequence non-target | IR700- | 27177 |
| strand | TGAAGCTGACAGCATTCGGGCCGAGATGTCTC | |
| | GCTCCGTGGCCTTAGCTGTGCTCGCGCT | |
| 7.37 target sequence target | IR800- | 27176 |
| strand | AGCGCGAGCACAGCTAAGGCCACGGAGCGAGA | |
| | CATCTCGGCCCGAATGCTGTCAGCTTCA | |
| 20-nt spacer sequence | GGCCGAGATGTCTCGCTCCG | 27191 |
| 18-nt spacer sequence | GGCCGAGATGTCTCGCTC | 27193 |
| 19-nt spacer sequence | GGCCGAGATGTCTCGCTCC | 27194 |
| Scaffold 174 template fwd | GAAATTAATACGACTCACTATAACTGGCGCTT | 247 |
| | TTATCTGATTACTTTGAGAGCCATCACCAGCG | |
| | ACTATGTCGTAGTGGGTAAAGCT | |
| Scaffold 174 template rev | CTTTGATGCTCCCTCCGAAGAGGGAGCTTTAC | 248 |
| | CCACTACGACATAGTCGC | |
| T7 amplification primer | GAAATTAATACGACTCACTATA | 234 |
| Scaffold 174 20-nt spacer primer | CGGAGCGAGACATCTCGGCCCTTTGATGCTCC | 249 |
| | CTCC | |
| Scaffold 174 18-nt spacer primer | GAGCGAGACATCTCGGCCCTTTGATGCTCCCT | 27195 |
| | CC | |
| Scaffold 174 19-nt spacer primer | GGAGCGAGACATCTCGGCCCTTTGATGCTCCC | 27196 |
| | TCC | |

TABLE 15
Cleavage rates of RNPs with truncated spacers

| Spacer length | 515 k_{cleave}(min⁻¹) | 526 k_{cleave}(min⁻¹) |
|---|---|---|
| 18 | 0.215 | 0.427 |
| 19 | 0.182 | 0.282 |
| 20 | 0.150 | 0.143 |

### Example 10: Assessing Binding Affinity to the Guide RNA

Purified wild-type and improved CasX will be incubated with synthetic single-guide RNA containing a 3′ Cy7.5 moiety in low-salt buffer containing magnesium chloride as well as heparin to prevent non-specific binding and aggregation. The sgRNA will be maintained at a concentration of 10 pM, while the protein will be titrated from 1 pM to 100 μM in separate binding reactions. After allowing the reaction to come to equilibrium, the samples will be run through a vacuum manifold filter-binding assay with a nitrocellulose membrane and a positively charged nylon membrane, which bind protein and nucleic acid, respectively. The membranes will be imaged to identify guide RNA, and the fraction of bound vs unbound RNA will be determined by the amount of fluorescence on the nitrocellulose vs nylon membrane for each protein concentration to calculate the dissociation constant of the protein-sgRNA complex. The experiment will also be carried out with improved variants of the sgRNA to determine if these mutations also affect the affinity of the guide for the wild-type and mutant proteins. We will also perform electromobility shift assays to qualitatively compare to the filter-binding assay and confirm that soluble binding, rather than aggregation, is the primary contributor to protein-RNA association.

### Example 11: Assessing Binding Affinity to the Target DNA

Purified wild-type and improved CasX will be complexed with single-guide RNA bearing a targeting sequence complementary to the target nucleic acid. The RNP complex will be incubated with double-stranded target DNA containing a PAM and the appropriate target nucleic acid sequence with a 5′ Cy7.5 label on the target strand in low-salt buffer containing magnesium chloride as well as heparin to prevent non-specific binding and aggregation. The target DNA will be maintained at a concentration of 1 nM, while the RNP will be titrated from 1 μM to 100 μM in separate binding reactions. After allowing the reaction to come to equilibrium, the samples will be run on a native 5% polyacrylamide gel to separate bound and unbound target DNA. The gel will be imaged to identify mobility shifts of the target DNA, and the fraction of bound vs unbound DNA will be calculated for each protein concentration to determine the dissociation constant of the RNP-target DNA ternary complex. The experiments are expected to demonstrate the improved binding affinity of the RNP comprising a CasX variant and gRNA variant compared to an RNP comprising a reference CasX and reference gRNA.

### Example 12: Assessing Improved Expression and Solubility Characteristics of CasX Variants for RNP Production

Wild-type and modified CasX variants will be expressed in BL21 (DE3) E. coli under identical conditions. All proteins will be under the control of an IPTG-inducible T7 promoter. Cells will be grown to an OD of 0.6 in TB media at 37° C., at which point the growth temperature will be reduced to 16° C. and expression will be induced by the addition of 0.5 mM IPTG. Cells will be harvested following 18 hours of expression. Soluble protein fractions will be extracted and analyzed on an SDS-PAGE gel. The relative levels of soluble CasX expression will be identified by Coomassie staining. The proteins will be purified in parallel according to the protocol above, and final yields of pure protein will be compared. To determine the solubility of the purified protein, the constructs will be concentrated in storage buffer until the protein begins to precipitate. Precipitated protein will be removed by centrifugation and the final concentration of soluble protein will be measured to determine the maximum solubility for each variant. Finally, the CasX variants will be complexed with single guide RNA and concentrated until precipitation begins. Precipitated RNP will be removed by centrifugation and the final concentration of soluble RNP will be measured to determine the maximum solubility of each variant when bound to guide RNA.

### Example 13: Editing of GATA1 Binding Region in the BCL11A Erythroid Enhancer Locus in HEK293T Cells

Experiment were conducted to demonstrate the ability of CasX to edit the GATA1 binding region in the BCL11A erythroid enhancer locus using the CasX variant 438 and guide variant 174, and a spacer targeting the GATA1 binding region of the human BCL11A erythroid enhancer locus in HEK293T cells.

HEK293T cells were maintained at 37° C. and 5% CO2 in Fibroblast (FB) medium, consisting of Dulbecco's Modified Eagle Medium (DMEM; Corning Cellgro, #10-013-CV) supplemented with 10% fetal bovine serum (FBS; Seradigm, #1500-500), and 100 Units/mL penicillin and 100 mg/mL streptomycin (100×-Pen-Strep; GIBCO #15140-122), and can additionally include sodium pyruvate (100×, Thermofisher #11360070), non-essential amino acids (100× Thermofisher #11140050), HEPES buffer (100× Thermofisher #15630080), and 2-mercaptoethanol (1000× Thermofisher #21985023).

For this experiment, HEK293T cells were seeded at 20-40k cells/well in a 96 well plate in 100 μL of FB medium and cultured in a 37° C. incubator with 5% CO2. The following day, cells were transfected at ˜75% confluence. CasX and guide construct (see Table 16 for sequences) was transfected into the HEK293T cells at 100-500 ng per well using Lipofectamine 3000 following the manufacturer's protocol, using 3 wells per construct as replicates. A non-targeting plasmid was used as a negative control. SpyCas9 and guide construct targeting the same region was used as a benchmarking control. Cells were selected for successful transfection with puromycin at 0.3-3 μg/ml for 24-48 hours followed by recovery in FB medium. Subsequently, cells for each sample from the experiment were lysed, and the genome was extracted following the manufacturer's protocol and standard practices. Editing in cells from each experimental sample were assayed using NGS analysis. Briefly, genomic DNA was amplified via PCR with primers specific to the target genomic location of interest to form a target amplicon. These primers contain additional sequence at the 5′ ends to introduce Illumina read and 2 sequences. Further, they contain a 16 nt random sequence that functions as a unique molecular identifier (UMI). Quality and quantification of the amplicon was assessed using a Fragment Analyzer DNA analyzer kit (Agilent, dsDNA 35-1500 bp). Amplicons were sequenced on the Illumina Miseq according to the manufacturer's instructions. Raw fastq files from sequencing were processed as follows. (1) The sequences were trimmed for quality and for adapter sequences using the program cutadapt (v. 2.1). (2) The sequences from read 1 and read 2 were merged into a single insert sequence using the program flash2 (v2.2.00). (3) The consensus insert sequences were run through the program CRISPResso2 (v 2.0.29), along with the expected amplicon sequence and the spacer sequence. This program quantifies the percent of reads that were modified in a window around the 3′ end of the spacer (30 bp window centered at −3 bp from 3′ end of spacer). The activity of the CasX molecule was quantified as the total percent of reads that contain insertions and/or deletions anywhere within this window.

TABLE 16
Guide sequences

| | Spacer | SEQ | | SEQ | | SEQ |
|---|---|---|---|---|---|---|
| Spacer | Sequence | ID NO | 174 Guide Sequence | ID NO | Guide + Spacer Sequence | ID NO |
| 21.1 | UGGAGCC | 22 | ACUGGCGCUUUUAUCU | 2238 | ACUGGCGCUUUUAUCUGAUUA | 271 |
| | UGUGAUA | | GAUUACUUUGAGAGCC | | CUUUGAGAGCCAUCACCAGCG | |
| | AAAGCA | | AUCACCAGCGACUAUG | | ACUAUGUCGUAGUGGGUAAAG | |
| | | | UCGUAGUGGGUAAAGC | | CUCCCUCUUCGGAGGGAGCAU | |
| | | | UCCCUCUUCGGAGGGA | | CAAAGUGGAGCCUGUGAUAAA | |
| | | | GCAUCAAAG | | AGCA | |

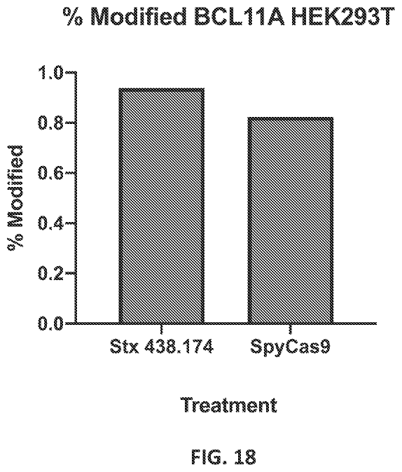

Results: The graph in FIG. 18 shows the results of NGS analysis of CasX-mediated editing of the GATA1 binding region at the BCL11A erythroid enhancer locus in HEK293T cells 5 days post-transfection. Each data point is an average measurement of NGS reads of editing outcomes generated by an individual treatment condition. The results indicate that CasX and guide was able to edit the BCL11A erythroid enhancer locus at an average editing level of 90%, while the SpyCas9 construct showed an average editing level of 80%. The construct with non-targeting spacer resulted in no editing (data not shown). This example demonstrates that CasX with an appropriate guide was able to edit the BCL11A erythroid enhancer locus in HEK293T cells. Experiments with CasX variants 668, 672, 676 and gRNA 235 would be performed under similar conditions and would be expected to result in similar editing efficiency.

### Example 14: Editing of GATA1 Binding Region in the BCL11A Erythroid Enhancer Locus in K562 Cells

Experiment were conducted to demonstrate the ability of CasX to edit the BCL11A erythroid enhancer locus using the CasX variants 119 and 491, scaffold variant 174, and a spacer targeting the GATA1 binding region of the human BCL11A erythroid enhancer locus in K562 cells.

K562 cells were maintained at 37° C. and 5% CO2 in medium consisting of RPMI (RPMI; Thermofisher, #11875119) supplemented with 10% fetal bovine serum (FBS; Seradigm, #1500-500), and 100 Units/mL penicillin and 100 mg/mL streptomycin (100×-Pen-Strep; GIBCO #15140-122), and can additionally include sodium pyruvate (100×, Thermofisher #11360070) and HEPES buffer (100× Thermofisher #15630080).

In this experiment, CasX and guide targeting the GATA1 binding region of the BCL11A locus were introduced into K562 cells using two different delivery modalities, RNPs and XDPs (the RNP packaged in a XDP).In the first experimental arm, CasX RNP targeting the GATA1 binding region of the BCL11A locus (see table for spacer sequence) was formulated using standard methods. Briefly, each CasX RNP (see table for sequences) was transduced into 100k-500k K562 cells at 10-100 pmol per condition using a Lonza nucleofector kit following the manufacturer's protocol, using 3 wells per construct as replicates. Cells were cultured in supplemented RPMI medium at 37° C. and 5% CO2.

In the second experimental arm, XDPs encapsulating CasX targeting the GATA1 binding region of the BCL11A locus were formulated as described below. Briefly, XDPs were produced using four structural plasmids: pXDP17, pSG0010, pGP2, and pXDP3. The plasmid pXDP17 expresses the HIV-1 gag sequence followed by CasX version 491. pSG0010 is scaffold 174 with spacer 21.1 (see below for sequence) targeting BC11A expressed under the U6 promoter. pGP2 expresses the VSV-G targeting moiety. pXDP3 expresses the HIV-1 gag polyprotein with no CasX molecule attached. For producing XDPs, LentiX cells from Takara were split and seeded 24 hours before plasmid DNA transfection. 89 μg of pSG0010, 366 μg of pXDP0017, 30 μg of pXDP0003, and 1.7 μg of pGP2 plasmids were mixed with Opti-MEM and PEI then added to cell culture. Media was changed to Opti-MEM 16 hours post transfection. 54 hours post transfection media was collected and concentrated through centrifugation. XDPs were resuspended in 150 mM NaCl buffer 1 and frozen at −150° C. On the day of the experiment, XDPs were thawed on ice and used immediately on cells.

K562 cells were seeded at 30-50k/well in a 96-well plate, transduced with XDPs at a range of different MOIs, and cultured in supplemented RPMI medium at 37° C. and 5% CO2.

Four days later, editing in cells from each experimental sample from RNP or XDP transduced samples were assayed using NGS analysis. Briefly, genomic DNA was amplified via PCR with primers specific to the target genomic location of interest to form a target amplicon. These primers contain additional sequence at the 5′ ends to introduce Illumina read and 2 sequences. Further, they contain a 16 nt random sequence that functions as a unique molecular identifier (UMI). Quality and quantification of the amplicon was assessed using a Fragment Analyzer DNA analyzer kit (Agilent, dsDNA 35-1500 bp). Amplicons were sequenced on the Illumina Miseq according to the manufacturer's instructions. Raw fastq files from sequencing were processed as follows. (1) The sequences were trimmed for quality and for adapter sequences using the program cutadapt (v. 2.1). (2) The sequences from read 1 and read 2 were merged into a single insert sequence using the program flash2 (v2.2.00). (3) The consensus insert sequences were run through the program CRISPResso2 (v 2.0.29), along with the expected amplicon sequence and the spacer sequence. This program quantifies the percent of reads that were modified in a window around the 3′ end of the spacer (30 bp window centered at −3 bp from 3′ end of spacer). The activity of the CasX molecule was quantified as the total percent of reads that contain insertions and/or deletions anywhere within this window.

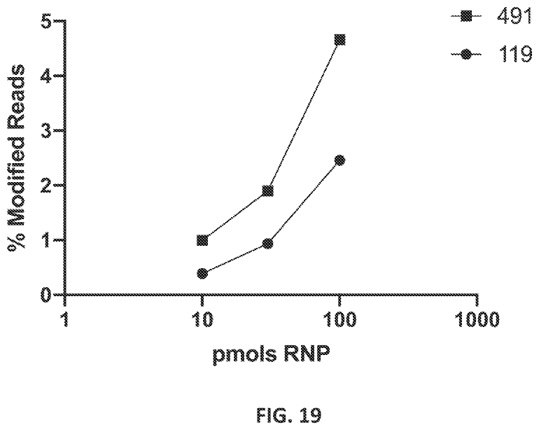

Results: The graph in FIG. 19 shows the results of NGS analysis of CasX-mediated editing of the GATA1 binding region at the BCL11A erythroid enhancer locus in K562 cells 4 days post RNP transduction. Each data point is an average measurement of NGS reads of editing outcomes generated by an individual treatment condition. The results indicate that CasX and guide was able to edit the BCL11A erythroid enhancer locus in a dose-dependent manner, with CasX variant 491 consistently showing a higher level of editing relative to CasX variant 119. This example demonstrates that, under the conditions of the assay, CasX with an appropriate guide was able to edit the BCL11A erythroid enhancer locus in K562 cells.

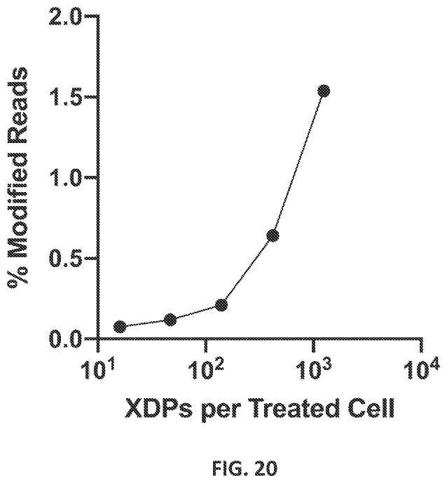

The graph in FIG. 20 shows the results of NGS analysis of CasX-mediated editing of the GATA1 binding region at the BCL11A erythroid enhancer locus in K562 cells 4 days post XDP transduction. Each data point is an average measurement of NGS reads of editing outcomes generated by an individual treatment condition. The results indicate that CasX and guide was able to edit the BCL11A erythroid enhancer locus in a dose-dependent manner. This example demonstrates that CasX with an appropriate guide was able to edit the BCL11A erythroid enhancer locus in K562 cells. Experiments with CasX variants 668, 672, 676 and gRNA 235 would be performed under similar conditions and would be expected to result in similar editing efficiency.

### Example 15: Editing of GATA1 Binding Region in the BCL11A Erythroid Enhancer Locus in Hematopoietic Stem Cells

Experiments were conducted to demonstrate the ability of CasX to edit the BCL11A erythroid enhancer locus using the CasX variants 119 and 491, scaffold variant 174, and a spacer targeting the GATA1 binding region of the human BCL11A erythroid enhancer locus in CD34+ Hematopoietic stem cells (HSCs).

HSCs were cultured in StemSpan SFEM II medium (Stem Cell #9605) supplemented with CC100 (Stem Cell #2697), and maintained at 37° C. and 5% CO2. In this experiment, CasX and guide targeting the GATA1 binding region of the BCL11A locus were introduced into HSCs using two different delivery modalities, RNPs and XDPs. In the first experimental arm, CasX RNP targeting the GATA1 binding region of the BCL11A locus (see table for spacer sequence) was formulated using standard methods. Each CasX RNP (see table for sequences) was transduced into 100k-500k HSCs at 10-100 pmol per condition using a Lonza nucleofector kit following the manufacturer's protocol, using 3 wells per construct as replicates. Cells were cultured in supplemented SFEM II medium at 37° C. and 5% CO2.

In the second experimental arm, XDPs encapsulating CasX targeting the GATA1 binding region of the BCL11A locus were formulated as described below. Briefly, XDPs were produced using four structural plasmids: pXDP17, pSG0010, pGP2, and pXDP3. The plasmid pXDP17 expresses the HIV-1 gag sequence followed by CasX version 491. pSG0010 is scaffold 174 with spacer 21.1 (see below for sequence) targeting BC11A expressed under the U6 promoter. pGP2 expresses the VSV-G targeting moiety. pXDP3 expresses the HIV-1 gag polyprotein with no CasX molecule attached. For producing XDPs, LentiX cells from Takara were split and seeded 24 hours before plasmid DNA transfection. 89 μg of pSG0010, 366 μg of pXDP0017, 30 μg of pXDP0003, and 1.7 μg of pGP2 plasmids were mixed with Opti-MEM and PEI then added to cell culture. Media was changed to Opti-MEM 16 hours post transfection. 54 hours post transfection media was collected and concentrated through centrifugation. XDPs were resuspended in 150 mM NaCl buffer 1 and frozen at −150° C. On the day of the experiment, XDPs were thawed on ice and used immediately on cells.

HSCs were seeded at 30-50k/well in a 96-well plate, transduced with XDPs at a range of different MOIs, and cultured in supplemented SFEM II medium at 37° C. and 5% CO2. Four days later, editing in cells from each experimental condition from RNP or XDP transduced samples were assayed using NGS analysis. Briefly, cells for each sample from the experiment were lysed, and the genome was extracted following the manufacturer's protocol and standard practices. Editing in cells from each experimental sample were assayed using NGS analysis. Briefly, genomic DNA was amplified via PCR with primers specific to the target genomic location of interest to form a target amplicon. These primers contain additional sequence at the 5′ ends to introduce Illumina read and 2 sequences. Further, they contain a 16 nt random sequence that functions as a unique molecular identifier (UMI). Quality and quantification of the amplicon was assessed using a Fragment Analyzer DNA analyzer kit (Agilent, dsDNA 35-1500 bp). Amplicons were sequenced on the Illumina Miseq according to the manufacturer's instructions. Raw fastq files from sequencing were processed as follows. (1) The sequences were trimmed for quality and for adapter sequences using the program cutadapt (v. 2.1). (2) The sequences from read 1 and read 2 were merged into a single insert sequence using the program flash2 (v2.2.00). (3) The consensus insert sequences were run through the program CRISPResso2 (v 2.0.29), along with the expected amplicon sequence and the spacer sequence. This program quantifies the percent of reads that were modified in a window around the 3′ end of the spacer (30 bp window centered at −3 bp from 3′ end of spacer). The activity of the CasX molecule was quantified as the total percent of reads that contain insertions and/or deletions anywhere within this window.

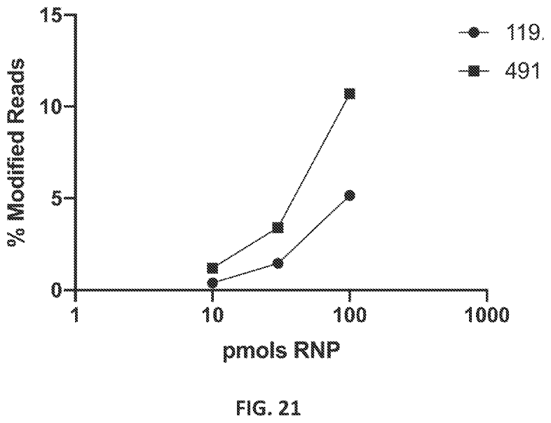

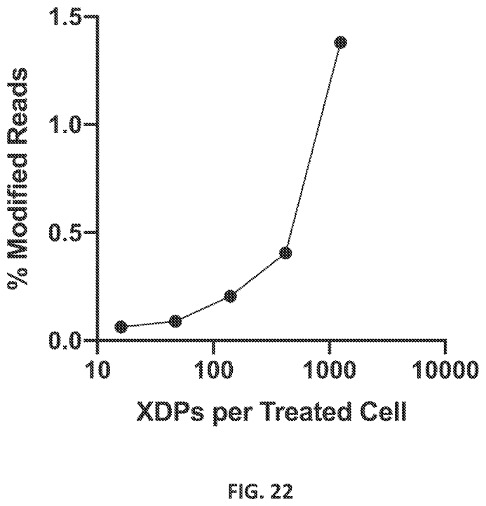

Results: The graph in FIG. 21 shows the results of NGS analysis of CasX-mediated editing of the GATA1 binding region at the BCL11A erythroid enhancer locus in HSCs 4 days post RNP transduction. Each data point is an average measurement of NGS reads of editing outcomes generated by an individual treatment condition. The results indicate that CasX and guide was able to edit the BCL11A erythroid enhancer locus in a dose-dependent manner, with CasX variant 491 consistently showing a higher level of editing relative to CasX variant 119. This example demonstrates that, under the conditions of the assay, CasX with an appropriate guide was able to edit the BCL11A erythroid enhancer locus in HSCs. The graph in FIG. 22 shows the results of NGS analysis of CasX-mediated editing of the GATA1 binding region at the BCL11A erythroid enhancer locus in HSCs 4 days post XDP transduction. Each data point is an average measurement of NGS reads of editing outcomes generated by an individual treatment condition. The results indicate that CasX and guide was able to edit the BCL11A erythroid enhancer locus in a dose-dependent manner. This example demonstrates that CasX with an appropriate guide was able to edit the BCL11A erythroid enhancer locus in HSCs. Experiments with CasX variants 668, 672, 676 and gRNA 235 would be performed under similar conditions and would be expected to result in similar editing efficiency.

What is claimed is:

1. A system comprising a Class 2 Type V CRISPR protein and a first guide ribonucleic acid (gRNA), wherein the gRNA comprises a targeting sequence complementary to a target nucleic acid sequence comprising a polypyrimidine tract-binding protein 1 (BCL11A) gene.

2. The system of claim 1, wherein the gRNA comprises a targeting sequence complementary to a target nucleic acid sequence selected from the group consisting of:
a. a BCL11A intron;
b. a BCL11A exon;
c. a BCL11A intron-exon junction;
d. a BCL11A regulatory element; and
e. an intergenic region.

3. The system of claim 1 or claim 2, wherein the BCL11A gene comprises a wild-type sequence.

4. The system of any one of claims 1-3, wherein the gRNA is a single-molecule gRNA (sgRNA).

5. The system of any one of claims 1-4, wherein the gRNA is a dual-molecule gRNA (dgRNA).

6. The system of any one of claims 1-5, wherein the targeting sequence of the gRNA comprises a sequence selected from the group consisting of SEQ ID NOS: 272-2100 and 2286-26789, or a sequence having at least about 65%, at least about 75%, at least about 85%, or at least about 95% identity thereto.

7. The system of any one of claims 1-5, wherein the targeting sequence of the gRNA comprises a sequence selected from the group consisting of SEQ ID NOS: 272-2100 and 2286-26789.

8. The system of claim 7, wherein the targeting sequence has a single nucleotide removed from the 3′ end of the sequence.

9. The system of claim 7, wherein the targeting sequence has two nucleotides removed from the 3′ end of the sequence.

10. The system of claim 7, wherein the targeting sequence has three nucleotides removed from the 3′ end of the sequence.

11. The system of claim 7, wherein the targeting sequence has four nucleotides removed from the 3′ end of the sequence.

12. The system of claim 7, wherein the targeting sequence has five nucleotides removed from the 3′ end of the sequence.

13. The system of any one of claims 1-12, wherein the targeting sequence of the gRNA is complementary to a sequence of a BCL11A exon.

14. The system of claim 13, wherein the targeting sequence of the gRNA is complementary to a sequence selected from the group consisting of a BCL11A exon 1 sequence, BCL11A exon 2 sequence, BCL11A exon 3 sequence, BCL11A exon 4 sequence, BCL11A exon 5 sequence, BCL11A exon 6 sequence, BCL11A exon 7 sequence, BCL11A exon 8 sequence, and a BCL11A exon 9 sequence.

15. The system of claim 14, wherein the targeting sequence of the gRNA is complementary to a sequence selected from the group consisting of a BCL11A exon 1 sequence, BCL11A exon 2 sequence, and a BCL11A exon 3 sequence.

16. The system of any one of claims 1-12, wherein the targeting sequence of the gRNA is complementary to a sequence of a BCL11A regulatory element.

17. The system of claim 16, wherein the targeting sequence of the gRNA is complementary to a sequence of a promoter of the BCL11A gene.

18. The system of claim 16, wherein the targeting sequence of the gRNA is complementary to a sequence of an enhancer regulatory element.

19. The system of claim 18, wherein the targeting sequence of the gRNA is complementary to a sequence that comprises a GATA1 erythroid-specific enhancer binding site (GATA1) of the BCL11A gene.

20. The system of claim 16, wherein the targeting sequence of the gRNA is complementary to a sequence that is 5′ to the GATA1 binding site of the BCL11A gene.

21. The system of claim 19 or claim 20, wherein the targeting sequence of the gRNA comprises a sequence of UGGAGCCUGUGAUAAAAGCA (SEQ ID NO: 22), or a sequence having at least 90% or 95% sequence identity thereto.

22. The system of claim 19, wherein the targeting sequence of the gRNA consists of a sequence of UGGAGCCUGUGAUAAAAGCA (SEQ ID NO: 22).

23. The system of claim 18, wherein the targeting sequence of the gRNA comprises a sequence of UGCUUUUAUCACAGGCUCCA (SEQ ID NO: 23), or a sequence having at least 90% or 95% sequence identity thereto.

24. The system of claim 18, wherein the targeting sequence of the gRNA consists of a sequence of UGCUUUUAUCACAGGCUCCA (SEQ ID NO: 23).

25. The system of claim 19 or claim 20, wherein the targeting sequence of the gRNA comprises a sequence of CAGGCUCCAGGAAGGGUUUG (SEQ ID NO: 2949), or a sequence having at least 90% or 95% sequence identity thereto.

26. The system of claim 19 or claim 20, wherein the targeting sequence of the gRNA consists of a sequence of CAGGCUCCAGGAAGGGUUUG (SEQ ID NO: 2949).

27. The system of claim 19 or claim 20, wherein the targeting sequence of the gRNA comprises a sequence of GAGGCCAAACCCUUCCUGGA (SEQ ID NO: 2948), or a sequence having at least 90% or 95% sequence identity thereto.

28. The system of claim 19 or claim 20, wherein the targeting sequence of the gRNA consists of a sequence of CAGGCUCCAGGAAGGGUUUG (SEQ ID NO: 2948).

29. The system of claim 19 or claim 20, wherein the targeting sequence of the gRNA comprises a sequence of AGUGCAAGCUAACAGUUGCU (SEQ ID NO: 15747), or a sequence having at least 90% or 95% sequence identity thereto.

30. The system of claim 19 or claim 20, wherein the targeting sequence of the gRNA consists of a sequence of AGUGCAAGCUAACAGUUGCU (SEQ ID NO: 15747).

31. The system of claim 19 or claim 20, wherein the targeting sequence of the gRNA comprises a sequence of AUACAACUUUGAAGCUAGUC (SEQ ID NO: 15748), or a sequence having at least 90% or 95% sequence identity thereto.

32. The system of claim 19 or claim 20, wherein the targeting sequence of the gRNA consists of a sequence of AUACAACUUUGAAGCUAGUC (SEQ ID NO: 15748).

33. The system of any one of claims 1-32, further comprising a second gRNA, wherein the second gRNA has a targeting sequence complementary to a different or overlapping portion of the BCL11A target nucleic acid compared to the targeting sequence of the gRNA of the first gRNA.

34. The system of claim 33, wherein the targeting sequence of the second gRNA is complementary to a sequence of the target nucleic acid that is 5′ or 3′ to the GATA1 binding site sequence.

35. The system of claim 33, wherein the first and the second gRNA each have a targeting sequence complementary to a sequence within the promoter of the BCL11A gene.

36. The system of any one of claims 1-35, wherein the first or second gRNA has a scaffold comprising a sequence selected from the group consisting of SEQ ID NOS: 2238-2285, 26794-26839 and 27219-27265, or a sequence having at least about 50%, at least about 60%, at least about 70%, at least about 80%, at least about 90%, at least about 95%, at least about 96%, at least about 97%, at least about 98%, at least about 99% sequence identity thereto.

37. The system of any one of claims 1-36, wherein the first or second gRNA has a scaffold comprising a sequence selected from the group consisting of SEQ ID NOs: 2238-2285, 26794-26839 and 27219-27265.

38. The system of any one of claims 1-36, wherein the first or second gRNA has a scaffold consisting of a sequence selected from the group consisting of SEQ ID NOs: 2238-2285, 26794-26839 and 27219-27265.

39. The system of claim 38, wherein the first or second gRNA has a scaffold consisting of the sequence of SEQ ID NO: 2238 or SEQ ID NO: 26800.

40. The system of any one of claims 36-39, wherein targeting sequence is linked to the 3′ end of the scaffold of the gRNA.

41. The system of any one of claims 1-40, wherein the Class 2 Type V CRISPR protein is a CasX variant protein comprising a sequence selected from the group consisting of SEQ ID NOS: 59, 72-99, 101-148, and 26908-27154, or a sequence having at least about 50%, at least about 60%, at least about 70%, at least about 80%, at least about 90%, or at least about 95%, or at least about 95%, or at least about 96%, or at least about 97%, or at least about 98%, or at least about 99% sequence identity thereto.

42. The system of claim 41, wherein the Class 2 Type V CRISPR protein is a CasX variant protein comprising a sequence selected from the group consisting of SEQ ID NOS: 59, 72-99, 101-148, and 26908-27154.

43. The system of claim 41, wherein the CasX variant protein consists of a sequence selected from the group consisting of SEQ ID NOS: 59, 72-99, 101-148, and 26908-27154.

44. The system of claim 42, wherein the CasX variant protein consists of a sequence selected from the group consisting of SEQ ID NOS: 126, 27043, 27046, 27050.

45. The system of claim 41, wherein the CasX variant protein comprises at least one modification relative to a reference CasX protein having a sequence selected from SEQ ID NOS:1-3.

46. The system of claim 45, wherein the at least one modification comprises at least one amino acid substitution, deletion, or substitution in a domain of the CasX variant protein relative to the reference CasX protein.

47. The system of claim 46, wherein the domain is selected from the group consisting of a non-target strand binding (NTSB) domain, a target strand loading (TSL) domain, a helical I domain, a helical II domain, an oligonucleotide binding domain (OBD), and a RuvC DNA cleavage domain.

48. The system of any one of claims 41-47, wherein the CasX variant protein does not comprise an HNH domain.

49. The system of any one of claims 41-48, wherein the CasX variant protein further comprises one or more nuclear localization signals (NLS).

50. The system of claim 49, wherein the one or more NLS are selected from the group of sequences consisting of PKKKRKV (SEQ ID NO: 168), KRPAATKKAGQAKKKK (SEQ ID NO: 169), PAAKRVKLD (SEQ ID NO: 170), RQRRNELKRSP (SEQ ID NO: 171), NQSSNFGPMKGGNFGGRSSGPYGGGGQYFAKPRNQGGY (SEQ ID NO: 172), RMRIZFKNKGKDTAELRRRRVEVSVELRKAKKDEQILKRRNV (SEQ ID NO: 173), VSRKRPRP (SEQ ID NO: 174), PPKKARED (SEQ ID NO: 175), PQPKKKPL (SEQ ID NO: 176), SALIKKKKKMAP (SEQ ID NO: 177), DRLRR (SEQ ID NO: 178), PKQKKRK (SEQ ID NO: 179), RKLKKKIKKL (SEQ ID NO: 180), REKKKFLKRR (SEQ ID NO: 181), KRKGDEVDGVDEVAKKKSKK (SEQ ID NO: 182), RKCLQAGMNLEARKTKK (SEQ ID NO: 183), PRPRKIPR (SEQ ID NO: 184), PPRKKRTVV (SEQ ID NO: 185), NLSKKKKRKREK (SEQ ID NO: 186), RRPSRPFRKP (SEQ ID NO: 187), KRPRSPSS (SEQ ID NO: 188), KRGINDRNFWRGENERKTR (SEQ ID NO: 189), PRPPKMARYDN (SEQ ID NO: 190), KRSFSKAF (SEQ ID NO: 191), KLKIKRPVK (SEQ ID NO: 192), PKKKRKVPPPPAAKRVKLD (SEQ ID NO: 193), PKTRRRPRRSQRKRPPT (SEQ ID NO:26792), SRRRKANPTKLSENAKKLAKEVEN (SEQ ID NO: 194), KTRRRPRRSQRKRPPT (SEQ ID NO: 195), RRKKRRPRRKKRR (SEQ ID NO: 196), PKKKSRKPKKKSRK (SEQ ID NO: 197), HKKKHPDASVNFSEFSK (SEQ ID NO: 198), QRPGPYDRPQRPGPYDRP (SEQ ID NO: 199), LSPSLSPLLSPSLSPL (SEQ ID NO: 200), RGKGGKGLGKGGAKRHRK (SEQ ID NO: 201), PKRGRGRPKRGRGR (SEQ ID NO: 202), PKKKRKVPPPPAAKRVKLD (SEQ ID NO: 203), PKKKRKVPPPPKKKRKV (SEQ ID NO: 204), PAKRARRGYKC (SEQ ID NO: 27199), KLGPRKATGRW (SEQ ID NO: 27200), PRRKREE (SEQ ID NO: 27201), PYRGRKE (SEQ ID NO: 27202), PLRKRPRR (SEQ ID NO: 27203), PLRKRPRRGSPLRKRPRR (SEQ ID NO: 27204), PAAKRVKLDGGKRTADGSEFESPKKKRKV (SEQ ID NO: 27205), PAAKRVKLDGGKRTADGSEFESPKKKRKVGIHGVPAA (SEQ ID NO: 27206), PAAKRVKLDGGKRTADGSEFESPKKKRKVAEAAAKEAAAKEAAAKA (SEQ ID NO: 207), PAAKRVKLDGGKRTADGSEFESPKKKRKVPG (SEQ ID NO: 27208), KRKGSPERGERKRHW (SEQ ID NO: 27209), KRTADSQHSTPPKTKRKVEFEPKKKRKV (SEQ ID NO: 27210), and PKKKRKVGGSKRTADSQHSTPPKTKRKVEFEPKKKRKV (SEQ ID NO: 27211), wherein the one or more NLS are linked to the CRISPR protein or to adjacent NLS with a linker peptide wherein the linker peptide is selected from the group consisting of RS, (G)n (SEQ ID NO: 27212), (GS)n (SEQ ID NO: 27213), (GSGGS)n (SEQ ID NO: 214), (GGSGGS)n (SEQ ID NO: 215), (GGGS)n (SEQ ID NO: 216), GGSG (SEQ ID NO: 217), GGSGG (SEQ ID NO: 218), GSGSG (SEQ ID NO: 219), GSGGG (SEQ ID NO: 220), GGGSG (SEQ ID NO: 221), GSSSG (SEQ ID NO: 222), GPGP (SEQ ID NO: 223), GGP, PPP, PPAPPA (SEQ ID NO: 224), PPPG (SEQ ID NO: 27214), PPPGPPP (SEQ ID NO: 225), PPP(GGGS)n (SEQ ID NO: 27215), (GGGS)nPPP (SEQ ID NO: 27216), AEAAAKEAAAKEAAAKA (SEQ ID NO: 27217), and TPPKTKRKVEFE (SEQ ID NO: 27218), wherein n is 1 to 5.

51. The system of claim 49 or claim 50, wherein the one or more NLS are located at or near the C-terminus of the CasX variant protein.

52. The system of claim 49 or claim 50, wherein the one or more NLS are located at or near the N-terminus of the CasX variant protein.

53. The system of claim 49 or claim 50, comprising one or more NLS located at or near the N-terminus and at or near the C-terminus of the CasX variant protein.

54. The system of any one of claims 41-53, wherein the CasX variant is capable of forming a ribonuclear protein complex (RNP) with a guide nucleic acid (gRNA).

55. The system of claim 54, wherein an RNP of the CasX variant protein and the gRNA variant exhibit at least one or more improved characteristics as compared to an RNP of a reference CasX protein of SEQ ID NO: 1, SEQ ID NO: 2, or SEQ ID NO: 3 and a gRNA comprising a sequence of any one of SEQ ID NOs: 4-16.

56. The system of claim 55, wherein the improved characteristic is selected from one or more of the group consisting of improved folding of the CasX variant; improved binding affinity to a guide nucleic acid (gRNA); improved binding affinity to a target DNA; improved ability to utilize a greater spectrum of one or more protospacer adjacent motif (PAM) sequences, including ATC, CTC, GTC, or TTC, in the editing of target DNA; improved unwinding of the target DNA; increased editing activity; improved editing efficiency; improved editing specificity; increased nuclease activity; improved target nucleic acid sequence cleavage rate; increased target strand loading for double strand cleavage; decreased target strand loading for single strand nicking; decreased off-target cleavage; improved binding of non-target DNA strand; improved protein stability; improved protein solubility; improved ribonuclear protein complex (RNP) formation; higher percentage of cleavage-competent RNP; improved protein:gRNA complex (RNP) stability; improved protein:gRNA complex solubility; improved protein yield; improved protein expression; and improved fusion characteristics.

57. The system of claim 55 or claim 56, wherein the improved characteristic of the RNP of the CasX variant protein and the gRNA variant is at least about 1.1 to about 100-fold or more improved relative to the RNP of the reference CasX protein of SEQ ID NO: 1, SEQ ID NO: 2, or SEQ ID NO: 3 and the gRNA comprising a sequence of any one of SEQ ID NOs: 4-16.

58. The system of claim 55 or claim 56, wherein the improved characteristic of the CasX variant protein is at least about 1.1, at least about 2, at least about 10, at least about 100-fold or more improved relative to the reference CasX protein of SEQ ID NO: 1, SEQ ID NO: 2, or SEQ ID NO: 3 and the gRNA comprising a sequence of any one of SEQ ID NOs: 4-16.

59. The system of claim 55 or claim 56, wherein the improved characteristic of the CasX variant protein is at least about 1.1, at least about 2, at least about 10, at least about 100-fold or more improved relative to the reference CasX protein of SEQ ID NO: 1, SEQ ID NO: 2, or SEQ ID NO: 3 and the gNA comprising a sequence of any one of SEQ ID NOS: 4-16.

60. The system of any one of claims 55-59, wherein the improved characteristic comprises editing efficiency, and the RNP of the CasX variant protein and the gRNA variant comprises a 1.1 to 100-fold improvement in editing efficiency compared to the RNP of the reference CasX protein of SEQ ID NO: 2 and the gRNA of any one of SEQ ID NOs: 4-16.

61. The system of any one of claims 54-59, wherein the RNP comprising the CasX variant and the gRNA variant exhibits greater editing efficiency and/or binding of a target nucleic acid sequence when any one of the PAM sequences TTC, ATC, GTC, or CTC is located 1 nucleotide 5′ to the non-target strand of a protospacer having identity with the targeting sequence of the gRNA in a cellular assay system compared to the editing efficiency and/or binding of an RNP comprising a reference CasX protein and a reference gRNA in a comparable assay system.

62. The system of claim 61, wherein the PAM sequence is TTC.

63. The system of claim 62, wherein the targeting sequence of the gRNA comprises a sequence selected from the group consisting of SEQ ID NOS: 17904-26789.

64. The system of claim 61, wherein the PAM sequence is ATC.

65. The system of claim 64, wherein the targeting sequence of the gRNA comprises a sequence selected from the group consisting of SEQ ID NOS: 272-2100 and 2286-5625.

66. The system of claim 61, wherein the PAM sequence is CTC.

67. The system of claim 66, wherein the targeting sequence of the gRNA comprises a sequence selected from the group consisting of SEQ ID NOS: 5626-13616.

68. The system of claim 61, wherein the PAM sequence is GTC.

69. The system of claim 66, wherein the targeting sequence of the gRNA comprises a sequence selected from the group consisting of SEQ ID NOS: 13617-17903.

70. The system of any one of claims 61-68, wherein the increased binding affinity for the one or more PAM sequences is at least 1.5-fold greater compared to the binding affinity of any one of the reference CasX proteins of SEQ ID NOS: 1-3 for the PAM sequences.

71. The system of any one of claims 54-70, wherein the RNP has at least a 5%, at least a 10%, at least a 15%, or at least a 20% higher percentage of cleavage-competent RNP compared to an RNP of the reference CasX protein and the gRNA of SEQ ID NOs: 4-16.

72. The system of any one of claims 41-71, wherein the CasX variant protein comprises a RuvC DNA cleavage domain having nickase activity.

73. The system of any one of claims 41-71, wherein the CasX variant protein comprises a RuvC DNA cleavage domain having double-stranded cleavage activity.

74. The system of any one of claims 1-54, wherein the CasX protein is a catalytically inactive CasX (dCasX) protein, and wherein the dCasX and the gRNA retain the ability to bind to the BCL11A target nucleic acid.

75. The system of claim 74, wherein the dCasX comprises a mutation at residues:
a. D672, E769, and/or D935 corresponding to the CasX protein of SEQ ID NO:1; or
b. D659, E756 and/or D922 corresponding to the CasX protein of SEQ ID NO: 2.

76. The system of claim 75, wherein the mutation is a substitution of alanine for the residue.

77. The system of any one of claims 1-73, further comprising a donor template nucleic acid.

78. The system of claim 77, wherein the donor template comprises a nucleic acid comprising at least a portion of a BCL11A gene selected from the group consisting of a BCL11A exon, a BCL11A intron, a BCL11A intron-exon junction, a BCL11A regulatory element, and the GATA1 binding site sequence.

79. The system of claim 78, wherein the donor template sequence comprises one or more mutations relative to a corresponding portion of a wild-type BCL11A gene.

80. The system of claim 78 or claim 79, wherein the donor template comprises a nucleic acid comprising at least a portion of a BCL11A exon selected from the group consisting of BCL11A exon 1, BCL11A exon 2, BCL11A exon 3, BCL11A exon 4, BCL11A exon 5, BCL11A exon 6, BCL11A exon 7, BCL11A exon 8, and BCL11A exon 9.

81. The system of claim 80, wherein the donor template comprises a nucleic acid comprising at least a portion of a BCL11A exon selected from the group consisting of BCL11A exon 1, BCL11A exon 2, and BCL11A exon 3.

82. The system of any one of claims 77-81, wherein the donor template ranges in size from 10-15,000 nucleotides.

83. The system of any one of claims 77-82, wherein the donor template is a single-stranded DNA template or a single stranded RNA template.

84. The system of any one of claims 77-82, wherein the donor template is a double-stranded DNA template.

85. The system of any one of claims 77-84, wherein the donor template comprises homologous arms at or near the 5′ and 3′ ends of the donor template that are complementary to sequences flanking cleavage sites in the BCL11A target nucleic acid introduced by the Class 2 Type V CRISPR protein.

86. A nucleic acid comprising the donor template of any one of claims 77-85.

87. A nucleic acid comprising a sequence that encodes the CasX of any one of claims 41-76.

88. A nucleic acid comprising a sequence that encodes the gRNA of any one of claims 1-40.

89. The nucleic acid of claim 87, wherein the sequence that encodes the CasX protein is codon optimized for expression in a eukaryotic cell.

90. A vector comprising the gRNA of any one of claims 1-40, the CasX protein of any one of claims 41-76, or the nucleic acid of any one of claims 86-89.

91. The vector of claim 90, wherein the vector further comprises one or more promoters.

92. The vector of claim 90 or claim 91, wherein the vector is selected from the group consisting of a retroviral vector, a lentiviral vector, an adenoviral vector, an adeno-associated viral (AAV) vector, a herpes simplex virus (HSV) vector, a virus-like particle (VLP), a CasX delivery particle (XDP), a plasmid, a minicircle, a nanoplasmid, a DNA vector, and an RNA vector.

93. The vector of claim 92, wherein the vector is an AAV vector.

94. The vector of claim 93, wherein the AAV vector is selected from AAV1, AAV2, AAV3, AAV4, AAV5, AAV6, AAV7, AAV8, AAV9, AAV10, AAV-Rh74, or AAVRh10.

95. The vector of claim 94, wherein the AAV vector is selected from AAV1, AAV2, AAV5, AAV8, or AAV9.

96. The vector of claim 94 or claim 95, wherein the AAV vector comprises a nucleic acid comprising the following components:
a. 5′ ITR;
b. a 3′ ITR; and
c. a transgene comprising the nucleic acid of claim 87 operably linked to a first promoter and the nucleic acid of claim 88 operably linked to a second promoter.

97. The vector of claim 96, wherein the nucleic acid further comprises a poly(A) sequence 3′ to the sequence encoding the CasX protein.

98. The vector of claim 96 or claim 97, wherein the nucleic acid further comprises one or more enhancer elements.

99. The vector of any one of claims 96-98, wherein a single AAV particle is capable of containing the nucleic acid, wherein the AAV particle has all the components necessary to transduce and effectively modify a target nucleic in a target cell.

100. The vector of claim 92, wherein the vector is a retroviral vector.

101. The vector of claim 92, wherein the vector is a XDP comprising one or more components of a gag polyprotein.

102. The vector of claim 101, wherein the one or more components of the gag polyprotein are selected from the group consisting of a matrix protein (MA), a nucleocapsid protein (NC), a capsid protein (CA), a p1 peptide, a p6 peptide, a P2A peptide, a P2B peptide, a P10 peptide, a p12 peptide, a PP 21/24 peptide, a P12/P3/P8 peptide, and a P20 peptide.

103. The vector of claim 101 or claim 102, wherein the XDP comprises the one or more components of the gag polyprotein, the CasX variant protein, and the gRNA.

104. The vector of claim 103, wherein the CasX variant protein and the gRNA are associated together in an RNP.

105. The vector of any one of claims 101-104, further comprising the donor template.

106. The vector of any one of claims 101-104, further comprising a pseudotyping viral envelope glycoprotein or antibody fragment that provides for binding and fusion of the XDP to a target cell.

107. The vector of claim 106, wherein the target cell is selected from the group consisting of a hematopoietic stem cell (HSC), a hematopoietic progenitor cell (HPC), a CD34+ cell, a mesenchymal stem cell (MSC), an embryonic stem (ES) cell, an induced pluripotent stem cell (iPSC), a common myeloid progenitor cell, a proerythroblast cell, and an erythroblast cell.

108. A host cell comprising the vector of any one of claims 90-107.

109. The host cell of claim 108, wherein the host cell is selected from the group consisting of Baby Hamster Kidney fibroblast (BHK) cells, human embryonic kidney 293 (HEK293) cells, human embryonic kidney 293T (HEK293T) cells, NS0 cells, SP2/0 cells, YO myeloma cells, P3X63 mouse myeloma cells, PER cells, PER.C6 cells, hybridoma cells, NIH3T3 cells, CV-1 (simian) in Origin with SV40 genetic material (COS) cells, HeLa, Chinese hamster ovary (CHO) cells, and yeast cells.

110. A method of modifying a BCL11A target nucleic acid sequence in a population of cells, the method comprising introducing into cells of the population:
a. the system of any one of claims 1-85;
b. the nucleic acid of any one of claims 86-89;
c. the vector as in any one of claims 90-100;
d. the XDP of any one of claims 101-107; or
e. combinations of two or more of (a)-(d),
wherein the BCL11A gene target nucleic acid sequence of the cells targeted by the first gRNA is modified by the CasX variant protein.

111. The method of claim 110, wherein the modifying comprises introducing a single-stranded break in the BCL11A gene target nucleic acid sequence of the cells of the population.

112. The method of claim 110, wherein the modifying comprises introducing a double-stranded break in the BCL11A gene target nucleic acid sequence of the cells of the population.

113. The method of any one of claims 110-112, further comprising introducing into the cells of the population a second gRNA or a nucleic acid encoding the second gRNA, wherein the second gRNA has a targeting sequence complementary to a different or overlapping portion of the BCL11A gene target nucleic acid compared to the first gRNA, resulting in an additional break in the BCL11A target nucleic acid of the cells of the population.

114. The method of any one of claims 110-113, wherein the modifying comprises introducing an insertion, deletion, substitution, duplication, or inversion of one or more nucleotides in the BCL11A gene of the cells of the population.

115. The method of claim 110-114, wherein a GATA1 binding site sequence of the target nucleic acid is modified.

116. The method of any one of claims 110-113, wherein the method comprises insertion of the donor template into the break site(s) of the BCL11A gene target nucleic acid sequence of the cells of the population.

117. The method of claim 114, wherein the insertion of the donor template is mediated by homology-directed repair (HDR) or homology-independent targeted integration (HITI).

118. The method of claim 116 or claim 117, wherein the GATA1 binding site sequence of the target nucleic acid is modified.

119. The method of any one of claims 116-118, wherein insertion of the donor template results in a knock-down or knock-out of the BCL11A gene in the cells of the population.

120. The method of any one of claims 110-119, wherein the BCL11A gene of the cells of the population is modified such that expression of the BCL11A protein is reduced by at least about 10%, at least about 20%, at least about 30%, at least about 40%, at least about 50%, at least about 60%, at least about 70%, at least about 80%, or at least about 90% in comparison to cells in which the BCL11A gene has not been modified.

121. The method of any one of claims 110-119, wherein the BCL11A gene of the cells of the population is modified such that at least about 10%, at least about 20%, at least about 30%, at least about 40%, at least about 50%, at least about 60%, at least about 70%, at least about 80%, or at least about 90% of the modified cells do not express a detectable level of BCL11A protein.

122. The method of any one of claims 110-121, wherein the cells are eukaryotic.

123. The method of claim 122, wherein the eukaryotic cells are selected from the group consisting of rodent cells, mouse cells, rat cells, and non-human primate cells.

124. The method of claim 122, wherein the eukaryotic cells are human cells.

125. The method of any one of claims 122-124, wherein the eukaryotic cell is selected from the group consisting of a hematopoietic stem cell (HSC), a hematopoietic progenitor cell (HPC), a CD34+ cell, a mesenchymal stem cell (MSC), induced pluripotent stem cell (iPSC), a common myeloid progenitor cell, a proerythroblast cell, and an erythroblast cell.

126. The method of any one of claim 110-125, wherein the modification of the BCL11A gene target nucleic acid sequence of the population of cells occurs in vitro or ex vivo.

127. The method of any one of claim 110-125, wherein the modification of the BCL11A gene target nucleic acid sequence of the population of cells occurs in vivo in a subject.

128. The method of claim 127, wherein the subject is selected from the group consisting of a rodent, a mouse, a rat, and a non-human primate.

129. The method of claim 127, wherein the subject is a human.

130. The method of any one of claims 127-129, wherein the method comprises administering a therapeutically effective dose of the AAV vector to the subject.

131. The method of claim 130, wherein the AAV vector is administered to the subject at a dose of at least about 1×10⁵ vector genomes/kg (vg/kg), at least about 1×10⁶ vg/kg, at least about 1×10⁷ vg/kg, at least about 1×10⁸ vg/kg, at least about 1×10⁹ vg/kg, at least about 1×10¹⁰ vg/kg, at least about 1×10¹¹ vg/kg, at least about 1×10¹² vg/kg, at least about 1×10¹³ vg/kg, at least about 1×10¹⁴ vg/kg, at least about 1×10¹⁵ vg/kg, or at least about 1×10¹⁶ vg/kg.

132. The method of claim 130, wherein the AAV vector is administered to the subject at a dose of at least about 1×10⁵ vg/kg to about 1×10¹⁶ vg/kg, at least about 1×10⁶ vg/kg to about 1×10¹⁵ vg/kg, or at least about 1×10⁷ vg/kg to about 1×10¹⁴ vg/kg.

133. The method of any one of claims 127-129, wherein the method comprises administering a therapeutically effective dose of a XDP to the subject.

134. The method of claim 133, wherein the XDP is administered to the subject at a dose of at least about 1×10⁵ particles/kg, at least about 1×10⁶ particles/kg, at least about 1×10⁷ particles/kg at least about 1×10⁸ particles/kg, at least about 1×10⁹ particles/kg, at least about 1×10¹⁰ particles/kg, at least about 1×10¹¹ particles/kg, at least about 1×10¹² particles/kg, at least about 1×10¹³ particles/kg, at least about 1×10¹⁴ particles/kg, at least about 1×10¹⁵ particles/kg, at least about 1×10¹⁶ particles/kg.

135. The method of claim 133, wherein the XDP is administered to the subject at a dose of at least about 1×10⁵ particles/kg to about 1×10¹⁶ particles/kg, or at least about 1×10⁶ particles/kg to about 1×10¹⁵ particles/kg, or at least about 1×10⁷ particles/kg to about 1×10¹⁴ particles/kg

136. The method of any one of claims 128-135, wherein the vector or XDP is administered to the subject by a route of administration selected from transplantation, local injection, systemic infusion, or combinations thereof.

137. The method of any one of claims 128-136, wherein the method results in an increased levels of hemoglobin F (HbF) in circulating blood of the subject of at least about 5%, at least about 10%, at least about 20%, at least about 30%, at least about 40%, or at least about 50% compared to the levels of HbF in the subject prior to treatment.

138. The method of any one of claims 128-137, wherein the method results in a ratio of HbF to hemoglobin S (HbS) in circulating blood of the subject of at least 0.01:1.0, at least 0.025:1.0, at least 0.05:1.0, at least 0.075:1.0 at least 0.1:1.0, at least 0.2:1.0, at least 0.3:1.0, at least 0.4:1.0, at least 0.5:1:0, at least 0.75:1.0, at least 1.0:1.0, at least 1.25:1.0, at least 1.5:1.0, or at least 1.75:1.0.

139. The method of any one of claims 128-138, wherein the method results in HbF levels of at least about 5%, or at least about 10%, or at least about 20%, or at least about 30% of total hemoglobin in circulating blood of the subject.

140. The method of any one of claims 110-139, further comprising contacting the BCL11A gene target nucleic acid sequence of the population of cells with:
a. an additional CRISPR nuclease and a gRNA targeting a different or overlapping portion of the BCL11A target nucleic acid compared to the first gRNA;
b. a polynucleotide encoding the additional CRISPR nuclease and the gRNA of (a);
c. a vector comprising the polynucleotide of (b); or
d. a XDP comprising the additional CRISPR nuclease and the gRNA of (a) wherein the contacting results in modification of the BCL11A gene at a different location in the sequence compared to the sequence targeted by the first gRNA.

141. The method of claim 140, wherein the additional CRISPR nuclease is a CasX protein having a sequence different from the CasX protein of any of the preceding claims.

142. The method of claim 140, wherein the additional CRISPR nuclease is not a CasX protein.

143. The method of claim 142, wherein the additional CRISPR nuclease is selected from the group consisting of Cas9, Cas12a, Cas12b, Cas12c, Cas12d (CasY), Cas12j, Cas12k, Cas13a, Cas13b, Cas13c, Cas13d, Cas14, Cpf1, C2cl, Csn2, and sequence variants thereof.

144. A population of cells modified by the method of any one of claims 110-143, wherein the cells have been modified such that at least 70%, at least 75%, at least 80%, at least 85%, at least 90%, or at least 95% of the modified cells do not express a detectable level of BCL11A protein.

145. A population of cells modified by the method of any one of claims 110-143, wherein the cells have been modified such that the expression of BCL11A protein is reduced by at least 70%, at least 75%, at least 80%, at least 85%, at least 90%, or at least 95% compared to cells where the BCL11A gene has not been modified.

146. A method of treating a hemoglobinopathy in a subject in need thereof, the method comprising administering to the subject a therapeutically effective amount of the cells of claim 144 or claim 145.

147. The method of claim 146, wherein the hemoglobinopathy is a sickle cell disease or beta-thalassemia.

148. The method of claim 146 or claim 147, wherein the cells are autologous with respect to the subject to be administered the cells.

149. The method of claim 146 or claim 147, wherein the cells are allogeneic with respect to the subject to be administered the cells.

150. The method of any one of claims 146-149, wherein the cells or their progeny persist in the subject for at least one month, two month, three months, four months, five months, six months, seven months, eight months, nine months, ten months, eleven months, twelve months, thirteen months, fourteen month, fifteen months, sixteen months, seventeen months, eighteen months, nineteen months, twenty months, twenty-one months, twenty-two months, twenty-three months, two years, three years, four years, or five years after administration of the modified cells to the subject.

151. The method of any one of claims 146-150, wherein the method results in an increased levels of hemoglobin F (HbF) in circulating blood of at least about 5%, at least about 10%, at least about 20%, at least about 30%, at least about 40%, or at least about 50% compared to the levels of HbF in the subject prior to treatment.

152. The method of any one of claims 146-150, wherein the method results in a ratio of HbF to hemoglobin S (HbS) in the subject of at least 0.01:1.0, at least 0.025:1.0, at least 0.05:1.0, at least 0.075:1.0 at least 0.1:1.0, at least 0.2:1.0, at least 0.3:1.0, at least 0.4:1.0, at least 0.5:1:0, at least 0.75:1.0, at least 1.0:1.0, at least 1.25:1.0, at least 1.5:1.0, or at least 1.75:1.0.

153. The method of any one of claims 146-150, wherein the method results in HbF levels of at least about 5%, or at least about 10%, or at least about 20%, or at least about 30% of total circulating hemoglobin in the subject.

154. The method of any one of claims 146-153, wherein the subject is selected from the group consisting of a rodent, a mouse, a rat, and a non-human primate.

155. The method of any one of claims 146-153, wherein the subject is a human.

156. A method of treating a hemoglobinopathy in a subject in need thereof, comprising modifying a BCL11A gene in cells of the subject, the modifying comprising contacting said cells with a therapeutically effective dose of:
a. the system of any one of claims 1-85;
b. the nucleic acid of any one of claims 86-89;
c. the vector as in any one of claims 90-100;
d. the XDP of any one of claims 101-104; or
e. combinations of two or more of (a)-(d),
wherein the BCL11A gene of the cells targeted by the first gRNA is modified by the CasX protein.

157. The method of claim 156, wherein the hemoglobinopathy is sickle cell disease or beta-thalassemia.

158. The method of any one of claim 156 or claim 157, wherein the cell is selected from the group consisting of hematopoietic stem cells (HSC), hematopoietic progenitor cells (HPC), CD34+ cells, mesenchymal stem cells (MSC), induced pluripotent stem cells (iPSC), common myeloid progenitor cells, proerythroblast cells, and erythroblast cells.

159. The method of any one of claims 156-158, wherein the modifying comprises introducing a single-stranded break in the BCL11A gene of the cells.

160. The method of any one of claims 156-158, wherein the modifying comprises introducing a double-stranded break in the BCL11A gene of the cells.

161. The method of any one of claims 156-160, further comprising introducing into the cells of the subject a second gRNA or a nucleic acid encoding the second gRNA, wherein the second gRNA has a targeting sequence complementary to a different or overlapping portion of the target nucleic acid compared to the first gRNA, resulting in an additional break in the BCL11A target nucleic acid of the cells of the subject.

162. The method of any one of claims 156-161, wherein the modifying comprises introducing an insertion, deletion, substitution, duplication, or inversion of one or more nucleotides in the BCL11A gene of the cells.

163. The method of claim 162, wherein the modifying results in a knock-down or knock-out of the BCL11A gene in the modified cells of the subject.

164. The method of any one of claims 156-163, wherein the BCL11A gene of the cells are modified such that expression of the BCL11A protein by the modified cells is reduced by at least about 10%, at least about 20%, at least about 30%, at least about 40%, at least about 50%, at least about 60%, at least about 70%, at least about 80%, or at least about 90% in comparison to cells that have not been modified.

165. The method of any one of claims 156-163, wherein the BCL11A gene of the cells of the subject are modified such that at least 70%, at least 75%, at least 80%, at least 85%, at least 90%, or at least 95% of the modified cells do not express a detectable level of BCL11A protein.

166. The method of any one of claims 156-165, wherein the method results in an increased levels of hemoglobin F (HbF) in circulating blood of the subject of at least about 5%, at least about 10%, at least about 20%, at least about 30%, at least about 40%, or at least about 50% compared to the levels of HbF in the subject prior to treatment.

167. The method of any one of claims 156-166, wherein the method results in a ratio of HbF to hemoglobin S (HbS) in circulating blood of the subject of at least 0.01:1.0, at least 0.025:1.0, at least 0.05:1.0, at least 0.075:1.0 at least 0.1:1.0, at least 0.2:1.0, at least 0.3:1.0, at least 0.4:1.0, at least 0.5:1:0, at least 0.75:1.0, at least 1.0:1.0, at least 1.25:1.0, at least 1.5:1.0, or at least 1.75:1.0.

168. The method of any one of claims 156-165, wherein the method results in HbF levels of at least about 5%, or at least about 10%, or at least about 20%, or at least about 30% of total hemoglobin in circulating blood of the subject.

169. The method of any one of claims 156-161, wherein the method comprises insertion of the donor template into the break site(s) of the BCL11A gene target nucleic acid sequence of the cells.

170. The method of claim 168, wherein the insertion of the donor template is mediated by homology-directed repair (HDR) or homology-independent targeted integration (HITI).

171. The method of claim 168 or claim 170, wherein insertion of the donor template results in a knock-down or knock-out of the BCL11A gene in the modified cells of the subject.

172. The method of any one of claims 166-171, wherein the BCL11A gene of the cells are modified such that expression of the BCL11A protein by the modified cells is reduced by at least about 10%, at least about 20%, at least about 30%, at least about 40%, at least about 50%, at least about 60%, at least about 70%, at least about 80%, or at least about 90% in comparison to cells that have not been modified.

173. The method of any one of claims 166-171, wherein the BCL11A gene of the cells of the subject are modified such that at least 70%, at least 75%, at least 80%, at least 85%, at least 90%, or at least 95% of the modified cells do not express a detectable level of BCL11A protein.

174. The method of any one of claims 166-173, wherein the method results in an increased levels of hemoglobin F (HbF) in circulating blood of the subject of at least about 5%, at least about 10%, at least about 20%, at least about 30%, at least about 40%, or at least about 50% compared to the levels of HbF in the subject prior to treatment.

175. The method of any one of claims 166-173, wherein the method results in a ratio of HbF to hemoglobin S (HbS) in circulating blood of the subject of at least 0.01:1.0, at least 0.025:1.0, at least 0.05:1.0, at least 0.075:1.0 at least 0.1:1.0, at least 0.2:1.0, at least 0.3:1.0, at least 0.4:1.0, at least 0.5:1:0, at least 0.75:1.0, at least 1.0:1.0, at least 1.25:1.0, at least 1.5:1.0, or at least 1.75:1.0.

176. The method of any one of claims 166-173, wherein the method results in HbF levels of at least about 5%, or at least about 10%, or at least about 20%, or at least about 30% of total hemoglobin in circulating blood of the subject.

177. The method of any one of claims 156-175, wherein the subject is selected from the group consisting of rodent, mouse, rat, and non-human primate.

178. The method of any one of claims 156-175, wherein the subject is a human.

179. The method of any one of claims 156-178, wherein the vector is AAV and is administered to the subject at a dose of at least about 1×10⁵ vector genomes/kg (vg/kg), at least about 1×10⁶ vg/kg, at least about 1×10⁷ vg/kg, at least about 1×10⁸ vg/kg, at least about 1×109 vg/kg, at least about 1×10¹⁰ vg/kg, at least about 1×10¹¹ vg/kg, at least about 1×10¹² vg/kg, at least about 1×10¹³ vg/kg, at least about 1×10¹⁴ vg/kg, at least about 1×10¹⁵ vg/kg, or at least about 1×10¹⁶ vg/kg.

180. The method of any one of claims 156-178, wherein the vector is AAV and is administered to the subject at a dose of at least about 1×10⁵ vg/kg to about 1×10¹⁶ vg/kg, at least about 1×10⁶ vg/kg to about 1×10¹⁵ vg/kg, or at least about 1×10⁷ vg/kg to about 1×10¹⁴ vg/kg.

181. The method of any one of claims 156-178, wherein the XDP is administered to the subject at a dose of at least about 1×10⁵ particles/kg, at least about 1×10⁶ particles/kg, at least about 1×10⁷ particles/kg at least about 1×10⁸ particles/kg, at least about 1×10⁹ particles/kg, at least about 1×10¹⁰ particles/kg, at least about 1×10¹¹ particles/kg, at least about 1×10¹² particles/kg, at least about 1×10¹³ particles/kg, at least about 1×10¹⁴ particles/kg, at least about 1×10¹⁵ particles/kg, at least about 1×10¹⁶ particles/kg.

182. The method of any one of claims 156-178, wherein the XDP is administered to the subject at a dose of at least about 1×10⁵ particles/kg to about 1×10¹⁶ particles/kg, or at least about 1×10⁶ particles/kg to about 1×10¹⁵ particles/kg, or at least about 1×10⁷ particles/kg to about 1×10¹⁴ particles/kg.

183. The method of any one of claims 156-182, wherein the vector or XDP is administered to the subject by a route of administration selected from intraparenchymal, intravenous, intra-arterial, intraperitoneal, intracapsular, subcutaneously, intramuscularly, intraabdominally, or combinations thereof, wherein the administering method is injection, transfusion, or implantation.

184. The method of any one of claims 156-183, wherein the method results in improvement in at least one clinically-relevant endpoint in the subject.

185. The method of claim 184, wherein the method results in improvement in at least one clinically-relevant parameter selected from the group consisting of occurrence of end-organ disease, albuminuria, hypertension, hyposthenia, hyposthenuria, diastolic dysfunction, functional exercise capacity, acute coronary syndrome, pain events, pain severity, anemia, hemolysis, tissue hypoxia, organ dysfunction, abnormal hematocrit values, childhood mortality, incidence of strokes, hemoglobin levels compared to baseline, HbF levels, reduced incidence of pulmonary embolisms, incidence of vaso-occlusive crises, concentration of hemoglobin S in erythrocytes, rate of hospitalizations, liver iron concentration, required blood transfusions, and quality of life score.

186. The method of claim 184, wherein the method results in improvement in at least two clinically-relevant parameters selected from the group consisting of occurrence of end-organ disease, albuminuria, hypertension, hyposthenia, hyposthenuria, diastolic dysfunction, functional exercise capacity, acute coronary syndrome, pain events, pain severity, anemia, hemolysis, tissue hypoxia, organ dysfunction, abnormal hematocrit values, childhood mortality, incidence of strokes, hemoglobin levels compared to baseline, HbF levels, reduced incidence of pulmonary embolisms, incidence of vaso-occlusive crises, concentration of hemoglobin S in erythrocytes, rate of hospitalizations, liver iron concentration, required blood transfusions, and quality of life score.

187. A method for treating a subject with a hemoglobinopathy, the method comprising:
a. isolating induced pluripotent stem cells (iPSC) or hematopoietic stem cells (HSC) from a subject;
b. modifying the BCL11A target nucleic acid of the iPSC or HSC by the method of any one of claims 110-126;
c. differentiating the modified iPSC or HSC into a hematopoietic progenitor cell; and
d. implanting the hematopoietic progenitor cell into the subject with the hemoglobinopathy,
wherein the method results in an increased levels of hemoglobin F (HbF) in circulating blood of the subject of at least about 5%, at least about 10%, at least about 20%, at least about 30%, at least about 40%, or at least about 50% compared to the levels of HbF in the subject prior to treatment.

188. The method of claim 187, wherein the iPSC or HSC is autologous and is isolated from the subject's bone marrow or peripheral blood.

189. The method of claim 187, wherein the iPSC or HSC is allogeneic and is isolated from a different subject's bone marrow or peripheral blood.

190. The method of any one of claims 187-189, wherein the implanting comprises administering the hematopoietic progenitor cell to the subject by transplantation, local injection, systemic infusion, or combinations thereof.

191. The method of any one of claims 187-190, wherein the hemoglobinopathy is sickle cell disease or beta-thalassemia.

192. A method of increasing fetal hemoglobin (HbF) in a subject by genome editing, the method comprising:
a. administering to the subject an effective dose of the vector of any one of claims 90-100 or the XDP of any one of claims 101-107, wherein the vector or XDP delivers the CasX:gRNA system to cells of the subject;
b. the BCL11A target nucleic acid of cells of the subject are edited by the CasX targeted by the first gRNA;
c. the editing comprises introducing an insertion, deletion, substitution, duplication, or inversion of one or more nucleotides in the target nucleic acid sequence such that expression of BCL11A protein is reduced or eliminated,
wherein the method results in an increased levels of hemoglobin F (HbF) in circulating blood of the subject of at least about 5%, at least about 10%, at least about 20%, at least about 30%, at least about 40%, or at least about 50% compared to the levels of HbF in the subject prior to treatment.

193. The method of claim 192, wherein the method results in a ratio of HbF to hemoglobin S (HbS) in the subject of at least 0.01:1.0, at least 0.025:1.0, at least 0.05:1.0, at least 0.075:1.0 at least 0.1:1.0, at least 0.2:1.0, at least 0.3:1.0, at least 0.4:1.0, at least 0.5:1:0, at least 0.75:1.0, at least 1.0:1.0, at least 1.25:1.0, at least 1.5:1.0, or at least 1.75:1.0.

194. The method of claim 192 or claim 193, wherein the method results in HbF levels of at least about 5%, or at least about 10%, or at least about 20%, or at least about 30% of total circulating hemoglobin in the subject.

195. The method of any one of claims 192-194, wherein the cells are selected from the group consisting of hematopoietic stem cells (HSC), hematopoietic progenitor cells (HPC), CD34+ cells, mesenchymal stem cells (MSC), induced pluripotent stem cells (iPSC), common myeloid progenitor cells, proerythroblast cells, and erythroblast cells.

196. The method of any one of claims 192-195, wherein the target nucleic acid of the cells has been edited such that expression of the BCL11A protein is reduced by at least about 10%, at least about 20%, at least about 30%, at least about 40%, at least about 50%, at least about 60%, at least about 70%, at least about 80%, or at least about 90% in comparison to target nucleic acid of cells that have not been edited.

197. The method of any one of claims 192-196, wherein the subject is selected from the group consisting of mouse, rat, pig, and non-human primate.

198. The method of any one of claims 192-196, wherein the subject is a human.

199. The method of any one of claims 192-198, wherein the vector is administered at a dose of at least about 1×10⁵ vector genomes/kg (vg/kg), at least about 1×10⁶ vg/kg, at least about 1×10⁷ vg/kg, at least about 1×10⁸ vg/kg, at least about 1×10⁹ vg/kg, at least about 1×10¹⁰ vg/kg at least about 1×10¹¹ vg/kg, at least about 1×10¹² vg/kg, at least about 1×10¹³ vg/kg, at least about 1×10¹⁴ vg/kg, at least about 1×10¹⁵ vg/kg, or at least about 1×10¹⁶ vg/kg.

200. The method of any one of claims 192-198, wherein the XDP is administered at a dose of at least about 1×10⁵ particles/kg, at least about 1×10⁶ particles/kg, at least about 1×10⁷ particles/kg, at least about 1×10⁸ particles/kg, at least about 1×10⁹ particles/kg, at least about 1×10¹⁰ particles/kg at least about 1×10¹¹ particles/kg, at least about 1×10¹² particles/kg, at least about 1×10¹³ particles/kg, at least about 1×10¹⁴ particles/kg, at least about 1×10¹⁵ particles/kg, or at least about 1×10¹⁶ particles/kg.

201. The method of any one of claims 192-200, wherein the vector or XDP is administered by a route of administration selected from transplantation, local injection, systemic infusion, or combinations thereof.

202. The system of any one of claims 1-85, the nucleic acid of any one of claims 86-89, the vector of any one of 90-95, the XDP of any one of claims 101-104, the host cell of claim 108 or claim 109, or the population of cells of claim 144 or claim 145, for use as a medicament for the treatment of a hemoglobinopathy.

203. The system of claim 1, wherein the target nucleic acid sequence is complementary to a non-target strand sequence located 1 nucleotide 3′ of a protospacer adjacent motif (PAM) sequence.

204. The system of claim 203, wherein the PAM sequence comprises a TC motif.

205. The system of claim 204, wherein the PAM sequence comprises ATC, GTC, CTC or TTC.

206. The system of any one of claims 203-205, wherein the Class 2 Type V CRISPR protein comprises a RuvC domain.

207. The system of claim 206, wherein the RuvC domain generates a staggered double-stranded break in the target nucleic acid sequence.

208. The system of any one of claims 203-207, wherein the Class 2 Type V CRISPR protein does not comprise an HNH nuclease domain.
